(19) United States
(12) Patent Application Publication
(10) Pub. No.: US 2007/0190533 A1
(43) Pub. Date: Aug. 16, 2007

(54) Effectors of innate immunity

(76) Inventors: Robert E.W. Hancock, Vancouver (CA); B. Brett Finlay, Richmond (CA); Monisha Gough Scott, Vancouver (CA); Dawn Bowdish, Oxford (GB); Carrie Melissa Rosenberger, Seattle, WA (US); Jon-Paul Steven Powers, Vancouver (CA)

Correspondence Address: DLA PIPER US LLP, 4365 EXECUTIVE DRIVE, SUITE 1100, SAN DIEGO, CA, 92121-2133, US

(21) Appl. No.: 11/241,882

(22) Filed: Sep. 29, 2005

Related U.S. Application Data
(63) Continuation-in-part of application No. 10/661,471, filed on Sep. 12, 2003.
Application No. 10/661,471 is a continuation-in-part of application No. 10/308,905, filed on Dec. 2, 2002.
(60) Provisional application No. 60/336,632, filed on Dec. 3, 2001.

(51) Int. Cl. C12Q 1/68; G01N 33/567

(52) U.S. Cl. 435/6; 435/7.2

(57) ABSTRACT

The present invention provides a method of identifying agents that enhance innate immunity in a subject. The invention further provides a method of selectively supresses sepsis by suppressing expression of a proinflammatory gene while maintaining expression of an anti-inflammatory gene. Also provided are methods of identifying a polynucleotide or pattern of polynucleotides regulated by one or more sepsis or inflammatory inducing agents and inhibited by a peptide is described, methods of identifying a pattern of polynucleotide expression for inhibition of an inflammatory or septic response, and compounds and agents identified by the methods of the invention.

### RELATED APPLICATION DATA

This application claims priority under 35 U.S.C. § 120 to U.S. patent application Ser. No. 10/661,471, filed Sep. 12, 2003, which is a continuation-in-part of U.S. patent application Ser. No. 10/308,905, filed Dec. 2, 2002, which claims priority under 35 U.S.C. § 119(e) to U.S. Patent Application Ser. No. 60/336,632, filed Dec. 3, 2001, herein incorporated by reference in their entirety.

### FIELD OF THE INVENTION

The present invention relates generally to peptides and specifically to peptides effective as therapeutics and for drug discovery related to pathologies resulting from microbial infections and for modulating innate immunity or inflammation.

### BACKGROUND OF THE INVENTION

Infectious diseases are the leading cause of death worldwide. According to a 1999 World Health Organization study, over 13 million people die from infectious diseases each year. Infectious diseases are the third leading cause of death in North America, accounting for 20% of deaths annually and increasing by 50% since 1980. The success of many medical and surgical treatments also hinges on the control of infectious diseases. The discovery and use of antibiotics has been one of the great achievements of modem medicine. Without antibiotics, physicians would be unable to perform complex surgery, chemotherapy or most medical interventions such as catheterization.

Current sales of antibiotics are US$26 billion worldwide. However, the overuse and sometimes unwarranted use of antibiotics have resulted in the evolution of new antibiotic-resistant strains of bacteria. Antibiotic resistance has become part of the medical landscape. Bacteria such as vancomycin-resistant Enterococcus, VRE, and methicillin-resistant Staphylococcus aureus and MRSA strains cannot be treated with antibiotics and often, patients suffering from infections with such bacteria die. Antibiotic discovery has proven to be one of the most difficult areas for new drug development and many large pharmaceutical companies have cut back or completely halted their antibiotic development programs. However, with the dramatic rise of antibiotic resistance, including the emergence of untreatable infections, there is a clear unmet medical need for novel types of anti-microbial therapies, and agents that impact on innate immunity would be one such class of agents.

The innate immune system is a highly effective and evolved general defense system. Elements of innate immunity are always present at low levels and are activated very rapidly when stimulated. Stimulation can include interaction of bacterial signaling molecules with pattern recognition receptors on the surface of the body's cells or other mechanisms of disease. Every day, humans are exposed to tens of thousands of potential pathogenic microorganisms through the food and water we ingest, the air we breathe and the surfaces, pets and people that we touch. The innate immune system acts to prevent these pathogens from causing disease. The innate immune system differs from so-called adaptive immunity (which includes antibodies and antigen-specific B- and T-lymphocytes) because it is always present, effective immediately, and relatively non-specific for any given pathogen. The adaptive immune system requires amplification of specific recognition elements and thus takes days to weeks to respond. Even when adaptive immunity is pre-stimulated by vaccination, it may take three days or more to respond to a pathogen whereas innate immunity is immediately or rapidly (hours) available. Innate immunity involves a variety of effector functions including phagocytic cells, complement, etc, but is generally incompletely understood. Generally speaking many known innate immune responses are “triggered” by the binding of microbial signaling molecules with pattern recognition receptors such as Toll-like receptors (TLR) on the surface of host cells. We now know that Toll/Interleukin-1 Receptor (TIR) domain-containing proteins play a pivotal role in initiating aspects of the inflammatory responses. Many of these effector functions are grouped together in the inflammatory response. However, too severe an inflammatory response can result in responses that are harmful to the body, and, in an extreme case, sepsis and potentially death can occur. Thus, a therapeutic intervention to boost innate immunity, which is based on stimulation of TLR signaling (for example using a TLR agonist), has the potential disadvantage that it could stimulate a potentially harmful inflammatory response and/or exacerbate the natural inflammatory response to infection.

Early responses to infection, collectively termed innate immunity and/or acute inflammation, are substantially orchestrated by various mechanisms, for example, the interaction of bacterial molecules with TLR. It has been shown that a breakdown in the appropriate regulation of the TLR pathway can cause common chronic inflammatory diseases including inflammatory bowel disease (IBD), cardiovascular disease, arthritis, and chronic interstitial nephritis. Further, TLR engagement by conserved microbial molecules results in the translocation of the pivotal transcription factor NFκB and the transcription of ‘early-response’ genes encoding, for example, cytokines, chemokines, selected antimicrobial/host defense peptides, acute phase proteins, cell adhesion molecules, co-stimulatory molecules and proteins required for negative feedback to suppress these responses. Alternatively, an exaggerated response to bacterial stimuli underlies a clinical condition called Systemic Inflammatory Response Syndrome, or sepsis, in which high levels of cytokines and inflammatory mediators become destructive, causing organ failure, cardiovascular shock and/or death.

Sepsis occurs in approximately 780,000 patients in North America annually. Sepsis may develop as a result of infections acquired in the community such as pneumonia, or it may be a complication of the treatment of trauma, cancer or major surgery. Severe sepsis occurs when the body is overwhelmed by the inflammatory response and body organs begin to fail. Up to 120,000 deaths occur annually in the United Stated due to sepsis. Sepsis may also involve pathogenic microorganisms or toxins in the blood (e.g., septicemia), which is a leading cause of death among humans. Gram-negative bacteria are the organisms most commonly associated with such diseases. However, gram-positive bacteria are an increasing cause of infections. Gram-negative and Gram-positive bacteria and their components can all cause sepsis.

The presence of microbial components induces the release of pro-inflammatory cytokines of which tumor necrosis factor-α (TNF-α) is of extreme importance. TNF-α and other pro-inflammatory cytokines can then cause the release of other pro-inflammatory mediators and lead to an inflammatory cascade. Gram-negative sepsis is usually caused by the release of the bacterial outer membrane component, lipopolysaccharide (LPS; also referred to as endotoxin). Endotoxin in the blood, called endotoxemia comes primarily from a bacterial infection, and may be released during treatment with antibiotics. Gram-positive sepsis can be caused by the release of bacterial cell wall components such as lipoteichoic acid (LTA), peptidoglycan (PG), rhamnose-glucose polymers made by Streptococci, or capsular polysaccharides made by Staphylococci. Bacterial or other non-mammalian DNA that, unlike mammalian DNA, frequently contains unmethylated cytosine-guanosine dimers (CpG DNA) has also been shown to induce septic conditions including the production of TNF-α. Mammalian DNA contains CpG dinucleotides at a much lower frequency, often in a methylated form. In addition to their natural release during bacterial infections, antibiotic treatment can also cause release of the bacterial cell wall components LPS and LTA and probably also bacterial DNA. This can then hinder recovery from infection or even cause sepsis.

In humans, inhalation of the Gram-negative bacterial component lipopolysaccharide (LPS, also termed endotoxin), a TLR4 ligand, results in increased cytokine and chemokine (TNFα, IL1β, IL6, IL8) mRNA and protein expression within 4-6 hr of inhalation. In mutant mice lacking responsiveness to LPS animals do not develop septic shock, demonstrating that the response to endotoxin is sufficient to promote sepsis. Other TLRs exist in humans and can be engaged by other pathogen molecules to drive septic responses. For example, TLR2 is engaged by the signature cell wall-associated molecule lipoteichoic acid (LTA) from Gram positive bacteria, while DNA containing the signature dinucleotide pair unmethylated CpG engages TLR9 and can also stimulate proinflammatory cytokine production. The nature, duration and intensity of inflammatory/septic responses are considered to involve the interplay between TLR and other receptors, different adaptor molecules such as MyD88, TIRAP/Mal and TRIF, and different signalling pathways. An ideal therapeutic regulator of the inflammatory response would be antagonistic to potentially lethal conditions such as septic shock by interacting with inflammatory signaling pathways but maintain innate immune defenses against bacterial infections, thus sustaining a balance between the protective and destructive components of inflammation.

Cationic host defense peptides (also known as antimicrobial peptides) are crucial molecules in host defense against pathogenic microbe challenge. These peptides have been demonstrated to have a wide range of functions ranging from direct antimicrobial activity to a broad range of immunomodulatory functions. They are widely distributed in nature, existing in organisms from insects to plants to mammals. The family includes defensins, cathelicidins, and histatins. Cathelicidins are small (12 to around 50 amino acids) cationic peptides and are amphipathic in nature with ˜50% hydrophobic residues. Mammalian cathelicidins are synthesized in a precursor pro-form that requires (generally-extracellular) proteolytic processing to generate the mature peptide. The only endogenous cathelicidin in humans is hCAP-18 (SEQ ID NO:1) which is found at high concentrations in its unprocessed form (hCAP-18) in the granules of neutrophils and is processed upon degranulation and release. It is also produced by epithelial cells and keratinocytes, etc., as the hCAP-18 precursor form, and is found as the processed 37-amino acid peptide SEQ ID NO: 1 in a number of tissues and bodily fluids including gastric juices, saliva, semen, sweat, plasma, airway surface liquid and breast milk.

Cationic peptides are being increasingly recognized as a form of defense against infection, and although the major effects recognized in the scientific and patent literature were the antimicrobial effects (Hancock, R. E. W., and R. Lehrer. 1998. Cationic peptides: a new source of antibiotics. Trends in Biotechnology 16: 82-88.), it is now becoming increasingly clear that they are effectors in other aspects of innate immunity (Hancock, R. E. W. and G. Diamond. 2000. The role of cationic peptides in innate host defenses. Trends in Microbiology 8:402-410.; Hancock, R. E. W. 2001. Cationic peptides: effectors in innate immunity and novel antimicrobials. Lancet Infectious Diseases 1:156-164).

Some cationic peptides have an affinity for binding bacterial products such as LPS and LTA. Such cationic peptides can suppress cytokine production in response to LPS, and to varying extents can prevent lethal shock. However it has not been proven as to whether such effects are due to binding of the peptides to LPS and LTA, or due to a direct interaction of the peptides with host cells. Cationic peptides are induced, in response to challenge by microbes or microbial signaling molecules like LPS, by a regulatory pathway similar to that used by the mammalian immune system (involving Toll receptors and the transcription factor; NFκB). Cationic peptides therefore appear to have a key role in innate immunity. Mutations that affect the induction of antibacterial peptides can reduce survival in response to bacterial challenge. As well, mutations of the Toll pathway of Drosophila that lead to decreased antifungal peptide expression result in increased susceptibility to lethal fungal infections. In humans, patients with specific granule deficiency syndrome, completely lacking in α-defensins, suffer from frequent and severe bacterial infections. Other evidence includes the inducibility of some peptides by infectious agents, and the very high concentrations of such peptides that have been recorded at sites of inflammation. Cationic peptides may also regulate cell migration, to promote the ability of leukocytes to combat bacterial infections. For example, two human α-defensin peptides, HNP-1 and HNP-2, have been indicated to have direct chemotactic activity for murine and human T cells and monocytes, and human β-defensins appear to act as chemoattractants for immature dendritic cells and memory T cells through interaction with CCR6. Similarly, the porcine cationic peptide, PR-39 was found to be chemotactic for neutrophils. It is unclear however as to whether peptides of different structures and compositions share these properties.

The single known cathelicidin from humans, SEQ ID NO: 1, is produced by myeloid precursors, testis, human keratinocytes during inflammatory disorders and airway epithelium. The characteristic feature of cathelicidin peptides is a high level of sequence identity at the N-terminus prepro regions termed the cathelin domain. Cathelicidin peptides are stored as inactive propeptide precursors that, upon stimulation, are processed into active peptides.

### SUMMARY OF THE INVENTION

The present invention is based on the seminal discovery that based on patterns of polynucleotide expression regulated by endotoxic lipopolysaccharide, lipoteichoic acid, CpG DNA, or other cellular components (e.g., microbe or their cellular components), and affected by cationic peptides, one can screen for novel compounds that block or reduce sepsis and/or inflammation in a subject. Further, based on the use of cationic peptides as a tool, one can identify selective enhancers of innate immunity that do not trigger the sepsis reaction and that can block/dampen inflammatory and/or septic responses.

Thus, in one embodiment, a method of identifying a polynucleotide or pattern of polynucleotides regulated by one or more sepsis or inflammatory inducing agents and inhibited by a cationic peptide, is provided. The method of the invention includes contacting the polynucleotide or polynucleotides with one or more sepsis or inflammatory inducing agents and contacting the polynucleotide or polynucleotides with a cationic peptide either simultaneously or immediately thereafter. Differences in expression are detected in the presence and absence of the cationic peptide, and a change in expression, either up- or down-regulation, is indicative of a polynucleotide or pattern of polynucleotides that is regulated by a sepsis or inflammatory inducing agent and inhibited by a cationic peptide. In another aspect the invention provides a polynucleotide or polynucleotides identified by the above method. Examples of sepsis or inflammatory regulatory agents include LPS, LTA or CpG DNA or microbial components (or any combination thereof), or related agents.

In another embodiment, the invention provides a method of identifying an agent that blocks sepsis or inflammation including combining a polynucleotide identified by the method set forth above with an agent wherein expression of the polynucleotide in the presence of the agent is modulated as compared with expression in the absence of the agent and wherein the modulation in expression affects an inflammatory or septic response.

In another embodiment, the invention provides a method of identifying a pattern of polynucleotide expression for inhibition of an inflammatory or septic response by 1) contacting cells with LPS, LTA and/or CpG DNA in the presence or absence of a cationic peptide and 2) detecting a pattern of polynucleotide expression for the cells in the presence and absence of the peptide. The pattern obtained in the presence of the peptide represents inhibition of an inflammatory or septic response. In another aspect the pattern obtained in the presence of the peptide is compared to the pattern of a test compound to identify a compound that provides a similar pattern. In another aspect the invention provides a compound identified by the foregoing method.

In another embodiment, the invention provides a method of identifying an agent that selectively enhances innate immunity by contacting cells containing a polynucleotide or polynucleotides that encode a polypeptide involved in innate immunity, with an agent of interest, wherein expression of the polynucleotide in the presence of the agent is modulated as compared with expression of the polynucleotide in the absence of the agent and wherein the modulated expression results in enhancement of innate immunity. Preferably, the agent does not stimulate a sepsis reaction in a subject. In one aspect, the agent increases the expression of an anti-inflammatory polynucleotide. Exemplary, but non-limiting anti-inflammatory polynucleotides encode proteins such as IL-1 R antagonist homolog 1 (AI167887), IL-10 R beta (AA486393), IL-10 R alpha (U00672) TNF Receptor member 1B (AA150416), TNF receptor member 5 (H98636), TNF receptor member 11b (AA194983), IK cytokine down-regulator of HLA II (R39227), TGF-B inducible early growth response 2 (AI473938), CD2 (AA927710), IL-19 (NM_{—}013371) or IL-10 (M57627). In one aspect, the agent decreases the expression of polynucleotides encoding proteasome subunits involved in NF-κB activation such as proteasome subunit 26S (D78151). In one aspect, the agent may act as an antagonist of protein kinases. In one aspect, the agent is a peptide selected from SEQ ID NO:4-54.

In another embodiment, the invention provides a method of identifying an agent that selectively suppresses the proinflammatory response of cells containing a polynucleotide or polynucleotides that encode a polypeptide involved in innate immunity. The method includes contacting the cells with microbes, or the TLR ligands and agonists derived from those microbes, and further contacting the cells with an agent of interest, wherein the agent decreases the expression of a proinflammatory gene encoding the polynucleotide as compared with expression of the proinflammatory gene in the absence of the agent. In one aspect, the modulated expression results in suppression of proinflammatory and septic responses. Preferably, the agent does not stimulate a sepsis reaction in a subject. Exemplary, but non-limiting proinflammatory genes include TNFα, TNFAIP2, IL1β. IL6, NFKB1 and RELA.

In another embodiment, the invention provides a method of identifying an agent that enhances innate immunity by contacting cells containing a polynucleotide or polynucleotides that encode a polypeptide involved in innate immunity, with an agent of interest, wherein the agent suppresses inflammation and sepsis while increasing the expression of an anti-inflammatory gene encoding the polynucleotide as compared with expression of the anti-inflammatory gene in the absence of the agent and wherein the modulated expression results in enhancement of innate immunity. In one aspect, the agent inhibits the expression of proinflammatory molecules such as TNFα, IL1-β, IL-6, TNFα, TNFAIP2, or the p50 or p65 subunits of transcription factor NFκB. In another aspect, inflammation is induced by a microbe or a microbial ligand acting on a Toll-like receptor such as Toll-like receptor-2, Toll-like receptor-4, or Toll-like receptor-9. Microbial ligands include, but are not limited to a bacterial endotoxin, lipopolysaccharide, lipoteichoic acid or CpG DNA. Exemplary, but non-limiting anti-inflammatory genes include ZNF83, NFKBIA, Q9P188, INVS, DIAPH1, IER3, Q9H640, GBP2, NANS, Q86XN7, Q9H9M1, TNFAIP3, Q96MJ8, Q9BSE2, Q9H753, NTNG1, INHBE, BCL6, CXCL1, EHD1, RELB, HRK, CCL4, SESN2, NAB1, EBI3, DDX21, XBP1, SLURP1, ARS, HDAC10, MEP1A, RAP2C, GYS1, RARRES3, PPY, NFKB1, MTL4_HUMAN, Q9H040, and Q9NUP6.

In another embodiment, the invention provides a method of identifying an agent that selectively suppresses sepsis by contacting cells containing a polynucleotide or polynucleotides that encode a polypeptide involved in innate immunity, with an agent of interest, wherein the agent suppresses expression of a proinflammatory gene while maintaining expression of an anti-inflammatory gene encoding the polynucleotide as compared with expression of the anti-inflammatory gene in the absence of the agent. In one aspect, the agent inhibits the expression of proinflammatory molecules such as TNFα, IL1-β, IL-6, TNFα, TNFAIP2, or the p50 or p65 subunits of transcription factor NFκB. In another aspect, inflammation is induced by a microbe or a microbial ligand acting on a Toll-like receptor such as Toll-like receptor-2, Toll-like receptor-4, or Toll-like receptor-9. Microbial ligands include, but are not limited to a bacterial endotoxin, lipopolysaccharide, lipoteichoic acid or CpG DNA. Exemplary, but non-limiting anti-inflammatory genes include ZNF83, NFKBIA, Q9P188, INVS, DIAPH1, IER3, Q9H640, GBP2, NANS, Q86XN7, Q9H9M1, TNFAIP3, Q96MJ8, Q9BSE2, Q9H753, NTNG1, INHBE, BCL6, CXCL1, EHD1, RELB, HRK, CCL4, SESN2, NAB1, EBI3, DDX21, XBP1, SLURP1, ARS, HDAC10, MEP1A, RAP2C, GYS1, RARRES3, PPY, NFKB1, MTL4_HUMAN, Q9H040, and Q9NUP6. Exemplary, but non-limiting proinflammatory genes include LC2A6, SLC4A5, MCL1, Q86XN7, Q9H9M1, Q86UU3, Q8NAA1, C15orf2, TNFRSF5, FACL6, Q8IW99, Q96AU7, PRB4, Q9NWP0, Q8NF24, Q8TEE5, PDE4DIP, NUDT4, DUSP2, LMAN2, RELB, SNF1LK, TNFα, GHRHR, TNFSF6, ENSG00000181873, IRAK2, CKB, CASR, KRTAP4-10, ARHGEF3, CYP3A4, CYP3A7, GPR27, PAX8, GAP43, Q96M75, Q9H568, AGTRL1, C1 or f22, EHD1, ADRA1B, SSTR2, SYNE1, ENSG00000139977, PTPRK, O15059, Q9NZ16, N4BP3, KIAA0341, Q8IVT2, Q9NV39, HIP1R, HIP12, KIAA0655, IL6, TNFAIP2, RCV1, FBLN2, TWIST2, PARD6B, DCK, TULP4, LK10, SPAP1, IBRDC2, JAM2, NRG2, CBARA1, DLG2, PRKCBP1, MGLL, Q9BYE1, MARCKS, Q96N98, Q8NBY1, Q96AF2, Q9BS16, PPP2CA, RAB38, VCAM1, TTTY8, HTR2A, SERPINB10, O75121, Q9BVE1, ZCCHC2, CXCL2, GADD45B, KARS, SCG2, SLC17A2, FLT4, Q9NXT0, Q96L19, BICD1, HCK, Q8N9T8, Q9H978, PPP1R1A, PAX7, EBI3, THRA, SLC16A10, INPP5E, Q9H967, NFKB1, MKL1, SS18L2, TNFRSF9, TNFAIP6, Q9Y2K2, ING5, IL1A, TMH, HDAC4, KPTN, SEC61G, Q9Y484, FRAS1, IER5, Q8N137, Q8NCB8, Q96HQ0, Q9H5P0, TXNRD1, CAV2, SCARB1, MAP3K5, PDHX, TCEB3, C21orf55, MPHOSPH10, PDE8A, TFR2, FARP1, SERPINA1, MYO15A, RABGGTA, KCNMB4, Q9BR02, APOB, MYC, FARP2, TFAP2BL1, Q86U90, Q9H5F8, USH1C, IL8, SOX2, Q9NVC3, NEIL2, TNIP1, ADRA1D, PCDHB9, Q12987, TNFRSF6, C20orf72, DNAJA3, MAB21L1, BIRC2, MYST1, CNN3, CXCL3, CD80, CSRP2, RAD51L1, ADARB1, TNFSF8, Q8IW74, UXS1, ENSG00000182364, TNFRSF7, MYBL2, RAB33A, ATIC, CAMK1, CCNT1, KCNE4, BOK, NF2, PDP2, and KIAA1348.

In another embodiment, the invention provides a method of identifying a pattern of polynucleotide expression for identification of a compound that selectively enhances innate immunity. The invention includes detecting a pattern of polynucleotide expression for cells contacted in the presence and absence of a cationic peptide, wherein the pattern in the presence of the peptide represents stimulation of innate immunity; detecting a pattern of polynucleotide expression for cells contacted in the presence of a test compound, wherein a pattern with the test compound that is similar to the pattern observed in the presence of the cationic peptide, is indicative of a compound that enhances innate immunity. It is preferred that the compound does not stimulate a septic reaction in a subject.

In another embodiment, the invention provides a method for inferring a state of infection in a mammalian subject from a nucleic acid sample of the subject by identifying in the nucleic acid sample a polynucleotide expression pattern exemplified by an increase in polynucleotide expression of at least 2 polynucleotides in Table 50, 51 and or 52, as compared to a non-infected subject. Also included is a polynucleotide expression pattern obtained by any of the methods described above.

In another aspect a cationic peptide that is an antagonist of CXCR-4 is provided. In still another aspect, a method of identifying a cationic peptide that is an antagonist of CXCR-4 by contacting T cells with SDF-1 in the presence of absence of a test peptide and measuring chemotaxis is provided. A decrease in chemotaxis in the presence of the test peptide is indicative of a peptide that is an antagonist of CXCR-4. Cationic peptide also acts to reduce the expression of the SDF-1 receptor polynucleotide (NM_{—}012428).

In all of the above described methods, the compounds or agents of the invention include but are not limited to peptides, cationic peptides, peptidomimetics, chemical compounds, polypeptides, nucleic acid molecules and the like.

In still another aspect the invention provides an isolated cationic peptide. An isolated cationic peptide of the invention is represented by one of the following general formulas and the single letter amino acid code:
X₁X₂X₃IX₄PX₄IPX₅X₂X₁ (SEQ ID NO: 4), where X₁ is one or two of R, L or K, X₂ is one of C, S or A, X₃ is one of R or P, X₄ is one of A or V and X₅ is one of V or W;
X₁LX₂X₃KX₄X₂X₅X₃PX₃X₁ (SEQ ID NO: 11), where X₁ is one or two of D, E, S, T or N, X2 is one or two of P, G or D, X₃ is one of G, A, V, L, I or Y, X₄ is one of R, K or H and X₅ is one of S, T, C, M or R;
X₁X₂X₃X₄WX₄WX₄X₅K (SEQ ID NO: 18), where X₁ is one to four chosen from A, P or R, X₂ is one or two aromatic amino acids (F, Y and W), X₃ is one of P or K, X₄ is one, two or none chosen from A, P, Y or W and X₅ is one to three chosen from R or P;
X₁X₂X₃X₄X₁VX₃X₄RGX₄X₃X₄X₁X₃X₁ (SEQ ID NO: 25) where X₁ is one or two of R or K, X₂ is a polar or charged amino acid (S, T, M, N, Q, D, E, K, R and H), X₃ is C, S, M, D or A and X₄ is F, I, V, M or R;
X₁X₂X₃X₄X₁VX₅X₄RGX₄X₅X₄X₁X₃X₁(SEQ ID NO: 32), where X₁ is one or two of R or K, X₂ is a polar or charged amino acid (S, T, M, N, Q, D, E, K, R and H), X₃ is one of C, S, M, D or A, X₄ is one of F, I, V, M or R and X₅ is one of A, I, S, M, D or R; and
KX₁KX₂FX₂KMLMX₂ALKKX₃ (SEQ ID NO: 39), where X₁ is a polar amino acid (C, S, T, M, N and Q); X₂ is one of A, L, S or K and X₃ is 1-17 amino acids chosen from G, A, V, L, I, P, F, S, T, K and H;
KWKX₂X₁X₁X₂X₂X₁X₂X₂X₁X₁X₂X₂IFHTALKPISS (SEQ ID NO: 46), where X₁ is a hydrophobic amino acid and X₂ is a hydrophilic amino acid.

Additionally, in another aspect the invention provides isolated cationic peptides KWKSFLRTFKSPVRTVFHTALKPISS (SEQ ID NO: 53) and KWKSYAHTIMSPVRLVFHTALKPISS (SEQ ID NO: 54).

Also provided are nucleic acid sequences encoding the cationic peptides of the invention, vectors including such polynucleotides and host cells containing the vectors.

In another embodiment, the invention provides methods for stimulating or enhancing innate immunity in a subject comprising administering to the subject a peptide of the invention, for example, peptides set forth in SEQ ID NO: 1-4, 11, 18, 25, 32, 39, 46, 53 or 54. As shown in the Examples herein, innate immunity can be evidenced by monocyte activation, proliferation, differentiation or MAP kinase pathway activation just by way of example. In one aspect, the method includes further administering a serum factor such as GM-CSF to the subject. The subject is preferably any mammal and more particularly a human subject.

In another embodiment, the invention provides a method of stimulating innate immunity in a subject having or at risk of having an infection including administering to the subject a sub-optimal concentration of an antibiotic in combination with a peptide of the invention. In one aspect, the peptide is SEQ ID NO:1 or SEQ ID NO:7.

### BRIEF DESCRIPTION OF THE FIGURES

FIG. 1 demonstrates the synergy of Seq ID No: 7 with cefepime in curing S. aureus infections. CD-1 mice (8/group) were given 1×10⁷ S. aureus in 5% porcine mucin via IP injection. Test compound (50 μg-2.5 mg/kg) was given via a separate IP injection 6 hours after S. aureus. At this time Cefepime was also given at a dose of 0.1 mg/kg. Mice were euthanized 24 hr later, blood removed and plated for viable counts. The average±standard error is shown. This experiment was repeated twice.

FIG. 2 shows exposure to SEQ ID NO: 1 induces phosphorylation of ERK1/2 and p38. Lysates from human peripheral blood derived monocytes were exposed to 50 μg/ml of SEQ ID NO: 1 for 15 minutes. A) Antibodies specific for the phosphorylated forms of ERK and p38 were used to detect activation of ERK1/2 and p38. All donors tested showed increased phosphorylation of ERK1/2 and p38 in response to SEQ ID NO: 1 treatment. One representative donor of eight. Relative amounts of phosphorylation of ERK (B) and p38(C) were determined by dividing the intensities of the phosphorylated bands by the intensity of the corresponding control band as described in the Materials and Methods.

FIG. 3 shows SEQ ID NO: 1 induced phosphorylation of ERK1/2 does not occur in the absence of serum and the magnitude of phosphorylation is dependent upon the type of serum present. Human blood derived monocytes were treated with 50 μg/ml of SEQ ID NO: 1 for 15 minutes. Lysates were run on a 12% acrylamide gel then transferred to nitrocellulose membrane and probed with antibodies specific for the phosphorylated (active) form of the kinase. To normalize for protein loading, the blots were reprobed with β-actin. Quantification was done with ImageJ software. The FIG. 3 inset demonstrates that SEQ ID NO: 1 is unable to induce MAPK activation in human monocytes under serum free conditions. Cells were exposed to 50 mg/ml of SEQ ID NO: 1 (+), or endotoxin free water (−) as a vehicle control, for 15 minutes. (A) After exposure to SEQ ID NO: 1 in media containing 10% fetal calf serum, phosphorylated ERK1/2 was detectable, however, no phosphorylation of ERK1/2 was detected in the absence of serum (n=3). (B) Elk-1, a transcription factor downstream of ERK1/2, was activated (phosphorylated) upon exposure to 50 μg/ml of SEQ ID NO: 1 in media containing 10% fetal calf serum, but not in the absence of serum (n=2).

FIG. 4 shows SEQ ID NO: 1 induced activation of ERK1/2 occurs at lower concentrations and is amplified in the presence of certain cytokines. When freshly isolated monocytes were stimulated in media containing both GM-CSF (100 ng/ml) and IL-4 (100 ng/ml) SEQ ID NO: 1 induced phosphorylation of ERK1/2 was apparent at concentrations as low as 5 μg/ml. This synergistic activation of ERK1/2 seems to be due primarily to GM-CSF.

FIG. 5 shows peptide affects both transcription of various cytokine genes and release of IL-8 in the 16HBE4o-human bronchial epithelial cell line. Cells were grown to confluency on a semi-permeable membrane and stimulated on the apical surface with 50 μg/ml of SEQ ID NO: 1 for four hours. A) SEQ ID NO: 1 treated cells produced significantly more IL-8 than controls, as detected by ELISA in the supematant collected from the apical surface, but not from the basolateral surface. Mean±SE of three independent experiments shown, asterisk indicates p=0.002. B) RNA was collected from the above experiments and RT-PCR was performed. A number of cytokine genes known to be regulated by either ERK1/2 or p38 were up-regulated upon stimulation with peptide. The average of two independent experiments is shown.

FIG. 6 is a graphical representation showing that SEQ ID NO: 1 suppresses LPS-induced secretion of TNF-α. The concentration of the pro-inflammatory cytokine TNFα (Y-axis) was monitored in the tissue culture supernatant or cytoplasmic extracts of cells by ELISA. The results are an average (±standard deviation) of three independent experiments. (A) THP-1 cells were stimulated with 10 ng/ml (-●-) or 100 ng/ml (-▪-) of LPS in the presence of increasing concentrations of SEQ ID NO: 1 (X-axis) for 4 hr. (B) PBMCs were stimulated with 100 ng/ml of LPS in presence or absence of 20 μg/ml SEQ ID NO: 1 for 4 hrs. The anti-endotoxin effect of SEQ ID NO: 1 demonstrated in PBMC was statistically significant with p-value of <0.05 (**). (C) THP-1 cells were treated with LPS, SEQ ID NO: 1 or LPS+SEQ ID NO: 1 for 4 hr in the absence (white bar) or presence of actinomycin D (black bar), the effect of actinomycin D on LPS-induced TNFα secretion was statistical significant with p-value <0.001 (***). (D) Cytoplasmic extracts of THP-1 cells treated with LPS, SEQ ID NO: 1 or LPS+SEQ ID NO: 1 for 60 mins in the absence (black bar) or presence of monensin (white bar) were monitored by ELISA.

FIG. 7 is a graphical representation showing the anti-endotoxic effect of SEQ ID NO: 1 involves pre- and post-transcriptional events. Tissue culture supernatants were screened for TNFα by ELISA following stimulation of cells with 100 ng/ml of LPS in the absence (-▪-) or in the presence of 20 μg/ml SEQ ID NO: 1 (-●-) for 1, 2, 4 and 24 hr of treatment. In each case, the control indicates un-stimulated cells (-▴-), the y-axis represents TNFα concentration and the x-axis indicates time (hr). SEQ ID NO: 1 (20 ug/ml) was added (A) simultaneously with LPS, (B) after 30 min of LPS treatment, or (C) 30 min prior to LPS treatment. See materials and method for details. The results are an average (±standard deviation) of 3 independent experiments.

FIG. 8 is a graphical representation showing that SEQ ID NO: 1 modifies inflammatory agent-induced cytokine secretion by PBMC. PBMC were incubated alone or with TLR agonists (LPS, LTA, CpG) or inflammatory cytokines (TNFα, IL1β) for 4 or 24 hr in the presence (black bars) or absence (white bars) of SEQ ID NO: 1. See materials and method for details. The concentration (y-axis) of IL1β, IL6, IL8 and TNFα (x-axis) were measured in the tissue culture supernatants by multiplex bead ELISA. The results are an average (±standard deviation) of 3 independent experiments. The effect of SEQ ID NO: 1 on agonist induced cytokine production was statistical significant with p-value <0.05 (***), p<0.1 (**) or p<0.15 (*).

FIG. 9 a graphical representation showing an LPS-induced gene transcription profile in monocytes is altered by the presence of host defense peptide SEQ ID NO: 1. (A) THP-1 cells were stimulated with 100 ng/ml LPS in the absence (top panel) or presence (lower panel) of 20 ug/ml SEQ ID NO: 1 for 1, 2, 4 or 24 hr. Using microarray analysis, the gene expression in response to stimuli was calculated relative to that in unstimulated cells at each time point. The relative gene expression is overlayed on the TLR-4 protein network using the supervised clustering tool Cytoscape. The colour code for the fold change and identification of proteins are in the left panel. (B) Cluster analysis of the differentially expressed genes as measured using log ratio (y-axis) of microarray spot intensity, with NFκB binding sites in response to 100 ng/ml of LPS in the absence (top) or presence of 20 ug/ml of SEQ ID NO: 1 (bottom) based on similar temporal expression profiles over the time course of 1 to 24 hr (x-axis) using K-means, a no-hierarchical algorithm with an affinity threshold of 85%. The table indicates the total number of differentially expressed genes, total number of clusters, number of clusters containing genes with NFκB binding sites and the NFκB target genes found in the clusters.

FIG. 10 is a graphical representation showing that SEQ ID NO: 1 selectively modulates the transcription of LPS-induced pro-inflammatory genes. qPCR of gene expression in LPS-stimulated cells (-▪-), cells treated with SEQ ID NO: 1 alone (-▴-) or cells treated with a combination of LPS and SEQ ID NO: 1 (-●-) for 1,2,4, and 24 hr (x-axis). Results shown are an average (±standard error) of three independent experiments. Fold changes (y-axis, log scale) for each gene were normalized to GAPDH and are relative to the gene expression in un-stimulated cells (normalized to 1) using the comparative Ct method (see materials and methods for details).

FIG. 11 is a pictorial diagram and a graphical representation showing that SEQ ID NO: 1 suppresses LPS-induced translocation of NFκB subunits p50 and p65. (A) Western blot of NFκB subunits (identified on the right) in the nuclear extract of THP-1 cells following incubation in the absence (−) or presence (+) of 100 ng/ml LPS or LPS and 20 μg/ml SEQ ID NO: 1 for 60 mins. Pre-stained molecular mass markers are indicated on the left. (B) ELISA for NFκB subunit p50 (upper panel) and NFκB subunit p65 (lower panel) detected in the nuclear extracts of THP-1 cells stimulated for 60 min as described in (A). The y-axis represents relative light units (luminescence). See materials and methods for details. Results are representative of 3 independent experiments.

FIG. 12 is a pictorial diagram of a model describing mechanisms of anti-endotoxin activity of SEQ ID NO: 1. Based on the data presented herein, SEQ ID NO: 1 regulates LPS-induced gene transcription and cytokine production, by one or more of several mechanisms. (1) SEQ ID NO: 1 can interact directly with LPS to reduce its binding to LBP, MD2 or another component of the TLR4 receptor complex, thus reducing activation of the downstream pathway. (2) SEQ ID NO: 1 partially inhibits the TLR4→NFκB pathway and LPS-induced p50/p65 translocation probably by the action of certain negative regulators of NFκB (TNFAIP3, NFKBIA), the expression of which is relatively unaffected by SEQ ID NO: 1. (3) SEQ ID NO: 1 selectively modulates gene transcription; completely inhibiting certain pro-inflammatory genes (NF↓B-1 (p50), TNFAIP2) and reducing the expression of others (TNFα). (4) SEQ ID NO: 1 directly triggers MAP kinase pathways that can impact on pro-inflammatory pathways. (5) SEQ ID NO: 1 has a stronger effect on e.g. TNFα protein production than on TNFα gene expression, and thus may directly or indirectly influence protein translation, stabilization, or processing. Points of intervention by SEQ ID NO: 1 are indicated by activation (z,900 ,z,901 ), inhibition (⊥), or suppression (z,902 ). Other abbreviations used are phosphorylation (P) and ubiquitination (U).

### DETAILED DESCRIPTION OF THE INVENTION

The present invention provides novel cationic peptides, characterized by a group of generic formulas, which have ability to modulate (e.g., up- and/or down regulate) polynucleotide expression, thereby regulating sepsis and inflammatory responses and/or innate immunity.

“Innate immunity” as used herein refers to the natural ability of an organism to defend itself against invasions by pathogens. Pathogens or microbes as used herein, may include, but are not limited to bacteria, fungi, parasite, and viruses. Innate immunity is contrasted with acquired/adaptive immunity in which the organism develops a defensive mechanism based substantially on antibodies and/or immune lymphocytes that is characterized by specificity, amplifiability and self vs. non-self dsicrimination. With innate immunity, broad, nonspecific immunity is provided and there is no immunologic memory of prior exposure. The hallmarks of innate immunity are effectiveness against a broad variety of potential pathogens, independence of prior exposure to a pathogen, and immediate effectiveness (in contrast to the specific immune response which takes days to weeks to be elicited). In addition, innate immunity includes immune responses that affect other diseases, such as cancer, inflammatory diseases, multiple sclerosis, various viral infections, and the like.

As used herein, the term “cationic peptide” refers to a sequence of amino acids from about 5 to about 50 amino acids in length. In one aspect, the cationic peptide of the invention is from about 10 to about 35 amino acids in length. A peptide is “cationic” if it possesses sufficient positively charged amino acids to have a pI greater than about 9.0, where pI (isoelectric point)=pH when the net charge of the peptide is neutral. Typically, at least two of the amino acid residues of the cationic peptide will be positively charged, for example, lysine or arginine. “Positively charged” refers to the side chains of the amino acid residues which have a net positive charge at pH 7.0. Examples of naturally occurring cationic antimicrobial peptides which can be recombinantly produced according to the invention include defensins, cathelicidins, magainins, melittin, and cecropins, bactenecins, indolicidins, polyphemusins, tachyplesins, and analogs thereof. A variety of organisms make cationic peptides, molecules used as part of a non-specific defense mechanism against microorganisms. When isolated, these peptides are toxic to a wide variety of microorganisms, including bacteria, fungi, and certain enveloped viruses. While cationic peptides act against many pathogens, notable exceptions and varying degrees of toxicity exist. However this patent reveals additional cationic peptides with no toxicity towards microorganisms but an ability to protect against infections through stimulation of innate immunity, and this invention is not limited to cationic peptides with antimicrobial activity. In fact, many peptides useful in the present invention do not have antimicrobial activity.

Cationic peptides known in the art include for example, the human cathelicidin LL-37, and the bovine neutrophil peptide indolicidin and the bovine variant of bactenecin, Bac2A.
LL-37 LLGDFFRKSKEKIGKEFKRIVQRIKDFLRNLVPRTES (SEQ ID NO: 1)
Indolicidin ILPWKWPWWPWRR-NH₂ (SEQ ID NO: 2)
Bac2A RLARIVVIRVAR-NH₂ (SEQ ID NO: 3)

Although SEQ ID NO: 1 is often defined as an antimicrobial (direct killing) peptide it has been suggested that at physiological salt conditions, this peptide is not antimicrobial at the concentrations (1-5 μg/ml) normally found in adults at mucosal surfaces (Bowdish, D. M. E., D. J. Davidson, Y. E. Lau, K. Lee, M. G. Scott, and R. E. W. Hancock. 2005. Impact of LL-37 on anti-infective immunity. J. Leukocyte Biol. 77:451-459). Moreover under these conditions and at these concentrations, SEQ ID NO: 1 exhibits a variety of immunomodulatory functions. This could help to explain why SEQ ID NO: 1 administration can protect mice against certain bacterial infections, due to its ability to modulate immunity. SEQ ID NO: 1 is also able to protect mice and rats against endotoxemia/sepsis induced by pure LPS indicating that SEQ ID NO: 1 can suppress potentially harmful pro-inflammatory responses.

Accordingly, the present invention provides evidence that human host defense peptide SEQ ID NO: 1 has potent anti-endotoxin properties, at very low (≦1 μg/ml) concentrations and physiological salt conditions reflecting those found in vivo. It is further demonstrated here that SEQ ID NO: 1 had a general anti-inflammatory effect on TLR stimulation, inhibiting pro-inflammatory cytokine release from human monocytic cells stimulated with TLR2, TLR4 and TLR9 agonists. The suppression of inflammatory responses by SEQ ID NO: 1 in LPS-stimulated cells is selective, as SEQ ID NO: 1 does not block the expression of certain (pro-inflammatory) genes required for cell recruitment and movement, yet abrogates pro-inflammatory cytokine responses that can potentially lead to sepsis. The anti-inflammatory activity of SEQ ID NO: 1 is apparently mediated through a diversity of mechanisms.

In innate immunity, the immune response is not dependent upon antigens. The innate immunity process may include the production of secretory molecules and cellular components as set forth above. In innate immunity, the pathogens are recognized by receptors (for example, Toll-like receptors) that have broad specificity, are capable of recognizing many pathogens, and are encoded in the germline. These Toll-like receptors have broad specificity and are capable of recognizing many pathogens. When cationic peptides are present in the immune response, they aid in the host response to pathogens. This change in the immune response induces the release of chemokines, which promote the recruitment of immune cells to the site of infection.

Chemokines, or chemoattractant cytokines, are a subgroup of immune factors that mediate chemotactic and other pro-inflammatory phenomena (See, Schall, 1991, Cytokine 3:165-183). Chemokines are small molecules of approximately 70-80 residues in length and can generally be divided into two subgroups, a which have two N-terminal cysteines separated by a single amino acid (CxC) and β which have two adjacent cysteines at the N terminus (CC). RANTES, MIP-1α and MIP-1β are members of the β subgroup (reviewed by Horuk, R., 1994, Trends Pharmacol. Sci, 15:159-165; Murphy, P. M., 1994, Annu. Rev. Immunol., 12:593-633). The amino terminus of the β chemokines RANTES, MCP-1, and MCP-3 have been implicated in the mediation of cell migration and inflammation induced by these chemokines. This involvement is suggested by the observation that the deletion of the amino terminal 8 residues of MCP-1, amino terminal 9 residues of MCP-3, and amino terminal 8 residues of RANTES and the addition of a methionine to the amino terminus of RANTES, antagonize the chemotaxis, calcium mobilization and/or enzyme release stimulated by their native counterparts (Gong et al., 1996 J Biol. Chem. 271:10521-10527; Proudfoot et al., 1996 J Biol. Chem. 271:2599-2603). Additionally, a chemokine-like chemotactic activity has been introduced into MCP-1 via a double mutation of Tyr 28 and Arg 30 to leucine and valine, respectively, indicating that internal regions of this protein also play a role in regulating chemotactic activity (Beall et al., 1992, J Biol. Chem. 267:3455-3459).

The monomeric forms of all chemokines characterized thus far share significant structural homology, although the quaternary structures of α and β groups are distinct. While the monomeric structures of the β and α chemokines are very similar, the dimeric structures of the two groups are completely different. An additional chemokine, lymphotactin, which has only one N terminal cysteine has also been identified and may represent an additional subgroup (γ) of chemokines (Yoshida et al., 1995, FEBS Lett. 360:155-159; and Kelner et al., 1994, Science 266:1395-1399).

Receptors for chemokines belong to the large family of G-protein coupled, 7 transmembrane domain receptors (GCR's) (See, reviews by Horuk, R., 1994, Trends Pharmacol. Sci. 15:159-165; and Murphy, P. M., 1994, Annu. Rev. Immunol. 12:593-633). Competition binding and cross-desensitization studies have shown that chemokine receptors exhibit considerable promiscuity in ligand binding. Examples demonstrating the promiscuity among β chemokine receptors include: CC CKR-1, which binds RANTES and MIP-1α (Neote et al., 1993, Cell 72: 415-425), CC CKR-4, which binds RANTES, MIP-1α , and MCP-1 (Power et al., 1995, J. Biol Chem. 270:19495-19500), and CC CKR-5, which binds RANTES, MIP-1α, and MIP-1β (Alkhatib et al., 1996, Science, in press and Dragic et al., 1996, Nature 381:667-674). Erythrocytes possess a receptor (known as the Duffy antigen) which binds both α and β chemokines (Horuk et al., 1994, J. Biol. Chem. 269:17730-17733; Neote et al., 1994, Blood 84:44-52; and Neote et al., 1993, J Biol. Chem. 268:12247-12249). Thus the sequence and structural homologies evident among chemokines and their receptors allows some overlap in receptor-ligand interactions.

In one aspect, the present invention provides the use of compounds including peptides of the invention to reduce sepsis and inflammatory responses by acting directly on host cells. In this aspect, a method of identification of a polynucleotide or polynucleotides that are regulated by one or more sepsis or inflammatory inducing agents is provided, where the regulation is altered by a cationic peptide. Such sepsis or inflammatory inducing agents include, but are not limited to endotoxic lipopolysaccharide (LPS), lipoteichoic acid (LTA) and/or CpG DNA or intact bacteria or other bacterial components. The identification is performed by contacting the polynucleotide or polynucleotides with the sepsis or inflammatory inducing agents and further contacting with a cationic peptide either simultaneously or immediately after. The expression of the polynucleotide in the presence and absence of the cationic peptide is observed and a change in expression is indicative of a polynucleotide or pattern of polynucleotides that is regulated by a sepsis or inflammatory inducing agent and inhibited by a cationic peptide. In another aspect, the invention provides a polynucleotide identified by the method.

Once identified, such polynucleotides will be useful in methods of screening for compounds that can block sepsis or inflammation by affecting the expression of the polynucleotide. Such an effect on expression may be either up regulation or down regulation of expression. By identifying compounds that do not trigger the sepsis reaction and that can block or dampen inflammatory or septic responses, the present invention also presents a method of identifying enhancers of innate immunity. Additionally, the present invention provides compounds that are used or identified in the above methods.

Candidate compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, and the like to produce structural analogs. Candidate agents are also found among biomolecules including, but not limited to: peptides, peptidiomimetics, saccharides, fatty acids, steroids, purines, pyrimidines, polypeptides, polynucleotides, chemical compounds, derivatives, structural analogs or combinations thereof.

Incubating components of a screening assay includes conditions which allow contact between the test compound and the polynucleotides of interest. Contacting includes in solution and in solid phase, or in a cell. The test compound may optionally be a combinatorial library for screening a plurality of compounds. Compounds identified in the method of the invention can be further evaluated, detected, cloned, sequenced, and the like, either in solution or after binding to a solid support, by any method usually applied to the detection of a compound.

Generally, in the methods of the invention, a cationic peptide is utilized to detect and locate a polynucleotide that is essential in the process of sepsis or inflammation. Once identified, a pattern of polynucleotide expression may be obtained by observing the expression in the presence and absence of the cationic peptide. The pattern obtained in the presence of the cationic peptide is then useful in identifying additional compounds that can inhibit expression of the polynucleotide and therefore block sepsis or inflammation. It is well known to one of skill in the art that non-peptidic chemicals and peptidomimetics can mimic the ability of peptides to bind to receptors and enzyme binding sites and thus can be used to block or stimulate biological reactions. Where an additional compound of interest provides a pattern of polynucleotide expression similar to that of the expression in the presence of a cationic peptide, that compound is also useful in the modulation of sepsis or an innate immune response. In this manner, the cationic peptides of the invention, which are known inhibitors of sepsis and inflammation and enhancers of innate immunity are useful as tools in the identification of additional compounds that inhibit sepsis and inflammation and enhance innate immunity.

As can be seen in the Examples below, peptides of the invention have a widespread ability to reduce the expression of polynucleotides regulated by LPS. High levels of endotoxin in the blood are responsible for many of the symptoms seen during a serious infection or inflammation such as fever and an elevated white blood cell count. Endotoxin is a component of the cell wall of Gram-negative bacteria and is a potent trigger of the pathophysiology of sepsis. The basic mechanisms of inflammation and sepsis are related. In Example 1, polynucleotide arrays were utilized to determine the effect of cationic peptides on the transcriptional response of epithelial cells. Specifically, the effects on over 14,000 different specific polynucleotide probes induced by LPS were observed. The tables show the changes seen with cells treated with peptide compared to control cells. The resulting data indicated that the peptides have the ability to reduce the expression of polynucleotides induced by LPS.

Example 2, similarly, shows that peptides of the invention are capable of neutralizing the stimulation of immune cells by Gram positive and Gram negative bacterial products. Additionally, it is noted that certain pro-inflammatory polynucleotides are down-regulated by cationic peptides, as set forth in table 24 such as TLR1 (AI339155), TLR2 (T57791), TLR5 (N41021), TNF receptor-associated factor 2 (T55353), TNF receptor-associated factor 3 (AA504259), TNF receptor superfamily, member 12 (W71984), TNF receptor superfamily, member 17 (AA987627), small inducible cytokine subfamily B, member 6 (AI889554), IL-12R beta 2 (AA977194), IL-18 receptor 1 (AA482489), while anti-inflammatory polynucleotides are up-regulated by cationic peptides, as seen in table 25 such as IL-1 R antagonist homolog 1 (AI167887), IL-10 R beta (AA486393), TNF Receptor member 1B (AA150416), TNF receptor member 5 (H98636), TNF receptor member 11b (AA194983), IK cytokine down-regulator of HLA II (R39227), TGF-B inducible early growth response 2 (AI473938), or CD2 (AA927710). The relevance and application of these results are confirmed by an in vivo application to mice.

In another aspect, the invention provides a method of identifying an agent that enhances innate immunity. In the method, a polynucleotide or polynucleotides that encode a polypeptide involved in innate immunity is contacted with an agent of interest. Expression of the polynucleotide is determined, both in the presence and absence of the agent. The expression is compared and of the specific modulation of expression was indicative of an enhancement of innate immunity. In another aspect, the agent does not stimulate a septic reaction as revealed by the lack of upregulation of the pro-inflammatory cytokine TNF-α. In still another aspect the agent reduces or blocks the inflammatory or septic response. In yet another aspect, the agent reduces the expression of TNF-α and/or interleukins including, but not limited to, IL-1β, IL-6, IL-12 p40, IL-12 p70, and IL-8.

In another aspect, the invention provides methods of direct polynucleotide regulation by cationic peptides and the use of compounds including cationic peptides to stimulate elements of innate immunity. In this aspect, the invention provides a method of identification of a pattern of polynucleotide expression for identification of a compound that enhances innate immunity. In the method of the invention, an initial detection of a pattern of polynucleotide expression for cells contacted in the presence and absence of a cationic peptide is made. The pattern resulting from polynucleotide expression in the presence of the peptide represents stimulation of innate immunity. A pattern of polynucleotide expression is then detected in the presence of a test compound, where a resulting pattern with the test compound that is similar to the pattern observed in the presence of the cationic peptide is indicative of a compound that enhances innate immunity. In another aspect, the invention provides compounds that are identified in the above methods. In another aspect, the compound of the invention stimulates chemokine or chemokine receptor expression. Chemokine or chemokine receptors may include, but are not limited to CXCR4, CXCR1, CXCR2, CCR2, CCR4, CCR5, CCR6, MIP-1 alpha, MDC, MIP-3 alpha, MCP-1, MCP-2, MCP-3, MCP-4, MCP-5, and RANTES. In still another aspect, the compound is a peptide, peptidomimetic, chemical compound, or a nucleic acid molecule.

In still another aspect the polynucleotide expression pattern includes expression of pro-inflammatory polynucleotides. Such pro-inflammatory polynucleotides may include, but are not limited to, ring finger protein 10 (D8745 1), serine/threonine protein kinase MASK (AB040057), KIAA0912 protein (AB020719), KIAA0239 protein (D87076), RAP1, GTPase activating protein 1 (M64788), FEM-1-like death receptor binding protein (AB007856), cathepsin S (M90696), hypothetical protein FLJ20308 (AK000315), pim-1 oncogene (M54915), proteasome subunit beta type 5 (D29011), KIAA0239 protein (D87076), mucin 5 subtype B tracheobronchial (AJ001403), cAMP response element-binding protein CREBPa, integrin alpha M (J03925), Rho-associated kinase 2 (NM_{—}004850), PTD017 protein (AL050361) unknown genes (AK001143, AK034348, AL049250, AL161991, AL031983) and any combination thereof. In still another aspect the polynucleotide expression pattern includes expression of cell surface receptors that may include but is not limited to retinoic acid receptor (X06614), G protein-coupled receptors (Z94155, X81892, U52219, U22491, AF015257, U66579) chemokine (C-C motif) receptor 7 (L31584), tumor necrosis factor receptor superfamily member 17 (Z29575), interferon gamma receptor 2 (U05875), cytokine receptor-like factor 1 (AF059293), class I cytokine receptor (AF053004), coagulation factor II (thrombin) receptor-like 2 (U92971), leukemia inhibitory factor receptor (NM_{—}002310), interferon gamma receptor 1 (AL050337).

In Example 4 it can be seen that the cationic peptides of the invention alter polynucleotide expression in macrophage and epithelial cells. The results of this example show that pro-inflammatory polynucleotides are down-regulated by cationic peptides (Table 24) whereas anti-inflammatory polynucleotides are up-regulated by cationic peptides (Table 25).

It is shown below, for example, in tables 1-15, that cationic peptides can neutralize the host response to the signaling molecules of infectious agents as well as modify the transcriptional responses of host cells, mainly by down-regulating the pro-inflammatory response and/or up-regulating the anti-inflammatory response. Example 5 shows that the cationic peptides can aid in the host response to pathogens by inducing the release of chemokines, which promote the recruitment of immune cells to the site of infection. The results are confirmed by an in vivo application to mice.

It is seen from the examples below that cationic peptides have a substantial influence on the host response to pathogens in that they assist in regulation of the host immune response by inducing selective pro-inflammatory responses that for example promote the recruitment of immune cells to the site of infection but not inducing potentially harmful pro-inflammatory cytokines. Sepsis appears to be caused in part by an overwhelming pro-inflammatory response to infectious agents. Peptides can aid the host in a “balanced” response to pathogens by inducing an anti-inflammatory response and suppressing certain potentially harmful pro-inflammatory responses.

In Example 7, the activation of selected MAP kinases was examined, to study the basic mechanisms behind the effects of interaction of cationic peptides with cells. Macrophages activate MEK/ERK kinases in response to bacterial infection. MEK is a MAP kinase kinase that when activated, phosphorylates the downstream kinase ERK (extracellular regulated kinase), which then dimerizes and translocates to the nucleus where it activates transcription factors such as Elk-1 to modify polynucleotide expression. MEK/ERK kinases have been shown to impair replication of Salmonella within macrophages. Signal transduction by MEK kinase and NADPH oxidase may play an important role in innate host defense against intracellular pathogens. By affecting the MAP kinases as shown below the cationic peptides have an effect on bacterial infection. The cationic peptides can directly affect kinases. Table 21 demonstrates but is not limited to MAP kinase polynucleotide expression changes in response to peptide. The kinases include MAP kinase kinase 6 (H070920), MAP kinase kinase 5 (W69649), MAP kinase 7 (H39192), MAP kinase 12 (AI936909) and MAP kinase-activated protein kinase 3 (W68281).

In another method, the methods of the invention may be used in combination, to identify an agent with multiple characteristics, i.e. a peptide with anti-inflammatory/anti-sepsis activity, and the ability to enhance innate immunity, in part by inducing chemokines in vivo.

In another aspect, the invention provides a method for inferring a state of infection in a mammalian subject from a nucleic acid sample of the subject by identifying in the nucleic acid sample a polynucleotide expression pattern exemplified by an increase in polynucleotide expression of at least 2 polynucleotides in Table 55 as compared to a non-infected subject. In another aspect the invention provides a method for inferring a state of infection in a mammalian subject from a nucleic acid sample of the subject by identifying in the nucleic acid sample a polynucleotide expression pattern exemplified by a polynucleotide expression of at least 2 polynucleotides in Table 56 or Table 57 as compared to a non-infected subject. In one aspect of the invention, the state of infection is due to infectious agents or signaling molecules derived therefrom, such as, but not limited to, Gram negative bacteria and Gram positive bacteria, viral, fungal or parasitic agents. In still another aspect the invention provides a polynucleotide expression pattern of a subject having a state of infection identified by the above method. Once identified, such polynucleotides will be useful in methods of diagnosis of a condition associated with the activity or presence of such infectious agents or signaling molecules.

Example 10 below demonstrates this aspect of the invention. Specifically, table 61 demonstrates that both MEK and the NADPH oxidase inhibitors can limit bacterial replication (infection of IFN-γ-primed macrophages by S. typhimurium triggers a MEK kinase). This is an example of how bacterial survival can be impacted by changing host cell signaling molecules.

In still another aspect of the invention, compounds are presented that inhibit stromal derived factor-1 (SDF-1) induced chemotaxis of T cells. Compounds are also presented which decrease expression of SDF-1 receptor. Such compounds also may act as an antagonist or inhibitor of CXCR-4. In one aspect the invention provides a cationic peptide that is an antagonist of CXCR-4. In another aspect the invention provides a method of identifying a cationic peptide that is an antagonist of CXCR-4. The method includes contacting T cells with SDF-1 in the presence of absence of a test peptide and measuring chemotaxis. A decrease in chemotaxis in the presence of the test peptide is then indicative of a peptide that is an antagonist of CXCR-4. Such compounds and methods are useful in therapeutic applications in HIV patients. These types of compounds and the utility thereof is demonstrated, for example, in Example 11 (see also Tables 62, 63). In that example, cationic peptides are shown to inhibit cell migration and therefore antiviral activity.

In one embodiment, the invention provides an isolated cationic peptides having an amino acid sequence of the general formula (Formula A): X₁X₂X₃IX₄PX₄IPX₅X₂X₁ (SEQ ID NO: 4), wherein X₁is one or two of R, L or K, X₂ is one of C, S or A, X₃ is one of R or P, X₄ is one of A or V and X₅ is one of V or W. Examples of the peptides of the invention include, but are not limited to: LLCRIVPVIPWCK (SEQ ID NO: 5), LRCPIAPVIPVCKK (SEQ ID NO: 6), KSRIVPAIPVSLL (SEQ ID NO: 7), KKSPIAPAIPWSR (SEQ ID NO: 8), RRARIVPAIPVARR (SEQ ID NO: 9) and LSRIAPAIPWAKL (SEQ ID NO: 10).

In another embodiment, the invention provides an isolated linear cationic peptide having an amino acid sequence of the general formula (Formula B):
X₁LX₂X₃KX₄X₂X₅X₃PX₃X₁ (SEQ ID NO: 11), wherein X₁ is one or two of D, E, S, T or N, X₂ is one or two of P, G or D, X₃ is one of G, A, V, L, I or Y, X₄ is one of R, K or H and X₅ is one of S, T, C, M or R. Examples of the peptides of the invention include, but are not limited to:
DLPAKRGSAPGST (SEQ ID NO: 12), SELPGLKHPCVPGS (SEQ ID NO: 13),
TTLGPVKRDSIPGE (SEQ ID NO: 14), SLPIKHDRLPATS (SEQ ID NO: 15),
ELPLKRGRVPVE (SEQ ID NO: 16) and NLPDLKKPRVPATS (SEQ ID NO: 17).

In another embodiment, the invention provides an isolated linear cationic peptide having an amino acid sequence of the general formula (Formula C): X₁X₂X₃X₄WX₄WX₄X₅K (SEQ ID NO: 18) (this formula includes CP₁₂a and CP12d), wherein X₁ is one to four chosen from A, P or R, X₂ is one or two aromatic amino acids (F, Y and W), X₃ is one of P or K, X₄ is one, two or none chosen from A, P, Y or W and X₅ is one to three chosen from R or P. Examples of the peptides of the invention include, but are not limited to:
RPRYPWWPWWPYRPRK (SEQ ID NO: 19), RRAWWKAWWARRK (SEQ ID NO: 20),
RAPYWPWAWARPRK (SEQ ID NO: 21), RPAWKYWWPWPWPRRK (SEQ ID NO: 22),
RAAFKWAWAWWRRK (SEQ ID NO: 23) and RRRWKWAWPRRK (SEQ ID NO: 24).

In another embodiment, the invention provides an isolated hexadecameric cationic peptide having an amino acid sequence of the general formula (Formula D):
X₁X₂X₃X₄X₁VX₃X₄RGX₄X₃X₄X₁X₃X₁(SEQ ID NO: 25) wherein X₁ is one or two of R or K,
X₂ is a polar or charged amino acid (S, T, M, N, Q, D, E, K, R and H), X₃ is C, S, M, D or A and X₄ is F, I, V, M or R. Examples of the peptides of the invention include, but are not limited to: RRMCIKVCVRGVCRRKCRK (SEQ ID NO: 26), KRSCFKVSMRGVSRRRCK (SEQ ID NO: 27), KKDAIKKVDIRGMDMRRAR (SEQ ID NO: 28), RKMVKVDVRGIMIRKDRR (SEQ ID NO: 29), KQCVKVAMRGMALRRCK (SEQ ID NO: 30) and
RREAIRRVAMRGRDMKRMRR (SEQ ID NO: 31).

In still another embodiment, the invention provides an isolated hexadecameric cationic peptide having an amino acid sequence of the general formula (Formula E):
X₁X₂X₃X₄X₁VX₅X₄RGX₄X₅X₄X₁X₃X₁(SEQ. ID NO: 32), wherein X₁ is one or two of R or K, X₂ is a polar or charged amino acid (S, T, M, N, Q, D, E, K, R and H), X₃ is one of C, S, M, D or A, X₄ is one of F, I, V, M or R and X₅ is one of A, I, S, M, D or R. Examples of the peptides of the invention include, but are not limited to: RTCVKRVAMRGIIRKRCR (SEQ ID NO: 33), KKQMMKRVDVRGISVKRKR (SEQ ID NO: 34), KESIKVIIRGMMVRMKK (SEQ ID NO: 35), RRDCRRVMVRGIDIKAK (SEQ ID NO: 36), KRTAIKKVSRRGMSVKARR (SEQ ID NO: 37) and RHCIRRVSMRGIIMRRCK (SEQ ID NO: 38).

In another embodiment, the invention provides an isolated longer cationic peptide having an amino acid sequence of the general formula (Formula F):
KX₁KX₂FX₂KMLMX₂ALKKX₃ (SEQ ID NO: 39), wherein X₁ is a polar amino acid (C, S, T, M, N and Q); X₂ is one of A, L, S or K and X₃ is 1-17 amino acids chosen from G, A, V, L, I, P, F, S, T, K and H. Examples of the peptides of the invention include, but are not limited to:
KCKLFKKMLMLALKKVLTTGLPALKLTK (SEQ ID NO: 40),
KSKSFLKMLMKALKKVLTTGLPALIS (SEQ ID NO: 41),
KTKKFAKMLMMALKKVVSTAKPLAILS (SEQ ID NO: 42),
KMKSFAKMLMLALKKVLKVLTTALTLKAGLPS (SEQ ID NO: 43),
KNKAFAKMLMKALKKVTTAAKPLTG (SEQ ID NO: 44) and
KQKLFAKMLMSALKKKTLVTTPLAGK (SEQ ID NO: 45).

In yet another embodiment, the invention provides an isolated longer cationic peptide having an amino acid sequence of the general formula (Formula G):
KWKX₂X₁X₁X₂X₂X₁X₂X₂X₁X₁X₂X₂IFHTALKPISS (SEQ ID NO: 46), wherein X₁ is a hydrophobic amino acid and X₂ is a hydrophilic amino acid. Examples of the peptides of the invention include, but are not limited to: KWKSFLRTFKSPVRTIFHTALKPISS (SEQ ID NO: 47), KWKSYAHTIMSPVRLIFHTALKPISS (SEQ ID NO: 48),
KWKRGAHRFMKFLSTIFHTALKPISS (SEQ ID NO: 49),
KWKKWAHSPRKVLTRIFHTALKPISS (SEQ ID NO: 50),
KWKSLVMMFKKPARRIFHTALKPISS (SEQ ID NO: 51) and
KWKHALMKAHMLWHMIFHTALKPISS (SEQ ID NO: 52).

In still another embodiment, the invention provides an isolated cationic peptide having an amino acid sequence of the formula: KWKSFLRTFKSPVRTVFHTALKPISS (SEQ ID NO: 53) or KWKSYAHTIMSPVRLVFHTALKPISS (SEQ ID NO: 54).

The term “isolated” as used herein refers to a peptide that is substantially free of other proteins, lipids, and nucleic acids (e.g., cellular components with which an in vivo-produced peptide would naturally be associated). Preferably, the peptide is at least 70%, 80%, or most preferably 90% pure by weight.

The invention also includes analogs, derivatives, conservative variations, and cationic peptide variants of the enumerated polypeptides, provided that the analog, derivative, conservative variation, or variant has a detectable activity in which it enhances innate immunity or has anti-inflammatory activity. It is not necessary that the analog, derivative, variation, or variant have activity identical to the activity of the peptide from which the analog, derivative, conservative variation, or variant is derived.

A cationic peptide “variant” is an peptide that is an altered form of a referenced cationic peptide. For example, the term “variant” includes a cationic peptide in which at least one amino acid of a reference peptide is substituted in an expression library. The term “reference” peptide means any of the cationic peptides of the invention (e.g., as defined in the above formulas), from which a variant, derivative, analog, or conservative variation is derived. Included within the term “derivative” is a hybrid peptide that includes at least a portion of each of two cationic peptides (e.g., 30-80% of each of two cationic peptides). Also included are peptides in which one or more amino acids are deleted from the sequence of a peptide enumerated herein, provided that the derivative has activity in which it enhances innate immunity or has anti-inflammatory activity. This can lead to the development of a smaller active molecule which would also have utility. For example, amino or carboxy terminal amino acids which may not be required for enhancing innate immunity or anti-inflammatory activity of a peptide can be removed. Likewise, additional derivatives can be produced by adding one or a few (e.g., less than 5) amino acids to a cationic peptide without completely inhibiting the activity of the peptide. In addition, C-terminal derivatives, e.g., C-terminal methyl esters, and N-terminal derivatives can be produced and are encompassed by the invention. Peptides of the invention include any analog, homolog, mutant, isomer or derivative of the peptides disclosed in the present invention, so long as the bioactivity as described herein remains. Also included is the reverse sequence of a peptide encompassed by the general formulas set forth above. Additionally, an amino acid of “D” configuration may be substituted with an amino acid of “L” configuration and vice versa. Alternatively the peptide may be cyclized chemically or by the addition of two or more cysteine residues within the sequence and oxidation to form disulphide bonds.

The invention also includes peptides that are conservative variations of those peptides exemplified herein. The term “conservative variation” as used herein denotes a polypeptide in which at least one amino acid is replaced by another, biologically similar residue. Examples of conservative variations include the substitution of one hydrophobic residue, such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic for aspartic acid, or glutamine for asparagine, and the like. Neutral hydrophilic amino acids that can be substituted for one another include asparagine, glutamine, serine and threonine. The term “conservative variation” also encompasses a peptide having a substituted amino acid in place of an unsubstituted parent amino acid. Such substituted amino acids may include amino acids that have been methylated or amidated. Other substitutions will be known to those of skill in the art. In one aspect, antibodies raised to a substituted polypeptide will also specifically bind the unsubstituted polypeptide.

Peptides of the invention can be synthesized by commonly used methods such as those that include t-BOC or FMOC protection of alpha-amino groups. Both methods involve stepwise synthesis in which a single amino acid is added at each step starting from the C-terminus of the peptide (See, Coligan, et al., Current Protocols in Immunology, Wiley Interscience, 1991, Unit 9). Peptides of the invention can also be synthesized by the well known solid phase peptide synthesis methods such as those described by Merrifield, J. Am. Chem. Soc., 85:2149, 1962) and Stewart and Young, Solid Phase Peptides Synthesis, Freeman, San Francisco, 1969, pp. 27-62) using a copoly(styrene-divinylbenzene) containing 0.1-1.0 mMol amines/g polymer. On completion of chemical synthesis, the peptides can be deprotected and cleaved from the polymer by treatment with liquid HF-10% anisole for about 1/4-1 hours at 0° C. After evaporation of the reagents, the peptides are extracted from the polymer with a 1% acetic acid solution, which is then lyophilized to yield the crude material. The peptides can be purified by such techniques as gel filtration on Sephadex G-15 using 5% acetic acid as a solvent. Lyophilization of appropriate fractions of the column eluate yield homogeneous peptide, which can then be characterized by standard techniques such as amino acid analysis, thin layer chromatography, high performance liquid chromatography, ultraviolet absorption spectroscopy, molar rotation, or measuring solubility. If desired, the peptides can be quantitated by the solid phase Edman degradation.

The invention also includes isolated nucleic acids (e.g., DNA, cDNA, or RNA) encoding the peptides of the invention. Included are nucleic acids that encode analogs, mutants, conservative variations, and variants of the peptides described herein. The term “isolated” as used herein refers to a nucleic acid that is substantially free of proteins, lipids, and other nucleic acids with which an in vivo-produced nucleic acids naturally associated. Preferably, the nucleic acid is at least 70%, 80%, or preferably 90% pure by weight, and conventional methods for synthesizing nucleic acids in vitro can be used in lieu of in vivo methods. As used herein, “nucleic acid” refers to a polymer of deoxyribo-nucleotides or ribonucleotides, in the form of a separate fragment or as a component of a larger genetic construct (e.g., by operably linking a promoter to a nucleic acid encoding a peptide of the invention). Numerous genetic constructs (e.g., plasmids and other expression vectors) are known in the art and can be used to produce the peptides of the invention in cell-free systems or prokaryotic or eukaryotic (e.g., yeast, insect, or mammalian) cells. By taking into account the degeneracy of the genetic code, one of ordinary skill in the art can readily synthesize nucleic acids encoding the polypeptides of the invention. The nucleic acids of the invention can readily be used in conventional molecular biology methods to produce the peptides of the invention.

DNA encoding the cationic peptides of the invention can be inserted into an “expression vector.” The term “expression vector” refers to a genetic construct such as a plasmid, virus or other vehicle known in the art that can be engineered to contain a nucleic acid encoding a polypeptide of the invention. Such expression vectors are preferably plasmids that contain a promoter sequence that facilitates transcription of the inserted genetic sequence in a host cell. The expression vector typically contains an origin of replication, and a promoter, as well as polynucleotides that allow phenotypic selection of the transformed cells (e.g., an antibiotic resistance polynucleotide). Various promoters, including inducible and constitutive promoters, can be utilized in the invention. Typically, the expression vector contains a replicon site and control sequences that are derived from a species compatible with the host cell.

Transformation or transfection of a recipient with a nucleic acid of the invention can be carried out using conventional techniques well known to those skilled in the art. For example, where the host cell is E. coli, competent cells that are capable of DNA uptake can be prepared using the CaCl₂, MgCl₂ or RbCl methods known in the art. Alternatively, physical means, such as electroporation or microinjection can be used. Electroporation allows transfer of a nucleic acid into a cell by high voltage electric impulse. Additionally, nucleic acids can be introduced into host cells by protoplast fusion, using methods well known in the art. Suitable methods for transforming eukaryotic cells, such as electroporation and lipofection, also are known.

“Host cells” or “Recipient cells” encompassed by of the invention are any cells in which the nucleic acids of the invention can be used to express the polypeptides of the invention. The term also includes any progeny of a recipient or host cell. Preferred recipient or host cells of the invention include E. coli, S. aureus and P. aeruginosa, although other Gram-negative and Gram-positive bacterial, fungal and mammalian cells and organisms known in the art can be utilized as long as the expression vectors contain an origin of replication to permit expression in the host.

The cationic peptide polynucleotide sequence used according to the method of the invention can be isolated from an organism or synthesized in the laboratory. Specific DNA sequences encoding the cationic peptide of interest can be obtained by: 1) isolation of a double-stranded DNA sequence from the genomic DNA; 2) chemical manufacture of a DNA sequence to provide the necessary codons for the cationic peptide of interest; and 3) in vitro synthesis of a double-stranded DNA sequence by reverse transcription of mRNA isolated from a donor cell. In the latter case, a double-stranded DNA complement of mRNA is eventually formed which is generally referred to as cDNA.

The synthesis of DNA sequences is frequently the method of choice when the entire sequence of amino acid residues of the desired peptide product is known. In the present invention, the synthesis of a DNA sequence has the advantage of allowing the incorporation of codons which are more likely to be recognized by a bacterial host, thereby permitting high level expression without difficulties in translation. In addition, virtually any peptide can be synthesized, including those encoding natural cationic peptides, variants of the same, or synthetic peptides.

When the entire sequence of the desired peptide is not known, the direct synthesis of DNA sequences is not possible and the method of choice is the formation of cDNA sequences. Among the standard procedures for isolating cDNA sequences of interest is the formation of plasmid or phage containing cDNA libraries which are derived from reverse transcription of mRNA which is abundant in donor cells that have a high level of genetic expression. When used in combination with polymerase chain reaction technology, even rare expression products can be cloned. In those cases where significant portions of the amino acid sequence of the cationic peptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single stranded form (Jay, et al., Nuc. Acid Res., 11:2325, 1983).

The peptide of the invention can be administered parenterally by injection or by gradual infusion over time. Preferably the peptide is administered in a therapeutically effective amount to enhance or to stimulate an innate immune response. Innate immunity has been described herein, however examples of indicators of stimulation of innate immunity include but are not limited to monocyte activation, proliferation, differentiation or MAP kinase pathway activation.

The peptide can be administered intravenously, intraperitoneally, intramuscularly, subcutaneously, intracavity, or transdermally. Preferred methods for delivery of the peptide include orally, by encapsulation in microspheres or proteinoids, by aerosol delivery to the lungs, or transdermally by iontophoresis or transdermal electroporation. Other methods of administration will be known to those skilled in the art.

Preparations for parenteral administration of a peptide of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

In one embodiment, the invention provides a method for synergistic therapy. For example, peptides as described herein can be used in synergistic combination with sub-inhibitory concentrations of antibiotics. Examples of particular classes of antibiotics useful for synergistic therapy with the peptides of the invention include aminoglycosides (e.g., tobramycin), penicillins (e.g., piperacillin), cephalosporins (e.g., ceftazidime), fluoroquinolones (e.g., ciprofloxacin), carbapenems (e.g., imipenem), tetracyclines and macrolides (e.g., erythromycin and clarithromycin). Further to the antibiotics listed above, typical antibiotics include aminoglycosides (amikacin, gentamicin, kanamycin, netilmicin, tobramycin, s-treptomycin, azithromycin, clarithromycin, erythromycin, erythromycin estolate/ethyl-succinate/gluceptate/lactobionate/stearate), beta-lactams such as penicillins (e.g., penicillin G, penicillin V, methicillin, nafcillin, oxacillin, cloxacillin, dicloxacillin, ampicillin, amoxicillin, ticarcillin, carbenicillin, mezlocillin, azlocillin and piperacillin), or cephalosporins (e.g., cephalothin, cefazolin, cefaclor, cefamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, and cefsulodin). Other classes of antibiotics include carbapenems (e.g., imipenem), monobactams (e.g., aztreonam), quinolones (e.g., fleroxacin, nalidixic acid, norfloxacin, ciprofloxacin, ofloxacin, enoxacin, lomefloxacin and cinoxacin), tetracyclines (e.g., doxycycline, minocycline, tetracycline), and glycopeptides (e.g., vancomycin, teicoplanin), for example. Other antibiotics include chloramphenicol, clindamycin, trimethoprim, sulfamethoxazole, nitrofurantoin, rifampin, mupirocin and the cationic peptides.

The efficacy of peptides was evaluated therapeutically alone and in combination with sub-optimal concentrations of antibiotics in models of infection. S. aureus is an important Gram positive pathogen and a leading cause of antibiotic resistant infections. Briefly, peptides were tested for therapeutic efficacy in the S. aureus infection model by injecting them alone and in combination with sub-optimal doses of antibiotics 6 hours after the onset of infection. This would simulate the circumstances of antibiotic resistance developing during an infection, such that the MIC of the resistant bacterium was too high to permit successful therapy (i.e the antibiotic dose applied was sub-optimal). It was demonstrated that the combination of antibiotic and peptide resulted in improved efficacy and suggests the potential for combination therapy (see Example 12).

The invention will now be described in greater detail by reference to the following non-limiting examples. While the invention has been described in detail with reference to certain preferred embodiments thereof, it will be understood that modifications and variations are within the spirit and scope of that which is described and claimed.

### EXAMPLE 1

### Anti-Sepsis/Anti-Inflammatory Activity

Polynucleotide arrays were utilized to determine the effect of cationic peptides on the transcriptional response of epithelial cells. The A549 human epithelial cell line was maintained in DMEM (Gibco) supplemented with 10% fetal bovine serum (FBS, Medicorp). The A549 cells were plated in 100 mm tissue culture dishes at 2.5×10⁶ cells/dish, cultured overnight and then incubated with 100 ng/ml E. coli O111:B4 LPS (Sigrna), without (control) or with 50 μg/ml peptide or medium alone for 4 h. After stimulation, the cells were washed once with diethyl pyrocarbonate-treated phosphate buffered saline (PBS), and detached from the dish using a cell scraper. Total RNA was isolated using RNAqueous (Ambion, Austin, TX). The RNA pellet was resuspended in RNase-free water containing Superase-In (RNase inhibitor; Ambion). DNA contamination was removed with DNA-free kit, Ambion). The quality of the RNA was assessed by gel electrophoresis on a 1% agarose gel.

The polynucleotide arrays used were the Human Operon arrays (identification number for the genome is PRHU04-S1), which consist of about 14,000 human oligos spotted in duplicate. Probes were prepared from 10 μg of total RNA and labeled with Cy3 or Cy5 labeled dUTP. The probes were purified and hybridized to printed glass slides overnight at 42° C. and washed. After washing, the image was captured using a Perkin Elmer array scanner. The image processing software (Imapolynucleotide 5.0, Marina Del Rey, Calif.) determines the spot mean intensity, median intensities, and background intensities. A “homemade” program was used to remove background. The program calculates the bottom 10% intensity for each subgrid and subtracts this for each grid. Analysis was performed with Genespring software (Redwood City, Calif.). The intensities for each spot were normalized by taking the median spot intensity value from the population of spot values within a slide and comparing this value to the values of all slides in the experiment. The relative changes seen with cells treated with peptide compared to control cells can be found in Tables 1 and 2. These tables 2 reflect only those polynucleotides that demonstrated significant changes in expression of the 14,000 polynucleotides that were tested for altered expression. The data indicate that the peptides have a widespread ability to reduce the expression of polynucleotides that were induced by LPS.

In Table 1, the peptide, SEQ ID NO: 27 is shown to potently reduce the expression of many of the polynucleotides up-regulated by E. coli O111:B4 LPS as studied by polynucleotide microarrays. Peptide (50 μg/ml) and LPS (0.1 μg/ml) or LPS alone was incubated with the A549 cells for 4 h and the RNA was isolated. Five μg total RNA was used to make Cy3/Cy5 labeled cDNA probes and hybridized onto Human Operon arrays (PRHU04). The intensity of unstimulated cells is shown in the third column of Table 1. The “Ratio: LPS/control” column refers to the intensity of polynucleotide expression in LPS simulated cells divided by in the intensity of unstimulated cells. The “Ratio: LPS+ID 27/control” column refers to the intensity of polynucleotide expression in cells stimulated with LPS and peptide divided by unstimulated cells.

TABLE 1
Reduction, by peptide SEQ ID 27, of A549 human epithelial cell polynucleotide
expression up-regulated by E. coli O111:B4 LPS

| | | Control: | | |
|---|---|---|---|---|
| Accession | Polynucleotide | Media only | Ratio: | Ratio: LPS + ID |
| Number^{a} | Gene Function | Intensity | LPS/control | 27/control |
| AL031983 | Unknown | 0.032 | 302.8 | 5.1 |
| L04510 | ADP- | 0.655 | 213.6 | 1.4 |
| | ribosylation | | | |
| | factor | | | |
| D87451 | ring finger | 3.896 | 183.7 | 2.1 |
| | protein 10 | | | |
| AK000869 | hypothetical | 0.138 | 120.1 | 2.3 |
| | protein | | | |
| U78166 | Ric-like | 0.051 | 91.7 | 0.2 |
| | expressed in | | | |
| | neurons | | | |
| AJ001403 | mucin 5 subtype B | 0.203 | 53.4 | 15.9 |
| | tracheobronchial | | | |
| AB040057 | serine/threonine | 0.95 | 44.3 | 15.8 |
| | protein kinase | | | |
| | MASK | | | |
| Z99756 | Unknown | 0.141 | 35.9 | 14.0 |
| L42243 | interferon | 0.163 | 27.6 | 5.2 |
| | receptor 2 | | | |
| NM_016216 | RNA lariat | 6.151 | 22.3 | 10.9 |
| | debranching | | | |
| | enzyme | | | |
| AK001589 | hypothetical | 0.646 | 19.2 | 1.3 |
| | protein | | | |
| AL137376 | Unknown | 1.881 | 17.3 | 0.6 |
| AB007856 | FEM-1-like | 2.627 | 15.7 | 0.6 |
| | death receptor | | | |
| | binding protein | | | |
| AB007854 | growth arrest- | 0.845 | 14.8 | 2.2 |
| | specific 7 | | | |
| AK000353 | cytosolic ovarian | 0.453 | 13.5 | 1.0 |
| | carcinoma | | | |
| | antigen 1 | | | |
| D14539 | myeloid/lymphoid | 2.033 | 11.6 | 3.1 |
| | or mixed- | | | |
| | lineage leukemia | | | |
| | translocated to 1 | | | |
| X76785 | integration site | 0.728 | 11.6 | 1.9 |
| | for Epstein-Barr | | | |
| | virus | | | |
| M54915 | pim-1 oncogene | 1.404 | 11.4 | 0.6 |
| NM_006092 | caspase | 0.369 | 11.0 | 0.5 |
| | recruitment | | | |
| | domain 4 | | | |
| J03925 | integrin_alpha M | 0.272 | 9.9 | 4.2 |
| NM_001663 | ADP- | 0.439 | 9.7 | 1.7 |
| | ribosylation | | | |
| | factor 6 | | | |
| M23379 | RAS p21 protein | 0.567 | 9.3 | 2.8 |
| | activator | | | |
| K02581 | thymidine kinase | 3.099 | 8.6 | 3.5 |
| | 1 soluble | | | |
| U94831 | transmembrane 9 | 3.265 | 7.1 | 1.5 |
| | superfamily | | | |
| | member 1 | | | |
| X70394 | zinc finger | 1.463 | 6.9 | 1.7 |
| | protein 146 | | | |
| AL137614 | hypothetical | 0.705 | 6.8 | 1.0 |
| | protein | | | |
| U43083 | guanine | 0.841 | 6.6 | 1.6 |
| | nucleotide | | | |
| | binding protein | | | |
| AL137648 | DKFZp434J1813 | 1.276 | 6.5 | 0.8 |
| | protein | | | |
| AF085692 | ATP-binding | 3.175 | 6.5 | 2.4 |
| | cassette sub- | | | |
| | family C | | | |
| | (CFTR/MRP) | | | |
| | member 3 | | | |
| AK001239 | hypothetical | 2.204 | 6.4 | 1.3 |
| | protein | | | |
| | FLJ10377 | | | |
| NM_001679 | ATPase Na+/K+ | 2.402 | 6.3 | 0.9 |
| | transporting beta | | | |
| | 3 polypeptide | | | |
| L24804 | unactive | 3.403 | 6.1 | 1.1 |
| | progesterone | | | |
| | receptor | | | |
| U15932 | dual specificity | 0.854 | 6.1 | 2.1 |
| | phosphatase 5 | | | |
| M36067 | ligase I DNA_ATP- | 1.354 | 6.1 | 2.2 |
| | dependent | | | |
| AL161951 | Unknown | 0.728 | 5.8 | 1.9 |
| M59820 | colony | 0.38 | 5.7 | 2.0 |
| | stimulating | | | |
| | factor 3 receptor | | | |
| AL050290 | spermidine/ | 2.724 | 5.6 | 1.4 |
| | spermine N1- | | | |
| | acetyltransferase | | | |
| NM_002291 | laminin_beta 1 | 1.278 | 5.6 | 1.8 |
| X06614 | retinoic acid | 1.924 | 5.5 | 0.8 |
| | receptor_alpha | | | |
| AB007896 | putative L-type | 0.94 | 5.3 | 1.8 |
| | neutral amino | | | |
| | acid transporter | | | |
| AL050333 | DKFZP564B116 | 1.272 | 5.3 | 0.6 |
| | protein | | | |
| AK001093 | hypothetical | 1.729 | 5.3 | 2.0 |
| | protein | | | |
| NM_016406 | hypothetical | 1.314 | 5.2 | 1.2 |
| | protein | | | |
| M86546 | pre-B-cell | 1.113 | 5.2 | 2.2 |
| | leukemia transcription | | | |
| | factor 1 | | | |
| X56777 | zona pellucida | 1.414 | 5.0 | 1.4 |
| | glycoprotein 3A | | | |
| NM_013400 | replication | 1.241 | 4.9 | 2.0 |
| | initiation region | | | |
| | protein | | | |
| NM_002309 | leukemia | 1.286 | 4.8 | 1.9 |
| | inhibitory factor | | | |
| NM_001940 | dentatorubral- | 2.034 | 4.7 | 1.2 |
| | pallidoluysian | | | |
| | atrophy | | | |
| U91316 | cytosolic acyl | 2.043 | 4.7 | 1.4 |
| | coenzyme A | | | |
| | thioester | | | |
| | hydrolase | | | |
| X76104 | death-associated | 1.118 | 4.6 | 1.8 |
| | protein kinase 1 | | | |
| AF131838 | Unknown | 1.879 | 4.6 | 1.4 |
| AL050348 | Unknown | 8.502 | 4.4 | 1.7 |
| D42085 | KIAA0095 gene | 1.323 | 4.4 | 1.2 |
| | product | | | |
| X92896 | Unknown | 1.675 | 4.3 | 1.5 |
| U26648 | syntaxin 5A | 1.59 | 4.3 | 1.4 |
| X85750 | monocyte to | 1.01 | 4.3 | 1.1 |
| | macrophage | | | |
| | differentiation- | | | |
| | associated | | | |
| D14043 | CD164 antigen_sialomucin | 1.683 | 4.2 | 1.0 |
| J04513 | fibroblast growth | 1.281 | 4.0 | 0.9 |
| | factor 2 | | | |
| U19796 | melanoma- | 1.618 | 4.0 | 0.6 |
| | associated | | | |
| | antigen | | | |
| AK000087 | hypothetical | 1.459 | 3.9 | 1.0 |
| | protein | | | |
| AK001569 | hypothetical | 1.508 | 3.9 | 1.2 |
| | protein | | | |
| AF189009 | ubiquilin 2 | 1.448 | 3.8 | 1.3 |
| U60205 | sterol-C4-methyl | 1.569 | 3.7 | 0.8 |
| | oxidase-like | | | |
| AK000562 | hypothetical | 1.166 | 3.7 | 0.6 |
| | protein | | | |
| AL096739 | Unknown | 3.66 | 3.7 | 0.5 |
| AK000366 | hypothetical | 15.192 | 3.5 | 1.0 |
| | protein | | | |
| NM_006325 | RAN member | 1.242 | 3.5 | 1.4 |
| | RAS oncogene | | | |
| | family | | | |
| X51688 | cyclin A2 | 1.772 | 3.3 | 1.0 |
| U34252 | aldehyde | 1.264 | 3.3 | 1.2 |
| | dehydrogenase 9 | | | |
| NM_013241 | FH1/FH2 | 1.264 | 3.3 | 0.6 |
| | domain- | | | |
| | containing | | | |
| | protein | | | |
| AF112219 | esterase | 1.839 | 3.3 | 1.1 |
| | D/formylglutathione | | | |
| | hydrolase | | | |
| NM_016237 | anaphase- | 2.71 | 3.2 | 0.9 |
| | promoting | | | |
| | complex subunit 5 | | | |
| AB014569 | KIAA0669 gene | 2.762 | 3.2 | 0.2 |
| | product | | | |
| AF151047 | hypothetical | 3.062 | 3.1 | 1.0 |
| | protein | | | |
| X92972 | protein | 2.615 | 3.1 | 1.1 |
| | phosphatase 6 | | | |
| | catalytic subunit | | | |
| AF035309 | proteasome 26S | 5.628 | 3.1 | 1.3 |
| | subunit ATPase 5 | | | |
| U52960 | SRB7 homolog | 1.391 | 3.1 | 0.8 |
| J04058 | electron-transfer- | 3.265 | 3.1 | 1.2 |
| | flavoprotein | | | |
| | alpha | | | |
| | polypeptide | | | |
| M57230 | interleukin 6 | 0.793 | 3.1 | 1.0 |
| | signal transducer | | | |
| U78027 | galactosidase_alpha | 3.519 | 3.1 | 1.1 |
| AK000264 | Unknown | 2.533 | 3.0 | 0.6 |
| X80692 | mitogen- | 2.463 | 2.9 | 1.3 |
| | activated protein | | | |
| | kinase 6 | | | |
| L25931 | lamin B receptor | 2.186 | 2.7 | 0.7 |
| X13334 | CD14 antigen | 0.393 | 2.5 | 1.1 |
| M32315 | tumor necrosis | 0.639 | 2.4 | 0.4 |
| | factor receptor | | | |
| | superfamily | | | |
| | member 1B | | | |
| NM_004862 | LPS-induced | 6.077 | 2.3 | 1.1 |
| | TNF-alpha factor | | | |
| AL050337 | interferon | 2.064 | 2.1 | 1.0 |
| | gamma receptor 1 | | | |

^{a}All Accession Numbers in Table 1 through Table 64 refer to GenBank Accession Numbers.

In Table 2, the cationic peptides at a concentration of 50 μg/ml were shown to potently reduce the expression of many of the polynucleotides up-regulated by 100 ng/ml E. coli O111:B4 LPS as studied by polynucleotide microarrays. Peptide and LPS or LPS alone was incubated with the A549 cells for 4 h and the RNA was isolated. 5 μg total RNA was used to make Cy3/Cy5 labeled cDNA probes and hybridized onto Human Operon arrays (PRHU04). The intensity of unstimulated cells is shown in the third column of Table 2. The “Ratio: LPS/control” column refers to the intensity of polynucleotide expression in LPS-simulated cells divided by in the intensity of unstimulated cells. The other columns refer to the intensity of polynucleotide expression in cells stimulated with LPS and peptide divided by unstimulated cells.

TABLE 2
Human A549 Epithelial Cell Polynucleotide Expression up-regulated
by E. coli O111:B4 LPS and reduced by Cationic Peptides.

| | | Ctrl: | | Ratio: | Ratio: | Ratio: |
|---|---|---|---|---|---|---|
| Accession | | Media only | Ratio: | LPS + ID | LPS + ID | LPS + ID |
| Number | Gene | Intensity | LPS/Ctrl | 27/Ctrl | 16/Ctrl | 22/Ctrl |
| AL031983 | Unknown | 0.03 | 302.8 | 5.06 | 6.91 | 0.31 |
| L04510 | ADP- | 0.66 | 213.6 | 1.4 | 2.44 | 3.79 |
| | ribosylation | | | | | |
| | factor | | | | | |
| D87451 | ring finger | 3.90 | 183.7 | 2.1 | 3.68 | 4.28 |
| | protein | | | | | |
| AK000869 | hypothetical | 0.14 | 120.1 | 2.34 | 2.57 | 2.58 |
| | protein | | | | | |
| U78166 | Ric like | 0.05 | 91.7 | 0.20 | 16.88 | 21.37 |
| X03066 | MHC class II | 0.06 | 36.5 | 4.90 | 12.13 | 0.98 |
| | DO beta | | | | | |
| AK001904 | hypothetical | 0.03 | 32.8 | 5.93 | 0.37 | 0.37 |
| | protein | | | | | |
| AB037722 | Unknown | 0.03 | 21.4 | 0.30 | 0.30 | 2.36 |
| AK001589 | hypothetical | 0.65 | 19.2 | 1.26 | 0.02 | 0.43 |
| | protein | | | | | |
| AL137376 | Unknown | 1.88 | 17.3 | 0.64 | 1.30 | 1.35 |
| L19185 | thioredoxin- | 0.06 | 16.3 | 0.18 | 2.15 | 0.18 |
| | dependent peroxide | | | | | |
| | reductase 1 | | | | | |
| J05068 | transcobalamin I | 0.04 | 15.9 | 1.78 | 4.34 | 0.83 |
| AB007856 | FEM-1-like | 2.63 | 15.7 | 0.62 | 3.38 | 0.96 |
| | death receptor | | | | | |
| | binding protein | | | | | |
| AK000353 | cytosolic | 0.45 | 13.5 | 1.02 | 1.73 | 2.33 |
| | ovarian | | | | | |
| | carcinoma ag 1 | | | | | |
| X16940 | smooth muscle | 0.21 | 11.8 | 3.24 | 0.05 | 2.26 |
| | enteric actin γ2 | | | | | |
| M54915 | pim-1 oncogene | 1.40 | 11.4 | 0.63 | 1.25 | 1.83 |
| AL122111 | hypothetical | 0.37 | 10.9 | 0.21 | 1.35 | 0.03 |
| | protein | | | | | |
| M95678 | phospholipase | 0.22 | 7.2 | 2.38 | 0.05 | 1.33 |
| | C beta 2 | | | | | |
| AK001239 | hypothetical | 2.20 | 6.4 | 1.27 | 1.89 | 2.25 |
| | protein | | | | | |
| AC004849 | Unknown | 0.14 | 6.3 | 0.07 | 2.70 | 0.07 |
| X06614 | retinoic acid | 1.92 | 5.5 | 0.77 | 1.43 | 1.03 |
| | receptor_alpha | | | | | |
| AB007896 | putative L-type | 0.94 | 5.3 | 1.82 | 2.15 | 2.41 |
| | neutral amino | | | | | |
| | acid transporter | | | | | |
| AB010894 | BAI1- | 0.69 | 5.0 | 1.38 | 1.03 | 1.80 |
| | associated | | | | | |
| | protein | | | | | |
| U52522 | partner of | 1.98 | 2.9 | 1.35 | 0.48 | 1.38 |
| | RAC1 | | | | | |
| AK001440 | hypothetical | 1.02 | 2.7 | 0.43 | 1.20 | 0.01 |
| | protein | | | | | |
| NM_001148 | ankyrin 2_neuronal | 0.26 | 2.5 | 0.82 | 0.04 | 0.66 |
| X07173 | inter-alpha | 0.33 | 2.2 | 0.44 | 0.03 | 0.51 |
| | inhibitor H2 | | | | | |
| AF095687 | brain and | 0.39 | 2.1 | 0.48 | 0.03 | 0.98 |
| | nasopharyngeal | | | | | |
| | carcinoma | | | | | |
| | susceptibility | | | | | |
| | protein | | | | | |
| NM_016382 | NK cell | 0.27 | 2.1 | 0.81 | 0.59 | 0.04 |
| | activation | | | | | |
| | inducing ligand | | | | | |
| | NAIL | | | | | |
| AB023198 | KIAA0981 | 0.39 | 2.0 | 0.43 | 0.81 | 0.92 |
| | protein | | | | | |

### EXAMPLE 2

### Neutralization of the Stimulation of Immune Cells

The ability of compounds to neutralize the stimulation of immune cells by both Gram-negative and Gram-positive bacterial products was tested. Bacterial products stimulate cells of the immune system to produce inflammatory cytokines and when unchecked this can lead to sepsis. Initial experiments utilized the murine macrophage cell line RAW 264.7, which was obtained from the American Type Culture Collection, (Manassas, Va.), the human epithelial cell line, A549, and primary macrophages derived from the bone marrow of BALB/c mice (Charles River Laboratories, Wilmington, Mass.). The cells from mouse bone marrow were cultured in 150-mm plates in Dulbecco's modified Eagle medium (DMEM; Life Technologies, Burlington, ON) supplemented with 20% FBS (Sigma Chemical Co,St. Louis, Mo.) and 20% L cell-conditioned medium as a source of M-CSF. Once macrophages were 60-80% confluent, they were deprived of L cell-conditioned medium for 14-16 h to render the cells quiescent and then were subjected to treatments with 100 ng/ml LPS or 100 ng/ml LPS+20 μg/ml peptide for 24 hours. The release of cytokines into the culture supernatant was determined by ELISA (R&D Systems, Minneapolis, Minn.). The cell lines, RAW 264.7 and A549, were maintained in DMEM supplemented with 10% fetal calf serum. RAW 264.7 cells were seeded in 24 well plates at a density of 10⁶ cells per well in DMEM and A549 cells were seeded in 24 well plates at a density of 10⁵ cells per well in DMEM and both were incubated at 37° C. in 5% CO₂ overnight. DMEM was aspirated from cells grown overnight and replaced with fresh medium. In some experiments, blood from volunteer human donors was collected (according to procedures accepted by UBC Clinical Research Ethics Board, certificate C00-0537) by venipuncture into tubes (Becton Dickinson, Franklin Lakes, N.J.) containing 14.3 USP units heparin/ml blood. The blood was mixed with LPS with or without peptide in polypropylene tubes at 37° C. for 6 h. The samples were centrifuged for 5 min at 2000×g, the plasma was collected and then stored at −20° C. until being analyzed for IL-8 by ELISA (R&D Systems). In the experiments with cells, LPS or other bacterial products were incubated with the cells for 6-24 hr at 37° C. in 5% C0₂. S. typhimurium LPS and E. coli 0111:B4 LPS were purchased from Sigma. Lipoteichoic acid (LTA) from S. aureus (Sigma) was resuspended in endotoxin free water (Sigma). The Limulus amoebocyte lysate assay (Sigma) was performed on LTA preparations to confirm that lots were not significantly contaminated by endotoxin. Endotoxin contamination was less than 1 ng/ml, a concentration that did not cause significant cytokine production in the RAW 264.7 cells. Non-capped lipoarabinomannan (AraLAM ) was a gift from Dr. John T. Belisle of Colorado State University. The AraLAM from Mycobacterium was filter sterilized and the endotoxin contamination was found to be 3.75 ng per 1.0 mg of LAM as determined by Limulus Amebocyte assay. At the same time as LPS addition (or later where specifically described), cationic peptides were added at a range of concentrations. The supernatants were removed and tested for cytokine production by ELISA (R&D Systems). All assays were performed at least three times with similar results. To confirm the anti-sepsis activity in vivo, sepsis was induced by intraperitoneal injection of 2 or 3 μg of E. coli O111:B4 LPS in phosphate-buffered saline (PBS; pH 7.2) into galactosamine-sensitized 8- to 10-week-old female CD-1 or BALB/c mice. In experiments involving peptides, 200 μg in 100 μl of sterile water was injected at separate intraperitoneal sites within 10 min of LPS injection. In other experiments, CD-1 mice were injected with 400 μg E. coli O111:B4 LPS and 10 min later peptide (200 μg) was introduced by intraperitoneal injection. Survival was monitored for 48 hours post injection.

Hyperproduction of TNF-α has been classically linked to development of sepsis. The three types of LPS, LTA or AraLAM used in this example represented products released by both Gram-negative and Gram-positive bacteria. Peptide, SEQ ID NO: 1, was able to significantly reduce TNF-α production stimulated by S. typhimurium, B. cepacia, and E. coli O111:B4 LPS, with the former being affected to a somewhat lesser extent (Table 3). At concentrations as low as 1 μg/ml of peptide (0.25 nM) substantial reduction of TNF-α production was observed in the latter two cases. A different peptide, SEQ ID NO: 3 did not reduce LPS-induced production of TNF-α in RAW macrophage cells, demonstrating that this is not a uniform and predictable property of cationic peptides. Representative peptides from each Formula were also tested for their ability to affect TNF-α production stimulated by E. coli O111:B4 LPS (Table 4). The peptides had a varied ability to reduce TNF-α production although many of them lowered TNF-α by at least 60%.

At certain concentrations peptides SEQ ID NO: 1 and SEQ ID NO: 2, could also reduce the ability of bacterial products to stimulate the production of IL-8 by an epithelial cell line. LPS is a known potent stimulus of IL-8 production by epithelial cells. Peptides, at low concentrations (1-20 μg/ml), neutralized the IL-8 induction responses of epithelial cells to LPS (Table 5-7). Peptide SEQ ID 2 also inhibited LPS-induced production of IL-8 in whole human blood (Table 4). Conversely, high concentrations of peptide SEQ ID NO: 1 (50 to 100 μg/ml) actually resulted in increased levels of IL-8 (Table 5). This suggests that the peptides have different effects at different concentrations.

The effect of peptides on inflammatory stimuli was also demonstrated in primary murine cells, in that peptide SEQ ID NO: 1 significantly reduced TNF-α production (>90%) by bone marrow-derived macrophages from BALB/c mice that had been stimulated with 100 ng/ml E. coli 0111:B4 LPS (Table 8). These experiments were performed in the presence of serum, which contains LPS-binding protein (LBP), a protein that can mediate the rapid binding of LPS to CD14. Delayed addition of SEQ ID NO: 1 to the supernatants of macrophages one hour after stimulation with 100 ng/ml E. coli LPS still resulted in substantial reduction (70%) of TNF-α production (Table 9).

Consistent with the ability of SEQ ID NO: 1 to prevent LPS-induced production of TNF-α in vitro, certain peptides also protected mice against lethal shock induced by high concentrations of LPS. In some experiments, CD-1 mice were sensitized to LPS with a prior injection of galactosamine. Galactosamine-sensitized mice that were injected with 3 μg of E. coli 0111:B4 LPS were all killed within 4-6 hours. When 200 μg of SEQ ID NO: 1 was injected 15 min after the LPS, 50% of the mice survived (Table 10). In other experiments when a higher concentration of LPS was injected into BALB/c mice with no D-galactosamine, peptide protected 100% compared to the control group in which there was no survival (Table 13). Selected other peptides were also found to be protective in these models (Tables 11,12).

Cationic peptides were also able to lower the stimulation of macrophages by Gram-positive bacterial products such as Mycobacterium non-capped lipoarabinomannan (AraLAM) and S. aureus LTA. For example, SEQ ID NO: 1 inhibited induction of TNF-α in RAW 264.7 cells by the Gram-positive bacterial products, LTA (Table 14) and to a lesser extent AraLAM (Table 15). Another peptide, SEQ ID NO: 2, was also found to reduce LTA-induced TNF-α production by RAW 264.7 cells. At a concentration of 1 μg/ml SEQ ID NO: 1 was able to substantially reduce (>75%) the induction of TNF-α production by 1 μg/ml S. aureus LTA. At 20 μ/ml SEQ ID NO: 1, there was >60% inhibition of AraLAM induced TNF-α. Polymyxin B (PMB) was included as a control to demonstrate that contaminating endotoxin was not a significant factor in the inhibition by SEQ ID NO: 1 of AraLAM induced TNF-α. These results demonstrate that cationic peptides can reduce the pro-inflammatory cytokine response of the immune system to bacterial products.

Background levels of TNF-α production by the RAW 264.7 cells cultured with no stimuli for 6 hours resulted in TNF-α levels ranging from 0.037-0.192 ng/ml. The data is from duplicate samples and presented as the mean of three experiments+standard error.

TABLE 3
Reduction by SEQ ID 1 of LPS induced TNF-α production
in RAW 264.7 cells.

| Amount of SEQ | Inhibition of TNF-α (%)* | | |
|---|---|---|---|
| ID NO: 1 (μg/ml) | B. cepacia LPS | E. coli LPS | S. typhimurium LPS |
| 0.1 | 8.5 ± 2.9 | 0.0 ± 0.6 | 0.0 ± 0 |
| 1 | 23.0 ± 11.4 | 36.6 ± 7.5 | 9.8 ± 6.6 |
| 5 | 55.4 ± 8 | 65.0 ± 3.6 | 31.1 ± 7.0 |
| 10 | 63.1 ± 8 | 75.0 ± 3.4 | 37.4 ± 7.5 |
| 20 | 71.7 ± 5.8 | 81.0 ± 3.5 | 58.5 ± 10.5 |
| 50 | 86.7 ± 4.3 | 92.6 ± 2.5 | 73.1 ± 9.1 |
| RAW 264.7 mouse macrophage cells were stimulated with 100 ng/ml S. typhimurium LPS, 100 ng/ml B. cepacia LPS and 100 ng/ml E. coli 0111:B4 LPS in the presence of the indicated concentrations of SEQ ID 1 for 6 hr. The concentrations of TNF-α | | | |
| # released into the culture supernatants were determined by ELISA. 100% represents the amount of TNF-α resulting from RAW 264.7 cells incubated with LPS alone for 6 hours (S. typhimurium LPS = 34.5 ± 3.2 ng/ml, B. cepacia LPS = 11.6 ± 2.9 ng/ml, and E.coli 0111:B4 | | | |
| # LPS = 30.8 ± 2.4 ng/ml). Background levels of TNF-α production by the RAW 264.7 cells cultured with no stimuli for 6 hours resulted in TNF-α levels ranging from 0.037-0.192 ng/ml. The data is from duplicate samples and presented as the mean of three experiments + standard error. | | | |

TABLE 4
Reduction by Cationic Peptides of E. coli LPS induced
TNF-α production in RAW 264.7 cells.

| Peptide (20 μg/ml) | Inhibition of TNF-α (%) |
|---|---|
| SEQ ID NO: 5 | 65.6 ± 1.6 |
| SEQ ID NO: 6 | 59.8 ± 1.2 |
| SEQ ID NO: 7 | 50.6 ± 0.6 |
| SEQ ID NO: 8 | 39.3 ± 1.9 |
| SEQ ID NO: 9 | 58.7 ± 0.8 |
| SEQ ID NO: 10 | 55.5 ± 0.52 |
| SEQ ID NO: 12 | 52.1 ± 0.38 |
| SEQ ID NO: 13 | 62.4 ± 0.85 |
| SEQ ID NO: 14 | 50.8 ± 1.67 |
| SEQ ID NO: 15 | 69.4 ± 0.84 |
| SEQ ID NO: 16 | 37.5 ± 0.66 |
| SEQ ID NO: 17 | 28.3 ± 3.71 |
| SEQ ID NO: 19 | 69.9 ± 0.09 |
| SEQ ID NO: 20 | 66.1 ± 0.78 |
| SEQ ID NO: 21 | 67.8 ± 0.6 |
| SEQ ID NO: 22 | 73.3 ± 0.36 |
| SEQ ID NO: 23 | 83.6 ± 0.32 |
| SEQ ID NO: 24 | 60.5 ± 0.17 |
| SEQ ID NO: 26 | 54.9 ± 1.6 |
| SEQ ID NO: 27 | 51.1 ± 2.8 |
| SEQ ID NO: 28 | 56 ± 1.1 |
| SEQ ID NO: 29 | 58.9 ± 0.005 |
| SEQ ID NO: 31 | 60.3 ± 0.6 |
| SEQ ID NO: 33 | 62.1 ± 0.08 |
| SEQ ID NO: 34 | 53.3 ± 0.9 |
| SEQ ID NO: 35 | 60.7 ± 0.76 |
| SEQ ID NO: 36 | 63 ± 0.24 |
| SEQ ID NO: 37 | 58.9 ± 0.67 |
| SEQ ID NO: 38 | 54 ± 1 |
| SEQ ID NO: 40 | 75 ± 0.45 |
| SEQ ID NO: 41 | 86 ± 0.37 |
| SEQ ID NO: 42 | 80.5 ± 0.76 |
| SEQ ID NO: 43 | 88.2 ± 0.65 |
| SEQ ID NO: 44 | 44.9 ± 1.5 |
| SEQ ID NO: 45 | 44.7 ± 0.39 |
| SEQ ID NO: 47 | 36.9 ± 2.2 |
| SEQ ID NO: 48 | 64 ± 0.67 |
| SEQ ID NO: 49 | 86.9 ± 0.69 |
| SEQ ID NO: 53 | 46.5 ± 1.3 |
| SEQ ID NO: 54 | 64 ± 0.73 |
| RAW 264.7 mouse macrophage cells were stimulated with 100 ng/ml E. coli 0111:B4 LPS in the presence of the indicated concentrations of cationic peptides for 6 h. The concentrations of TNF-α released into the culture supernatants were determined by ELISA. | |
| # Background levels of TNF-α production by the RAW 264.7 cells cultured with no stimuli for 6 hours resulted in TNF-α levels ranging from 0.037-0.192 ng/ml. The data is from duplicate samples | |
| # and presented as the mean of three experiments + standard deviation. | |

TABLE 5
Reduction by SEQ ID NO: 1 of LPS induced IL-8 production in A549
cells.

| SEQ ID NO: 1 (μg/ml) | Inhibition of IL-8 (%) |
|---|---|
| 0.1 | 1 ± 0.3 |
| 1 | 32 ± 10 |
| 10 | 60 ± 9 |
| 20 | 47 ± 12 |
| 50 | 40 ± 13 |
| 100 | 0 |
| A549 cells were stimulated with increasing concentrations of SEQ ID 1 in the presence of LPS (100 ng/ml E. coli 0111 :B4) for 24 hours. The concentration of IL-8 in the culture supernatants was determined by ELISA. The background levels of IL-8 from cells alone was | |
| # 0.172 ± 0.029 ng/ml. The data is presented as the mean of three experiments + standard error. | |

TABLE 6
Reduction by SEQ ID NO: 2 of E. coli LPS induced IL-8 production in
A549 cells.

| Concentration of SEQ ID NO: 2 (μg/ml) | Inhibition of IL-8 (%) |
|---|---|
| 0.1 | 6.8 ± 9.6 |
| 1 | 12.8 ± 24.5 |
| 10 | 29.0 ± 26.0 |
| 50 | 39.8 ± 1.6 |
| 100 | 45.0 ± 3.5 |
| Human A549 epithelial cells were stimulated with increasing concentrations of SEQ ID NO: 2 in the presence of LPS (100 ng/ml E. coli 0111:B4) for 24 hours. The concentration of IL-8 in the culture supernatants was determined by ELISA. The data is | |
| # presented as the mean of three experiments + standard error. | |

TABLE 7
Reduction by SEQ ID NO: 2 of E. coli LPS induced IL-8 in human blood.

| SEQ ID NO: 2 (μg/ml) | IL-8 (pg/ml) |
|---|---|
| 0 | 3205 |
| 10 | 1912 |
| 50 | 1458 |

Whole human blood was stimulated with increasing concentrations of peptide and E.coli 0111:B4 LPS for 4 hr. The human blood samples were centrifuged and the serum was removed and tested for IL-8 by ELISA. The data is presented as the average of 2 donors.

TABLE 8
Reduction by SEQ ID NO: 1 of E. coli LPS induced TNF-α production
in murine bone marrow macrophages.

Production of
TNF-α (ng/ml)

| SEQ ID NO: 1 (μg/ml) | 6 hours | 24 hours |
|---|---|---|
| LPS alone | 1.1 | 1.7 |
| 1 | 0.02 | 0.048 |
| 10 | 0.036 | 0.08 |
| 100 | 0.033 | 0.044 |
| No LPS control | 0.038 | 0.06 |
| BALB/c Mouse bone marrow-derived macrophages were cultured for either 6 h or 24 h with 100 ng/ml E. coli 0111:B4 LPS in the presence or absence of 20 μg/ml of peptide. The supernatant was collected and tested for levels of TNF-α by ELISA. The data represents the amount of TNF-α resulting from duplicate wells of bone marrow-derived macrophages incubated with LPS alone for 6 h (1.1 ± 0.09 ng/ml) or 24 h (1.7 ± 0.2 ng/ml). | | |
| # Background levels of TNF-α were 0.038 ± 0.008 ng/ml for 6 h and 0.06 ± 0.012 ng/ml for 24 h. | | |

TABLE 9
Inhibition of E. coli LPS-induced TNF-α production by delayed
addition of SEQ ID NO: 1 to A549 cells.

| Time of addition of SEQ ID NO: 1 | |
|---|---|
| after LPS (min) | Inhibition of TNF-α (%) |
| 0 | 98.3 ± 0.3 |
| 15 | 89.3 ± 3.8 |
| 30 | 83 ± 4.6 |
| 60 | 68 ± 8 |
| 90 | 53 ± 8 |
| Peptide (20 μg/ml) was added at increasing time points to wells already containing A549 human epithelial cells and 100 ng/ml E. coli 0111:B4 LPS. | |
| # The supernatant was collected after 6 hours and tested for levels of TNF-α by ELISA. The data is presented as the mean of three experiments + standard error. | |

TABLE 10
Protection against lethal endotoxemia in galactosamine-sensitized
CD-1 mice by SEQ ID NO: 1.

| | | | | Survival |
|---|---|---|---|---|
| D- | | | | post |
| Galactosamine | E. coli | Peptide or | | endotoxin |
| treatment | 0111:B4 LPS | buffer | Total mice | shock |
| 0 | 3 μg | PBS | 5 | 5 (100%) |
| 20 mg | 3 μg | PBS | 12 | 0 (0%) |
| 20 mg | 3 μg | SEQ ID NO: 1 | 12 | 6 (50%) |
| CD-1 mice (9 weeks-old) were sensitized to endotoxin by three intraperitoneal injections of galactosamine (20 mg in 0.1 ml sterile PBS). Then endotoxic shock was induced by intraperitoneal injection of E. coli 0111:B4 LPS (3 μg in 0.1 ml PBS). Peptide, | | | | |
| # SEQ ID NO: 1, (200 μg/mouse = 8 mg/kg) was injected at a separate intraperitoneal site 15 min after injection of LPS. The mice were monitored for 48 hours and the results were recorded. | | | | |

TABLE 11
Protection against lethal endotoxemia in galactosamine-
sensitized CD-1 mice by Cationic Peptides.

| | E. coli 0111:B4 | Number | |
|---|---|---|---|
| Peptide Treatment | LPS added | of Mice | Survival (%) |
| Control (no peptide) | 2 μg | 5 | 0 |
| SEQ ID NO: 6 | 2 μg | 5 | 40 |
| SEQ ID NO: 13 | 2 μg | 5 | 20 |
| SEQ ID NO: 17 | 2 μg | 5 | 40 |
| SEQ ID NO: 24 | 2 μg | 5 | 0 |
| SEQ ID NO: 27 | 2 μg | 5 | 20 |
| CD-1 mice (9 weeks-old) were sensitized to endotoxin by intraperitoneal injection of galactosamine (20 mg in 0.1 ml sterile PBS). Then endotoxic shock was induced by intraperitoneal injection of E. coli 0111:B4 LPS (2 μg in 0.1 ml PBS). Peptide | | | |
| # (200 μg/mouse = 8mg/kg) was injected at a separate intraperitoneal site 15 min after injection of LPS. The mice were monitored for 48 hours and the results were recorded. | | | |

TABLE 12
Protection against lethal endotoxemia in galactosamine-sensitized
BALB/c mice by Cationic Peptides.

| | E. coli | | |
|---|---|---|---|
| Peptide Treatment | 0111:B4 LPS added | Number of Mice | Survival (%) |
| No peptide | 2 μg | 10 | 10 |
| SEQ ID NO: 1 | 2 μg | 6 | 17 |
| SEQ ID NO: 3 | 2 μg | 6 | 0 |
| SEQ ID NO: 5 | 2 μg | 6 | 17 |
| SEQ ID NO: 6 | 2 μg | 6 | 17 |
| SEQ ID NO: 12 | 2 μg | 6 | 17 |
| SEQ ID NO: 13 | 2 μg | 6 | 33 |
| SEQ ID NO: 15 | 2 μg | 6 | 0 |
| SEQ ID NO: 16 | 2 μg | 6 | 0 |
| SEQ ID NO: 17 | 2 μg | 6 | 17 |
| SEQ ID NO: 23 | 2 μg | 6 | 0 |
| SEQ ID NO: 24 | 2 μg | 6 | 17 |
| SEQ ID NO: 26 | 2 μg | 6 | 0 |
| SEQ ID NO: 27 | 2 μg | 6 | 50 |
| SEQ ID NO: 29 | 2 μg | 6 | 0 |
| SEQ ID NO: 37 | 2 μg | 6 | 0 |
| SEQ ID NO: 38 | 2 μg | 6 | 0 |
| SEQ ID NO: 41 | 2 μg | 6 | 0 |
| SEQ ID NO: 44 | 2 μg | 6 | 0 |
| SEQ ID NO: 45 | 2 μg | 6 | 0 |

BALB/c mice (8 weeks-old) were sensitized to endotoxin by intraperitoneal injection of galactosamine (20 mg in 0.1 ml sterile PBS). Then endotoxic shock was induced by intraperitoneal injection of E. coli 0111:B4 LPS (2 μg in 0.1 ml PBS). Peptide (200 μg/mouse = 8 mg/kg) was injected at a separate intraperitoneal site 15 mm after injection of LPS. The mice were monitored for 48 hours and the results were recorded.

TABLE 13
Protection against lethal endotoxemia in BALB/c mice by SEQ ID
NO: 1.

| | E. coli | Number | |
|---|---|---|---|
| Peptide Treatment | 0111:B4 LPS | of Mice | Survival (%) |
| No peptide | 400 μg | 5 | 0 |
| SEQ ID NO: 1 | 400 μg | 5 | 100 |

BALB/c mice were injected intraperitoneal with 400 μg E. coli 0111:B4 LPS. Peptide (200 μg/mouse = 8 mg/kg) was injected at a separate intraperitoneal site and the mice were monitored for 48 hours and the results were recorded.

TABLE 14
Peptide inhibition of TNF-cz production induced by S. aureus LTA.

| SEQ ID NO: 1 added (μg/ml) | Inhibition of TNF-α (%) |
|---|---|
| 0.1 | 44.5 ± 12.5 |
| 1 | 76.7 ± 6.4 |
| 5 | 91 ± 1 |
| 10 | 94.5 ± 1.5 |
| 20 | 96 ± 1 |

RAW 264.7 mouse macrophage cells were stimulated with 1 μg/ml S. aureus LTA in the absence and presence of increasing concentrations of peptide. The supernatant was collected and tested for levels of TNF-α by ELISA. Background levels of TNF-α production by the RAW 264.7 cells cultured with no stimuli for 6 hours resulted in TNF-α levels ranging from 0.037-0.192 ng/ml. The data is presented as the mean of three or more experiments + standard error.

TABLE 15
Peptide inhibition of TNF-α production induced by Mycobacterium
non-capped lipoarabinomannan.

| Peptide (20 μg/ml) | Inhibition of TNF-α (%) |
|---|---|
| No peptide | 0 |
| SEQ ID NO: 1 | 64 ± 5.9 |
| Polymyxin B | 15 ± 2 |

RAW 264.7 mouse macrophage cells were stimulated with 1 μg/ml AraLAM in the absence and presence of 20 μg/ml peptide or Polymyxin B. The supernatant was collected and tested for levels of TNF-α by ELISA. Background levels of TNF-α production by the RAW 264.7 cells cultured with no stimuli for 6 hours resulted in TNF-α levels ranging from 0.037-0.192 ng/ml. The data is presented as the mean inhibition of three or more experiments + standard error.

### EXAMPLE 3

### Assessment of Toxicity of the Cationic Peptides

The potential toxicity of the peptides was measured in two ways. First, the Cytotoxicity Detection Kit (Roche) (Lactate dehydrogenase—LDH) Assay was used. It is a colorimetric assay for the quantification of cell death and cell lysis, based on the measurement of LDH activity released from the cytosol of damaged cells into the supernatant. LDH is a stable cytoplasmic enzyme present in all cells and it is released into the cell culture supernatant upon damage of the plasma membrane. An increase in the amount of dead or plasma membrane-damaged cells results in an increase of the LDH enzyme activity in the culture supernatant as measured with an ELISA plate reader, OD₄₉₀nm (the amount of color formed in the assay is proportional to the number of lysed cells). In this assay, human bronchial epithelial cells (16HBEo14, HBE) cells were incubated with 100 μg of peptide for 24 hours, the supernatant removed and tested for LDH. The other assay used to measure toxicity of the cationic peptides was the WST-1 assay (Roche). This assay is a colorimetric assay for the quantification of cell proliferation and cell viability, based on the cleavage of the tetrazolium salt WST-1 by mitochondrial dehydrogenases in viable cells (a non-radioactive alternative to the [³H]-thymidine incorporation assay). In this assay, HBE cells were incubated with 100 μg of peptide for 24 hours, and then 10 μl/well Cell Proliferation Reagent WST-1 was added. The cells are incubated with the reagent and the plate is then measured with an ELISA plate reader, OD₄₉₀nm.

The results shown below in Tables 16 and 17 demonstrate that most of the peptides are not toxic to the cells tested. However, four of the peptides from Formula F (SEQ ID NOS: 40, 41, 42 and 43) did induce membrane damage as measured by both assays.

TABLE 16
Toxicity of the Cationic Peptides as Measured by the LDH Release
Assay.

| Treatment | LDH Release (OD₄₉₀ nm) |
|---|---|
| No cells Control | 0.6 ± 0.1 |
| Triton X-100 Control | 4.6 ± 0.1 |
| No peptide control | 1.0 ± 0.05 |
| SEQ ID NO: 1 | 1.18 ± 0.05 |
| SEQ ID NO: 3 | 1.05 ± 0.04 |
| SEQ ID NO: 6 | 0.97 ± 0.02 |
| SEQ ID NO: 7 | 1.01 ± 0.04 |
| SEQ ID NO: 9 | 1.6 ± 0.03 |
| SEQ ID NO: 10 | 1.04 ± 0.04 |
| SEQ ID NO: 13 | 0.93 ± 0.06 |
| SEQ ID NO: 14 | 0.99 ± 0.05 |
| SEQ ID NO: 16 | 0.91 ± 0.04 |
| SEQ ID NO: 17 | 0.94 ± 0.04 |
| SEQ ID NO: 19 | 1.08 ± 0.02 |
| SEQ ID NO: 20 | 1.05 ± 0.03 |
| SEQ ID NO: 21 | 1.06 ± 0.04 |
| SEQ ID NO: 22 | 1.29 ± 0.12 |
| SEQ ID NO: 23 | 1.26 ± 0.46 |
| SEQ ID NO: 24 | 1.05 ± 0.01 |
| SEQ ID NO: 26 | 0.93 ± 0.04 |
| SEQ ID NO: 27 | 0.91 ± 0.04 |
| SEQ ID NO: 28 | 0.96 ± 0.06 |
| SEQ ID NO: 29 | 0.99 ± 0.02 |
| SEQ ID NO: 31 | 0.98 ± 0.03 |
| SEQ ID NO: 33 | 1.03 ± 0.05 |
| SEQ ID NO: 34 | 1.02 ± 0.03 |
| SEQ ID NO: 35 | 0.88 ± 0.03 |
| SEQ ID NO: 36 | 0.85 ± 0.04 |
| SEQ ID NO: 37 | 0.96 ± 0.04 |
| SEQ ID NO: 38 | 0.95 ± 0.02 |
| SEQ ID NO: 40 | 2.8 ± 0.5 |
| SEQ ID NO: 41 | 3.3 ± 0.2 |
| SEQ ID NO: 42 | 3.4 ± 0.2 |
| SEQ ID NO: 43 | 4.3 ± 0.2 |
| SEQ ID NO: 44 | 0.97 ± 0.03 |
| SEQ ID NO: 45 | 0.98 ± 0.04 |
| SEQ ID NO: 47 | 1.05 ± 0.05 |
| SEQ ID NO: 48 | 0.95 ± 0.05 |
| SEQ ID NO: 53 | 1.03 ± 0.06 |
| Polymyxin B | 1.21 ± 0.03 |

Human HBE bronchial epithelial cells were incubated with 100 μg/ml peptide or Polymyxin B for 24 hours. LDH activity was assayed in the supernatant of the cell cultures. As a control for 100% LDH release, Triton X-100 was added. The data is presented as the mean ± standard deviation. Only peptides SEQ ID 40, 41, 42 and 43 showed any significant toxicity.

TABLE 17
Toxicity of the Cationic Peptides as Measured by the WST-1 Assay.

| Treatment | OD₄₉₀ nm |
|---|---|
| No cells Control | 0.24 ± 0.01 |
| Triton X-100 Control | 0.26 ± 0.01 |
| No peptide control | 1.63 ± 0.16 |
| SEQ ID NO: 1 | 1.62 ± 0.34 |
| SEQ ID NO: 3 | 1.35 ± 0.12 |
| SEQ ID NO: 10 | 1.22 ± 0.05 |
| SEQ ID NO: 6 | 1.81 ± 0.05 |
| SEQ ID NO: 7 | 1.78 ± 0.10 |
| SEQ ID NO: 9 | 1.69 ± 0.29 |
| SEQ ID NO: 13 | 1.23 ± 0.11 |
| SEQ ID NO: 14 | 1.25 ± 0.02 |
| SEQ ID NO: 16 | 1.39 ± 0.26 |
| SEQ ID NO: 17 | 1.60 ± 0.46 |
| SEQ ID NO: 19 | 1.42 ± 0.15 |
| SEQ ID NO: 20 | 1.61 ± 0.21 |
| SEQ ID NO: 21 | 1.28 ± 0.07 |
| SEQ ID NO: 22 | 1.33 ± 0.07 |
| SEQ ID NO: 23 | 1.14 ± 0.24 |
| SEQ ID NO: 24 | 1.27 ± 0.16 |
| SEQ ID NO: 26 | 1.42 ± 0.11 |
| SEQ ID NO: 27 | 1.63 ± 0.03 |
| SEQ ID NO: 28 | 1.69 ± 0.03 |
| SEQ ID NO: 29 | 1.75 ± 0.09 |
| SEQ ID NO: 31 | 1.84 ± 0.06 |
| SEQ ID NO: 33 | 1.75 ± 0.21 |
| SEQ ID NO: 34 | 0.96 ± 0.05 |
| SEQ ID NO: 35 | 1.00 ± 0.08 |
| SEQ ID NO: 36 | 1.58 ± 0.05 |
| SEQ ID NO: 37 | 1.67 ± 0.02 |
| SEQ ID NO: 38 | 1.83 ± 0.03 |
| SEQ ID NO: 40 | 0.46 ± 0.06 |
| SEQ ID NO: 41 | 0.40 ± 0.01 |
| SEQ ID NO: 42 | 0.39 ± 0.08 |
| SEQ ID NO: 43 | 0.46 ± 0.10 |
| SEQ ID NO: 44 | 1.49 ± 0.39 |
| SEQ ID NO: 45 | 1.54 ± 0.35 |
| SEQ ID NO: 47 | 1.14 ± 0.23 |
| SEQ ID NO: 48 | 0.93 ± 0.08 |
| SEQ ID NO: 53 | 1.51 ± 0.37 |
| Polymyxin B | 1.30 ± 0.13 |

HBE cells were incubated with 100 μg/ml peptide or Polymyxin B for 24 hours and cell viability was tested. The data is presented as the mean ± standard deviation. As a control for 100% LDH release, Triton X-100 was added. Only peptides SEQ ID NOS: 40, 41, 42 and 43 showed any significant toxicity.

### EXAMPLE 4

### Polynucleotide Regulation by Cationic Peptides

Polynucleotide arrays were utilized to determine the effect of cationic peptides by themselves on the transcriptional response of macrophages and epithelial cells. Mouse macrophage RAW 264.7, Human Bronchial cells (HBE), or A549 human epithelial cells were plated in 150 mm tissue culture dishes at 5.6×10⁶ cells/dish, cultured overnight and then incubated with 50 μg/ml peptide or medium alone for 4 h. After stimulation, the cells were washed once with diethyl pyrocarbonate-treated PBS, and detached from the dish using a cell scraper. Total RNA was isolated using Trizol (Gibco Life Technologies). The RNA pellet was resuspended in RNase-free water containing RNase inhibitor (Ambion, Austin, Tex.). The RNA was treated with DNaseI (Clontech, Palo Alto, Calif.) for 1 h at 37° C. After adding termination mix (0.1 M EDTA [pH 8.0], 1 mg/ml glycogen), the samples were extracted once with phenol: chloroform: isoamyl alcohol (25:24:1), and once with chloroform. The RNA was then precipitated by adding 2.5 volumes of 100% ethanol and 1/10^{th} volume sodium acetate, pH 5.2. The RNA was resuspended in RNase-free water with RNase inhibitor (Ambion) and stored at −70° C. The quality of the RNA was assessed by gel electrophoresis on a 1% agarose gel. Lack of genomic DNA contamination was assessed by using the isolated RNA as a template for PCR amplification with β-actin-specific primers (5′-GTCCCTGTATGCCTCTGGTC-3′(SEQ ID NO: 55) and 5′-GATGTCACGCACGATTTCC-3′(SEQ ID NO: 56)). Agarose gel electrophoresis and ethidium bromide staining confirmed the absence of an amplicon after 35 cycles.

Atlas cDNA Expression Arrays (Clontech, Palo Alto, Calif.), which consist of 588 selected mouse cDNAs spotted in duplicate on positively charged membranes were used for early polynucleotide array studies (Tables 18 and 19). ³²P-radiolabeled cDNA probes prepared from 5 μg total RNA were incubated with the arrays overnight at 71° C. The filters were washed extensively and then exposed to a phosphoimager screen (Molecular Dynamics, Sunnyvale, Calif.) for 3 days at 4° C. The image was captured using a Molecular Dynamics PSI phosphoimager. The hybridization signals were analyzed using AtlasImage 1.0 Image Analysis software (Clontech) and Excel (Microsoft, Redmond, Wash.). The intensities for each spot were corrected for background levels and normalized for differences in probe labeling using the average values for 5 polynucleotides observed to vary little between the stimulation conditions: β-actin, ubiquitin, ribosomal protein S29, glyceraldehyde-3-phosphate dehydrogenase (GAPDH), and Ca²⁺ binding protein. When the normalized hybridization intensity for a given cDNA was less than 20, it was assigned a value of 20 to calculate the ratios and relative expression.

The next polynucleotide arrays used (Tables 21-26) were the Resgen Human cDNA arrays (identification number for the genome is PRHU03-S3), which consist of 7,458 human cDNAs spotted in duplicate. Probes were prepared from 15-20 μg of total RNA and labeled with Cy3 labeled dUTP. The probes were purified and hybridized to printed glass slides overnight at 42° C. and washed. After washing, the image was captured using a Virtek slide reader. The image processing software (Imagene 4.1, Marina Del Rey, Calif.) determines the spot mean intensity, median intensities, and background intensities. Normalization and analysis was performed with Genespring software (Redwood City, Calif.). Intensity values were calculated by subtracting the mean background intensity from the mean intensity value determined by Imagene. The intensities for each spot were normalized by taking the median spot intensity value from the population of spot values within a slide and comparing this value to the values of all slides in the experiment. The relative changes seen with cells treated with peptide compared to control cells can be found in the Tables below.

The other polynucleotide arrays used (Tables 27-35) were the Human Operon arrays (identification number for the genome is PRHU04-S1), which consist of about 14,000 human oligos spotted in duplicate. Probes were prepared from 10 μg of total RNA and labeled with Cy3 or Cy5 labeled dUTP. In these experiments, A549 epithelial cells were plated in 100 mm tissue culture dishes at 2.5×10⁶ cells/dish. Total RNA was isolated using RNAqueous (Ambion). DNA contamination was removed with DNA-free kit (Ambion). The probes prepared from total RNA were purified and hybridized to printed glass slides overnight at 42° C. and washed. After washing, the image was captured using a Perkin Elmer array scanner. The image processing software (Imagene 5.0, Marina Del Rey, Calif.) determines the spot mean intensity, median intensities, and background intensities. An “in house” program was used to remove background. The program calculates the bottom 10% intensity for each subgrid and subtracts this for each grid. Analysis was performed with Genespring software (Redwood City, Calif.). The intensities for each spot were normalized by taking the median spot intensity value from the population of spot values within a slide and comparing this value to the values of all slides in the experiment. The relative changes seen with cells treated with peptide compared to control cells can be found in the Tables below.

Semi-quantitative RT-PCR was performed to confirm polynucleotide array results. 1 μg RNA samples were incubated with 1 μl oligodT (500 μg/ml) and 1 μl mixed dNTP stock at 1 mM, in a 12 μl volume with DEPC treated water at 65° C. for 5 min in a thermocycler. 4 μl 5× First Strand buffer, 2 μl 0.1M DTT, and 1 μl RNaseOUT recombinant ribonuclease inhibitor (40 units/μl) were added and incubated at 42° C. for 2 min, followed by the addition of 1 μl (200 units) of Superscript II (Invitrogen, Burlington, ON). Negative controls for each RNA source were generated using parallel reactions in the absence of Superscript II. cDNAs were amplified in the presence of 5′ and 3′ primers (1.0 μM), 0.2 mM dNTP mixture, 1.5 mM MgCl, 1 U of Taq DNA polymerase (New England Biolabs, Missisauga, ON), and 1× PCR buffer. Each PCR was performed with a thermal cycler by using 30-40 cycles consisting of 30s of denaturation at 94° C., 30s of annealing at either 52° C. or 55° C. and 40s of extension at 72° C. The number of cycles of PCR was optimized to lie in the linear phase of the reaction for each primer and set of RNA samples. A housekeeping polynucleotide β-actin was amplified in each experiment to evaluate extraction procedure and to estimate the amount of RNA. The reaction product was visualized by electrophoresis and analyzed by densitometry, with relative starting RNA concentrations calculated with reference to β-actin amplification.

Table 18 demonstrates that SEQ ID NO: 1 treatment of RAW 264.7 cells up-regulated the expression of more than 30 different polynucleotides on small Atlas microarrays with selected known polynucleotides. The polynucleotides up-regulated by peptide, SEQ ID NO: 1, were mainly from two categories: one that includes receptors (growth, chemokine, interleukin, interferon, hormone, neurotransmitter), cell surface antigens and cell adhesion and another one that includes cell-cell communication (growth factors, cytokines, chemokines, interleukin, interferons, hormones), cytoskeleton, motility, and protein turnover. The specific polynucleotides up-regulated included those encoding chemokine MCP-3, the anti-inflammatory cytokine IL-10, macrophage colony stimulating factor, and receptors such as IL-1R-2 (a putative antagonist of productive IL-1 binding to IL-1R1), PDGF receptor B, NOTCH4, LIF receptor, LFA-1, TGFβ receptor 1, G-CSF receptor, and IFNγ receptor. The peptide also up-regulated polynucleotides encoding several metalloproteinases, and inhibitors thereof, including the bone morphogenetic proteins BMP-1, BMP-2, BMP-8a, TIMP2 and TIMP3. As well, the peptide up-regulated specific transcription factors, including JunD, and the YY and LIM-1 transcription factors, and kinases such as Etk1 and Csk demonstrating its widespread effects. It was also discovered from the polynucleotide array studies that SEQ ID NO: 1 down-regulated at least 20 polynucleotides in RAW 264.7 macrophage cells (Table 19). The polynucleotides down-regulated by peptide included DNA repair proteins and several inflammatory mediators such as MIP-1α, oncostatin M and IL-12. A number of the effects of peptide on polynucleotide expression were confirmed by RT-PCR (Table 20). The peptides, SEQ ID NO: 2, SEQ ID NO: 3, SEQ ID NO: 19, and SEQ ID NO: 1, and representative peptides from each of the formulas also altered the transcriptional responses in a human epithelial cell line using mid-sized microarrays (7835 polynucleotides). The effect of SEQ ID NO: 1 on polynucleotide expression was compared in 2 human epithelial cell lines, A549 and HBE. Polynucleotides related to the host immune response that were up-regulated by 2 peptides or more by a ratio of 2-fold more than unstimulated cells. are described in Table 21. Polynucleotides that were down-regulated by 2 peptides or more by a ratio of 2-fold more than unstimulated cells are described in Table 22. In Table 23 and Table 24, the human epithelial pro-inflammatory polynucleotides that are up- and down-regulated respectively are shown. In Table 25 and Table 26 the anti-inflammatory polynucleotides affected by cationic peptides are shown. The trend becomes clear that the cationic peptides up-regulate the anti-inflammatory response and down-regulate the pro-inflammatory response. It was very difficult to find a polynucleotide related to the anti-inflammatory response that was down-regulated (Table 26). The pro-inflammatory polynucleotides upregulated by cationic peptides were mainly polynucleotides related to migration and adhesion. Of the down-regulated pro-inflammatory polynucleotides, it should be noted that all the cationic peptides affected several toll-like receptor (TLR) polynucleotides, which are very important in signaling the host response to infectious agents. An important anti-inflammatory polynucleotide that was up-regulated by all the peptides is the IL-10 receptor. IL-10 is an important cytokine involved in regulating the pro-inflammatory cytokines. These polynucleotide expression effects were also observed using primary human macrophages as observed for peptide SEQ ID NO: 6 in Tables 27 and 28. The effect of representative peptides from each of the formulas on human epithelial cell expression of selected polynucleotides (out of 14,000 examined) is shown in Tables 31-37 below. At least 6 peptides from each formula were tested for their ability to alter human epithelial polynucleotide expression and indeed they had a wide range of stimulatory effects. In each of the formulas there were at least 50 polynucleotides commonly up-regulated by each of the peptides in the group.

TABLE 18
Polynucleotides up-regulated by peptide, SEQ ID NO: 1, treatment of RAW macrophage cells^{a}.

| Polynucleotide/ | | Unstimulated | Ratio | Accession |
|---|---|---|---|---|
| Protein | Polynucleotide Function | Intensity | peptide:Unstimulated^{b} | Number |
| Etk1 | Tyrosine-protein kinase | 20 | 43 | M68513 |
| | receptor | | | |
| PDGFRB | Growth factor receptor | 24 | 25 | X04367 |
| | Corticotropin releasing | 20 | 23 | X72305 |
| | factor receptor | | | |
| NOTCH4 | proto-oncopolynucleotide | 48 | 18 | M80456 |
| IL-1R2 | Interleukin receptor | 20 | 16 | X59769 |
| MCP-3 | Chemokine | 56 | 14 | S71251 |
| BMP-1 | Bone | 20 | 14 | L24755 |
| | morphopolynucleotidetic | | | |
| | protein | | | |
| Endothelin | Receptor | 20 | 14 | U32329 |
| b receptor | | | | |
| c-ret | Oncopolynucleotide | 20 | 13 | X67812 |
| | precursor | | | |
| LIFR | Cytokine receptor | 20 | 12 | D26177 |
| BMP-8a | Bone | 20 | 12 | M97017 |
| | morphopolynucleotidetic | | | |
| | protein | | | |
| Zfp92 | Zinc finger protein 92 | 87 | 11 | U47104 |
| MCSF | Macrophage colony | 85 | 11 | X05010 |
| | stimulating factor 1 | | | |
| GCSFR | Granulocyte colony- | 20 | 11 | M58288 |
| | stimulating factor receptor | | | |
| IL-8RB | Chemokine receptor | 112 | 10 | D17630 |
| IL-9R | Interleukin receptor | 112 | 6 | M84746 |
| Cas | Crk-associated substrate | 31 | 6 | U48853 |
| p58/GTA | Kinase | 254 | 5 | M58633 |
| CASP2 | Caspase precursor | 129 | 5 | D28492 |
| IL-1β | Interleukin precursor | 91 | 5 | M15131 |
| precursor | | | | |
| SPI2-2 | Serine protease inhibitor | 62 | 5 | M64086 |
| C5AR | Chemokine receptor | 300 | 4 | S46665 |
| L-myc | Oncopolynucleotide | 208 | 4 | X13945 |
| IL-10 | Interleukin | 168 | 4 | M37897 |
| p19ink4 | cdk4 and cdk6 inhibitor | 147 | 4 | U19597 |
| ATOH2 | Atonal homolog 2 | 113 | 4 | U29086 |
| DNAse1 | DNase | 87 | 4 | U00478 |
| CXCR-4 | Chemokine receptor | 36 | 4 | D87747 |
| Cyclin D3 | Cyclin | 327 | 3 | U43844 |
| IL-7Rα | Interleukin receptor | 317 | 3 | M29697 |
| POLA | DNA polymerase_{α} | 241 | 3 | D17384 |
| Tie-2 | Oncopolynucleotide | 193 | 3 | S67051 |
| DNL1 | DNA ligase I | 140 | 3 | U04674 |
| BAD | Apoptosis protein | 122 | 3 | L37296 |
| GADD45 | DNA-damage-inducible | 88 | 3 | L28177 |
| | protein | | | |
| Sik | Src-related kinase | 82 | 3 | U16805 |
| integrin_{α}4 | Integrin | 2324 | 2 | X53176 |
| TGFβR1 | Growth factor receptor | 1038 | 2 | D25540 |
| LAMR1 | Receptor | 1001 | 2 | J02870 |
| Crk | Crk adaptor protein | 853 | 2 | S72408 |
| ZFX | Chromosomal protein | 679 | 2 | M32309 |
| Cyclin E1 | Cylcin | 671 | 2 | X75888 |
| POLD1 | DNA polymerase subunit | 649 | 2 | Z21848 |
| Vav | proto-oncopolynucleotide | 613 | 2 | X64361 |
| YY (NF-E1) | Transcription factor | 593 | 2 | L13968 |
| JunD | Transcription factor | 534 | 2 | J050205 |
| Csk | c-src kinase | 489 | 2 | U05247 |
| Cdk7 | Cyclin-dependent kinase | 475 | 2 | U11822 |
| MLC1A | Myosin light subunit | 453 | 2 | M19436 |
| | isoform | | | |
| ERBB-3 | Receptor | 435 | 2 | L47240 |
| UBF | Transcription factor | 405 | 2 | X60831 |
| TRAIL | Apoptosis ligand | 364 | 2 | U37522 |
| LFA-1 | Cell adhesion receptor | 340 | 2 | X14951 |
| SLAP | Src-like adaptor protein | 315 | 2 | U29056 |
| IFNGR | Interferon gamma receptor | 308 | 2 | M28233 |
| LIM-1 | Transcription factor | 295 | 2 | Z27410 |
| ATF2 | Transcription factor | 287 | 2 | S76657 |
| FST | Follistatin precursor | 275 | 2 | Z29532 |
| TIMP3 | Protease inhibitor | 259 | 2 | L19622 |
| RU49 | Transcription factor | 253 | 2 | U41671 |
| IGF-1Rα | Insulin-like growth factor | 218 | 2 | U00182 |
| | receptor | | | |
| Cyclin G2 | Cyclin | 214 | 2 | U95826 |
| fyn | Tyrosine-protein kinase | 191 | 2 | U70324 |
| BMP-2 | Bone | 186 | 2 | L25602 |
| | morphopolynucleotidetic | | | |
| | protein | | | |
| Brn-3.2 | Transcription factor | 174 | 2 | S68377 |
| POU | | | | |
| KIF1A | Kinesin family protein | 169 | 2 | D29951 |
| MRC1 | Mannose receptor | 167 | 2 | Z11974 |
| PAI2 | Protease inhibitor | 154 | 2 | X19622 |
| BKLF | CACCC Box-binding | 138 | 2 | U36340 |
| | protein | | | |
| TIMP2 | Protease inhibitor | 136 | 2 | X62622 |
| Mas | Proto-oncopolynucleotide | 131 | 2 | X67735 |
| NURR-1 | Transcription factor | 129 | 2 | S53744 |
| The cationic peptides at a concentration of 50 μg/ml were shown to potently induce the expression of several polynucleotides. Peptide was incubated with the RAW cells for 4 h and the RNA was isolated, converted into labeled cDNA probes and hybridized to Atlas arrays. The intensity of unstimulated cells is shown in the third column. The “Ratio Peptide:Unstimulated” column refers to the intensity of polynucleotide expression in peptide-simulated cells divided by the intensity of | | | | |
| # unstimulated cells. | | | | |
| The changes in the normalized intensities of the housekeeping polynucleotides ranged from 0.8-1.2 fold, validating the use of these polynucleotides for normalization. When the normalized hybridization intensity for a given cDNA was less than 20, it was assigned a value of 20 to calculate the ratios and relative expression. The array experiments were repeated 3 times with different RNA preparations and the average fold change is shown above. Polynucleotides with a two fold or greater | | | | |
| # change in relative expression levels are presented. | | | | |

TABLE 19
Polynucleotides down-regulated by SEQ ID NO: 1 treatment of RAW macrophage cells^{a}.

| Polynucleotide/ | | Unstimulated | Ratio | Accession |
|---|---|---|---|---|
| Protein | Polynucleotide Function | Intensity | peptide:Unstimulated | Number |
| sodium channel | Voltage-gated ion channel | 257 | 0.08 | L36179 |
| XRCC1 | DNA repair protein | 227 | 0.09 | U02887 |
| ets-2 | Oncopolynucleotide | 189 | 0.11 | J04103 |
| XPAC | DNA repair protein | 485 | 0.12 | X74351 |
| EPOR | Receptor precursor | 160 | 0.13 | J04843 |
| PEA 3 | Ets-related protein | 158 | 0.13 | X63190 |
| orphan receptor | Nuclear receptor | 224 | 0.2 | U11688 |
| N-cadherin | Cell adhesion receptor | 238 | 0.23 | M31131 |
| OCT3 | Transcription factor | 583 | 0.24 | M34381 |
| PLCβ | phospholipase | 194 | 0.26 | U43144 |
| KRT18 | Intermediate filament | 318 | 0.28 | M11686 |
| | proteins | | | |
| THAM | Enzyme | 342 | 0.32 | X58384 |
| CD40L | CD40 ligand | 66 | 0.32 | X65453 |
| CD86 | T-lymphocyte antigen | 195 | 0.36 | L25606 |
| oncostatin M | Cytokine | 1127 | 0.39 | D31942 |
| PMS2 DNA | DNA repair protein | 200 | 0.4 | U28724 |
| IGFBP6 | Growth factor | 1291 | 0.41 | X81584 |
| MIP-1β | Cytokine | 327 | 0.42 | M23503 |
| ATBF1 | AT motif-binding factor | 83 | 0.43 | D26046 |
| nucleobindin | Golgi resident protein | 367 | 0.43 | M96823 |
| bcl-x | Apoptosis protein | 142 | 0.43 | L35049 |
| uromodulin | glycoprotein | 363 | 0.47 | L33406 |
| IL-12 p40 | Interleukin | 601 | 0.48 | M86671 |
| MmRad52 | DNA repair protein | 371 | 0.54 | Z32767 |
| Tob1 | Antiproliferative factor | 956 | 0.5 | D78382 |
| Ung1 | DNA repair protein | 535 | 0.51 | X99018 |
| KRT19 | Intermediate filament | 622 | 0.52 | M28698 |
| | proteins | | | |
| PLCγ | phospholipase | 251 | 0.52 | X95346 |
| Integrin α₆ | Cell adhesion receptor | 287 | 0.54 | X69902 |
| GLUT1 | Glucose transporter | 524 | 0.56 | M23384 |
| CTLA4 | immunoglobin | 468 | 0.57 | X05719 |
| | superfamily | | | |
| FRA2 | Fos-related antigen | 446 | 0.57 | X83971 |
| MTRP | Lysosome-associated | 498 | 0.58 | U34259 |
| | protein | | | |
| The cationic peptides at a concentration of 50 μg/m1 were shown to reduce the expression of several polynucleotides. Peptide was incubated with the RAW cells for 4 h and the RNA was isolated, converted into labeled cDNA probes and hybridized to Atlas arrays. The intensity of unstimulated cells is shown in the third column. The “Ratio Peptide:Unstimulated” column refers to the intensity of polynucleotide expression in peptide-simulated cells divided by experiments were | | | | |
| # repeated 3 times with different cells and the average fold change is shown below. Polynucleotides with an approximately two fold or greater change in relative expression levels are presented. | | | | |

TABLE 20
Polynucleotide Expression changes in response to peptide,
SEQ ID NO: 1, could be confirmed by RT-PCR.

| Polynucleotide | Array Ratio-* | RT-PCR Ratio-* |
|---|---|---|
| CXCR-4 | 4.0 ± 1.7 | 4.1 ± 0.9 |
| IL-8RB | 9.5 ± 7.6 | 7.1 ± 1.4 |
| MCP-3 | 13.5 ± 4.4 | 4.8 ± 0.88 |
| IL-10 | 4.2 ± 2.1 | 16.6 ± 6.1 |
| CD14 | 0.9 ± 0.1 | 0.8 ± 0.3 |
| MIP-1B | 0.42 ± 0.09 | 0.11 ± 0.04 |
| XRCC1 | 0.12 ± 0.01 | 0.25 ± 0.093 |
| MCP-1 | Not on array | 3.5 ± 1.4 |
| RAW 264.7 macrophage cells were incubated with 50 μg/ml of peptide or media only for 4 hours and total RNA isolated and subjected to semi-quantitative RT-PCR. Specific primer pairs for each polynucleotide were used for amplification of RNA. Amplification of β-actin was used as a positive control and for standardization. Densitometric analysis of RT-PCR products was used. The results refer to the relative fold change in polynucleotide expression of peptide treated cells | | |
| # compared to cells incubated with media alone. The data is presented as the mean ± standard error of three experiments. | | |

TABLE 21
Polynucleotides up-regulated by peptide treatment of A549 epithelial cells^{a}.

| | Unstimulated | Ratio Peptide:Unstimulated | | | | Accession |
|---|---|---|---|---|---|---|
| Polynucleotide/Protein | Intensity | ID 2 | ID 3 | ID 19 | ID 1 | Number |
| IL-1 R antagonist homolog 1 | 0.00 | 3086 | 1856 | 870 | | AI167887 |
| IL-10 R beta | 0.53 | 2.5 | 1.6 | 1.9 | 3.1 | AA486393 |
| IL-11 R alpha | 0.55 | 2.4 | 1.0 | 4.9 | 1.8 | AA454657 |
| IL-17 R | 0.54 | 2.1 | 2.0 | 1.5 | 1.9 | AW029299 |
| TNF R superfamily, member | 0.28 | 18 | 3.0 | 15 | 3.6 | AA150416 |
| 1B | | | | | | |
| TNF R superfamily, member 5 | 33.71 | 3.0 | 0.02 | | | H98636 |
| (CD40LR) | | | | | | |
| TNF R superfamily, member | 1.00 | 5.3 | 4.50 | 0.8 | | AA194983 |
| 11b | | | | | | |
| IL-8 | 0.55 | 3.6 | 17 | 1.8 | 1.1 | AA102526 |
| interleukin enhancer binding | 0.75 | 1.3 | 2.3 | 0.8 | 4.6 | AA894687 |
| factor 2 | | | | | | |
| interleukin enhancer binding | 0.41 | 2.7 | | 5.3 | 2.5 | R56553 |
| factor 1 | | | | | | |
| cytokine inducible SH2- | 0.03 | 33 | 44 | 39 | 46 | AA427521 |
| containing protein | | | | | | |
| IK cytokine, down-regulator of | 0.50 | 3.1 | 2.0 | 1.7 | 3.3 | R39227 |
| HLA II | | | | | | |
| cytokine inducible SH2- | 0.03 | 33 | 44 | 39 | 46 | AA427521 |
| containing protein | | | | | | |
| IK cytokine, down-regulator of | 0.50 | 3.1 | 2.0 | 1.7 | 3.3 | R39227 |
| HLA II | | | | | | |
| small inducible cytokine | 1.00 | 3.9 | | | 2.4 | AI922341 |
| subfamily A (Cys—Cys), | | | | | | |
| member 21 | | | | | | |
| TGFB inducible early growth | 0.90 | 2.4 | 2.1 | 0.9 | 1.1 | AI473938 |
| response 2 | | | | | | |
| NK cell R | 1.02 | 2.5 | 0.7 | 0.3 | 1.0 | AA463248 |
| CCR6 | 0.14 | 4.5 | 7.8 | 6.9 | 7.8 | N57964 |
| cell adhesion molecule | 0.25 | 4.0 | 3.9 | 3.9 | 5.1 | R40400 |
| melanoma adhesion molecule | 0.05 | 7.9 | 20 | 43 | 29.1 | AA497002 |
| CD31 | 0.59 | 2.7 | 3.1 | 1.0 | 1.7 | R22412 |
| integrin, alpha 2 (CD49B, | 1.00 | 0.9 | 2.4 | 3.6 | 0.9 | AA463257 |
| alpha 2 subunit of VLA-2 | | | | | | |
| receptor | | | | | | |
| integrin, alpha 3 (antigen | 0.94 | 0.8 | 2.5 | 1.9 | 1.1 | AA424695 |
| CD49C, alpha 3 subunit of | | | | | | |
| VLA-3 receptor) | | | | | | |
| integrin, alpha E | 0.01 | 180 | 120 | 28 | 81 | AA425451 |
| integrin, beta 1 | 0.47 | 2.1 | 2.1 | 7.0 | 2.6 | W67174 |
| integrin, beta 3 | 0.55 | 2.7 | 2.8 | 1.8 | 1.0 | AA037229 |
| integrin, beta 3 | 0.57 | 2.6 | 1.4 | 1.8 | 2.0 | AA666269 |
| integrin, beta 4 | 0.65 | 0.8 | 2.2 | 4.9 | 1.5 | AA485668 |
| integrin beta 4 binding protein | 0.20 | 1.7 | 5.0 | 6.6 | 5.3 | AI017019 |
| calcium and integrin binding | 0.21 | 2.8 | 4.7 | 9.7 | 6.7 | AA487575 |
| protein | | | | | | |
| disintegrin and | 0.46 | 3.1 | | 2.2 | 3.8 | AA279188 |
| metalloproteinase domain 8 | | | | | | |
| disintegrin and | 0.94 | 1.1 | 2.3 | 3.6 | 0.5 | H59231 |
| metalloproteinase domain 9 | | | | | | |
| disintegrin and | 0.49 | 1.5 | 2.1 | 3.3 | 2.2 | AA043347 |
| metalloproteinase domain 10 | | | | | | |
| disintegrin and | 0.44 | 1.9 | 2.3 | 2.5 | 4.6 | H11006 |
| metalloproteinase domain 23 | | | | | | |
| cadherin 1, type 1, E-cadherin | 0.42 | 8.1 | 2.2 | 2.4 | 7.3 | H97778 |
| (epithelial) | | | | | | |
| cadherin 12, type 2 (N- | 0.11 | 13 | 26 | 9.5 | | AI740827 |
| cadherin 2) | | | | | | |
| protocadherin 12 | 0.09 | 14.8 | 11.5 | 2.6 | 12.4 | AI652584 |
| protocadherin gamma | 0.34 | 3.0 | 2.5 | 4.5 | 9.9 | R89615 |
| subfamily C, 3 | | | | | | |
| catenin (cadherin-associated | 0.86 | 1.2 | 2.2 | 2.4 | | AA025276 |
| protein), delta 1 | | | | | | |
| laminin R 1 (67 kD, ribosomal | 0.50 | 0.4 | 2.0 | 4.4 | 3.0 | AA629897 |
| protein SA) | | | | | | |
| killer cell lectin-like receptor | 0.11 | 9.7 | 9.0 | 4.1 | 13.4 | AA190627 |
| subfamily C, member 2 | | | | | | |
| killer cell lectin-like receptor | 1.00 | 3.2 | 1.0 | 0.9 | 1.3 | W93370 |
| subfamily C, member 3 | | | | | | |
| killer cell lectin-like receptor | 0.95 | 2.3 | 1.7 | 0.7 | 1.1 | AI433079 |
| subfamily G, member 1 | | | | | | |
| C-type lectin-like receptor-2 | 0.45 | 2.1 | 8.0 | 2.2 | 5.3 | H70491 |
| CSF 3 R | 0.40 | 1.9 | 2.5 | 3.5 | 4.0 | AA458507 |
| macrophage stimulating 1 R | 1.00 | 1.7 | 2.3 | 0.4 | 0.7 | AA173454 |
| BMP R type IA | 0.72 | 1.9 | 2.8 | 0.3 | 1.4 | W15390 |
| formyl peptide receptor 1 | 1.00 | 3.1 | 1.4 | 0.4 | | AA425767 |
| CD2 | 1.00 | 2.6 | 0.9 | 1.2 | 0.9 | AA927710 |
| CD36 | 0.18 | 8.2 | 5.5 | 6.2 | 2.5 | N39161 |
| vitamin D R | 0.78 | 2.5 | 1.3 | 1.1 | 1.4 | AA485226 |
| Human proteinase activated R-2 | 0.54 | 6.1 | 1.9 | 2.2 | | AA454652 |
| prostaglandin E receptor 3 | 0.25 | 4.1 | 4.9 | 3.8 | 4.9 | AA406362 |
| (subtype EP3) | | | | | | |
| PDGF R beta polypeptide | 1.03 | 2.5 | 1.0 | 0.5 | 0.8 | R56211 |
| VIP R 2 | 1.00 | 3.1 | | | 2.0 | AI057229 |
| growth factor receptor-bound | 0.51 | 2.2 | 2.0 | 2.4 | 0.3 | AA449831 |
| protein 2 | | | | | | |
| Mouse Mammary Turmor | 1.00 | 6.9 | | 16 | | W93891 |
| Virus Receptor homolog | | | | | | |
| adenosine A2a R | 0.41 | 3.1 | 1.8 | 4.0 | 2.5 | N57553 |
| adenosine A3 R | 0.83 | 2.0 | 2.3 | 1.0 | 1.2 | AA863086 |
| T cell R delta locus | 0.77 | 2.7 | 1.3 | | 1.8 | AA670107 |
| prostaglandin E receptor 1 | 0.65 | 7.2 | | 6.0 | 1.5 | AA972293 |
| (subtype EP1) | | | | | | |
| growth factor receptor-bound | 0.34 | | 3.0 | 6.3 | 2.9 | R24266 |
| protein 14 | | | | | | |
| Epstein-Barr virus induced | 0.61 | 1.6 | 2.4 | | 8.3 | AA037376 |
| polynucleotide 2 | | | | | | |
| complement component | 0.22 | 26 | 4.5 | 2.6 | 18.1 | AA521362 |
| receptor 2 | | | | | | |
| endothelin receptor type A | 0.07 | 12 | 14 | 14 | 16 | AA450009 |
| v-SNARE R | 0.56 | 11 | 12 | 1.8 | | AA704511 |
| tyrosine kinase, non-receptor, 1 | 0.12 | 7.8 | 8.5 | 10 | 8.7 | AI936324 |
| receptor tyrosine kinase-like | 0.40 | 7.3 | 5.0 | 1.6 | 2.5 | N94921 |
| orphan receptor 2 | | | | | | |
| protein tyrosine phosphatase, | 1.02 | 1.0 | 13.2 | 0.5 | 0.8 | AA682684 |
| non-receptor type 3 | | | | | | |
| protein tyrosine phosphatase, | 0.28 | 3.5 | 4.0 | 0.9 | 5.3 | AA434420 |
| non-receptor type 9 | | | | | | |
| protein tyrosine phosphatase, | 0.42 | 2.9 | 2.4 | 2.2 | 3.0 | AA995560 |
| non-receptor type 11 | | | | | | |
| protein tyrosine phosphatase, | 1.00 | 2.3 | 2.2 | 0.8 | 0.5 | AA446259 |
| non-receptor type 12 | | | | | | |
| protein tyrosine phosphatase, | 0.58 | 1.7 | 2.4 | 3.6 | 1.7 | AA679180 |
| non-receptor type 13 | | | | | | |
| protein tyrosine phosphatase, | 0.52 | 3.2 | 0.9 | 1.9 | 6.5 | AI668897 |
| non-receptor type 18 | | | | | | |
| protein tyrosine phosphatase, | 0.25 | 4.0 | 2.4 | 16.8 | 12.8 | H82419 |
| receptor type, A | | | | | | |
| protein tyrosine phosphatase, | 0.60 | 3.6 | 3.2 | 1.6 | 1.0 | AA045326 |
| receptor type, J | | | | | | |
| protein tyrosine phosphatase, | 0.73 | 1.2 | 2.8 | 3.0 | 1.4 | R52794 |
| receptor type, T | | | | | | |
| protein tyrosine phosphatase, | 0.20 | 6.1 | 1.2 | 5.6 | 5.0 | AA644448 |
| receptor type, U | | | | | | |
| protein tyrosine phosphatase, | 1.00 | 5.1 | | | 2.4 | AA481547 |
| receptor type, C-associated | | | | | | |
| protein | | | | | | |
| phospholipase A2 receptor 1 | 0.45 | 2.8 | 2.2 | 1.9 | 2.2 | AA086038 |
| MAP kinase-activated protein | 0.52 | 2.1 | 2.7 | 1.1 | 1.9 | W68281 |
| kinase 3 | | | | | | |
| MAP kinase kinase 6 | 0.10 | 18 | 9.6 | | 32 | H07920 |
| MAP kinase kinase 5 | 1.00 | 3.0 | 5.2 | 0.8 | 0.2 | W69649 |
| MAP kinase 7 | 0.09 | | 11.5 | 12 | 33 | H39192 |
| MAP kinase 12 | 0.49 | 2.1 | 1.7 | 2.2 | 2.0 | AI936909 |
| G protein-coupled receptor 4 | 0.40 | 3.7 | 3.0 | 2.4 | 2.5 | AI719098 |
| G protein-coupled receptor 49 | 0.05 | | 19 | 19 | 27 | AA460530 |
| G protein-coupled receptor 55 | 0.08 | 19 | 15 | 12 | | N58443 |
| G protein-coupled receptor 75 | 0.26 | 5.2 | 3.1 | 7.1 | 3.9 | H84878 |
| G protein-coupled receptor 85 | 0.20 | 6.8 | 5.4 | 4.9 | 5.0 | N62306 |
| regulator of G-protein | 0.02 | 48 | 137 | 82 | | AI264190 |
| signalling 20 | | | | | | |
| regulator of G-protein | 0.27 | | 3.7 | 8.9 | 10.6 | R39932 |
| signalling 6 | | | | | | |
| BCL2-interacting killer | 1.00 | 1.9 | | 5.2 | | AA291323 |
| (apoptosis-inducing) | | | | | | |
| apoptosis inhibitor 5 | 0.56 | 2.8 | 1.6 | 2.4 | 1.8 | AI972925 |
| caspase 6, apoptosis-related | 0.79 | 0.7 | 2.6 | 1.3 | 2.8 | W45688 |
| cysteine protease | | | | | | |
| apoptosis-related protein | 0.46 | 2.2 | 1.4 | 2.3 | 2.9 | AA521316 |
| PNAS-1 | | | | | | |
| caspase 8, apoptosis-related | 0.95 | 2.2 | 1.0 | 0.6 | 2.0 | AA448468 |
| cysteine protease | | | | | | |

The cationic peptides at concentrations of 50 μg/ml were shown to increase the expression of several polynucleotides. Peptide was incubated with the human A549 epithelial cells for 4 h and the RNA was isolated, converted into labeled cDNA probes and hybridized to Human cDNA arrays ID#PRHU03-S3. The intensity of polynucleotides in unstimulated cells is shown in the second column.
The “Ratio Peptide:Unstimulated” columns refers to the intensity of polynucleotide expression in peptide-simulated cells divided by the intensity of unstimulated cells.

TABLE 22
Polynucleotides down-regulated by peptide treatment of A549 epithelial cells^{a}.

| | Unstimulated | Ratio Peptide:Unstimulated | | | | Accession |
|---|---|---|---|---|---|---|
| Polynucleotide/Protein | Intensity | ID 2 | ID 3 | ID 19 | ID 1 | Number |
| TLR 1 | 3.22 | 0.35 | 0.31 | 0.14 | 0.19 | AI339155 |
| TLR 2 | 2.09 | 0.52 | 0.31 | 0.48 | 0.24 | T57791 |
| TLR 5 | 8.01 | 0.12 | 0.39 | | | N41021 |
| TLR 7 | 5.03 | 0.13 | 0.11 | 0.20 | 0.40 | N30597 |
| TNF receptor-associated factor 2 | 0.82 | 1.22 | 0.45 | 2.50 | 2.64 | T55353 |
| TNF receptor-associated factor 3 | 3.15 | 0.15 | | 0.72 | 0.32 | AA504259 |
| TNF receptor superfamily, member 12 | 4.17 | 0.59 | 0.24 | | 0.02 | W71984 |
| TNF R superfamily, member 17 | 2.62 | | 0.38 | 0.55 | 0.34 | AA987627 |
| TRAF and TNF receptor-associated | 1.33 | 0.75 | 0.22 | 0.67 | 0.80 | AA488650 |
| protein | | | | | | |
| IL-1 receptor, type I | 1.39 | 0.34 | 0.72 | 1.19 | 0.34 | AA464526 |
| IL-2 receptor, alpha | 2.46 | 0.41 | 0.33 | 0.58 | | AA903183 |
| IL-2 receptor, gamma (severe | 3.34 | 0.30 | 0.24 | | 0.48 | N54821 |
| combined immunodeficiency) | | | | | | |
| IL-12 receptor, beta 2 | 4.58 | 0.67 | 0.22 | | | AA977194 |
| IL-18 receptor 1 | 1.78 | 0.50 | 0.42 | 0.92 | 0.56 | AA482489 |
| TGF beta receptor III | 2.42 | 0.91 | 0.24 | 0.41 | 0.41 | H62473 |
| leukotriene b4 receptor (chemokine | 1.00 | | 1.38 | 4.13 | 0.88 | AI982606 |
| receptor-like 1) | | | | | | |
| small inducible cytokine subfamily A | 2.26 | 0.32 | | 0.44 | 1.26 | AA495985 |
| (Cys—Cys), member 18 | | | | | | |
| small inducible cytokine subfamily A | 2.22 | 0.19 | 0.38 | 0.45 | 0.90 | AI285199 |
| (Cys—Cys), member 20 | | | | | | |
| small inducible cytokine subfamily A | 2.64 | 0.38 | 0.31 | 1.53 | | AA916836 |
| (Cys—Cys), member 23 | | | | | | |
| small inducible cytokine subfamily B | 3.57 | 0.11 | 0.06 | 0.28 | 0.38 | AI889554 |
| (Cys-X-Cys), member 6 (granulocyte | | | | | | |
| chemotactic protein 2) | | | | | | |
| small inducible cytokine subfamily B | 2.02 | 0.50 | 1.07 | 0.29 | 0.40 | AA878880 |
| (Cys-X-Cys), member 10 | | | | | | |
| small inducible cytokine A3 | 2.84 | 1.79 | 0.32 | 0.35 | | AA677522 |
| (homologous to mouse Mip-la) | | | | | | |
| cytokine-inducible kinase | 2.70 | 0.41 | 0.37 | 0.37 | 0.34 | AA489234 |
| complement component C1q receptor | 1.94 | 0.46 | 0.58 | 0.51 | 0.13 | AI761788 |
| cadherin 11, type 2, OB-cadherin | 2.00 | 0.23 | 0.57 | 0.30 | 0.50 | AA136983 |
| (osteoblast) | | | | | | |
| cadherin 3, type 1, P-cadherin | 2.11 | 0.43 | 0.53 | 0.10 | 0.47 | AA425217 |
| (placental) | | | | | | |
| cadherin, EGF LAG seven-pass G-type | 1.67 | 0.42 | 0.41 | 1.21 | 0.60 | H39187 |
| receptor 2, flamingo (Drosophila) | | | | | | |
| homolog | | | | | | |
| cadherin 13, H-cadherin (heart) | 1.78 | 0.37 | 0.40 | 0.56 | 0.68 | R41787 |
| selectin L (lymphocyte adhesion | 4.43 | 0.03 | 0.23 | 0.61 | | H00662 |
| molecule 1) | | | | | | |
| vascular cell adhesion molecule 1 | 1.40 | 0.20 | 0.72 | 0.77 | 0.40 | H16591 |
| intercellular adhesion molecule 3 | 1.00 | 0.12 | 0.31 | 2.04 | 1.57 | AA479188 |
| integrin, alpha 1 | 2.42 | 0.41 | 0.26 | | 0.56 | AA450324 |
| integrin, alpha 7 | 2.53 | 0.57 | 0.39 | 0.22 | 0.31 | AA055979 |
| integrin, alpha 9 | 1.16 | 0.86 | 0.05 | 0.01 | 2.55 | AA865557 |
| integrin, alpha 10 | 1.00 | 0.33 | 0.18 | 1.33 | 2.25 | AA460959 |
| integrin, beta 5 | 1.00 | 0.32 | 1.52 | 1.90 | 0.06 | AA434397 |
| integrin, beta 8 | 3.27 | 0.10 | 1.14 | 0.31 | 0.24 | W56754 |
| disintegrin and metalloproteinase | 2.50 | 0.40 | 0.29 | 0.57 | 0.17 | AI205675 |
| domain 18 | | | | | | |
| disintegrin-like and metalloprotease | 2.11 | 0.32 | 0.63 | 0.47 | 0.35 | AA398492 |
| with thrombospondin type 1 motif, 3 | | | | | | |
| disintegrin-like and metalloprotease | 1.62 | 0.39 | 0.42 | 1.02 | 0.62 | AI375048 |
| with thrombospondin type 1 motif, 5 | | | | | | |
| T-cell receptor interacting molecule | 1.00 | 0.41 | 1.24 | 1.41 | 0.45 | AI453185 |
| diphtheria toxin receptor (heparin- | 1.62 | 0.49 | 0.85 | 0.62 | 0.15 | R45640 |
| binding epidermal growth factor-like | | | | | | |
| growth factor) | | | | | | |
| vasoactive intestinal peptide receptor 1 | 2.31 | 0.43 | 0.31 | 0.23 | 0.54 | H73241 |
| Fc fragment of IgG, low affinity IIIb, | 3.85 | −0.20 | 0.26 | 0.76 | 0.02 | H20822 |
| receptor for (CD16) | | | | | | |
| Fc fragment of IgG, low affinity IIb, | 1.63 | 0.27 | 0.06 | 1.21 | 0.62 | R68106 |
| receptor for (CD32) | | | | | | |
| Fc fragment of IgE, high affinity I, | 1.78 | 0.43 | 0.00 | 0.56 | 0.84 | AI676097 |
| receptor for; alpha polypeptide | | | | | | |
| leukocyte immunoglobulin-like | 2.25 | 0.44 | 0.05 | 0.38 | 0.99 | N63398 |
| receptor, subfamily A | | | | | | |
| leukocyte immunoglobulin-like | 14.21 | | | 1.10 | 0.07 | AI815229 |
| receptor, subfamily B (with TM and | | | | | | |
| ITIM domains), member 3 | | | | | | |
| leukocyte immunoglobulin-like | 2.31 | 0.75 | 0.43 | 0.19 | 0.40 | AA076350 |
| receptor, subfamily B (with TM and | | | | | | |
| ITIM domains), member 4 | | | | | | |
| leukocyte immunoglobulin-like | 1.67 | 0.35 | 0.60 | 0.18 | 0.90 | H54023 |
| receptor, subfamily B | | | | | | |
| peroxisome proliferative activated | 1.18 | 0.38 | 0.85 | 0.87 | 0.26 | AI739498 |
| receptor, alpha | | | | | | |
| protein tyrosine phosphatase, receptor | 2.19 | 0.43 | | 1.06 | 0.46 | N49751 |
| type, f polypeptide (PTPRF), | | | | | | |
| interacting protein (liprin), α1 | | | | | | |
| protein tyrosine phosphatase, receptor | 1.55 | 0.44 | 0.64 | 0.30 | 0.81 | H74265 |
| type, C | | | | | | |
| protein tyrosine phosphatase, receptor | 2.08 | 0.23 | 0.37 | 0.56 | 0.48 | AA464542 |
| type, E | | | | | | |
| protein tyrosine phosphatase, receptor | 2.27 | 0.02 | 0.44 | | 0.64 | AA464590 |
| type, N polypeptide 2 | | | | | | |
| protein tyrosine phosphatase, receptor | 2.34 | 0.11 | 0.43 | 0.24 | 0.89 | AI924306 |
| type, H | | | | | | |
| protein tyrosine phosphatase, receptor- | 1.59 | 0.63 | 0.34 | 0.72 | 0.35 | AA476461 |
| type, Z polypeptide 1 | | | | | | |
| protein tyrosine phosphatase, non- | 1.07 | 0.94 | 0.43 | 0.25 | 1.13 | H03504 |
| receptor type 21 | | | | | | |
| MAP kinase 8 interacting protein 2 | 1.70 | 0.07 | 0.85 | 0.47 | 0.59 | AA418293 |
| MAP kinase kinase kinase 4 | 1.27 | 0.37 | 0.79 | 1.59 | −5.28 | AA402447 |
| MAP kinase kinase kinase 14 | 1.00 | 0.34 | 0.66 | 2.10 | 1.49 | W61116 |
| MAP kinase 8 interacting protein 2 | 2.90 | 0.16 | 0.35 | 0.24 | 0.55 | AI202738 |
| MAP kinase kinase kinase 12 | 1.48 | 0.20 | 0.91 | 0.58 | 0.68 | AA053674 |
| MAP kinase kinase kinase kinase 3 | 2.21 | 0.45 | 0.20 | 1.03 | 0.41 | AA043537 |
| MAP kinase kinase kinase 6 | 2.62 | 0.37 | 0.38 | | 0.70 | AW084649 |
| MAP kinase kinase kinase kinase 4 | 1.04 | 0.96 | 0.09 | 0.29 | 2.79 | AA417711 |
| MAP kinase kinase kinase 11 | 1.53 | 0.65 | 0.41 | 0.99 | 0.44 | R80779 |
| MAP kinase kinase kinase 10 | 1.32 | 1.23 | 0.27 | 0.50 | 0.76 | H01340 |
| MAP kinase 9 | 2.54 | 0.57 | 0.39 | 0.16 | 0.38 | AA157286 |
| MAP kinase kinase kinase 1 | 1.23 | 0.61 | 0.42 | 0.81 | 1.07 | AI538525 |
| MAP kinase kinase kinase 8 | 0.66 | 1.52 | 1.82 | 9.50 | 0.59 | W56266 |
| MAP kinase-activated protein kinase 3 | 0.52 | 2.13 | 2.68 | 1.13 | 1.93 | W68281 |
| MAP kinase kinase 2 | 0.84 | 1.20 | 3.35 | 0.02 | 1.31 | AA425826 |
| MAP kinase kinase kinase 7 | 1.00 | 0.97 | | 1.62 | 7.46 | AA460969 |
| MAP kinase 7 | 0.09 | | 11.45 | 11.80 | 33.43 | H39192 |
| MAP kinase kinase 6 | 0.10 | 17.83 | 9.61 | | 32.30 | H07920 |
| regulator of G-protein signalling 5 | 3.7397 | 0.27 | 0.06 | 0.68 | 0.18 | AA668470 |
| regulator of G-protein signalling 13 | 1.8564 | 0.54 | 0.45 | 0.07 | 1.09 | H70047 |
| G protein-coupled receptor | 1.04 | 1.84 | 0.16 | 0.09 | 0.96 | R91916 |
| G protein-coupled receptor 17 | 1.78 | 0.32 | 0.56 | 0.39 | 0.77 | AI953187 |
| G protein-coupled receptor kinase 7 | 2.62 | | 0.34 | 0.91 | 0.38 | AA488413 |
| orphan seven-transmembrane receptor, | 7.16 | 1.06 | 0.10 | 0.11 | 0.14 | AI131555 |
| chemokine related | | | | | | |
| apoptosis antagonizing transcription | 1.00 | 0.28 | 2.50 | 1.28 | 0.19 | AI439571 |
| factor | | | | | | |
| caspase 1, apoptosis-related cysteine | 2.83 | 0.44 | | 0.33 | 0.35 | T95052 |
| protease (interleukin 1, beta, | | | | | | |
| convertase) | | | | | | |
| programmed cell death 8 (apoptosis- | 1.00 | 1.07 | 0.35 | 1.94 | 0.08 | AA496348 |
| inducing factor) | | | | | | |

The cationic peptides at concentrations of 50 μg/ml were shown to decrease the expression of several polynucleotides. Peptide was incubated with the human A549 epithelial cells for 4 h and the RNA was isolated, converted into labeled cDNA probes and hybridized to Human cDNA arrays ID#PRHU03-S3. The intensity of polynucleotides in unstimulated cells is shown in the second colunm.
The “Ratio Peptide:Unstimulated” columns refers to the intensity of polynucleotide expression in peptide-simulated cells divided by the intensity of unstimulated cells.

TABLE 23
Pro-inflammatory polynucleotides up-regulated by peptide treatment of A549 cells.

| | Unstim. | Ratio Peptide:Unstimulated | | | | Accession |
|---|---|---|---|---|---|---|
| Polynucleotide/Protein and function | Intensity | ID 2 | ID 3 | ID 19 | ID 1 | Number |
| IL-11 Rα; Receptor for pro- | 0.55 | 2.39 | 0.98 | 4.85 | 1.82 | AA454657 |
| inflammatory cytokine, inflammation | | | | | | |
| IL-17 R; Receptor for IL-17, an inducer | 0.54 | 2.05 | 1.97 | 1.52 | 1.86 | AW029299 |
| of cytokine production in epithelial cells | | | | | | |
| small inducible cytokine subfamily A, | 1.00 | 3.88 | | | 2.41 | AI922341 |
| member 21; a chemokine | | | | | | |
| CD31; Leukocyte and cell to cell | 0.59 | 2.71 | 3.13 | 1.01 | 1.68 | R22412 |
| adhesion (PECAM) | | | | | | |
| CCR6; Receptor for chemokine MIP-3α | 0.14 | 4.51 | 7.75 | 6.92 | 7.79 | N57964 |
| integrin, alpha 2 (CD49B, alpha 2 | 1.00 | 0.89 | 2.44 | 3.62 | 0.88 | AA463257 |
| subunit of VLA-2 receptor; Adhesion to | | | | | | |
| leukocytes | | | | | | |
| integrin, alpha 3 (antigen CD49C, alpha | 0.94 | 0.79 | 2.51 | 1.88 | 1.07 | AA424695 |
| 3 subunit of VLA-3 receptor); Leukocyte | | | | | | |
| Adhesion | | | | | | |
| integrin, alpha E; Adhesion | 0.01 | 179.33 | 120.12 | 28.48 | 81.37 | AA425451 |
| integrin, beta 4; Leukocyte adhesion | 0.65 | 0.79 | 2.17 | 4.94 | 1.55 | AA485668 |
| C-type lectin-like receptor-2; Leukocyte | 0.45 | 2.09 | 7.92 | 2.24 | 5.29 | H70491 |
| adhesion | | | | | | |

The cationic peptides at concentrations of 50 μg/m1 were shown to increase the expression of certain pro-inflammatory polynucleotides (data is a subset of Table 21). Peptide was incubated with the human A549 epithelial cells for 4 h and the RNA was isolated, converted into labeled cDNA probes and hybridized to Human cDNA arrays ID#PRHU03-S3. The intensity of polynucleotides in unstimulated cells is shown in the second column.
The “Ratio Peptide:Unstimulated” columns refers to the intensity of polynucleotide expression in peptide-simulated cells divided by the intensity of unstimulated cells.

TABLE 24
Pro-inflammatory polynucleotides down-regulated by peptide treatment of A549 cells.

| | Unstim | Ratio Peptide:Unstimulated | | | | Accession |
|---|---|---|---|---|---|---|
| Polynucleotide/Protein; Function | Intensity | ID 2 | ID 3 | ID 19 | ID 1 | Number |
| Toll-like receptor (TLR) 1; Response to gram | 3.22 | 0.35 | 0.31 | 0.14 | 0.19 | AI339155 |
| positive bacteria | | | | | | |
| TLR 2; Response to gram positive bacteria and | 2.09 | 0.52 | 0.31 | 0.48 | 0.24 | T57791 |
| yeast | | | | | | |
| TLR 5; May augment other TLR responses, | 8.01 | 0.12 | 0.39 | | | N41021 |
| Responsive to flagellin | | | | | | |
| TLR 7: Putative host defense mechanism | 5.03 | 0.13 | 0.11 | 0.20 | 0.40 | N30597 |
| TNF receptor-associated factor 2; Inflammation | 0.82 | 1.22 | 0.45 | 2.50 | 2.64 | T55353 |
| TNF receptor-associated factor 3; Inflammation | 3.15 | 0.15 | | 0.72 | 0.32 | AA504259 |
| TNF receptor superfamily, member 12; | 4.17 | 0.59 | 0.24 | | 0.02 | W71984 |
| Inflammation | | | | | | |
| TNF R superfamily, member 17; Inflammation | 2.62 | | 0.38 | 0.55 | 0.34 | AA987627 |
| TRAF and TNF receptor-associated protein; | 1.33 | 0.75 | 0.22 | 0.67 | 0.80 | AA488650 |
| TNF signalling | | | | | | |
| small inducible cytokine subfamily A, member | 2.26 | 0.32 | | 0.44 | 1.26 | AA495985 |
| 18; Chemokine | | | | | | |
| small inducible cytokine subfamily A, member | 2.22 | 0.19 | 0.38 | 0.45 | 0.90 | AI285199 |
| 20; Chemokine | | | | | | |
| small inducible cytokine subfamily A, member | 2.64 | 0.38 | 0.31 | 1.53 | | AA916836 |
| 23; Chemokine | | | | | | |
| small inducible cytokine subfamily B, member 6 | 3.57 | 0.11 | 0.06 | 0.28 | 0.38 | AI889554 |
| (granulocyte chemotactic protein); Chemokine | | | | | | |
| small inducible cytokine subfamily B, member | 2.02 | 0.50 | 1.07 | 0.29 | 0.40 | AA878880 |
| 10; Chemokine | | | | | | |
| small inducible cytokine A3 (homologous to | 2.84 | 1.79 | 0.32 | 0.35 | | AA677522 |
| mouse Mip-1α); Chemokine | | | | | | |
| IL-12 receptor, beta 2; Interleukin and Interferon | 4.58 | 0.67 | 0.22 | | | AA977194 |
| receptor | | | | | | |
| IL-18 receptor 1; Induces IFN-γ | 1.78 | 0.50 | 0.42 | 0.92 | 0.56 | AA482489 |
| selectin L (lymphocyte adhesion molecule 1); | 4.43 | 0.03 | 0.23 | 0.61 | | H00662 |
| Leukocyte adhesion | | | | | | |
| vascular cell adhesion molecule 1; Leukocyte | 1.40 | 0.20 | 0.72 | 0.77 | 0.40 | H16591 |
| adhesion | | | | | | |
| intercellular adhesion molecule 3; Leukocyte | 1.00 | 0.12 | 0.31 | 2.04 | 1.57 | AA479188 |
| adhesion | | | | | | |
| integrin, alpha 1; Leukocyte adhesion | 2.42 | 0.41 | 0.26 | | 0.56 | AA450324 |

The cationic peptides at concentrations of 50 μg/ml were shown to decrease the expression of certain pro-inflammatory polynucleotides (data is a subset of Table 22). Peptide was incubated with the human A549 epithelial cells for 4 h and the RNA was isolated, converted into labeled cDNA probes and hybridized to Human cDNA arrays ID#PRHU03-S3. The intensity of polynucleotides in unstimulated cells is shown in the second column.
The “Ratio Peptide:Unstimulated” columns refers to the intensity of polynucleotide expression in peptide-simulated cells divided by the intensity of unstimulated cells.

TABLE 25
Anti-inflammatory polynucleotides up-regulated by peptide treatment of A549 cells.

| | Unstim | Ratio Peptide:Unstimulated | | | | Accession |
|---|---|---|---|---|---|---|
| Polynucleotide/Protein; Function | Intensity | ID 2 | ID 3 | ID 19 | ID 1 | Number |
| IL-1 R antagonist homolog 1; | 0.00 | 3085.96 | 1855.90 | 869.57 | | AI167887 |
| Inhibitor of septic shock | | | | | | |
| IL-10 R beta; Receptor for | 0.53 | 2.51 | 1.56 | 1.88 | 3.10 | AA486393 |
| cytokine synthesis inhibitor | | | | | | |
| TNF R, member 1B; Apoptosis | 0.28 | 17.09 | 3.01 | 14.93 | 3.60 | AA150416 |
| TNF R, member 5; Apoptosis | 33.71 | 2.98 | 0.02 | | | H98636 |
| (CD40L) | | | | | | |
| TNF R, member 11b; Apoptosis | 1.00 | 5.29 | 4.50 | 0.78 | | AA194983 |
| IK cytokine, down-regulator of | 0.50 | 3.11 | 2.01 | 1.74 | 3.29 | R39227 |
| HLA II; Inhibits antigen | | | | | | |
| presentation | | | | | | |
| TGFB inducible early growth | 0.90 | 2.38 | 2.08 | 0.87 | 1.11 | AI473938 |
| response 2; anti-inflammatory | | | | | | |
| cytokine | | | | | | |
| CD2; Adhesion molecule, binds | 1.00 | 2.62 | 0.87 | 1.15 | 0.88 | AA927710 |
| LFAp3 | | | | | | |

The cationic peptides at concentrations of 50 μg/ml were shown to increase the expression of certain anti-inflammatory polynucleotides (data is a subset of Table 21). Peptide was incubated with the human A549 epithelial cells for 4 h and the RNA was isolated, converted into labeled cDNA probes and hybridized to Human cDNA arrays ID#PRHU03-S3. The intensity of polynucleotides in unstimulated cells is shown in the second column.
The “Ratio Peptide:Unstimulated” columns refers to the intensity of polynucleotide expression in peptide-simulated cells divided by the intensity of unstimulated cells.

TABLE 26
Anti-inflammatory polynucleotides
down-regulated by peptide treatment of A549 cells.

| Polynucleotide/ | | | | | | |
|---|---|---|---|---|---|---|
| Protein; | Unstim | Ratio Peptide:Unstimulated | | | | Accession |
| Function | Intensity | ID 2 | ID 3 | ID 19 | ID 1 | Number |
| MAP kinase 9 | 2.54 | 0.57 | 0.39 | 0.16 | 0.38 | AA157286 |

The cationic peptides at concentrations of 50 μg/ml were shown to increase the expression of certain anti-inflammatory polynucleotides (data is a subset of Table 21). Peptide was incubated with the human A549 epithelial cells for 4 h and the RNA was isolated, converted into labeled cDNA probes and hybridized to Human cDNA arrays ID#PRHU03-S3. The intensity of polynucleotides in unstimulated cells is shown in the second column.
The “Ratio Peptide:Unstimulated” columns refers to the intensity of polynucleotide expression in peptide-simulated cells divided by the intensity of unstimulated cells.

TABLE 27
Polynucleotides up-regulated by SEQ ID NO: 6, in primary human macrophages.

| | Control: | Ratio peptide |
|---|---|---|
| Gene (Accession Number) | Unstimulated cells | treated:control |
| proteoglycan 2 (Z26248) | 0.69 | 9.3 |
| Unknown (AK001843) | 26.3 | 8.2 |
| phosphorylase kinase alpha 1 (X73874) | 0.65 | 7.1 |
| actinin, alpha 3 (M86407) | 0.93 | 6.9 |
| DKFZP586B2420 protein (AL050143) | 0.84 | 5.9 |
| Unknown (AL109678) | 0.55 | 5.6 |
| transcription factor 21 (AF047419) | 0.55 | 5.4 |
| Unknown (A433612) | 0.62 | 5.0 |
| chromosome condensation 1-like (AF060219) | 0.69 | 4.8 |
| Unknown (AL137715) | 0.66 | 4.4 |
| apoptosis inhibitor 4 (U75285) | 0.55 | 4.2 |
| TERF1 (TRF1)-interacting nuclear factor 2 | 0.73 | 4.2 |
| (NM_012461) | | |
| LINE retrotransposable element 1 (M22333) | 6.21 | 4.0 |
| 1-acylglycerol-3-phosphate O-acyltransferase 1 (U56417) | 0.89 | 4.0 |
| Vacuolar proton-ATPase, subunit D; V- | 1.74 | 4.0 |
| ATPase, subunit D (X71490) | | |
| KIAA0592 protein (AB011164) | 0.70 | 4.0 |
| potassium voltage-gated channel KQT-like | 0.59 | 3.9 |
| subfamily member 4 (AF105202) | | |
| CDC14 homolog A (AF000367) | 0.87 | 3.8 |
| histone fold proteinCHRAC17 (AF070640) | 0.63 | 3.8 |
| Cryptochrome 1 (D83702) | 0.69 | 3.8 |
| pancreatic zymogen granule membrane | 0.71 | 3.7 |
| associated protein (AB035541) | | |
| Sp3 transcription factor (X68560) | 0.67 | 3.6 |
| hypothetical protein FLJ20495 (AK000502) | 0.67 | 3.5 |
| E2F transcription factor 5, p130-binding | 0.56 | 3.5 |
| (U31556) | | |
| hypothetical protein FLJ20070 (AK000077) | 1.35 | 3.4 |
| glycoprotein IX (X52997) | 0.68 | 3.4 |
| KIAA1013 protein (AB023230) | 0.80 | 3.4 |
| eukaryotic translation initiation factor 4A, | 2.02 | 3.4 |
| isoform 2 (AL137681) | | |
| FYN-binding protein (AF198052) | 1.04 | 3.3 |
| guanine nucleotide binding protein, gamma | 0.80 | 3.3 |
| transducing activity polypeptide 1 (U41492) | | |
| glypican 1 (X54232) | 0.74 | 3.2 |
| mucosal vascular addressin cell adhesion | 0.65 | 3.2 |
| molecule 1 (U43628) | | |
| lymphocyte antigen (M38056) | 0.70 | 3.2 |
| H1 histone family, member 4 (M60748) | 0.81 | 3.0 |
| translational inhibitor protein p14.5 (X95384) | 0.78 | 3.0 |
| hypothetical protein FLJ20689 (AB032978) | 1.03 | 2.9 |
| KIAA1278 protein (AB03104) | 0.80 | 2.9 |
| unknown (AL031864) | 0.95 | 2.9 |
| chymotrypsin-like protease (X71877) | 3.39 | 2.9 |
| calumenin (NM_001219) | 2.08 | 2.9 |
| protein kinase, cAMP-dependent, regulatory, | 7.16 | 2.9 |
| type I, beta (M65066) | | |
| POU domain, class 4, transcription factor 2 | 0.79 | 2.8 |
| (U06233) | | |
| POU domain, class 2, associating factor 1 | 1.09 | 2.8 |
| (Z49194) | | |
| KIAA0532 protein (AB011104) | 0.84 | 2.8 |
| unknown (AF068289) | 1.01 | 2.8 |
| unknown (AL117643) | 0.86 | 2.7 |
| cathepsin E (M84424) | 15.33 | 2.7 |
| matrix metalloproteinase 23A (AF056200) | 0.73 | 2.7 |
| interferon receptor 2 (L42243) | 0.70 | 2.5 |
| MAP kinase kinase 1 (L11284) | 0.61 | 2.4 |
| protein kinase C, alpha (X52479) | 0.76 | 2.4 |
| c-Cbl-interacting protein (AF230904) | 0.95 | 2.4 |
| c-fos induced growth factor (Y12864) | 0.67 | 2.3 |
| cyclin-dependent kinase inhibitor 1B (S76988) | 0.89 | 2.2 |
| zinc finger protein 266 (X78924) | 1.67 | 2.2 |
| MAP kinase 14 (L35263) | 1.21 | 2.2 |
| KIAA0922 protein (AB023139) | 0.96 | 2.1 |
| bone morphogenetic protein 1 (NM_006129) | 1.10 | 2.1 |
| NADH dehydrogenase 1 alpha subcomplex, 10 | 1.47 | 2.1 |
| (AF087661) | | |
| bone morphogenetic protein receptor, type IB | 0.50 | 2.1 |
| (U89326) | | |
| interferon regulatory factor 2 (NM 002199) | 1.46 | 2.0 |
| protease, serine, 21 (AB031331) | 0.89 | 2.0 |
| The peptide SEQ ID NO: 6 at a concentration of 50 μg/ml was shown to increase the expression of many polynucleotides. Peptide was incubated with the human macrophages for 4 h and the RNA was isolated, converted into labeled cDNA probes and hybridized to Human Operon arrays (PRHU04). The intensity of polynucleotides in unstimulated cells is shown in the second column. The “Ratio peptide treated:Control” columns refer to the intensity of polynucleotide expression in | | |
| # peptide-simulated cells divided by the intensity of unstimulated cells. | | |

TABLE 28
Polynucleotides down-regulated by SEQ ID NO: 6, in primary human macrophages.

| | Control:Unstimulated | Ratio peptide |
|---|---|---|
| Gene (Accession Number) | cells | treated:control |
| Unknown (AL049263) | 17 | 0.06 |
| integrin-linked kinase (U40282) | 2.0 | 0.13 |
| KIAA0842 protein (AB020649) | 1.1 | 0.13 |
| Unknown (AB037838) | 13 | 0.14 |
| Granulin (AF055008) | 8.6 | 0.14 |
| glutathione peroxidase 3 (NM_002084) | 1.2 | 0.15 |
| KIAA0152 gene product (D63486) | 0.9 | 0.17 |
| TGFB1-induced anti-apoptotic factor 1 | 0.9 | 0.19 |
| (D86970) | | |
| disintegrin protease (Y13323) | 1.5 | 0.21 |
| proteasome subunit beta type 7 (D38048) | 0.7 | 0.22 |
| cofactor required for Sp1 transcriptional | 0.9 | 0.23 |
| activation subunit 3 (AB033042) | | |
| TNF receptor superfamily, member 14 (U81232) | 0.8 | 0.26 |
| proteasome 26S subunit non-ATPase 8 (D38047) | 1.1 | 0.28 |
| proteasome subunit beta type, 4 (D26600) | 0.7 | 0.29 |
| TNF receptor superfamily member 1B (M32315) | 1.7 | 0.29 |
| cytochrome c oxidase subunit Vic (X13238) | 3.3 | 0.30 |
| S100 calcium-binding protein A4 (M80563) | 3.8 | 0.31 |
| proteasome subunit alpha type, 6 (X59417) | 2.9 | 0.31 |
| proteasome 26S subunit non-ATPase, 10 | 1.0 | 0.32 |
| (AL031177) | | |
| MAP kinase kinase kinase 2 (NM_006609) | 0.8 | 0.32 |
| ribosomal protein L11 (X79234) | 5.5 | 0.32 |
| matrix metalloproteinase 14 (Z48481) | 1.0 | 0.32 |
| proteasome subunit beta type, 5 (D29011) | 1.5 | 0.33 |
| MAP kinase-activated protein kinase 2 (U12779) | 1.5 | 0.34 |
| caspase 3 (U13737) | 0.5 | 0.35 |
| jun D proto-oncogene (X56681) | 3.0 | 0.35 |
| proteasome 26S subunit, ATPase, 3 (M34079) | 1.3 | 0.35 |
| IL-1 receptor-like 1 (AB012701) | 0.7 | 0.35 |
| interferon alpha-inducible protein (AB019565) | 13 | 0.35 |
| SDF receptor 1 (NM_012428) | 1.6 | 0.35 |
| Cathepsin D (M63138) | 46 | 0.36 |
| MAP kinase kinase 3 (D87116) | 7.4 | 0.37 |
| TGF, beta-induced, (M77349) | 1.8 | 0.37 |
| TNF receptor superfamily, member 10b | 1.1 | 0.37 |
| (AF016266) | | |
| proteasome subunit beta type, 6 (M34079) | 1.3 | 0.38 |
| nuclear receptor binding protein (NM_013392) | 5.2 | 0.38 |
| Unknown (AL050370) | 1.3 | 0.38 |
| protease inhibitor 1 alpha-1-antitrypsin (X01683) | 0.7 | 0.40 |
| proteasome subunit alpha type, 7 (AF054185) | 5.6 | 0.40 |
| LPS-induced TNF-alpha factor (NM_004862) | 5.3 | 0.41 |
| transferrin receptor (X01060) | 14 | 0.42 |
| proteasome 26S subunit non-ATPase 13 | 1.8 | 0.44 |
| (AB009398) | | |
| MAP kinase kinase 5 (U25265) | 1.3 | 0.44 |
| Cathepsin L (X12451) | 15 | 0.44 |
| IL-1 receptor-associated kinase 1 (L76191) | 1.7 | 0.45 |
| MAP kinase kinase kinase kinase 2 (U07349) | 1.1 | 0.46 |
| peroxisome proliferative activated receptor delta | 2.2 | 0.46 |
| (AL022721) | | |
| TNF superfamily, member 15 (AF039390) | 16 | 0.46 |
| defender against cell death 1 (D15057) | 3.9 | 0.46 |
| TNF superfamily member 10 (U37518) | 287 | 0.46 |
| cathepsin H (X16832) | 14 | 0.47 |
| protease inhibitor 12 (Z81326) | 0.6 | 0.48 |
| proteasome subunit alpha type, 4 (D00763) | 2.6 | 0.49 |
| proteasome 26S subunit ATPase, 1 (L02426) | 1.8 | 0.49 |
| proteasome 26S subunit ATPase, 2 (D11094) | 2.1 | 0.49 |
| caspase 7 (U67319) | 2.4 | 0.49 |
| matrix metalloproteinase 7 (Z11887) | 2.5 | 0.49 |

The peptide SEQ ID NO: 6 at a concentration of 50 μg/ml was shown to increase the expression of many polynucleotides. Peptide was incubated with the human macrophages for 4 h and the RNA was isolated, converted into labeled cDNA probes and hybridized to Human Operon arrays (PRHU04). The intensity of polynucleotides in unstimulated cells is shown in the second column.
The “Ratio of Peptide:Control” columns refer to the intensity of polynucleotide expression in peptide-simulated cells divided by the intensity of unstimulated cells.

TABLE 29
Polynucleotides up-regulated by SEQ ID NO: 1, in HBE cells.

| Accession | | Control:Unstimulated | Ratio peptide |
|---|---|---|---|
| Number | Gene | cells | treated:control |
| AL110161 | Unknown | 0.22 | 5218.3 |
| AF131842 | Unknown | 0.01 | 573.1 |
| AJ000730 | solute carrier family | 0.01 | 282.0 |
| Z25884 | chloride channel 1 | 0.01 | 256.2 |
| M93426 | protein tyrosine phosphatase receptor- | 0.01 | 248.7 |
| | type, zeta | | |
| X65857 | olfactory receptor, family 1, subfamily | 0.01 | 228.7 |
| | D, member 2 | | |
| M55654 | TATA box binding protein | 0.21 | 81.9 |
| AK001411 | hypothetical protein | 0.19 | 56.1 |
| D29643 | dolichyl-diphosphooligosaccharide- | 1.56 | 55.4 |
| | protein glycosyltransferase | | |
| AF006822 | myelin transcription factor 2 | 0.07 | 55.3 |
| AL117601 | Unknown | 0.05 | 53.8 |
| AL117629 | DKFZP434C245 protein | 0.38 | 45.8 |
| M59465 | tumor necrosis factor, alpha-induced | 0.50 | 45.1 |
| | protein 3 | | |
| AB013456 | aquaporin 8 | 0.06 | 41.3 |
| AJ131244 | SEC24 related gene family, member A | 0.56 | 25.1 |
| AL110179 | Unknown | 0.87 | 24.8 |
| AB037844 | Unknwon | 1.47 | 20.6 |
| Z47727 | polymerase II polypeptide K | 0.11 | 20.5 |
| AL035694 | Unknown | 0.81 | 20.4 |
| X68994 | H. sapiens CREB gene | 0.13 | 19.3 |
| AJ238379 | hypothetical protein | 1.39 | 18.5 |
| NM_003519 | H2B histone family member | 0.13 | 18.3 |
| U16126 | glutamate receptor, ionotropic kainate 2 | 0.13 | 17.9 |
| U29926 | adenosine monophosphate deaminase | 0.16 | 16.3 |
| AK001160 | hypothetical protein | 0.39 | 14.4 |
| U18018 | ets variant gene 4 | 0.21 | 12.9 |
| D80006 | KIAA0184 protein | 0.21 | 12.6 |
| AK000768 | hypothetical protein | 0.30 | 12.3 |
| X99894 | insulin promoter factor 1, | 0.26 | 12.0 |
| AL031177 | Unknown | 1.09 | 11.2 |
| AF052091 | unknown | 0.28 | 10.9 |
| L38928 | 5,10-methenyltetrahydrofolate | 0.22 | 10.6 |
| | synthetase | | |
| AL117421 | unknown | 0.89 | 10.1 |
| AL133606 | hypothetical protein | 0.89 | 9.8 |
| NM_016227 | membrane protein CH1 | 0.28 | 9.6 |
| NM_006594 | adaptor-related protein complex 4 | 0.39 | 9.3 |
| U54996 | ZW10 homolog, protein | 0.59 | 9.3 |
| AJ007557 | potassium channel, | 0.28 | 9.0 |
| AF043938 | muscle RAS oncogene | 1.24 | 8.8 |
| AK001607 | unknown | 2.74 | 8.7 |
| AL031320 | peroxisomal biogenesis factor 3 | 0.31 | 8.4 |
| D38024 | unknown | 0.31 | 8.3 |
| AF059575 | LIM homeobox TF | 2.08 | 8.2 |
| AF043724 | hepatitis A virus cellular receptor 1 | 0.39 | 8.1 |
| AK002062 | hypothetical protein | 2.03 | 8.0 |
| L13436 | natriuretic peptide receptor | 0.53 | 7.8 |
| U33749 | thyroid transcription factor 1 | 0.36 | 7.6 |
| AF011792 | cell cycle progression 2 protein | 0.31 | 7.6 |
| AK000193 | hypothetical protein | 1.18 | 6.8 |
| AF039022 | exportin, tRNA | 0.35 | 6.8 |
| M17017 | interleukin 8 | 0.50 | 6.7 |
| AF044958 | NADH dehydrogenase | 0.97 | 6.5 |
| U35246 | vacuolar protein sorting | 0.48 | 6.5 |
| AK001326 | tetraspan 3 | 1.59 | 6.5 |
| M55422 | Krueppel-related zinc finger protein | 0.34 | 6.4 |
| U44772 | palmitoyl-protein thioesterase | 1.17 | 6.3 |
| AL117485 | hypothetical protein | 0.67 | 5.9 |
| AB037776 | unknown | 0.75 | 5.7 |
| AF131827 | unknown | 0.69 | 5.6 |
| AL137560 | unknown | 0.48 | 5.2 |
| X05908 | annexin A1 | 0.81 | 5.1 |
| X68264 | melanoma adhesion molecule | 0.64 | 5.0 |
| AL161995 | neurturin | 0.86 | 4.9 |
| AF037372 | cytochrome c oxidase | 0.48 | 4.8 |
| NM_016187 | bridging integrator 2 | 0.65 | 4.8 |
| AL137758 | unknown | 0.57 | 4.8 |
| U59863 | TRAF family member-associated NFKB | 0.46 | 4.7 |
| | activator | | |
| Z30643 | chloride channel Ka | 0.70 | 4.7 |
| D16294 | acetyl-Coenzyme A acyltransferase 2 | 1.07 | 4.6 |
| AJ132592 | zinc finger protein 281 | 0.55 | 4.6 |
| X82324 | POU domain TF | 1.73 | 4.5 |
| NM_016047 | CGI-110 protein | 1.95 | 4.5 |
| AK001371 | hypothetical protein | 0.49 | 4.5 |
| M60746 | H3 histone family member D | 3.05 | 4.5 |
| AB033071 | hypothetical protein | 4.47 | 4.4 |
| AB002305 | KIAA0307 gene product | 1.37 | 4.4 |
| X92689 | UDP-N-acetyl-alpha-D- | 0.99 | 4.4 |
| | galactosamine:polypeptide N- | | |
| | acetylgalactosaminyltransferase 3 | | |
| AL049543 | glutathione peroxidase 5 | 1.62 | 4.3 |
| U43148 | patched homolog | 0.96 | 4.3 |
| M67439 | dopamine receptor D5 | 2.61 | 4.2 |
| U09850 | zinc finger protein 143 | 0.56 | 4.2 |
| L20316 | glucagon receptor | 0.75 | 4.2 |
| AB037767 | a disintegrin-like and metalloprotease | 0.69 | 4.2 |
| NM_017433 | myosin IIIA | 99.20 | 4.2 |
| D26579 | a disintegrin and metalloprotease domain 8 | 0.59 | 4.1 |
| L10333 | reticulon 1 | 1.81 | 4.1 |
| AK000761 | unknown | 1.87 | 4.1 |
| U91540 | NK homeobox family 3, A | 0.80 | 4.1 |
| Z17227 | interleukin 10 receptor, beta | 0.75 | 4.0 |

The peptide SEQ ID NO: 1 at a concentration of 50 μg/ml was shown to increase the expression of many polynucleotides. Peptide was incubated with the human HBE epithelial cells for 4 h and the RNA was isolated, converted into labeled cDNA probes and hybridized to Human Operon arrays (PRHU04). The intensity of polynucleotides in unstimulated cells is shown in the second column.
The “Ratio Peptide:Control” columns refer to the intensity of polynucleotide expression in peptide-simulated cells divided by the intensity of unstimulated cells.

TABLE 30
Polynucleotides down-regulated by Peptide (50 μg/ml), SEQ ID NO: 1, in HBE cells.

| | | | Ratio of SEQ ID |
|---|---|---|---|
| Accession | | Control:Unstimulated | NO: 1- |
| Number | Gene | Cells | treated:control |
| AC004908 | Unknown | 32.4 | 0.09 |
| S70622 | G1 phase-specific gene | 43.1 | 0.10 |
| Z97056 | DEAD/H box polypeptide | 12.8 | 0.11 |
| AK002056 | hypothetical protein | 11.4 | 0.12 |
| L33930 | CD24 antigen | 28.7 | 0.13 |
| X77584 | thioredoxin | 11.7 | 0.13 |
| NM_014106 | PRO1914 protein | 25.0 | 0.14 |
| M37583 | H2A histone family member | 22.2 | 0.14 |
| U89387 | polymerase (RNA) II polypeptide D | 10.2 | 0.14 |
| D25274 | ras-related C3 botulinum toxin substrate 1 | 10.3 | 0.15 |
| J04173 | phosphoglycerate mutase 1 | 11.4 | 0.15 |
| U19765 | zinc finger protein 9 | 8.9 | 0.16 |
| X67951 | proliferation-associated gene A | 14.1 | 0.16 |
| AL096719 | profilin 2 | 20.0 | 0.16 |
| AF165217 | tropomodulin 4 | 14.6 | 0.16 |
| NM_014341 | mitochondrial carrier homolog 1 | 11.1 | 0.16 |
| AL022068 | Unknown | 73.6 | 0.17 |
| X69150 | ribosomal protein S18 | 42.8 | 0.17 |
| AL031577 | Unknown | 35.0 | 0.17 |
| AL031281 | Unknown | 8.9 | 0.17 |
| AF090094 | Human mRNA for ornithine decarboxylase | 10.3 | 0.17 |
| | antizyme, | | |
| AL022723 | HLA-G histocompatibility antigen, class I, G | 20.6 | 0.18 |
| U09813 | ATP synthase, H+ transporting mitochondrial | 9.8 | 0.18 |
| | F0 complex | | |
| AF000560 | Homo sapiens TTF-I interacting peptide 20 | 20.2 | 0.19 |
| NM_016094 | HSPC042 protein | 67.2 | 0.19 |
| AF047183 | NADH dehydrogenase | 7.5 | 0.19 |
| D14662 | anti-oxidant protein 2 (non-selenium | 8.1 | 0.19 |
| | glutathione peroxidase, acidic calcium- | | |
| | independent phospholipas | | |
| X16662 | annexin A8 | 8.5 | 0.19 |
| U14588 | paxillin | 11.3 | 0.19 |
| AL117654 | DKFZP586D0624 protein | 12.6 | 0.20 |
| AK001962 | hypothetical protein | 7.7 | 0.20 |
| L41559 | 6-pyruvoyl-tetrahydropterin | 9.1 | 0.20 |
| | synthase/dimerization cofactor of hepatocyte | | |
| | nuclear factor 1 alpha | | |
| NM_016139 | 16.7 Kd protein | 21.0 | 0.21 |
| NM_016080 | CGI-150 protein | 10.7 | 0.21 |
| U86782 | 26S proteasome-associated pad1 homolog | 6.7 | 0.21 |
| AJ400717 | tumor protein, translationally-controlled 1 | 9.8 | 0.21 |
| X07495 | homeo box C4 | 31.0 | 0.21 |
| AL034410 | Unknown | 7.3 | 0.22 |
| X14787 | thrombospondin 1 | 26.2 | 0.22 |
| AF081192 | purine-rich element binding protein B | 6.8 | 0.22 |
| D49489 | protein disulfide isomerase-related protein | 11.0 | 0.22 |
| NM_014051 | PTD011 protein | 9.3 | 0.22 |
| AK001536 | Unknown | 98.0 | 0.22 |
| X62534 | high-mobility group protein 2 | 9.5 | 0.22 |
| AJ005259 | endothelial differentiation-related factor 1 | 6.7 | 0.22 |
| NM_000120 | epoxide hydrolase 1, microsomal | 10.0 | 0.22 |
| M38591 | S100 calcium-binding protein A10 | 23.9 | 0.23 |
| AF071596 | immediate early response 3 | 11.5 | 0.23 |
| X16396 | methylene tetrahydrofolate dehydrogenase | 8.3 | 0.23 |
| AK000934 | ATPase inhibitor precursor | 7.6 | 0.23 |
| AL117612 | Unknown | 10.7 | 0.23 |
| AF119043 | transcriptional intermediary factor 1 gamma | 7.3 | 0.23 |
| AF037066 | solute carrier family 22 member 1-like | 7.6 | 0.23 |
| | antisense | | |
| AF134406 | cytochrome c oxidase subunit | 13.3 | 0.23 |
| AE000661 | Unknown | 9.2 | 0.24 |
| AL157424 | synaptojanin 2 | 7.2 | 0.24 |
| X56468 | tyrosine 3-monooxygenase/tryptophan 5- | 7.2 | 0.24 |
| | monooxygenase activation protein, | | |
| U39318 | ubiquitin-conjugating enzyme E2D 3 | 10.7 | 0.24 |
| AL034348 | Unknown | 24.4 | 0.24 |
| D26600 | proteasome subunit beta type 4 | 11.4 | 0.24 |
| AB032987 | Unknown | 16.7 | 0.24 |
| J04182 | lysosomal-associated membrane protein 1 | 7.4 | 0.24 |
| X78925 | zinc finger protein 267 | 16.1 | 0.25 |
| NM_000805 | gastrin | 38.1 | 0.25 |
| U29700 | anti-Mullerian hormone receptor, type II | 12.0 | 0.25 |
| Z98200 | Unknown | 13.4 | 0.25 |
| U07857 | signal recognition particle | 10.3 | 0.25 |
| L05096 | Homo sapiens ribosomal protein L39 | 25.3 | 0.25 |
| AK001443 | hypothetical protein | 7.5 | 0.25 |
| K03515 | glucose phosphate isomerase | 6.2 | 0.25 |
| X57352 | interferon induced transmembrane protein 3 | 7.5 | 0.26 |
| J02883 | colipase pancreatic | 5.7 | 0.26 |
| M24069 | cold shock domain protein | 6.3 | 0.26 |
| AJ269537 | chondroitin-4-sulfotransferase | 60.5 | 0.26 |
| AL137555 | Unknown | 8.5 | 0.26 |
| U89505 | RNA binding motif protein 4 | 5.5 | 0.26 |
| U82938 | CD27-binding protein | 7.5 | 0.26 |
| X99584 | SMT3 homolog 1 | 12.8 | 0.26 |
| AK000847 | Unknown | 35.8 | 0.27 |
| NM_014463 | Lsm3 protein | 7.8 | 0.27 |
| AL133645 | Unknown | 50.8 | 0.27 |
| X78924 | zinc finger protein 266 | 13.6 | 0.27 |
| NM_004304 | anaplastic lymphoma kinase | 15.0 | 0.27 |
| X57958 | ribosomal protein L7 | 27.9 | 0.27 |
| U63542 | Unknown | 12.3 | 0.27 |
| AK000086 | hypothetical protein | 8.3 | 0.27 |
| X57138 | H2A histone family member N | 32.0 | 0.27 |
| AB023206 | KIAA0989 protein | 6.5 | 0.27 |
| AB021641 | gonadotropin inducible transcriptn repressor-1, | 5.5 | 0.28 |
| AF050639 | NADH dehydrogenase | 5.5 | 0.28 |
| M62505 | complement component 5 receptor 1 | 7.5 | 0.28 |
| X64364 | basigin | 5.8 | 0.28 |
| AJ224082 | Unknown | 22.5 | 0.28 |
| AF042165 | cytochrome c oxidase | 20.4 | 0.28 |
| AK001472 | anillin | 10.9 | 0.28 |
| X86428 | protein phosphatase 2A subunit | 12.7 | 0.28 |
| AF227132 | candidate taste receptor T2R5 | 5.1 | 0.28 |
| Z98751 | Unknown | 5.3 | 0.28 |
| D21260 | clathrin heavy polypeptide | 8.3 | 0.28 |
| AF041474 | actin-like 6 | 15.1 | 0.28 |
| NM_005258 | GTP cyclohydrolase I protein | 7.6 | 0.28 |
| L20859 | solute carrier family 20 | 9.6 | 0.29 |
| Z80783 | H2B histone family member | 9.0 | 0.29 |
| AB011105 | laminin alpha 5 | 7.1 | 0.29 |
| AL008726 | protective protein for beta-galactosidase | 5.2 | 0.29 |
| D29012 | proteasome subunit | 12.6 | 0.29 |
| X63629 | cadherin 3 P-cadherin | 6.8 | 0.29 |
| X02419 | plasminogen activator urokinase | 12.9 | 0.29 |
| X13238 | cytochrome c oxidase | 8.0 | 0.29 |
| X59798 | cyclin D1 | 12.7 | 0.30 |
| D78151 | proteasome 26S subunit | 7.6 | 0.31 |
| AF054185 | proteasome subunit | 18.8 | 0.31 |
| J03890 | surfactant pulmonary-associated protein C | 5.5 | 0.32 |
| M34079 | proteasome 26S subunit, | 5.2 | 0.33 |

The peptide SEQ ID NO: 1 at a concentration of 50 μg/ml was shown to decrease the expression of many polynucleotides. Peptide was incubated with the human A549 epithelial cells for 4 h and the RNA was isolated, converted into labeled cDNA probes and hybridized to Human Operon arrays (PRHU04). The intensity of polynucleotides in unstimulated cells is shown in the third column.
The “Ratio Peptide:Control” columns refer to the intensity of polynucleotide expression in peptide-simulated cells divided by the intensity of unstimulated cells.

TABLE 31
Up-regulation of Polynucleotide expression in A549 cells induced by Formula A Peptides.

| Accession | | ctrl- | ctrl 1- | ID 5: | ID 6: | ID 7: | ID 8: | ID 9: | ID 10: |
|---|---|---|---|---|---|---|---|---|---|
| Number | Gene | Cy3 | Cy5 | ctrl | ctrl | ctrl | ctrl | ctrl | ctrl |
| U12472 | glutathione S- | 0.09 | 0.31 | 13.0 | 3.5 | 4.5 | 7.0 | 4.3 | 16.4 |
| | transferase | | | | | | | | |
| X66403 | cholinergic | 0.17 | 0.19 | 7.8 | 9.9 | 6.0 | 6.4 | 5.0 | 15.7 |
| | receptor | | | | | | | | |
| AK001932 | unknown | 0.11 | 0.25 | 19.4 | 4.6 | 9.9 | 7.6 | 8.1 | 14.5 |
| X58079 | S100 calcium- | 0.14 | 0.24 | 12.2 | 7.6 | 8.1 | 4.3 | 4.5 | 13.2 |
| | binding | | | | | | | | |
| | protein | | | | | | | | |
| U18244 | solute carrier | 0.19 | 0.20 | 6.1 | 9.7 | 11.9 | 5.0 | 3.7 | 10.6 |
| | family 1 | | | | | | | | |
| U20648 | zinc finger | 0.16 | 0.13 | 5.3 | 6.2 | 5.6 | 3.1 | 6.8 | 9.5 |
| | protein | | | | | | | | |
| AB037832 | unknown | 0.10 | 0.29 | 9.0 | 4.2 | 9.4 | 3.1 | 2.6 | 8.7 |
| AC002542 | unknown | 0.15 | 0.07 | 10.5 | 15.7 | 7.8 | 10.1 | 11.7 | 8.2 |
| M89796 | membrane- | 0.15 | 0.14 | 2.6 | 6.1 | 7.6 | 3.5 | 13.3 | 8.1 |
| | spanning 4- | | | | | | | | |
| | domains, | | | | | | | | |
| | subfamily A | | | | | | | | |
| AF042163 | cytochrome c | 0.09 | 0.19 | 3.9 | 3.2 | 7.6 | 6.3 | 4.9 | 7.9 |
| | oxidase | | | | | | | | |
| AL032821 | Vanin 2 | 0.41 | 0.23 | 2.5 | 5.2 | 3.2 | 2.1 | 4.0 | 7.9 |
| U25341 | melatonin | 0.04 | 0.24 | 33.1 | 5.1 | 23.3 | 6.6 | 4.1 | 7.6 |
| | receptor 1B | | | | | | | | |
| U52219 | G protein- | 0.28 | 0.20 | 2.1 | 6.2 | 6.9 | 2.4 | 3.9 | 7.1 |
| | coupled | | | | | | | | |
| | receptor | | | | | | | | |
| X04506 | apolipoprotein B | 0.29 | 0.32 | 7.9 | 3.4 | 3.3 | 4.8 | 2.6 | 7.0 |
| AB011138 | ATPase type | 0.12 | 0.07 | 3.5 | 12.9 | 6.6 | 6.4 | 21.3 | 6.9 |
| | IV | | | | | | | | |
| AF055018 | unknown | 0.28 | 0.22 | 3.8 | 6.9 | 5.0 | 2.3 | 3.1 | 6.8 |
| AK002037 | hypothetical | 0.08 | 0.08 | 2.9 | 7.9 | 14.1 | 7.9 | 20.1 | 6.5 |
| | protein | | | | | | | | |
| AK001024 | guanine | 0.16 | 0.11 | 7.7 | 11.9 | 5.0 | 10.3 | 6.0 | 6.3 |
| | nucleotide- | | | | | | | | |
| | binding | | | | | | | | |
| | protein | | | | | | | | |
| AF240467 | TLR-7 | 0.11 | 0.10 | 20.4 | 9.0 | 3.4 | 9.4 | 12.9 | 6.1 |
| AF105367 | glucagon-like | 0.15 | 0.35 | 23.2 | 2.6 | 3.0 | 10.6 | 2.9 | 5.7 |
| | peptide 2 | | | | | | | | |
| | receptor | | | | | | | | |
| AL009183 | TNFR | 0.46 | 0.19 | 10.6 | 4.7 | 3.7 | 2.8 | 6.5 | 5.7 |
| | superfamily, | | | | | | | | |
| | member 9 | | | | | | | | |
| X54380 | pregnancy- | 0.23 | 0.08 | 4.7 | 11.9 | 7.2 | 12.7 | 3.8 | 5.5 |
| | zone protein | | | | | | | | |
| AL137736 | unknown | 0.22 | 0.15 | 2.1 | 7.2 | 3.3 | 7.1 | 4.6 | 5.5 |
| X05615 | thyroglobulin | 0.28 | 0.42 | 6.3 | 2.7 | 7.7 | 2.4 | 3.1 | 5.4 |
| D28114 | myelin- | 0.24 | 0.08 | 2.5 | 15.9 | 13.0 | 7.1 | 13.7 | 5.4 |
| | associated | | | | | | | | |
| | protein | | | | | | | | |
| AK000358 | microfibrillar- | 0.28 | 0.28 | 8.7 | 4.2 | 7.2 | 3.2 | 2.4 | 5.3 |
| | associated | | | | | | | | |
| | protein 3 | | | | | | | | |
| AK001351 | unknown | 0.12 | 0.22 | 3.9 | 7.6 | 8.7 | 3.9 | 2.3 | 5.2 |
| U79289 | unknown | 0.14 | 0.27 | 2.5 | 2.7 | 2.8 | 2.0 | 4.3 | 5.1 |
| AB014546 | ring finger | 0.12 | 0.34 | 6.8 | 2.4 | 4.1 | 2.7 | 2.0 | 5.0 |
| | protein | | | | | | | | |
| AL117428 | DKFZP434A2 | 0.10 | 0.07 | 2.8 | 16.1 | 12.8 | 9.7 | 14.2 | 4.9 |
| | 36 protein | | | | | | | | |
| AL050378 | unknown | 0.41 | 0.14 | 3.5 | 8.7 | 11.7 | 3.5 | 7.0 | 4.9 |
| AJ250562 | transmembrane | 0.13 | 0.10 | 5.2 | 5.7 | 14.2 | 3.8 | 10.3 | 4.8 |
| | 4 superfamily | | | | | | | | |
| | member 2 | | | | | | | | |
| NM_001756 | corticosteroid | 0.28 | 0.13 | 4.0 | 7.9 | 6.5 | 14.9 | 5.6 | 4.8 |
| | binding | | | | | | | | |
| | globulin | | | | | | | | |
| AL137471 | hypothetical | 0.29 | 0.05 | 3.7 | 18.0 | 6.2 | 7.2 | 16.3 | 4.7 |
| | protein | | | | | | | | |
| M19684 | protease | 0.41 | 0.14 | 3.5 | 4.6 | 5.4 | 2.8 | 9.4 | 4.7 |
| | inhibitor 1 | | | | | | | | |
| NM_001963 | epidermal | 0.57 | 0.05 | 3.4 | 6.2 | 1.8 | 32.9 | 14.7 | 4.4 |
| | growth factor | | | | | | | | |
| NM_000910 | neuropeptide | 0.62 | 0.36 | 3.1 | 2.7 | 2.3 | 2.6 | 3.1 | 4.4 |
| | Y receptor | | | | | | | | |
| AF022212 | Rho GTPase | 0.19 | 0.02 | 9.0 | 45.7 | 25.6 | 12.4 | 72.2 | 4.4 |
| | activating | | | | | | | | |
| | protein 6 | | | | | | | | |
| AK001674 | cofactor | 0.11 | 0.13 | 8.4 | 6.5 | 7.9 | 4.5 | 7.4 | 4.3 |
| | required for | | | | | | | | |
| | Sp1 | | | | | | | | |
| U51920 | signal | 0.23 | 0.27 | 3.4 | 3.8 | 2.1 | 4.1 | 8.8 | 4.2 |
| | recognition | | | | | | | | |
| | particle | | | | | | | | |
| AK000576 | hypothetical | 0.27 | 0.06 | 4.4 | 14.7 | 7.4 | 14.1 | 8.6 | 4.2 |
| | protein | | | | | | | | |
| AL080073 | unknown | 0.17 | 0.20 | 21.6 | 3.9 | 4.3 | 8.8 | 2.6 | 4.1 |
| U59628 | paired box | 0.34 | 0.06 | 3.4 | 14.1 | 5.4 | 7.9 | 4.9 | 4.1 |
| | gene 9 | | | | | | | | |
| U90548 | butyrophilin, | 0.41 | 0.31 | 2.3 | 4.7 | 5.5 | 6.8 | 3.4 | 4.1 |
| | subfamily 3, | | | | | | | | |
| | member A3 | | | | | | | | |
| M19673 | cystatin SA | 0.43 | 0.26 | 2.3 | 8.5 | 4.5 | 2.5 | 4.1 | 3.8 |
| AL161972 | ICAM 2 | 0.44 | 0.37 | 2.0 | 3.6 | 2.0 | 2.7 | 5.5 | 3.8 |
| X54938 | inositol 1,4,5- | 0.32 | 0.22 | 3.9 | 3.3 | 6.2 | 3.1 | 4.4 | 3.7 |
| | trisphosphate | | | | | | | | |
| | 3-kinase A | | | | | | | | |
| AB014575 | KIAA0675 | 0.04 | 0.13 | 46.2 | 4.5 | 10.2 | 8.0 | 6.2 | 3.4 |
| | gene product | | | | | | | | |
| M83664 | MHC II, DP | 0.57 | 0.29 | 2.9 | 2.1 | 2.0 | 3.1 | 6.6 | 3.4 |
| | beta 1 | | | | | | | | |
| AK000043 | hypothetical | 0.34 | 0.14 | 2.7 | 7.1 | 3.7 | 9.4 | 8.8 | 3.3 |
| | protein | | | | | | | | |
| U60666 | testis specific | 0.21 | 0.11 | 9.9 | 9.0 | 4.1 | 5.5 | 13.0 | 3.3 |
| | leucine rich | | | | | | | | |
| | repeat protein | | | | | | | | |
| AK000337 | hypothetical | 0.49 | 0.19 | 4.3 | 5.1 | 4.7 | 10.6 | 7.1 | 3.3 |
| | protein | | | | | | | | |
| AF050198 | putative | 0.34 | 0.15 | 7.0 | 6.3 | 3.6 | 5.6 | 11.9 | 3.3 |
| | mitochondrial | | | | | | | | |
| | space protein | | | | | | | | |
| AJ251029 | odorant- | 0.28 | 0.12 | 4.4 | 9.4 | 7.2 | 8.8 | 7.1 | 3.2 |
| | binding | | | | | | | | |
| | protein 2A | | | | | | | | |
| X74142 | forkhead box | 0.12 | 0.33 | 19.5 | 4.5 | 8.4 | 6.4 | 4.4 | 3.2 |
| | G1B | | | | | | | | |
| AB029033 | KIAA1110 | 0.35 | 0.24 | 3.1 | 2.2 | 5.6 | 5.2 | 3.1 | 3.1 |
| | protein | | | | | | | | |
| D85606 | cholecystokinin | 0.51 | 0.14 | 4.3 | 3.9 | 4.6 | 3.5 | 7.2 | 3.1 |
| | A receptor | | | | | | | | |
| X84195 | acylphosphatase | 0.32 | 0.19 | 4.8 | 3.7 | 5.0 | 11.2 | 9.8 | 3.0 |
| | 2 muscle type | | | | | | | | |
| U57971 | ATPase Ca++ | 0.29 | 0.13 | 2.2 | 7.9 | 1.8 | 6.3 | 4.8 | 3.0 |
| | transporting | | | | | | | | |
| | plasma | | | | | | | | |
| | membrane 3 | | | | | | | | |
| J02611 | apolipoprotein D | 0.28 | 0.10 | 2.8 | 11.0 | 3.7 | 10.3 | 8.4 | 3.0 |
| AF071510 | lecithin retinol | 0.07 | 0.05 | 7.9 | 3.8 | 11.7 | 46.0 | 16.3 | 3.0 |
| | acyltransferase | | | | | | | | |
| AF131757 | unknown | 0.10 | 0.08 | 4.8 | 9.0 | 44.3 | 9.3 | 10.7 | 3.0 |
| L10717 | IL2-inducible | 0.45 | 0.21 | 2.5 | 4.9 | 2.8 | 10.9 | 4.5 | 2.9 |
| | T-cell kinase | | | | | | | | |
| L32961 | 4-aminobutyrate | 0.64 | 0.32 | 3.6 | 2.9 | 3.2 | 5.3 | 2.3 | 2.9 |
| | aminotransferase | | | | | | | | |
| NM_003631 | poly (ADP- | 0.46 | 0.41 | 9.7 | 3.9 | 4.1 | 3.8 | 2.8 | 2.7 |
| | ribose) | | | | | | | | |
| | glycohydrolase | | | | | | | | |
| AF098484 | pronapsin A | 0.28 | 0.14 | 3.7 | 3.7 | 5.6 | 11.6 | 3.7 | 2.5 |
| NM_009589 | arylsulfatase D | 0.73 | 0.16 | 3.2 | 5.6 | 6.0 | 48.6 | 7.2 | 2.4 |
| M14764 | TNFR | 0.49 | 0.15 | 2.3 | 3.5 | 10.6 | 13.6 | 6.8 | 2.2 |
| | superfamily, | | | | | | | | |
| | member 16 | | | | | | | | |
| AL035250 | endothelin 3 | 0.52 | 0.14 | 2.1 | 7.3 | 4.8 | 4.5 | 3.7 | 2.2 |
| M97925 | defensin, | 0.33 | 0.07 | 4.0 | 14.7 | 7.8 | 9.4 | 3.5 | 2.1 |
| | alpha 5, | | | | | | | | |
| | Paneth cell- | | | | | | | | |
| | specific | | | | | | | | |
| D43945 | transcription | 0.46 | 0.19 | 6.6 | 2.9 | 8.2 | 4.0 | 3.5 | 2.1 |
| | factor EC | | | | | | | | |
| D16583 | histidine | 0.46 | 0.09 | 3.2 | 13.8 | 4.2 | 8.8 | 13.7 | 2.1 |
| | decarboxylase | | | | | | | | |

The peptides at a concentration of 50 μg/ml were shown to increase the expression of many polynucleotides. Peptide was incubated with the human A549 epithelial cells for 4 h and the RNA was isolated, converted into labeled cDNA probes and hybridized to Human Operon arrays (PRHU04). The intensity of poIynueleotides in control, unstimulated cells are shown in the second and third columns for labeling of cDNA with the dyes Cy3 and Cy5 respectively.
The “ID#: Control” columns refer to the intensity of polynucleotide expression in peptidesimulated cells divided by the intensity of unstimulated cells.

TABLE 32
Up-regulation of Polynucleotide expression in A549 cells induced by Formula B Peptides.

| Accession | | ctrl- | ctrl- | ID 12: | ID 13: | ID 14: | ID 15: | ID 16: | ID 17: |
|---|---|---|---|---|---|---|---|---|---|
| Number | Gene | Cy3 | Cy5 | ctrl | ctrl | ctrl | ctrl | ctrl | ctrl |
| AL157466 | unknown | 0.05 | 0.06 | 18.0 | 21.4 | 16.7 | 5.2 | 6.8 | 8.6 |
| AB023215 | KIAA0998 | 0.19 | 0.07 | 14.8 | 10.6 | 7.9 | 14.4 | 6.6 | 16.1 |
| | protein | | | | | | | | |
| AL031121 | unknown | 0.24 | 0.09 | 14.1 | 5.7 | 3.8 | 5.5 | 2.8 | 4.6 |
| NM_016331 | zinc finger | 0.16 | 0.08 | 12.8 | 7.2 | 11.0 | 5.3 | 11.2 | 9.7 |
| | protein | | | | | | | | |
| M14565 | cytochrome | 0.16 | 0.12 | 10.6 | 12.5 | 5.0 | 3.6 | 10.1 | 6.3 |
| | P450 | | | | | | | | |
| U22492 | G protein- | 0.28 | 0.07 | 10.4 | 8.9 | 4.8 | 10.8 | 6.6 | 3.6 |
| | coupled | | | | | | | | |
| | receptor 8 | | | | | | | | |
| U76010 | solute carrier | 0.14 | 0.07 | 9.7 | 18.6 | 3.7 | 4.8 | 5.6 | 8.9 |
| | family 30 | | | | | | | | |
| AK000685 | unknown | 0.51 | 0.10 | 9.0 | 3.1 | 2.8 | 3.9 | 15.3 | 3.0 |
| AF013620 | Immunoglobulin | 0.19 | 0.18 | 8.5 | 2.6 | 6.2 | 5.7 | 8.2 | 3.8 |
| | heavy variable | | | | | | | | |
| | 4—4 | | | | | | | | |
| AL049296 | unknown | 0.61 | 0.89 | 8.1 | 3.2 | 2.7 | 3.2 | 2.7 | 2.0 |
| AB006622 | KIAA0284 | 0.47 | 0.28 | 7.5 | 5.0 | 2.8 | 11.1 | 5.5 | 4.6 |
| | protein | | | | | | | | |
| X04391 | CD5 antigen | 0.22 | 0.13 | 7.2 | 16.7 | 2.7 | 7.7 | 6.1 | 5.9 |
| AK000067 | hypothetical | 0.80 | 0.35 | 7.1 | 4.6 | 2.1 | 3.2 | 8.5 | 2.2 |
| | protein | | | | | | | | |
| AF053712 | TNF | 0.17 | 0.08 | 6.9 | 17.7 | 3.0 | 6.2 | 12.3 | 5.2 |
| | superfamily_member | | | | | | | | |
| | 11 | | | | | | | | |
| X58079 | S100 calcium- | 0.14 | 0.24 | 6.7 | 6.7 | 5.9 | 6.5 | 5.3 | 2.5 |
| | binding protein | | | | | | | | |
| | A1 | | | | | | | | |
| M91036 | hemoglobin_gamma A | 0.48 | 0.36 | 6.7 | 14.2 | 2.1 | 2.9 | 2.7 | 4.8 |
| AF055018 | unknown | 0.28 | 0.22 | 6.3 | 10.7 | 2.7 | 2.6 | 4.6 | 6.5 |
| L17325 | pre-T/NK cell | 0.19 | 0.29 | 6.1 | 4.4 | 6.5 | 4.7 | 4.0 | 4.0 |
| | associated | | | | | | | | |
| | protein | | | | | | | | |
| D45399 | phosphodiesterase | 0.21 | 0.18 | 6.1 | 4.6 | 5.0 | 2.8 | 10.8 | 4.0 |
| AB023188 | KIAA0971 | 0.29 | 0.13 | 5.9 | 10.6 | 3.6 | 3.4 | 10.6 | 7.2 |
| | protein | | | | | | | | |
| NM_012177 | F-box protein | 0.26 | 0.31 | 5.9 | 5.5 | 3.8 | 2.8 | 3.0 | 6.8 |
| D38550 | E2F TF 3 | 0.43 | 0.39 | 5.8 | 3.4 | 2.1 | 4.5 | 2.5 | 2.4 |
| AL050219 | unknown | 0.26 | 0.04 | 5.7 | 17.0 | 3.1 | 9.2 | 30.3 | 16.1 |
| AL137540 | unknown | 0.67 | 0.79 | 5.5 | 3.2 | 3.9 | 10.9 | 2.9 | 2.3 |
| D50926 | KIAA0136 | 0.57 | 0.21 | 5.4 | 5.6 | 2.0 | 3.3 | 4.4 | 3.2 |
| | protein | | | | | | | | |
| AL137658 | unknown | 0.31 | 0.07 | 5.4 | 12.1 | 2.6 | 10.8 | 3.9 | 8.6 |
| U21931 | fructose- | 0.48 | 0.14 | 5.4 | 4.1 | 2.9 | 3.6 | 6.0 | 3.2 |
| | bisphosphatase 1 | | | | | | | | |
| AK001230 | DKFZP586D211 | 0.43 | 0.26 | 5.0 | 4.6 | 2.1 | 2.2 | 2.5 | 2.7 |
| | protein | | | | | | | | |
| AL137728 | unknown | 0.67 | 0.47 | 5.0 | 5.9 | 2.2 | 6.8 | 5.9 | 2.1 |
| AB022847 | unknown | 0.39 | 0.24 | 4.5 | 2.2 | 3.5 | 4.3 | 3.8 | 3.7 |
| X75311 | mevalonate | 0.67 | 0.22 | 4.3 | 4.0 | 2.0 | 8.3 | 4.0 | 5.1 |
| | kinase | | | | | | | | |
| AK000946 | DKFZP566C243 | 0.36 | 0.29 | 4.1 | 3.8 | 3.9 | 5.4 | 25.8 | 2.7 |
| | protein | | | | | | | | |
| AB023197 | KIAA0980 | 0.25 | 0.30 | 4.0 | 8.3 | 2.1 | 8.8 | 2.2 | 4.9 |
| | protein | | | | | | | | |
| AB014615 | fibroblast | 0.19 | 0.07 | 3.9 | 3.3 | 7.0 | 3.4 | 2.2 | 7.7 |
| | growth factor 8 | | | | | | | | |
| X04014 | unknown | 0.29 | 0.16 | 3.8 | 2.5 | 2.2 | 3.0 | 5.5 | 3.1 |
| U76368 | solute carrier | 0.46 | 0.17 | 3.8 | 3.8 | 2.8 | 3.2 | 4.2 | 3.0 |
| | family 7 | | | | | | | | |
| AB032436 | unknown | 0.14 | 0.21 | 3.8 | 2.7 | 6.1 | 3.2 | 4.5 | 2.6 |
| AB020683 | KIAA0876 | 0.37 | 0.21 | 3.7 | 4.2 | 2.2 | 5.3 | 2.9 | 9.4 |
| | protein | | | | | | | | |
| NM_012126 | carbohydrate | 0.31 | 0.20 | 3.7 | 5.2 | 3.2 | 3.4 | 3.9 | 2.5 |
| | sulfotransferase 5 | | | | | | | | |
| AK002037 | hypothetical | 0.08 | 0.08 | 3.7 | 17.1 | 4.6 | 12.3 | 11.0 | 8.7 |
| | protein | | | | | | | | |
| X78712 | glycerol kinase | 0.17 | 0.19 | 3.6 | 2.5 | 4.5 | 5.3 | 2.2 | 3.3 |
| | pseudogene 2 | | | | | | | | |
| NM_014178 | HSPC156 | 0.23 | 0.12 | 3.5 | 8.4 | 2.9 | 6.9 | 14.4 | 5.5 |
| | protein | | | | | | | | |
| AC004079 | homeo box A2 | 0.31 | 0.11 | 3.5 | 7.0 | 2.1 | 2.0 | 7.3 | 9.1 |
| AL080182 | unknown | 0.51 | 0.21 | 3.4 | 3.5 | 2.2 | 2.1 | 2.9 | 2.4 |
| M91036 | hemoglobin | 0.22 | 0.02 | 3.4 | 26.3 | 5.8 | 6.8 | 30.4 | 21.6 |
| | gamma G | | | | | | | | |
| AJ000512 | serum/glucocort | 0.27 | 0.43 | 3.3 | 2.1 | 4.9 | 2.3 | 3.9 | 2.7 |
| | icoid regulated | | | | | | | | |
| | kinase | | | | | | | | |
| AK002140 | hypothetical | 0.28 | 0.14 | 3.3 | 9.9 | 2.8 | 2.1 | 16.6 | 7.2 |
| | protein | | | | | | | | |
| AL137284 | unknown | 0.22 | 0.04 | 3.3 | 7.2 | 4.1 | 6.0 | 12.2 | 3.7 |
| Z11898 | POU domain_class | 0.12 | 0.29 | 3.2 | 3.7 | 8.2 | 2.5 | 6.6 | 2.2 |
| | 5 TF 1 | | | | | | | | |
| AB017016 | brain-specific | 0.27 | 0.29 | 3.1 | 2.8 | 2.5 | 2.8 | 3.3 | 5.5 |
| | protein | | | | | | | | |
| X54673 | Solute-carrier | 0.34 | 0.08 | 2.9 | 12.0 | 2.2 | 10.4 | 7.4 | 5.9 |
| | family 6 | | | | | | | | |
| AL033377 | unknown | 0.40 | 0.22 | 2.6 | 2.6 | 2.6 | 2.3 | 4.5 | 2.2 |
| X85740 | CCR4 | 0.34 | 0.05 | 2.6 | 2.3 | 2.6 | 2.5 | 12.5 | 5.2 |
| AB010419 | core-binding | 0.59 | 0.20 | 2.5 | 12.8 | 2.0 | 2.8 | 2.9 | 5.9 |
| | factor | | | | | | | | |
| AL109726 | uknown | 0.14 | 0.15 | 2.3 | 9.0 | 4.3 | 4.4 | 2.6 | 3.7 |
| NM_012450 | sulfate | 0.15 | 0.10 | 2.2 | 3.1 | 8.2 | 9.9 | 4.7 | 5.9 |
| | transporter 1 | | | | | | | | |
| J04599 | biglycan | 0.39 | 0.30 | 2.1 | 3.3 | 6.6 | 2.2 | 2.7 | 5.4 |
| AK000266 | hypothetical | 0.49 | 0.35 | 2.1 | 3.5 | 3.5 | 6.6 | 4.3 | 4.0 |
| | protein | | | | | | | | |

The peptides at a concentration of 50 μg/ml were shown to increase the expression of many polynucleotides. Peptide was incubated with the human A549 epithelial cells for 4 h and the RNA was isolated, converted into labeled cDNA probes and hybridized to Human Operon arrays (PRHU04). The intensity of polynucleotides in control, unstimulated cells are shown in the second and third columns for labeling of cDNA with the dyes Cy3 and Cy5 respectively.
The “ID#: Control” columns refer to the intensity of polynucleotide expression in peptide-simulated cells divided by the intensity of unstimulated cells.

TABLE 33
Up-regulation of Polynucleotide expression in A549 cells induced by Formula C Peptides.

| Accession | | ctrl- | ctrl- | ID 19: | ID 20: | ID 21: | ID 22: | ID 23: | ID 24: |
|---|---|---|---|---|---|---|---|---|---|
| Number | Gene | Cy3 | Cy5 | ctrl | ctrl | ctrl | ctrl | ctrl | ctrl |
| NM_014139 | sodium | 0.04 | 0.05 | 31.6 | 25.2 | 18.0 | 9.7 | 22.2 | 11.2 |
| | channel | | | | | | | | |
| | voltage- | | | | | | | | |
| | gated, | | | | | | | | |
| X84003 | TATA box | 0.47 | 0.07 | 31.8 | 12.7 | 2.5 | 2.8 | 18.0 | 14.2 |
| | binding | | | | | | | | |
| | protein | | | | | | | | |
| AF144412 | lens epithelial | 0.25 | 0.07 | 23.9 | 8.0 | 6.8 | 3.4 | 16.2 | 3.5 |
| | cell protein | | | | | | | | |
| AL080107 | unknown | 0.11 | 0.06 | 17.8 | 34.4 | 12.4 | 6.2 | 5.4 | 7.9 |
| AF052116 | unknown | 0.34 | 0.07 | 15.5 | 3.9 | 9.2 | 3.0 | 6.9 | 2.7 |
| AB033063 | unknown | 0.46 | 0.13 | 15.2 | 10.3 | 4.0 | 2.6 | 7.2 | 11.2 |
| AK000258 | hypothetical | 0.27 | 0.07 | 13.9 | 8.0 | 3.5 | 3.4 | 26.5 | 11.5 |
| | protein | | | | | | | | |
| NM_006963 | zinc finger | 0.10 | 0.08 | 12.8 | 6.8 | 6.2 | 5.9 | 17.2 | 1241.2 |
| | protein | | | | | | | | |
| NM_014099 | PRO1768 | 0.30 | 0.06 | 12.3 | 17.4 | 5.4 | 5.4 | 19.5 | 3.4 |
| | protein | | | | | | | | |
| AK000996 | hypothetical | 0.17 | 0.07 | 10.0 | 8.0 | 9.7 | 7.4 | 20.7 | 16.3 |
| | protein | | | | | | | | |
| M81933 | cell division | 0.13 | 0.21 | 8.8 | 7.8 | 19.6 | 15.6 | 4.8 | 3.8 |
| | cycle 25A | | | | | | | | |
| AF181286 | unknown | 0.05 | 0.22 | 8.8 | 2.7 | 12.0 | 35.6 | 5.9 | 2.3 |
| AJ272208 | IL-1R | 0.22 | 0.17 | 8.8 | 2.9 | 5.0 | 3.2 | 9.8 | 7.3 |
| | accessory | | | | | | | | |
| | protein-like 2 | | | | | | | | |
| AF030555 | fatty-acid- | 0.10 | 0.39 | 8.7 | 2.2 | 11.3 | 9.9 | 3.0 | 2.1 |
| | Coenzyme A | | | | | | | | |
| | ligase | | | | | | | | |
| AL050125 | unknown | 0.23 | 0.07 | 8.6 | 14.3 | 5.2 | 2.8 | 18.7 | 8.3 |
| AB011096 | KIAA0524 | 0.21 | 0.08 | 8.5 | 24.4 | 4.7 | 6.8 | 10.4 | 7.5 |
| | protein | | | | | | | | |
| J03068 | N- | 0.54 | 0.21 | 8.3 | 2.4 | 2.2 | 4.1 | 3.0 | 6.0 |
| | acylaminoacyl- | | | | | | | | |
| | peptide | | | | | | | | |
| | hydrolase | | | | | | | | |
| M33906 | MHC class | 0.14 | 0.08 | 7.6 | 4.5 | 15.2 | 6.1 | 7.5 | 7.9 |
| | II, DQ alpha 1 | | | | | | | | |
| AJ272265 | secreted | 0.21 | 0.09 | 7.6 | 9.0 | 3.3 | 4.9 | 18.8 | 14.5 |
| | phosphoprotein | | | | | | | | |
| J00210 | interferon | 0.41 | 0.07 | 7.2 | 15.0 | 2.8 | 3.1 | 11.0 | 4.3 |
| | alpha 13 | | | | | | | | |
| AK001952 | hypothetical | 0.42 | 0.21 | 6.9 | 4.9 | 2.5 | 3.1 | 7.6 | 4.5 |
| | protein | | | | | | | | |
| X54131 | protein | 0.09 | 0.20 | 6.4 | 6.5 | 7.7 | 15.0 | 5.6 | 4.1 |
| | tyrosine | | | | | | | | |
| | phosphatase, | | | | | | | | |
| | receptor type, | | | | | | | | |
| AF064493 | LIM binding | 0.46 | 0.14 | 5.9 | 5.6 | 2.2 | 2.9 | 8.5 | 5.8 |
| | domain 2 | | | | | | | | |
| AL117567 | DKFZP566O | 0.44 | 0.22 | 5.8 | 3.3 | 2.9 | 2.3 | 5.7 | 14.9 |
| | 084 protein | | | | | | | | |
| L40933 | phosphoglucomutase 5 | 0.16 | 0.03 | 5.6 | 11.0 | 4.8 | 3.5 | 8.5 | 76.3 |
| M27190 | regenerating | 0.19 | 0.28 | 5.3 | 3.0 | 3.8 | 3.6 | 5.8 | 3.6 |
| | islet-derived | | | | | | | | |
| | 1 alpha | | | | | | | | |
| AL031121 | unknown | 0.24 | 0.09 | 5.3 | 3.8 | 3.2 | 3.9 | 3.0 | 27.9 |
| U27655 | regulator of | 0.24 | 0.29 | 5.0 | 9.0 | 4.5 | 8.3 | 4.2 | 4.5 |
| | G-protein | | | | | | | | |
| | signalling | | | | | | | | |
| AB037786 | unknown | 0.12 | 0.03 | 4.7 | 54.1 | 2.8 | 2.3 | 2.2 | 11.0 |
| X73113 | myosin- | 0.29 | 0.13 | 4.7 | 6.5 | 6.0 | 2.4 | 6.7 | 6.3 |
| | binding | | | | | | | | |
| | protein C | | | | | | | | |
| AB010962 | matrix | 0.08 | 0.12 | 4.7 | 6.2 | 2.4 | 4.7 | 10.9 | 4.2 |
| | metalloproteinase | | | | | | | | |
| AL096729 | unknown | 0.36 | 0.13 | 4.7 | 7.7 | 3.2 | 2.4 | 6.3 | 6.2 |
| AB018320 | Arg/Abl- | 0.16 | 0.18 | 4.6 | 7.1 | 3.0 | 3.3 | 5.8 | 8.9 |
| | interacting | | | | | | | | |
| | protein | | | | | | | | |
| AK001024 | guanine | 0.16 | 0.11 | 4.6 | 2.0 | 9.8 | 2.6 | 7.6 | 14.1 |
| | nucleotide- | | | | | | | | |
| | binding | | | | | | | | |
| | protein | | | | | | | | |
| AJ275355 | unknown | 0.15 | 0.08 | 4.6 | 17.3 | 5.4 | 9.2 | 5.1 | 5.5 |
| U21931 | fructose- | 0.48 | 0.14 | 4.6 | 4.3 | 2.6 | 2.1 | 8.4 | 9.6 |
| | bisphosphatase 1 | | | | | | | | |
| X66403 | cholinergic | 0.17 | 0.19 | 4.4 | 9.0 | 10.9 | 9.3 | 5.1 | 6.7 |
| | receptor | | | | | | | | |
| X67734 | contactin 2 | 0.25 | 0.09 | 4.3 | 6.8 | 3.1 | 5.8 | 7.9 | 8.4 |
| U92981 | unknown | 0.20 | 0.23 | 4.3 | 3.2 | 4.8 | 5.6 | 5.4 | 6.3 |
| X68879 | empty | 0.05 | 0.08 | 4.3 | 2.0 | 12.3 | 2.7 | 5.6 | 4.7 |
| | spiracles | | | | | | | | |
| | homolog 1 | | | | | | | | |
| AL137362 | unknown | 0.22 | 0.22 | 4.2 | 4.1 | 2.7 | 4.1 | 9.3 | 4.2 |
| NM_001756 | corticosteroid | 0.28 | 0.13 | 4.1 | 10.6 | 3.9 | 2.7 | 10.3 | 5.5 |
| | binding | | | | | | | | |
| | globulin | | | | | | | | |
| U80770 | unknown | 0.31 | 0.14 | 4.1 | 4.1 | 23.3 | 2.7 | 7.0 | 10.1 |
| AL109792 | unknown | 0.16 | 0.19 | 4.0 | 4.5 | 4.3 | 8.8 | 8.7 | 3.9 |
| X65962 | cytochrome | 0.33 | 0.05 | 3.8 | 25.3 | 5.7 | 5.1 | 19.8 | 12.0 |
| | P-450 | | | | | | | | |
| AK001856 | unknown | 0.40 | 0.21 | 3.8 | 7.0 | 2.6 | 3.1 | 2.9 | 7.8 |
| AL022723 | MHC, class I, F | 0.55 | 0.18 | 3.7 | 5.7 | 4.4 | 2.3 | 3.3 | 5.2 |
| D38449 | putative G | 0.18 | 0.09 | 3.5 | 11.1 | 13.3 | 5.8 | 4.8 | 5.2 |
| | protein | | | | | | | | |
| | coupled | | | | | | | | |
| | receptor | | | | | | | | |
| AL137489 | unknown | 0.74 | 0.26 | 3.3 | 2.9 | 2.6 | 3.3 | 2.5 | 5.4 |
| AB000887 | small | 0.76 | 0.18 | 3.3 | 5.0 | 2.6 | 2.4 | 5.9 | 10.3 |
| | inducible | | | | | | | | |
| | cytokine | | | | | | | | |
| | subfamily A | | | | | | | | |
| NM_012450 | sulfate | 0.15 | 0.10 | 3.3 | 9.0 | 10.0 | 10.9 | 4.6 | 8.7 |
| | transporter 1 | | | | | | | | |
| U86529 | glutathione | 0.55 | 0.15 | 3.2 | 6.8 | 4.4 | 2.3 | 9.3 | 5.1 |
| | S-transferase | | | | | | | | |
| | zeta 1 | | | | | | | | |
| AK001244 | unknown | 0.79 | 0.31 | 3.2 | 5.5 | 2.3 | 2.3 | 3.9 | 2.8 |
| AL133602 | unknown | 0.16 | 0.21 | 3.1 | 7.8 | 8.7 | 2.6 | 4.1 | 5.6 |
| AB033080 | cell cycle | 0.31 | 0.31 | 3.1 | 4.6 | 3.0 | 3.5 | 2.2 | 4.2 |
| | progression 8 | | | | | | | | |
| | protein | | | | | | | | |
| AF023466 | putative | 0.27 | 0.18 | 3.1 | 5.0 | 4.2 | 7.4 | 10.1 | 3.8 |
| | glycine-N- | | | | | | | | |
| | acyltransferase | | | | | | | | |
| AL117457 | cofilin 2 | 0.68 | 0.53 | 3.0 | 4.6 | 3.3 | 2.4 | 7.4 | 3.4 |
| AC007059 | unknown | 0.37 | 0.35 | 3.0 | 5.7 | 3.1 | 2.4 | 2.6 | 2.4 |
| U60179 | growth | 0.34 | 0.21 | 2.9 | 3.5 | 2.3 | 3.1 | 8.0 | 4.7 |
| | hormone | | | | | | | | |
| | receptor | | | | | | | | |
| M37238 | phospholipase | 0.60 | 0.36 | 2.9 | 2.0 | 3.2 | 2.1 | 2.9 | 4.6 |
| | C, gamma 2 | | | | | | | | |
| L22569 | cathepsin B | 0.32 | 0.12 | 2.9 | 2.1 | 6.2 | 3.0 | 13.1 | 16.7 |
| M80359 | MAP/microtubule | 0.37 | 0.76 | 2.9 | 3.1 | 6.1 | 7.6 | 2.1 | 3.3 |
| | affinity- | | | | | | | | |
| | regulating | | | | | | | | |
| | kinase 3 | | | | | | | | |
| S70348 | Integrin beta 3 | 0.58 | 0.31 | 2.6 | 4.8 | 4.1 | 2.6 | 2.6 | 2.6 |
| L13720 | growth | 0.36 | 0.26 | 2.4 | 2.5 | 6.8 | 4.8 | 3.9 | 3.7 |
| | arrest- | | | | | | | | |
| | specific 6 | | | | | | | | |
| AL049423 | unknown | 0.33 | 0.30 | 2.4 | 3.7 | 3.8 | 2.8 | 2.9 | 3.4 |
| AL050201 | unknown | 0.68 | 0.29 | 2.2 | 3.1 | 3.7 | 3.0 | 3.0 | 2.2 |
| AF050078 | growth arrest | 0.87 | 0.33 | 2.1 | 8.4 | 2.5 | 2.2 | 2.6 | 4.4 |
| | specific 11 | | | | | | | | |
| AK001753 | hypothetical | 0.53 | 0.28 | 2.1 | 5.0 | 2.2 | 2.8 | 3.6 | 4.6 |
| | protein | | | | | | | | |
| X05323 | unknown | 0.39 | 0.13 | 2.1 | 7.8 | 2.6 | 2.4 | 21.5 | 3.5 |
| AB014548 | KIAA0648 | 0.61 | 0.30 | 2.0 | 2.4 | 4.8 | 3.4 | 4.9 | 3.9 |
| | protein | | | | | | | | |

The peptides at a concentration of 50 μg/ml were shown to increase the expression of many polynucleotides. Peptide was incubated with the human A549 epithelial cells for 4 h and the RNA was isolated, converted into labeled cDNA probes and hybridized to Human Operon arrays (PRHU04). The intensity of polynucleotides in control, unstimulated cells are shown in the second and third columns for labeling of cDNA with the dyes Cy3 and Cy5 respectively.
The “ID#: Control” columns refer to the intensity of polynucleotide expression in peptide-simulated cells divided by the intensity of unstimulated cells.

TABLE 34
Up-regulation of Polynucleotide expression in A549 cells induced by Formula D Peptides.

| Accession | | ctrl- | ctrl- | | | | | | |
|---|---|---|---|---|---|---|---|---|---|
| Number | Gene | Cy3 | Cy5 | ID 26:ctrl | ID 27:ctrl | ID 28:ctrl | ID 29:ctrl | ID 30:ctrl | ID 31:ctrl |
| U68018 | MAD homolog 2 | 0.13 | 0.71 | 11.2 | 2.2 | 8.0 | 2.3 | 6.7 | 25.6 |
| NM_016015 | CGI-68 protein | 0.92 | 1.59 | 2.3 | 2.3 | 3.5 | 3.7 | 3.4 | 22.9 |
| AF071510 | lecithin retinol | 0.07 | 0.05 | 15.4 | 10.3 | 5.3 | 44.1 | 2.1 | 21.2 |
| | acyltransferase | | | | | | | | |
| AC005154 | unkown | 0.17 | 1.13 | 2.7 | 7.2 | 12.6 | 6.4 | 3.3 | 20.6 |
| M81933 | cell division | 0.13 | 0.21 | 4.3 | 3.1 | 3.2 | 4.3 | 5.6 | 18.2 |
| | cycle 25A | | | | | | | | |
| AF124735 | LIM HOX | 0.17 | 0.21 | 2.1 | 4.4 | 5.9 | 5.2 | 7.6 | 17.0 |
| | gene 2 | | | | | | | | |
| AL110125 | unknown | 0.30 | 0.08 | 5.0 | 2.7 | 6.8 | 10.2 | 2.8 | 12.0 |
| NM_004732 | potassium | 0.15 | 0.16 | 7.6 | 4.0 | 3.4 | 2.2 | 2.9 | 11.4 |
| | voltage-gated | | | | | | | | |
| | channel | | | | | | | | |
| AF030555 | fatty-acid- | 0.10 | 0.39 | 10.5 | 2.2 | 6.4 | 3.0 | 5.1 | 10.7 |
| | Coenzyme A | | | | | | | | |
| | ligase_long- | | | | | | | | |
| | chain 4 | | | | | | | | |
| AF000237 | 1-acylglycerol- | 1.80 | 2.37 | 3.4 | 2.5 | 2.4 | 2.1 | 3.7 | 9.9 |
| | 3-phosphate O- | | | | | | | | |
| | acyltransferase 2 | | | | | | | | |
| AL031588 | hypothetical | 0.40 | 0.26 | 5.8 | 20.2 | 2.8 | 4.7 | 5.6 | 9.1 |
| | protein | | | | | | | | |
| AL080077 | unknown | 0.15 | 0.21 | 2.4 | 2.0 | 11.9 | 3.8 | 2.3 | 8.7 |
| NM_014366 | putative | 0.90 | 2.52 | 2.4 | 4.3 | 2.4 | 2.6 | 3.0 | 8.6 |
| | nucleotide | | | | | | | | |
| | binding | | | | | | | | |
| | protein_estradiol- | | | | | | | | |
| | induced | | | | | | | | |
| AB002359 | phosphoribosyl | 0.81 | 2.12 | 3.2 | 2.7 | 5.5 | 2.5 | 2.8 | 6.9 |
| | formylglycinamidine | | | | | | | | |
| | synthase | | | | | | | | |
| U33547 | MHC class II | 0.14 | 0.16 | 2.5 | 5.3 | 4.5 | 5.0 | 3.1 | 6.6 |
| | antigen HLA- | | | | | | | | |
| | DRB6 mRNA_{—} | | | | | | | | |
| AL133051 | unknown | 0.09 | 0.07 | 7.7 | 6.3 | 5.4 | 23.1 | 5.4 | 6.5 |
| AK000576 | hypothetical | 0.27 | 0.06 | 7.1 | 9.3 | 5.0 | 6.9 | 2.9 | 6.2 |
| | protein | | | | | | | | |
| AF042378 | spindle pole | 0.36 | 0.39 | 3.3 | 3.0 | 9.5 | 4.5 | 3.4 | 6.2 |
| | body protein | | | | | | | | |
| AF093265 | Homer | 0.67 | 0.53 | 2.7 | 13.3 | 6.5 | 5.0 | 2.9 | 6.2 |
| | neuronal | | | | | | | | |
| | immediate | | | | | | | | |
| | early gene_3 | | | | | | | | |
| D80000 | Segregation of | 1.01 | 1.56 | 3.6 | 2.5 | 4.9 | 3.2 | 6.3 | 6.1 |
| | mitotic | | | | | | | | |
| | chromosomes 1 | | | | | | | | |
| AF035309 | proteasome | 3.61 | 4.71 | 2.7 | 6.6 | 5.2 | 4.9 | 2.7 | 6.0 |
| | 26S subunit | | | | | | | | |
| | ATPase 5 | | | | | | | | |
| M34175 | adaptor-related | 4.57 | 5.13 | 3.2 | 3.1 | 4.0 | 4.6 | 2.7 | 6.0 |
| | protein | | | | | | | | |
| | complex 2 beta | | | | | | | | |
| | 1 subunit | | | | | | | | |
| AB020659 | KIAA0852 | 0.18 | 0.37 | 4.1 | 7.6 | 5.7 | 4.8 | 2.5 | 5.7 |
| | protein | | | | | | | | |
| NM_004862 | LPS-induced | 2.61 | 3.36 | 3.8 | 4.8 | 4.1 | 4.9 | 3.2 | 5.6 |
| | TNF-alpha | | | | | | | | |
| | factor | | | | | | | | |
| U00115 | zinc finger | 0.51 | 0.07 | 18.9 | 2.2 | 3.5 | 7.2 | 21.2 | 5.6 |
| | protein 51 | | | | | | | | |
| AF088868 | fibrousheathin | 0.45 | 0.20 | 4.7 | 10.0 | 3.2 | 6.4 | 6.0 | 5.6 |
| | II | | | | | | | | |
| AK001890 | unknown | 0.42 | 0.55 | 2.4 | 3.5 | 3.6 | 2.3 | 2.2 | 5.6 |
| AL137268 | KIAA0759 | 0.49 | 0.34 | 3.8 | 2.3 | 5.0 | 3.5 | 3.3 | 5.4 |
| | protein | | | | | | | | |
| X63563 | polymerase II | 1.25 | 1.68 | 2.5 | 8.1 | 3.4 | 4.8 | 5.2 | 5.4 |
| | polypeptide B | | | | | | | | |
| D12676 | CD36 antigen | 0.35 | 0.39 | 2.9 | 3.4 | 2.6 | 2.2 | 3.5 | 5.3 |
| AK000161 | hypothetical | 1.06 | 0.55 | 3.4 | 8.7 | 2.1 | 6.7 | 2.9 | 5.1 |
| | protein | | | | | | | | |
| AF052138 | unknown | 0.64 | 0.51 | 2.9 | 2.8 | 2.7 | 5.2 | 3.6 | 5.0 |
| AL096803 | unknown | 0.36 | 0.03 | 20.1 | 18.3 | 3.7 | 19.3 | 16.1 | 4.9 |
| S49953 | DNA-binding | 0.70 | 0.15 | 3.7 | 4.0 | 2.1 | 6.6 | 4.0 | 4.8 |
| | transcriptional | | | | | | | | |
| | activator | | | | | | | | |
| X89399 | RAS p21 | 0.25 | 0.10 | 8.5 | 14.9 | 4.8 | 18.6 | 4.3 | 4.8 |
| | protein | | | | | | | | |
| | activator | | | | | | | | |
| AJ005273 | antigenic | 0.70 | 0.10 | 7.6 | 11.1 | 2.8 | 9.9 | 12.0 | 4.6 |
| | determinant of | | | | | | | | |
| | recA protein | | | | | | | | |
| AK001154 | hypothetical | 1.70 | 0.96 | 2.4 | 4.4 | 2.9 | 8.9 | 2.4 | 4.5 |
| | protein | | | | | | | | |
| AL133605 | unknown | 0.26 | 0.15 | 12.4 | 4.2 | 4.4 | 3.3 | 3.3 | 4.1 |
| U71092 | G protein- | 0.53 | 0.06 | 19.0 | 9.1 | 2.2 | 12.0 | 3.3 | 4.1 |
| | coupled | | | | | | | | |
| | receptor 24 | | | | | | | | |
| AF074723 | RNA | 0.67 | 0.54 | 4.0 | 3.2 | 3.1 | 3.4 | 6.0 | 4.0 |
| | polymerase II | | | | | | | | |
| | transcriptional | | | | | | | | |
| | regulation | | | | | | | | |
| | mediator | | | | | | | | |
| AL137577 | unknown | 0.32 | 0.12 | 31.4 | 6.2 | 5.3 | 10.1 | 25.3 | 3.9 |
| AF151043 | hypothetical | 0.48 | 0.35 | 2.6 | 2.2 | 2.0 | 3.3 | 2.2 | 3.8 |
| | protein | | | | | | | | |
| AF131831 | unknown | 0.67 | 0.81 | 2.1 | 7.0 | 3.5 | 3.2 | 3.9 | 3.7 |
| D50405 | histone | 1.52 | 2.62 | 3.1 | 7.2 | 2.9 | 4.1 | 2.8 | 3.7 |
| | deacetylase 1 | | | | | | | | |
| U78305 | protein | 1.21 | 0.20 | 4.7 | 13.0 | 3.5 | 5.9 | 4.2 | 3.7 |
| | phosphatase | | | | | | | | |
| | 1D | | | | | | | | |
| AL035562 | paired box | 0.24 | 0.01 | 30.2 | 81.9 | 5.6 | 82.3 | 6.2 | 3.7 |
| | gene 1 | | | | | | | | |
| U67156 | mitogen- | 1.15 | 0.30 | 6.6 | 3.0 | 2.2 | 2.3 | 2.5 | 3.6 |
| | activated | | | | | | | | |
| | protein kinase | | | | | | | | |
| | kinase kinase 5 | | | | | | | | |
| AL031121 | unknown | 0.24 | 0.09 | 5.2 | 3.7 | 2.3 | 6.5 | 9.1 | 3.6 |
| U13666 | G protein- | 0.34 | 0.14 | 3.8 | 5.4 | 3.1 | 3.3 | 2.8 | 3.6 |
| | coupled | | | | | | | | |
| | receptor 1 | | | | | | | | |
| AB018285 | KIAA0742 | 0.53 | 0.13 | 14.9 | 13.9 | 5.9 | 18.5 | 15.2 | 3.5 |
| | protein | | | | | | | | |
| D42053 | site-1 protease | 0.63 | 0.40 | 2.6 | 7.1 | 5.6 | 9.2 | 2.6 | 3.5 |
| AK001135 | Sec23- | 0.29 | 0.53 | 5.7 | 4.5 | 3.4 | 2.6 | 11.3 | 3.4 |
| | interacting | | | | | | | | |
| | protein p125 | | | | | | | | |
| AL137461 | unknown | 0.25 | 0.02 | 23.8 | 9.0 | 2.7 | 59.2 | 12.5 | 3.3 |
| NM_006963 | zinc finger | 0.10 | 0.08 | 3.2 | 7.6 | 3.7 | 7.9 | 11.2 | 3.2 |
| | protein 22 | | | | | | | | |
| AL137540 | unknown | 0.67 | 0.79 | 3.9 | 2.6 | 5.6 | 4.2 | 3.5 | 3.1 |
| AL137718 | unknown | 0.95 | 0.18 | 4.7 | 8.0 | 4.0 | 13.3 | 3.0 | 3.1 |
| AF012086 | RAN binding | 1.20 | 0.59 | 4.6 | 4.0 | 2.0 | 4.6 | 3.6 | 3.1 |
| | protein 2-like 1 | | | | | | | | |
| S57296 | HER2/neu | 0.59 | 0.17 | 7.3 | 12.1 | 2.3 | 20.0 | 22.2 | 3.0 |
| | receptor | | | | | | | | |
| NM_013329 | GC-rich | 0.16 | 0.08 | 6.9 | 14.3 | 9.7 | 3.3 | 7.2 | 3.0 |
| | sequence | | | | | | | | |
| | DNA-binding | | | | | | | | |
| | factor | | | | | | | | |
| | candidate | | | | | | | | |
| AF038664 | UDP-Gal:beta | 0.15 | 0.03 | 13.4 | 22.2 | 5.4 | 15.8 | 17.6 | 3.0 |
| | GlcNAc beta | | | | | | | | |
| | 1_4- | | | | | | | | |
| | galactosyltransferase | | | | | | | | |
| AF080579 | Homo sapiens | 0.34 | 1.03 | 3.3 | 3.0 | 6.7 | 2.1 | 2.9 | 2.9 |
| | integral | | | | | | | | |
| | membrane | | | | | | | | |
| | protein | | | | | | | | |
| AK001075 | hypothetical | 0.67 | 0.10 | 2.1 | 2.6 | 2.6 | 8.9 | 2.2 | 2.9 |
| | protein | | | | | | | | |
| AB011124 | KIAA0552 | 0.46 | 0.04 | 9.6 | 72.0 | 6.0 | 33.9 | 13.6 | 2.9 |
| | gene product | | | | | | | | |
| J03068 | N- | 0.54 | 0.21 | 2.2 | 5.0 | 2.4 | 5.2 | 3.6 | 2.8 |
| | acylaminoacyl- | | | | | | | | |
| | peptide | | | | | | | | |
| | hydrolase | | | | | | | | |
| D87120 | osteoblast | 0.87 | 0.87 | 2.2 | 2.0 | 4.7 | 2.3 | 2.0 | 2.8 |
| | protein | | | | | | | | |
| AB006537 | IL-1R | 0.17 | 0.07 | 2.9 | 7.0 | 14.5 | 5.3 | 6.6 | 2.8 |
| | accessory | | | | | | | | |
| | protein | | | | | | | | |
| L34587 | transcription | 2.49 | 1.23 | 2.2 | 16.3 | 5.0 | 15.8 | 5.5 | 2.7 |
| | elongation | | | | | | | | |
| | factor B | | | | | | | | |
| D31891 | SET domain_bifurcated_1 | 1.02 | 0.29 | 3.9 | 6.0 | 4.3 | 4.9 | 6.6 | 2.7 |
| D00760 | proteasome | 4.97 | 4.94 | 4.1 | 2.6 | 2.0 | 2.8 | 2.7 | 2.7 |
| | subunit_alpha | | | | | | | | |
| | type_2 | | | | | | | | |
| AC004774 | distal-less | 0.25 | 0.12 | 2.3 | 6.3 | 3.8 | 5.2 | 5.2 | 2.6 |
| | homeo box 5 | | | | | | | | |
| AL024493 | unknown | 1.46 | 0.54 | 4.8 | 13.5 | 2.1 | 11.6 | 6.8 | 2.6 |
| AB014536 | copine III | 1.80 | 1.29 | 3.2 | 9.5 | 3.8 | 6.8 | 2.6 | 2.6 |
| X59770 | IL-1R type II | 0.59 | 0.16 | 9.6 | 4.7 | 3.9 | 3.2 | 4.9 | 2.5 |
| AF052183 | unknown | 0.65 | 0.76 | 4.0 | 3.7 | 2.3 | 5.0 | 3.0 | 2.5 |
| AK000541 | hypothetical | 0.92 | 0.27 | 4.5 | 13.9 | 3.6 | 18.1 | 4.3 | 2.5 |
| | protein | | | | | | | | |
| U88528 | cAMP | 1.37 | 0.86 | 3.1 | 5.4 | 2.1 | 2.8 | 2.1 | 2.4 |
| | responsive | | | | | | | | |
| | element | | | | | | | | |
| | binding protein | | | | | | | | |
| M97925 | defensin alpha | 0.33 | 0.07 | 4.6 | 35.9 | 2.0 | 7.8 | 6.5 | 2.4 |
| | 5_Paneth cell- | | | | | | | | |
| | specific | | | | | | | | |
| NM_013393 | cell division | 1.38 | 0.94 | 3.1 | 5.8 | 2.1 | 4.2 | 2.6 | 2.3 |
| | protein FtsJ | | | | | | | | |
| X62744 | MHC class II | 0.86 | 0.32 | 4.0 | 4.7 | 2.3 | 2.9 | 6.1 | 2.3 |
| | DM alpha | | | | | | | | |
| AF251040 | putative | 0.64 | 0.30 | 6.7 | 3.4 | 2.9 | 3.9 | 5.7 | 2.2 |
| | nuclear protein | | | | | | | | |
| AK000227 | hypothetical | 1.49 | 0.43 | 3.4 | 7.1 | 2.3 | 3.3 | 9.1 | 2.1 |
| | protein | | | | | | | | |
| U88666 | SFRS protein | 1.78 | 0.37 | 3.4 | 5.9 | 2.6 | 8.4 | 6.1 | 2.0 |
| | kinase 2 | | | | | | | | |

The peptides at a concentration of 50 μg/ml were shown to increase the expression of many polynucleotides. Peptide was incubated with the human A549 epithelial cells for 4 h and the RNA was isolated, converted into labeled DNA probes and hybridized to Human Operon arrays (PRHU04). The intensity of polynucleotides in control, unstimulated cells are shown in the second and third columns for labeling of cDNA with the dyes Cy3 and Cy5 respectively.
The “ID #:Control” columns refer to the intensity of polynucleotide expression in peptide-simulated cells divided by the intensity of unstimulated cells.

TABLE 35
Up-regulation of Polynucleotide expression in A549 cells induced by Formula E Peptides.

| Accession | | ctrl- | ctrl- | | | | | | |
|---|---|---|---|---|---|---|---|---|---|
| Number | Gene | Cy3 | Cy5 | ID 33:ctrl | ID 34:ctrl | ID 35:ctrl | ID 36:ctrl | ID 37:ctrl | ID 38:ctrl |
| AL049689 | Novel human | 0.25 | 0.05 | 2.7 | 26.5 | 3.3 | 21.7 | 5.4 | 37.9 |
| | mRNA | | | | | | | | |
| AK000576 | hypothetical | 0.27 | 0.06 | 3.0 | 19.1 | 3.9 | 23.0 | 3.1 | 28.3 |
| | protein | | | | | | | | |
| X74837 | mannosidase, | 0.10 | 0.07 | 5.6 | 10.0 | 10.8 | 12.3 | 12.0 | 19.9 |
| | alpha class | | | | | | | | |
| | 1A member 1 | | | | | | | | |
| AK000258 | hypothetical | 0.27 | 0.07 | 14.0 | 11.1 | 7.9 | 16.1 | 6.2 | 18.9 |
| | protein | | | | | | | | |
| X89067 | transient | 0.20 | 0.14 | 3.7 | 2.2 | 2.4 | 2.6 | 8.0 | 18.1 |
| | receptor | | | | | | | | |
| AL137619 | unknown | 0.16 | 0.08 | 6.3 | 6.7 | 10.8 | 10.5 | 7.9 | 16.5 |
| NM_003445 | zinc finger | 0.17 | 0.07 | 4.0 | 23.6 | 2.9 | 13.6 | 4.3 | 14.4 |
| | protein | | | | | | | | |
| X03084 | complement | 0.36 | 0.15 | 2.4 | 3.1 | 2.9 | 7.7 | 3.4 | 13.7 |
| | component 1 | | | | | | | | |
| U27330 | fucosyltransferase 5 | 0.39 | 0.08 | 2.4 | 2.5 | 2.6 | 12.1 | 3.5 | 13.0 |
| AF070549 | unknown | 0.16 | 0.09 | 2.7 | 4.7 | 7.9 | 10.3 | 4.2 | 12.6 |
| AB020335 | sel-1-like | 0.19 | 0.24 | 2.9 | 2.6 | 2.0 | 7.3 | 4.7 | 12.4 |
| M26901 | renin | 0.09 | 0.12 | 14.9 | 2.2 | 7.3 | 12.0 | 20.8 | 12.0 |
| Y07828 | ring finger | 0.09 | 0.06 | 9.0 | 26.6 | 8.9 | 16.0 | 3.6 | 11.6 |
| | protein | | | | | | | | |
| AK001848 | hypothetical | 0.21 | 0.07 | 6.2 | 8.2 | 2.7 | 5.2 | 5.5 | 10.9 |
| | protein | | | | | | | | |
| NM_016331 | zinc finger | 0.16 | 0.08 | 7.6 | 5.1 | 7.0 | 25.5 | 5.5 | 10.9 |
| | protein | | | | | | | | |
| U75330 | neural cell | 0.42 | 0.08 | 2.5 | 3.6 | 2.0 | 5.8 | 6.2 | 9.9 |
| | adhesion | | | | | | | | |
| | molecule 2 | | | | | | | | |
| AB037826 | unknown | 0.16 | 0.11 | 3.8 | 6.0 | 3.4 | 13.4 | 6.0 | 9.8 |
| M34041 | adrenergic | 0.30 | 0.13 | 4.5 | 4.5 | 3.7 | 8.6 | 5.6 | 9.8 |
| | alpha-2B- | | | | | | | | |
| | receptor | | | | | | | | |
| D38449 | putative G | 0.18 | 0.09 | 2.3 | 25.8 | 11.7 | 2.3 | 3.2 | 9.5 |
| | protein | | | | | | | | |
| | coupled | | | | | | | | |
| | receptor | | | | | | | | |
| AJ250562 | transmembrane | 0.13 | 0.10 | 10.0 | 8.4 | 2.2 | 8.1 | 16.3 | 9.1 |
| | 4 superfamily | | | | | | | | |
| | member 2 | | | | | | | | |
| AK001807 | hypothetical | 0.18 | 0.12 | 4.2 | 5.3 | 4.6 | 3.2 | 4.0 | 8.3 |
| | protein | | | | | | | | |
| AL133051 | unknown | 0.09 | 0.07 | 5.1 | 13.6 | 6.0 | 9.1 | 2.2 | 8.2 |
| U43843 | Neuro-d4 | 0.61 | 0.10 | 2.0 | 6.4 | 2.3 | 16.6 | 2.2 | 8.1 |
| | homolog | | | | | | | | |
| NM_013227 | aggrecan 1 | 0.28 | 0.15 | 7.5 | 3.1 | 2.5 | 6.9 | 8.5 | 7.8 |
| AF226728 | somatostatin | 0.23 | 0.17 | 7.0 | 3.6 | 3.1 | 5.5 | 3.5 | 7.7 |
| | receptor- | | | | | | | | |
| | interacting | | | | | | | | |
| | protein | | | | | | | | |
| AK001024 | guanine | 0.16 | 0.11 | 3.9 | 12.3 | 2.7 | 7.4 | 3.3 | 7.0 |
| | nucleotide- | | | | | | | | |
| | binding | | | | | | | | |
| | protein | | | | | | | | |
| AC002302 | unknown | 0.13 | 0.14 | 16.1 | 5.8 | 5.8 | 2.6 | 9.6 | 6.2 |
| AB007958 | unknown | 0.17 | 0.27 | 2.0 | 2.3 | 11.3 | 3.3 | 3.0 | 6.1 |
| AF059293 | cytokine | 0.19 | 0.22 | 3.6 | 2.5 | 10.2 | 3.8 | 2.7 | 5.9 |
| | receptor-like | | | | | | | | |
| | factor 1 | | | | | | | | |
| V01512 | v-fos | 0.27 | 0.21 | 6.7 | 3.7 | 13.7 | 9.3 | 3.7 | 5.4 |
| U82762 | sialyltransferase 8 | 0.23 | 0.15 | 3.2 | 6.5 | 2.7 | 9.2 | 5.7 | 5.4 |
| U44059 | thyrotrophic | 0.05 | 0.13 | 22.9 | 7.1 | 12.5 | 7.4 | 9.7 | 5.4 |
| | embryonic | | | | | | | | |
| | factor | | | | | | | | |
| X05323 | antigen | 0.39 | 0.13 | 4.3 | 2.5 | 2.2 | 7.4 | 2.8 | 5.1 |
| | identified by | | | | | | | | |
| | monoclonal | | | | | | | | |
| | antibody | | | | | | | | |
| U72671 | ICAM 5, | 0.25 | 0.14 | 5.3 | 2.7 | 3.7 | 10.0 | 3.2 | 4.8 |
| AL133626 | hypothetical | 0.26 | 0.25 | 2.2 | 4.2 | 2.9 | 3.0 | 2.6 | 4.7 |
| | protein | | | | | | | | |
| X96401 | MAX | 0.31 | 0.29 | 6.9 | 2.3 | 4.9 | 3.1 | 2.9 | 4.6 |
| | binding | | | | | | | | |
| | protein | | | | | | | | |
| AL117533 | unknown | 0.05 | 0.26 | 8.2 | 2.7 | 11.1 | 2.5 | 11.9 | 4.5 |
| AK001550 | hypothetical | 0.10 | 0.30 | 8.0 | 2.0 | 4.9 | 2.1 | 7.8 | 4.5 |
| | protein | | | | | | | | |
| AB032436 | Homo | 0.14 | 0.21 | 5.1 | 2.2 | 9.1 | 4.5 | 6.4 | 4.4 |
| | sapiens BNPI | | | | | | | | |
| | mRNA | | | | | | | | |
| AL035447 | hypothetical | 0.28 | 0.23 | 4.3 | 3.7 | 8.7 | 5.2 | 3.7 | 4.2 |
| | protein | | | | | | | | |
| U09414 | zinc finger | 0.28 | 0.25 | 4.0 | 2.2 | 4.7 | 3.3 | 7.2 | 4.2 |
| | protein | | | | | | | | |
| AK001256 | unknown | 0.09 | 0.08 | 5.3 | 6.5 | 31.1 | 12.7 | 6.4 | 4.1 |
| L14813 | carboxyl | 0.64 | 0.21 | 2.7 | 6.2 | 3.1 | 2.1 | 3.4 | 3.9 |
| | ester lipase- | | | | | | | | |
| | like | | | | | | | | |
| AF038181 | unknowan | 0.06 | 0.18 | 34.1 | 6.4 | 4.5 | 8.7 | 11.3 | 3.9 |
| NM_001486 | glucokinase | 0.21 | 0.08 | 3.0 | 2.2 | 6.5 | 12.4 | 5.7 | 3.9 |
| AB033000 | hypothetical | 0.24 | 0.22 | 3.4 | 3.3 | 7.1 | 5.5 | 4.5 | 3.8 |
| | protein | | | | | | | | |
| AL117567 | DKFZP566O084 | 0.44 | 0.22 | 2.2 | 2.7 | 3.9 | 4.0 | 4.5 | 3.7 |
| | protein | | | | | | | | |
| NM_012126 | carbohydrate | 0.31 | 0.20 | 5.5 | 5.4 | 3.8 | 5.5 | 2.6 | 3.5 |
| | sulfotransferase 5 | | | | | | | | |
| AL031687 | unknown | 0.16 | 0.27 | 5.9 | 2.6 | 3.4 | 2.3 | 4.9 | 3.5 |
| X04506 | apolipoprotein B | 0.29 | 0.32 | 5.4 | 4.4 | 6.9 | 5.5 | 2.1 | 3.5 |
| NM_006641 | CCR9 | 0.35 | 0.11 | 3.3 | 3.3 | 2.2 | 16.5 | 2.3 | 3.5 |
| Y00970 | acrosin | 0.12 | 0.14 | 8.2 | 8.8 | 3.1 | 6.2 | 17.5 | 3.4 |
| X67098 | rTS beta | 0.19 | 0.26 | 2.4 | 3.1 | 7.8 | 3.5 | 4.4 | 3.3 |
| | protein | | | | | | | | |
| U51990 | pre-mRNA | 0.56 | 0.19 | 2.2 | 3.0 | 2.8 | 13.7 | 2.9 | 3.0 |
| | splicing | | | | | | | | |
| | factor | | | | | | | | |
| AF030555 | fatty-acid- | 0.10 | 0.39 | 3.5 | 6.9 | 13.3 | 4.4 | 7.5 | 2.9 |
| | Coenzyme A | | | | | | | | |
| AL009183 | TNFR | 0.46 | 0.19 | 6.0 | 4.1 | 2.8 | 8.6 | 2.6 | 2.8 |
| | superfamily, | | | | | | | | |
| | member 9 | | | | | | | | |
| AF045941 | sciellin | 0.16 | 0.21 | 11.6 | 2.4 | 2.8 | 2.2 | 4.1 | 2.8 |
| AF072756 | A kinase | 0.33 | 0.07 | 2.5 | 5.3 | 3.9 | 32.7 | 2.3 | 2.7 |
| | anchor | | | | | | | | |
| | protein 4 | | | | | | | | |
| X78678 | ketohexokinase | 0.10 | 0.20 | 18.0 | 3.5 | 4.1 | 2.5 | 14.6 | 2.6 |
| AL031734 | unknown | 0.03 | 0.39 | 43.7 | 2.3 | 41.7 | 4.0 | 10.8 | 2.5 |
| D87717 | KIAA0013 | 0.35 | 0.42 | 4.2 | 2.3 | 3.6 | 2.6 | 2.9 | 2.5 |
| | gene product | | | | | | | | |
| U01824 | solute carrier | 0.42 | 0.29 | 4.8 | 2.3 | 4.2 | 7.1 | 4.2 | 2.4 |
| | family 1 | | | | | | | | |
| AF055899 | solute carrier | 0.14 | 0.31 | 9.5 | 12.3 | 7.4 | 4.7 | 6.6 | 2.3 |
| | family 27 | | | | | | | | |
| U22526 | lanosterol | 0.09 | 0.45 | 4.1 | 3.4 | 10.4 | 2.2 | 17.9 | 2.3 |
| | synthase | | | | | | | | |
| AB032963 | unknown | 0.19 | 0.34 | 6.3 | 6.1 | 2.9 | 2.1 | 5.7 | 2.2 |
| NM_015974 | lambda- | 0.17 | 0.25 | 11.4 | 2.8 | 5.9 | 2.4 | 5.8 | 2.2 |
| | crystallin | | | | | | | | |
| X82200 | stimulated | 0.23 | 0.15 | 8.2 | 3.4 | 3.0 | 2.8 | 11.3 | 2.2 |
| | trans-acting | | | | | | | | |
| | factor | | | | | | | | |
| AL137522 | unknown | 0.12 | 0.26 | 12.1 | 3.7 | 12.6 | 6.9 | 4.3 | 2.2 |
| Z99916 | crystallin, | 0.28 | 0.65 | 2.5 | 2.1 | 3.6 | 2.2 | 2.6 | 2.1 |
| | beta B3 | | | | | | | | |
| AF233442 | ubiquitin | 0.41 | 0.31 | 2.6 | 3.6 | 3.6 | 4.5 | 3.4 | 2.1 |
| | specific | | | | | | | | |
| | protease 21 | | | | | | | | |
| AK001927 | hypothetical | 0.24 | 0.52 | 7.6 | 5.6 | 5.0 | 2.5 | 4.1 | 2.0 |
| | protein | | | | | | | | |

The peptides at a concentration of 50 μg/ml were shown to increase the expression of many polynucleotides. Peptide was incubated with the human A549 epithelial cells for 4 h and the RNA was isolated, converted into labeled cDNA probes and hybridized to Human Operon arrays (PRHU04). The intensity of polynucleotides in control, unstimulated cells are shown in the second and third columns for labeling of cDNA with the dyes Cy3 and Cy5 respectively.
The “ID #:Control” columns refer to the intensity of polynucleotide expression in peptidesimulated cells divided by the intensity of unstimulated cells.

TABLE 36
Up-regulation of Polynucleotide expression in A549 cells induced by Formula F Peptides.

| Accession | | ctrl- | ctrl- | Ratio | Ratio | Ratio | Ratio | Ratio |
|---|---|---|---|---|---|---|---|---|
| Number | Gene | Cy3 | Cy5 | ID 40:ctrl | ID 42:ctrl | ID 43:Ctrl | ID 44:Ctrl | ID 45:ctrl |
| AF025840 | polymerase | 0.34 | 0.96 | 3.4 | 2.0 | 2.0 | 2.1 | 4.3 |
| | epsilon 2 | | | | | | | |
| AF132495 | CGI-133 | 0.83 | 0.67 | 3.0 | 2.2 | 2.6 | 2.8 | 5.1 |
| | protein | | | | | | | |
| AL137682 | hypothetical | 0.73 | 0.40 | 2.0 | 5.3 | 4.8 | 2.9 | 8.2 |
| | protein | | | | | | | |
| U70426 | regulator of | 0.23 | 0.25 | 3.1 | 3.0 | 5.3 | 3.1 | 12.2 |
| | G-protein | | | | | | | |
| | signalling | | | | | | | |
| | 16 | | | | | | | |
| AK001135 | Sec23- | 0.29 | 0.53 | 3.2 | 2.6 | 3.3 | 14.4 | 5.2 |
| | interacting | | | | | | | |
| | protein | | | | | | | |
| | p125 | | | | | | | |
| AB023155 | KIAA0938 | 0.47 | 0.21 | 2.7 | 4.8 | 8.1 | 4.2 | 10.4 |
| | protein | | | | | | | |
| AB033080 | cell cycle | 0.31 | 0.31 | 4.4 | 2.2 | 5.9 | 4.3 | 6.9 |
| | progression | | | | | | | |
| | 8 protein | | | | | | | |
| AF061836 | Ras | 0.29 | 0.31 | 3.2 | 2.5 | 11.1 | 18.8 | 6.8 |
| | association | | | | | | | |
| | domain | | | | | | | |
| | family 1 | | | | | | | |
| AK000298 | hypothetical | 0.48 | 0.27 | 3.3 | 2.2 | 7.1 | 5.6 | 7.7 |
| | protein | | | | | | | |
| L75847 | zinc finger | 0.35 | 0.52 | 3.2 | 3.0 | 4.0 | 3.0 | 3.9 |
| | protein | | | | | | | |
| X97267 | protein | 0.19 | 0.24 | 4.1 | 9.3 | 2.4 | 4.2 | 8.3 |
| | tyrosine | | | | | | | |
| | phosphatase | | | | | | | |
| Z11933 | POU | 0.09 | 0.23 | 8.7 | 2.5 | 3.6 | 4.3 | 8.2 |
| | domain | | | | | | | |
| | class 3 TF 2 | | | | | | | |
| AB037744 | unknown | 0.37 | 0.57 | 2.6 | 2.9 | 2.7 | 3.0 | 3.1 |
| U90908 | unknown | 0.12 | 0.16 | 11.8 | 7.7 | 3.4 | 7.8 | 11.2 |
| AL050139 | unknown | 0.29 | 0.60 | 5.2 | 2.4 | 3.3 | 3.0 | 2.8 |
| AB014615 | fibroblast | 0.19 | 0.07 | 5.4 | 3.5 | 8.5 | 3.2 | 22.7 |
| | growth | | | | | | | |
| | factor 8 | | | | | | | |
| M28825 | CD1A | 0.51 | 0.36 | 4.1 | 2.6 | 2.0 | 4.6 | 4.4 |
| | antigen | | | | | | | |
| U27330 | fucosyltransferase 5 | 0.39 | 0.08 | 3.3 | 2.1 | 24.5 | 8.2 | 19.3 |
| NM_00696 | zinc finger | 0.10 | 0.08 | 10.4 | 12.6 | 12.3 | 29.2 | 20.5 |
| | protein | | | | | | | |
| AF093670 | peroxisomal | 0.44 | 0.53 | 4.0 | 2.6 | 2.6 | 4.3 | 2.9 |
| | biogenesis | | | | | | | |
| | factor | | | | | | | |
| AK000191 | hypothetical | 0.50 | 0.18 | 2.3 | 3.6 | 4.4 | 2.2 | 8.2 |
| | protein | | | | | | | |
| AB022847 | unknown | 0.39 | 0.24 | 2.1 | 6.9 | 4.5 | 2.8 | 6.2 |
| AK000358 | microfibrillar- | 0.28 | 0.28 | 5.7 | 2.0 | 3.5 | 5.2 | 5.2 |
| | associated | | | | | | | |
| | protein 3 | | | | | | | |
| X74837 | mannosidase_alpha | 0.10 | 0.07 | 13.1 | 18.4 | 23.6 | 16.3 | 20.8 |
| | class 1A | | | | | | | |
| AF053712 | TNF | 0.17 | 0.08 | 11.3 | 9.3 | 13.4 | 10.6 | 16.6 |
| | superfamily_member | | | | | | | |
| | 11 | | | | | | | |
| AL133114 | DKFZP586P2421 | 0.11 | 0.32 | 8.5 | 3.4 | 4.9 | 5.3 | 4.3 |
| | protein | | | | | | | |
| AF049703 | E74-like | 0.22 | 0.24 | 5.1 | 6.0 | 3.3 | 2.7 | 5.4 |
| | factor 5 | | | | | | | |
| AL137471 | hypothetical | 0.29 | 0.05 | 4.0 | 15.0 | 10.1 | 2.7 | 25.3 |
| | protein | | | | | | | |
| AL035397 | unknown | 0.33 | 0.14 | 2.3 | 2.8 | 10.6 | 4.6 | 9.3 |
| AL035447 | hypothetical | 0.28 | 0.23 | 3.8 | 6.8 | 2.7 | 3.0 | 5.7 |
| | protein | | | | | | | |
| X55740 | CD73 | 0.41 | 0.61 | 2.1 | 3.3 | 2.9 | 3.2 | 2.1 |
| NM_004909 | taxol | 0.20 | 0.22 | 3.9 | 2.9 | 6.5 | 3.2 | 5.6 |
| | resistance | | | | | | | |
| | associated | | | | | | | |
| | gene 3 | | | | | | | |
| AF233442 | ubiquitin | 0.41 | 0.31 | 2.9 | 4.7 | 2.7 | 3.5 | 3.9 |
| | specific | | | | | | | |
| | protease | | | | | | | |
| U92980 | unknown | 0.83 | 0.38 | 4.2 | 4.1 | 4.8 | 2.3 | 3.1 |
| AF105424 | myosin | 0.30 | 0.22 | 2.8 | 3.3 | 4.4 | 2.3 | 5.3 |
| | heavy | | | | | | | |
| | polypeptide- | | | | | | | |
| | like | | | | | | | |
| M26665 | histatin 3 | 0.29 | 0.26 | 7.9 | 3.5 | 4.6 | 3.5 | 4.5 |
| AF083898 | neuro- | 0.20 | 0.34 | 18.7 | 3.8 | 2.2 | 3.6 | 3.5 |
| | oncological | | | | | | | |
| | ventral | | | | | | | |
| | antigen 2 | | | | | | | |
| AJ009771 | ariadne_Drosophila_homolog | 0.33 | 0.06 | 2.3 | 17.6 | 15.9 | 2.5 | 20.3 |
| | of | | | | | | | |
| AL022393 | hypothetical | 0.05 | 0.33 | 32.9 | 2.4 | 3.0 | 69.4 | 3.4 |
| | protein P1 | | | | | | | |
| AF039400 | chloride | 0.11 | 0.19 | 8.4 | 2.9 | 5.1 | 18.1 | 5.9 |
| | channel_calcium | | | | | | | |
| | activated_family | | | | | | | |
| | member 1 | | | | | | | |
| AJ012008 | dimethylarginine | 0.42 | 0.43 | 5.1 | 3.3 | 3.2 | 6.2 | 2.6 |
| | dimethylaminohydrolase 2 | | | | | | | |
| AK000542 | hypothetical | 0.61 | 0.24 | 2.1 | 4.5 | 5.0 | 3.7 | 4.4 |
| | protein | | | | | | | |
| AL133654 | unknown | 0.27 | 0.40 | 2.8 | 2.1 | 2.5 | 2.5 | 2.6 |
| AL137513 | unknown | 0.43 | 0.43 | 6.4 | 3.2 | 3.8 | 2.3 | 2.3 |
| U05227 | GTP- | 0.38 | 0.36 | 5.0 | 3.1 | 3.1 | 2.2 | 2.8 |
| | binding | | | | | | | |
| | protein | | | | | | | |
| D38449 | putative G | 0.18 | 0.09 | 5.8 | 6.7 | 6.7 | 9.1 | 10.4 |
| | protein | | | | | | | |
| | coupled | | | | | | | |
| | receptor | | | | | | | |
| U80770 | unknown | 0.31 | 0.14 | 3.9 | 3.8 | 6.6 | 3.1 | 6.8 |
| X61177 | IL-5R alpha | 0.40 | 0.27 | 2.6 | 4.4 | 9.8 | 8.1 | 3.6 |
| U35246 | vacuolar | 0.15 | 0.42 | 5.8 | 2.8 | 2.6 | 4.5 | 2.2 |
| | protein | | | | | | | |
| | sorting 45A | | | | | | | |
| AB017016 | brain- | 0.27 | 0.29 | 6.0 | 2.6 | 3.4 | 3.1 | 3.1 |
| | specific | | | | | | | |
| | protein p25 | | | | | | | |
| | alpha | | | | | | | |
| X82153 | cathepsin K | 0.45 | 0.20 | 4.2 | 5.2 | 4.8 | 4.4 | 4.6 |
| AC005162 | probable | 0.12 | 0.28 | 11.9 | 3.4 | 6.8 | 18.7 | 3.2 |
| | carboxypeptidase | | | | | | | |
| | precursor | | | | | | | |
| AL137502 | unknown | 0.22 | 0.16 | 3.9 | 4.9 | 7.3 | 3.9 | 5.3 |
| U66669 | 3- | 0.30 | 0.40 | 10.3 | 3.5 | 5.2 | 2.3 | 2.1 |
| | hydroxyisobutyryl- | | | | | | | |
| | Coenzyme | | | | | | | |
| | A hydrolase | | | | | | | |
| AK000102 | unknown | 0.39 | 0.30 | 2.8 | 5.3 | 5.2 | 4.1 | 2.8 |
| AF034970 | docking | 0.28 | 0.05 | 3.3 | 8.5 | 15.7 | 4.0 | 17.3 |
| | protein 2 | | | | | | | |
| AK000534 | hypothetical | 0.13 | 0.29 | 6.8 | 2.3 | 4.0 | 20.6 | 2.9 |
| | protein | | | | | | | |
| J04599 | biglycan | 0.39 | 0.30 | 4.0 | 3.7 | 4.0 | 4.8 | 2.8 |
| AL133612 | unknown | 0.62 | 0.33 | 2.7 | 3.4 | 5.2 | 3.0 | 2.5 |
| D10495 | protein | 0.18 | 0.10 | 12.0 | 20.7 | 8.7 | 6.8 | 8.1 |
| | kinase C | | | | | | | |
| | delta | | | | | | | |
| X58467 | cytochrome | 0.07 | 0.24 | 15.4 | 4.7 | 7.9 | 34.4 | 3.4 |
| | P450 | | | | | | | |
| AF131806 | unknown | 0.31 | 0.25 | 2.6 | 3.4 | 5.7 | 7.0 | 3.2 |
| AK000351 | hypothetical | 0.34 | 0.13 | 4.0 | 6.9 | 5.5 | 2.8 | 6.3 |
| | protein | | | | | | | |
| AF075050 | hypothetical | 0.55 | 0.09 | 2.7 | 17.8 | 5.1 | 2.2 | 8.3 |
| | protein | | | | | | | |
| AK000566 | hypothetical | 0.15 | 0.35 | 6.7 | 2.2 | 6.8 | 6.4 | 2.1 |
| | protein | | | | | | | |
| | unknown | | | | | | | |
| U43328 | cartilage | 0.44 | 0.19 | 2.5 | 6.2 | 6.9 | 7.8 | 3.8 |
| | linking | | | | | | | |
| | protein 1 | | | | | | | |
| AF045941 | sciellin | 0.16 | 0.21 | 6.8 | 7.5 | 4.8 | 6.9 | 3.4 |
| U27655 | regulator of | 0.24 | 0.29 | 5.5 | 4.9 | 2.9 | 4.9 | 2.4 |
| | G-protein | | | | | | | |
| | signalling 3 | | | | | | | |
| AK000058 | hypothetical | 0.25 | 0.15 | 5.0 | 9.7 | 16.4 | 2.7 | 4.5 |
| | protein | | | | | | | |
| AL035364 | hypothetical | 0.32 | 0.26 | 4.4 | 4.2 | 7.3 | 2.8 | 2.6 |
| | protein | | | | | | | |
| AK001864 | unknown | 0.40 | 0.25 | 3.7 | 3.7 | 4.6 | 3.2 | 2.6 |
| AB015349 | unknown | 0.14 | 0.24 | 10.5 | 2.8 | 3.7 | 8.0 | 2.7 |
| V00522 | MHC class | 0.62 | 0.22 | 4.8 | 3.9 | 4.7 | 2.5 | 3.0 |
| | II DR beta 3 | | | | | | | |
| U75330 | neural cell | 0.42 | 0.08 | 2.1 | 9.6 | 13.2 | 3.3 | 7.8 |
| | adhesion | | | | | | | |
| | molecule 2 | | | | | | | |
| NM_007199 | IL-1R- | 0.15 | 0.25 | 8.7 | 7.8 | 8.6 | 16.1 | 2.5 |
| | associated | | | | | | | |
| | kinase M | | | | | | | |
| D30742 | calcium/cal | 0.28 | 0.09 | 6.2 | 28.7 | 7.4 | 2.4 | 6.8 |
| | modulin- | | | | | | | |
| | dependent | | | | | | | |
| | protein | | | | | | | |
| | kinase IV | | | | | | | |
| X05978 | cystatin A | 0.63 | 0.17 | 2.7 | 4.8 | 9.4 | 2.2 | 3.6 |
| AF240467 | TLR-7 | 0.11 | 0.10 | 13.8 | 13.3 | 4.7 | 7.7 | 4.9 |

The peptides at a concentration of 50 μg/ml were shown to increase the expression of many polynucleotides. Peptide was incubated with the human A549 epithelial cells for 4 h and the RNA was isolated, converted into labeled cDNA probes and hybridized to Human Operon arrays (PRHU04). The intensity of polynucleotides in control, unstimulated cells are shown in the second and third columns for labeling of eDNA with the dyes Cy3 and Cy5 respectively.
The “Ratio ID #:Control” columns refer to the intensity of polynucleotide expression in peptide-simulated cells divided by the intensity of unstimulated cells.

TABLE 37
Up-regulation of Polynucleotide expression in A549 cells induced by Formula G
and additional Peptides.

| Accession | ctrl- | ctrl- | | | | | | | | |
|---|---|---|---|---|---|---|---|---|---|---|
| Number | Cy3 | Cy5 | ID 53:ctrl | ID 54:ctrl | ID 47:ctrl | ID 48:ctrl | ID 49:ctrl | ID 50:ctrl | ID 51:ctrl | ID 52:ctrl |
| U00115 | 0.51 | 0.07 | 27.4 | 7.3 | 2.4 | 3.1 | 4.8 | 8.3 | 3.5 | 20.0 |
| M91036 | 0.22 | 0.02 | 39.1 | 32.5 | 5.2 | 2.2 | 37.0 | 6.0 | 16.2 | 18.0 |
| AK000070 | 0.36 | 0.18 | 3.8 | 7.6 | 2.6 | 15.1 | 12.2 | 9.9 | 17.2 | 15.3 |
| AF055899 | 0.14 | 0.31 | 6.7 | 3.7 | 9.7 | 10.0 | 2.2 | 16.7 | 5.4 | 14.8 |
| AK001490 | 0.05 | 0.02 | 14.1 | 35.8 | 3.2 | 28.6 | 25.0 | 20.2 | 56.5 | 14.1 |
| X97674 | 0.28 | 0.28 | 3.2 | 3.7 | 4.0 | 10.7 | 3.3 | 3.1 | 4.0 | 13.2 |
| AB022847 | 0.39 | 0.24 | 4.1 | 4.4 | 4.5 | 2.7 | 3.7 | 10.4 | 5.0 | 11.3 |
| AJ275986 | 0.26 | 0.35 | 5.8 | 2.3 | 5.7 | 2.2 | 2.5 | 9.7 | 4.3 | 11.1 |
| D10495 | 0.18 | 0.10 | 8.0 | 3.4 | 4.6 | 2.0 | 6.9 | 2.5 | 12.7 | 10.3 |
| L36642 | 0.26 | 0.06 | 5.8 | 14.2 | 2.6 | 4.1 | 8.9 | 3.4 | 6.5 | 6.6 |
| M31166 | 0.31 | 0.12 | 4.8 | 3.8 | 12.0 | 3.6 | 9.8 | 2.4 | 8.8 | 6.4 |
| AF176012 | 0.45 | 0.26 | 3.1 | 2.9 | 2.8 | 2.6 | 2.3 | 6.9 | 3.0 | 5.8 |
| AF072756 | 0.33 | 0.07 | 9.9 | 9.3 | 4.4 | 4.3 | 3.2 | 4.9 | 11.9 | 5.4 |
| NM_014439 | 0.47 | 0.07 | 12.0 | 7.1 | 3.3 | 3.3 | 4.7 | 5.9 | 5.0 | 5.4 |
| AJ271351 | 0.46 | 0.12 | 3.4 | 3.5 | 2.3 | 4.7 | 2.3 | 2.7 | 6.9 | 5.2 |
| AK000576 | 0.27 | 0.06 | 7.4 | 15.7 | 2.9 | 4.7 | 9.0 | 2.4 | 8.2 | 5.1 |
| AJ272265 | 0.21 | 0.09 | 6.2 | 7.9 | 2.3 | 3.7 | 10.3 | 4.5 | 4.6 | 4.7 |
| AL122038 | 0.46 | 0.06 | 6.7 | 4.5 | 2.6 | 4.3 | 16.4 | 6.5 | 26.6 | 4.6 |
| AK000307 | 0.23 | 0.09 | 3.7 | 4.0 | 4.3 | 3.2 | 5.3 | 2.9 | 13.1 | 4.4 |
| AB029001 | 0.52 | 0.21 | 14.4 | 4.3 | 4.6 | 4.4 | 4.8 | 21.9 | 3.2 | 4.2 |
| U62437 | 0.38 | 0.13 | 12.6 | 6.5 | 4.2 | 6.7 | 2.2 | 3.7 | 4.8 | 3.9 |
| AF064854 | 0.15 | 0.16 | 2.6 | 2.9 | 6.2 | 8.9 | 14.4 | 5.0 | 9.1 | 3.9 |
| AL031588 | 0.40 | 0.26 | 8.3 | 5.2 | 2.8 | 3.3 | 5.3 | 9.0 | 5.6 | 3.4 |
| X89399 | 0.25 | 0.10 | 15.8 | 12.8 | 7.4 | 4.2 | 16.7 | 6.9 | 12.7 | 3.3 |
| D45399 | 0.21 | 0.18 | 3.0 | 4.7 | 3.3 | 4.4 | 8.7 | 5.3 | 5.1 | 3.3 |
| AB037716 | 0.36 | 0.40 | 5.1 | 7.5 | 2.6 | 2.1 | 3.5 | 3.1 | 2.4 | 2.8 |
| X79981 | 0.34 | 0.10 | 4.7 | 7.2 | 3.2 | 4.6 | 6.5 | 5.1 | 5.8 | 2.7 |
| AF034208 | 0.45 | 0.24 | 2.7 | 10.9 | 2.1 | 3.7 | 2.3 | 5.9 | 2.2 | 2.5 |
| AL133355 | 0.22 | 0.23 | 2.3 | 3.4 | 7.3 | 2.7 | 3.3 | 4.3 | 2.8 | 2.5 |
| NM_016281 | 0.40 | 0.19 | 6.6 | 10.6 | 2.1 | 2.8 | 5.0 | 11.2 | 10.6 | 2.5 |
| AF023614 | 0.11 | 0.42 | 2.2 | 2.2 | 6.0 | 7.5 | 5.0 | 2.7 | 2.0 | 2.4 |
| AF056717 | 0.43 | 0.62 | 4.3 | 3.2 | 5.1 | 4.0 | 4.6 | 9.7 | 3.1 | 2.2 |
| AB029039 | 0.79 | 0.49 | 2.7 | 3.3 | 3.7 | 2.0 | 2.3 | 2.4 | 4.8 | 2.2 |
| J03634 | 0.40 | 0.12 | 3.7 | 2.3 | 2.3 | 4.0 | 10.5 | 4.1 | 9.1 | 2.2 |
| U80764 | 0.31 | 0.18 | 2.3 | 7.4 | 4.2 | 2.3 | 5.1 | 3.3 | 8.8 | 2.1 |
| AB032963 | 0.19 | 0.34 | 4.0 | 7.3 | 5.0 | 3.0 | 2.9 | 6.7 | 3.8 | 2.1 |
| X82835 | 0.25 | 0.38 | 2.0 | 2.7 | 2.9 | 7.7 | 3.3 | 3.1 | 3.5 | 2.0 |
| The peptides at a concentration of 50 μg/ml were shown to increase the expression of many polynucleotides. Peptide was incubated with the human A549 epithelial cells for 4 h and the RNA was isolated, converted into labelled cDNA probes and hybridised to Human Operon arrays (PRHUO4). The intensity of polynucleotides in control, unstimulated cells are shown in the second and third columns for labelling of cDNA with the dyes Cy3 and Cy5 respectively. | | | | | | | | | | |
| The “Ratio ID #:Control” columns refer to the intensity of polynucleotide expression in peptide-simulated cells divided by the intensity of unstimulated cells. | | | | | | | | | | |
| Accession numbers and gene designations are U00115, zinc finger protein; M91036, hemoglobin gamma G; K000070, hypothetical protein; AF055899, solute carrier family 27; AK001490, hypothetical protein; X97674, nuclear receptor coactivator 2; AB022847, unknown; AJ275986, transcription factor; D10495, protein kinase C, delta; L36642, EphA7; M31166, pentaxin-related gene; AF176012, unknown; AF072756, A kinase anchor protein 4; NM_014439, IL-i Superfamily z; AJ271351, putative | | | | | | | | | | |
| # transcriptional regulator; AK000576, hypothetical protein; AJ272265, secreted phosphoprotein 2; AL122038, hypothetical protein; AK000307, hypothetical protein; AB029001, KIAA1O78 protein; U62437, cholinergic receptor; AF064854, unknown; AL031588, hypothetical protein; X89399, RAS p21 protein activator; D45399, phosphodiesterase; AB037716, hypothetical protein; X79981, cadherin 5; AF034208, RIG-like 7-1; AL133355, chromosome 21 open reading frame 53; NM_016281, STE20-like kinase; | | | | | | | | | | |
| # AF023614, transmembrane activator and CAML interactor; AF056717, ash2-like; AB029039, KIAA1116 protein; J03634, inhibin, beta A; U80764, unknown; AB032963, unknown; X82835, sodium channel, voltage-gated, type IX. | | | | | | | | | | |

### EXAMPLE 5

### Induction of Chemokines in Cell Lines, Whole Human Blood, and in Mice by Peptides

The murine macrophage cell line RAW 264.7, THP-1 cells (human monocytes), a human epithelial cell line (A549), human bronchial epithelial cells (16HBEo14), and whole human blood were used. HBE cells were grown in MEM with Earle's. THP-1 cells were grown and maintained in RPMI 1640 medium. The RAW and A549 cell lines were maintained in DMEM supplemented with 10% fetal calf serum. The cells were seeded in 24 well plates at a density of 10⁶ cells per well in DMEM (see above) and A549 cells were seeded in 24 well plates at a density of 10⁵ cells per well in DMEM (see above) and both were incubated at 37° C. in 5% CO₂ overnight. DMEM was aspirated from cells grown overnight and replaced with fresh medium. After incubation of the cells with peptide, the release of chemokines into the culture supernatant was determined by ELISA (R&D Systems, Minneapolis, Minn.).

Animal studies were approved by the UBC Animal Care Committee (UBC ACC # A01-0008). BALB/c mice were purchased from Charles River Laboratories and housed in standard animal facilities. Age, sex and weight matched adult mice were anaesthetized with an intraperitoneal injection of Avertin (4.4 mM 2-2-2-tribromoethanol, 2.5% 2-methyl-2-butanol, in distilled water), using 200 μl per 10 g body weight. The instillation was performed using a non-surgical, intratracheal instillation method adapted from Ho and Furst 1973. Briefly, the anaesthetized mouse was placed with its upper teeth hooked over a wire at the top of a support frame with its jaw held open and a spring pushing the thorax forward to position the pharynx, larynx and trachea in a vertical straight line. The airway was illuminated externally and an intubation catheter was inserted into the clearly illuminated tracheal lumen. Twenty-μl of peptide suspension or sterile water was placed in a well at the proximal end of the catheter and gently instilled into the trachea with 200 μl of air. The animals were maintained in an upright position for 2 minutes after instillation to allow the fluid to drain into the respiratory tree. After 4 hours the mice were euthanaised by intraperitoneal injection of 300 mg/kg of pentobarbital. The trachea was exposed; an intravenous catheter was passed into the proximal trachea and tied in place with suture thread. Lavage was performed by introducing 0.75 ml sterile PBS into the lungs via the tracheal cannula and then after a few seconds, withdrawing the fluid. This was repeated 3 times with the same sample of PBS. The lavage fluid was placed in a tube on ice and the total recovery volume per mouse was approximately 0.5 ml. The bronchoalveolar lavage (BAL) fluid was centrifuged at 1200 rpm for 10 min, the clear supernatant removed and tested for TNF-α and MCP-1 by ELISA.

The up-regulation of chemokines by cationic peptides was confirmed in several different systems. The murine MCP-1, a homologue of the human MCP-1, is a member of the β(C-C) chemokine family. MCP-1 has been demonstrated to recruit monocytes, NK cells and some T lymphocytes. When RAW 264.7 macrophage cells and whole human blood from 3 donors were stimulated with increasing concentrations of peptide, SEQ ID NO: 1, they produced significant levels of MCP-1 in their supernatant, as judged by ELISA (Table 36). RAW 264.7 cells stimulated with peptide concentrations ranging from 20-50 μg/ml for 24 hr produced significant levels of MCP-1 (200-400 pg/ml above background). When the cells (24 h) and whole blood (4 h) were stimulated with 100 μg/ml of SEQ ID NO: 1, high levels of MCP-1 were produced.

The effect of cationic peptides on chemokine induction was also examined in a completely different cell system, A549 human epithelial cells. Interestingly, although these cells produce MCP-1 in response to LPS, and this response could be antagonized by peptide; there was no production of MCP-1 by A549 cells in direct response to peptide, SEQ ID NO: 1. Peptide SEQ ID NO: 1 at high concentrations, did however induce production of IL-8, a neutrophil specific chemokine (Table 37). Thus, SEQ ID NO: 1 can induce a different spectrum of responses from different cell types and at different concentrations. A number of peptides from each of the formula groups were tested for their ability to induce IL-8 in A549 cells (Table 38). Many of these peptides at a low concentration, 10 μg/ml induced IL-8 above background levels. At high concentrations (100 μg/ml) SEQ ID NO: 13 was also found to induce IL-8 in whole human blood (Table 39). Peptide SEQ ID NO: 2 also significantly induced IL-8 in HBE cells (Table 40) and undifferentiated THP-1 cells (Table 41).

BALB/c mice were given SEQ ID NO: 1 or endotoxin-free water by intratracheal instillation and the levels of MCP-1 and TNF-α examined in the bronchioalveolar lavage fluid after 3-4 hr. It was found that the mice treated with 50 μg/ml peptide, SEQ ID NO: 1 produced significantly increased levels of MCP-1 over mice given water or anesthetic alone (Table 42). This was not a pro-inflammatory response to peptide, SEQ ID NO: 1 since peptide did not significantly induce more TNF-α than mice given water or anesthetic alone. peptide, SEQ ID NO: 1 was also found not to significantly induce TNF-α production by RAW 264.7 cells and bone marrow-derived macrophages treated with peptide, SEQ ID NO: 1 (up to 100 μg/ml) (Table 43). Thus, peptide, SEQ ID NO: 1 selectively induces the production of chemokines without inducing the production of inflammatory mediators such as TNF-α. This illustrates the dual role of peptide, SEQ ID NO: 1 as a factor that can block bacterial product-induced inflammation while helping to recruit phagocytes that can clear infections.

TABLE 38
Induction of MCP-1 in RAW 264.7 cells and whole human blood.

| | |
|---|---|
| | Monocyte chemoattractant |
| Peptide, SEQ ID NO: 1 | protein (MCP)-1 (pg/ml)* |

| (μg/ml) | RAW cells | Whole blood |
|---|---|---|
| 0 | 135.3 ± 16.3 | 112.7 ± 43.3 |
| 10 | 165.7 ± 18.2 | 239.3 ± 113.3 |
| 50 | 367 ± 11.5 | 371 ± 105 |
| 100 | 571 ± 17.4 | 596 ± 248.1 |

RAW 264.7 mouse macrophage cells or whole human blood were stimulated with increasing concentrations of SEQ ID NO: 1 for 4 hr. The human blood samples were centrifuged and the serum was removed and tested for MCP-1 by ELISA along with the supernatants from the RAW 264.7 cells.
The RAW cell data presented is the mean of three or more experiments ± standard error and the human blood data represents the mean ± standard error from three separate donors.

TABLE 39
Induction of IL-8 in A549 cells and whole human blood.

| | |
|---|---|
| Peptide, SEQ ID NO: 1 | IL-8 (pg/ml) |

| (μg/ml) | A549 cells | Whole blood |
|---|---|---|
| 0 | 172 ± 29.1 | 660.7 ± 126.6 |
| 1 | 206.7 ± 46.1 | |
| 10 | 283.3 ± 28.4 | 945.3 ± 279.9 |
| 20 | 392 ± 31.7 | |
| 50 | 542.3 ± 66.2 | 1160.3 ± 192.4 |
| 100 | 1175.3 ± 188.3 | |

A549 cells or whole human blood were stimulated with increasing concentrations of peptide for 24 and 4 hr respectively, The human blood samples were centrifuged and the serum was removed and tested for IL-8 by ELISA along with the supernatants from the A549 cells.
The A549 cell data presented is the mean of three or more experiments ± standard error and the human blood data represents the mean ± standard error from three separate donors.

TABLE 40
Induction of IL-8 in A549 cells by Cationic peptides.

| Peptide (10 ug/ml) | IL-8 (ng/ml) |
|---|---|
| No peptide | 0.164 |
| LPS, no peptide | 0.26 |
| SEQ ID NO: 1 | 0.278 |
| SEQ ID NO: 6 | 0.181 |
| SEQ ID NO: 7 | 0.161 |
| SEQ ID NO: 9 | 0.21 |
| SEQ ID NO: 10 | 0.297 |
| SEQ ID NO: 13 | 0.293 |
| SEQ ID NO: 14 | 0.148 |
| SEQ ID NO: 16 | 0.236 |
| SEQ ID NO: 17 | 0.15 |
| SEQ ID NO: 19 | 0.161 |
| SEQ ID NO: 20 | 0.151 |
| SEQ ID NO: 21 | 0.275 |
| SEQ ID NO: 22 | 0.314 |
| SEQ ID NO: 23 | 0.284 |
| SEQ ID NO: 24 | 0.139 |
| SEQ ID NO: 26 | 0.201 |
| SEQ ID NO: 27 | 0.346 |
| SEQ ID NO: 28 | 0.192 |
| SEQ ID NO: 29 | 0.188 |
| SEQ ID NO: 30 | 0.284 |
| SEQ ID NO: 31 | 0.168 |
| SEQ ID NO: 33 | 0.328 |
| SEQ ID NO: 34 | 0.315 |
| SEQ ID NO: 35 | 0.301 |
| SEQ ID NO: 36 | 0.166 |
| SEQ ID NO: 37 | 0.269 |
| SEQ ID NO: 38 | 0.171 |
| SEQ ID NO: 40 | 0.478 |
| SEQ ID NO: 41 | 0.371 |
| SEQ ID NO: 42 | 0.422 |
| SEQ ID NO: 43 | 0.552 |
| SEQ ID NO: 44 | 0.265 |
| SEQ ID NO: 45 | 0.266 |
| SEQ ID NO: 47 | 0.383 |
| SEQ ID NO: 48 | 0.262 |
| SEQ ID NO: 49 | 0.301 |
| SEQ ID NO: 50 | 0.141 |
| SEQ ID NO: 51 | 0.255 |
| SEQ ID NO: 52 | 0.207 |
| SEQ ID NO: 53 | 0.377 |
| SEQ ID NO: 54 | 0.133 |

A549 human epithelial cells were stimulated with 10 μg of peptide for 24 hr. The supematant was removed and tested for IL-8 by ELISA.

TABLE 41
Induction by Peptide of IL-8 in human blood.

| SEQ ID NO: 3 (μg/ml) | IL-8 (pg/ml) |
|---|---|
| 0 | 85 |
| 10 | 70 |
| 100 | 323 |

Whole human blood was stimulated with increasing concentrations of peptide for 4 hr. The human blood samples were centrifuged and the serum was removed and tested for IL-8 by ELISA. The data shown is the average 2 donors.

TABLE 42
Induction of IL-8 in HBE cells.

| SEQ ID NO: 2 | |
|---|---|
| (μg/ml) | IL-8 (pg/ml) |
| 0 | 552 ± 90 |
| 0.1 | 670 ± 155 |
| 1 | 712 ± 205 |
| 10 | 941 ± 15 |
| 50 | 1490 ± 715 |

Increasing concentrations of the peptide were incubated with HBE cells for 8 h, the supernantant removed and tested for IL-8. The data is presented as the mean of three or more experiments ± standard error.

TABLE 43
Induction of IL-8 in undifferentiated THP-1 cells.

| SEQ ID NO: 3 | |
|---|---|
| (μg/ml) | IL-8 (pg/ml) |
| 0 | 10.6 |
| 10 | 17.2 |
| 50 | 123.7 |

The human monocyte THP-1 cells were incubated with indicated concentrations of peptide for 8 hr. The supernatant was removed and tested for IL-8 by ELISA.

TABLE 44
Induction of MCP-1 by Peptide, SEQ ID NO: 1 in mouse airway.

| Condition | MCP-1 (pg/ml) | TNF-α (pg/ml) |
|---|---|---|
| Water | 16.5 ± 5 | 664 ± 107 |
| peptide | 111 ± 30 | 734 ± 210 |
| Avertin | 6.5 ± 0.5 | 393 ± 129 |

BALB/c mice were anaesthetised with avertin and given intratracheal instillation of peptide or water or no instillation (no treatment). The mice were monitored for 4 hours, anaesthetised and the BAL fluid was isolated and analyzed for MCP-1 and TNF-α concentrations by ELISA. The data shown is the mean of 4 or 5 mice for each condjtion ± standard error.

TABLE 45
Lack of Significant TNF-α induction by the Cationic Peptides.

| Peptide Treatment | TNF-α (pg/ml) |
|---|---|
| Media background | 56 ± 8 |
| LPS treatment, No peptide | 15207 ± 186 |
| SEQ ID NO: 1 | 274 ± 15 |
| SEQ ID NO: 5 | 223 ± 45 |
| SEQ ID NO: 6 | 297 ± 32 |
| SEQ ID NO: 7 | 270 ± 42 |
| SEQ ID NO: 8 | 166 ± 23 |
| SEQ ID NO: 9 | 171 ± 33 |
| SEQ ID NO: 10 | 288 ± 30 |
| SEQ ID NO: 12 | 299 ± 65 |
| SEQ ID NO: 13 | 216 ± 42 |
| SEQ ID NO: 14 | 226 ± 41 |
| SEQ ID NO: 15 | 346 ± 41 |
| SEQ ID NO: 16 | 341 ± 68 |
| SEQ ID NO: 17 | 249 ± 49 |
| SEQ ID NO: 19 | 397 ± 86 |
| SEQ ID NO: 20 | 285 ± 56 |
| SEQ ID NO: 21 | 263 ± 8 |
| SEQ ID NO: 22 | 195 ± 42 |
| SEQ ID NO: 23 | 254 ± 58 |
| SEQ ID NO: 24 | 231 ± 32 |
| SEQ ID NO: 26 | 281 ± 34 |
| SEQ ID NO: 27 | 203 ± 42 |
| SEQ ID NO: 28 | 192 ± 26 |
| SEQ ID NO: 29 | 242 ± 40 |
| SEQ ID NO: 31 | 307 ± 71 |
| SEQ ID NO: 33 | 196 ± 42 |
| SEQ ID NO: 34 | 204 ± 51 |
| SEQ ID NO: 35 | 274 ± 76 |
| SEQ ID NO: 37 | 323 ± 41 |
| SEQ ID NO: 38 | 199 ± 38 |
| SEQ ID NO: 43 | 947 ± 197 |
| SEQ ID NO: 44 | 441 ± 145 |
| SEQ ID NO: 45 | 398 ± 90 |
| SEQ ID NO: 48 | 253 ± 33 |
| SEQ ID NO: 49 | 324 ± 38 |
| SEQ ID NO: 50 | 311 ± 144 |
| SEQ ID NO: 53 | 263 ± 40 |
| SEQ ID NO: 54 | 346 ± 86 |

RAW 264.7 macrophage cells were incubated with indicated peptides (40 μg/ml) for 6 hours. The supernatant was collected and tested for levels of TNF-α by ELISA. The data is presented as the mean of three or more experiments ± standard error.

### EXAMPLE 6

### Cationic Peptides Increase Surface Expression of Chemokine Receptors

To analyze cell surface expression of IL-8RB, CXCR-4, CCR2, and LFA-1, RAW macrophage cells were stained with 10 μg/ml of the appropriate primary antibody (Santa Cruz Biotechnology) followed by FITC-conjugated goat anti-rabbit IgG [IL-8RB and CXCR-4 (Jackson ImmunoResearch Laboratories, West Grove, Pa.)] or FITC-conjugated donkey anti-goat IgG (Santa Cruz). The cells were analyzed using a FACscan, counting 10,000 events and gating on forward and side scatter to exclude cell debris.

The polynucleotide array data suggested that some peptides up-regulate the expression of the chemokine receptors IL-8RB, CXCR-4 and CCR2 by 10, 4 and 1.4 fold above unstimulated cells respectively. To confirm the polynucleotide array data, the surface expression was examined by flow cytometry of these receptors on RAW cells stimulated with peptide for 4 hr. When 50 μg/ml of peptide was incubated with RAW cells for 4 hr, IL-8RB was upregulated an average of 2.4-fold above unstimulated cells, CXCR-4 was up-regulated an average of 1.6-fold above unstimulated cells and CCR2 was up-regulated 1.8-fold above unstimulated cells (Table 46). As a control CEMA was demonstrated to cause similar up-regulation. SEQ ID NO: 3 was the only peptide to show significant up-regulation of LFA-1 (3.8 fold higher than control cells).

TABLE 46
Increased surface expression of CXCR-4, IL-8RB and
CCR2 in response to peptides.

| | Concentration | Fold Increase in Protein Expression | | |
|---|---|---|---|---|
| Peptide | (μg/ml) | IL-8RB | CXCR-4 | CCR2 |
| SEQ ID NO: 1 | 10 | 1.0 | 1.0 | 1.0 |
| SEQ ID NO: 1 | 50 | 1.3 ± 0.05 | 1.3 ± 0.03 | 1.3 ± 0.03 |
| SEQ ID NO: 1 | 100 | 2.4 ± 0.6 | 1.6 ± 0.23 | 1.8 ± 0.15 |
| SEQ ID NO: 3 | 100 | 2.0 ± 0.6 | Not Done | 4.5 |
| CEMA | 50 | 1.6 ± 0.1 | 1.5 ± 0.2 | 1.5 ± 0.15 |
| CEMA | 100 | 3.6 ± 0.8 | Not Done | 4.7 ± 1.1 |

RAW macrophage cells were stimulated with peptide for 4 hr. The cells were washed and stained with the appropriate primary and FITC-labeled secondary antibodies. The data shown represents the average (fold change of RAW cells stimulated with peptide from media) ± standard error.

### EXAMPLE 7

### Phosphorylation of Map Kinases by Cationic Peptides

The cells were seeded at 2.5×10⁵-5×10⁵ cells/ml and left overnight. They were washed once in media, serum starved in the morning (serum free media -4 hrs). The media was removed and replaced with PBS, then sat at 37° C. for 15 minutes and then brought to room temp for 15 minutes. Peptide was added (concentrations 0.1 ug/ml-50 ug/ml) or H₂O and incubated 10 min. The PBS was very quickly removed and replaced with ice-cold radioimmunoprecipitation (RIPA) buffer with inhibitors (NaF, B-glycerophosphate, MOL, Vanadate, PMSF, Leupeptin Aprotinin). The plates were shaken on ice for 10-15 min or until the cells were lysed and the lysates collected. The procedure for THP-1 cells was slightly different; more cells (2×10⁶) were used. They were serum starved overnight, and to stop the reaction 1 ml of ice-cold PBS was added then they sat on ice 5-10 min, were spun down then resuspended in RIPA. Protein concentrations were determined using a protein assay (Pierce, Rockford, Ill.). Cell lysates (20 μg of protein) were separated by SDS-PAGE and transferred to nitrocellulose filters. The filters were blocked for 1 h with 10 mM Tris-HCl, pH 7.5, 150 mM NaCl (TBS)/5% skim milk powder and then incubated overnight in the cold with primary antibody in TBS/0.05% Tween 20. After washing for 30 min with TBS/0.05% Tween 20, the filters were incubated for 1 h at room temperature with 1 μg/ml secondary antibody in TBS. The filters were washed for 30 min with TBS/0.05% Tween 20 and then incubated 1 h at room temperature with horseradish peroxidase-conjugated sheep anti-mouse IgG (1:10,000 in TBS/0.05% Tween 20). After washing the filters for 30 min with TBS/0.1% Tween 20, immunoreactive bands were visualized by enhanced chemiluminescence (ECL) detection. For experiments with peripheral blood mononuclear cells: The peripheral blood (50-100 ml) was collected from all subjects. Mononuclear cells were isolated from the peripheral blood by density gradient centrifugation on Ficoll-Hypaque. Interphase cells (mononuclear cells) were recovered, washed and then resuspended in recommended primary medium for cell culture (RPMI-1640) with 10% fetal calf serum (FCS) and 1% L-glutamine. Cells were added to 6 well culture plates at 4×10⁶ cells/well and were allowed to adhere at 37° C. in 5% CO₂ atmosphere for 1 hour. The supernatant medium and non-adherent cells were washed off and the appropriate media with peptide was added. The freshly harvested cells were consistently >99% viable as assessed by their ability to exclude trypan blue. After stimulation with peptide, lysates were collected by lysing the cells in RIPA buffer in the presence of various phosphatase- and kinase-inhibitors. Protein content was analyzed and approximately 30 μg of each sample was loaded in a 12% SDS-PAGE gel. The gels were blotted onto nitrocellulose, blocked for 1 hour with 5% skim milk powder in Tris buffered saline (TBS) with 1% Triton X 100. Phosphorylation was detected with phosphorylation-specific antibodies.

The results of peptide-induced phosphorylation are summarized in Table 46. SEQ ID NO: 2 was found to cause dose dependent phosphorylation of p38 and ERK1/2 in the mouse macrophage RAW cell line and the HBE cells. SEQ ID NO: 3 caused phosphorylation of MAP kinases in THP-1 human monocyte cell line and phosphorylation of ERK1/2 in the mouse RAW cell line.

TABLE 47
Phosphorylation of MAP kinases in response to peptides.

MAP kinase
phosphorylated

| Cell Line | Peptide | p38 | ERK½ |
|---|---|---|---|
| RAW 264.7 | SEQ ID NO: 3 | − | + |
| | SEQ ID NO: 2 | + | + |
| HBE | SEQ ID NO: 3 | | + |
| | SEQ ID NO: 2 | + | + |
| THP-1 | SEQ ID NO: 3 | + | + |
| | SEQ ID NO: 2 | | |

TABLE 48
Peptide Phosphorylation of MAP kinases in human blood monocytes.
SEQ ID NO: 1 at 50 μg/ml) was used to promote phosphorylation.

| | |
|---|---|
| p38 phosphorylation | ERK½ phosphorylation |

| 15 minutes | 60 minutes | 15 minutes | 60 minutes |
|---|---|---|---|
| + | − | + | + |

### EXAMPLE 8

### Cationic Peptides Protect Against Bacterial Infection by Enhancing the Immune Response

BALB/c mice were given 1×10⁵ Salmonella and cationic peptide (200 μg) by intraperitoneal injection. The mice were monitored for 24 hours at which point they were euthanized, the spleen removed, homogenized and resuspended in PBS and plated on Luria Broth agar plates with Kanamycin (50 μg/ml). The plates were incubated overnight at 37° C. and counted for viable bacteria (Table 49 and 50). CD-1 mice were given 1×10⁸ S. aureus in 5% porcine mucin and cationic peptide (200 μg) by intraperitoneal injection (Table 51). The mice were monitored for 3 days at which point they were euthanized, blood removed and plated for viable counts. CD-1 male mice were given 5.8×10⁶ CFU EHEC bacteria and cationic peptide (200 μg) by intraperitoneal (IP) injection and monitored for 3 days (Table 52). In each of these animal models a subset of the peptides demonstrated protection against infections. The most protective peptides in the Salmonella model demonstrated an ability to induce a common subset of genes in epithelial cells (Table 53) when comparing the protection assay results in Tables 50 and 51 to the gene expression results in Tables 31-37. This clearly indicates that there is a pattern of gene expression that is consistent with the ability of a peptide to demonstrate protection. Many of the cationic peptides were shown not to be directly antimicrobial as tested by the Minimum Inhibitory Concentration (MIC) assay (Table 54). This demonstrates that the ability of peptides to protect against infection relies on the ability of the peptide to stimulate host innate immunity rather than on direct antimicrobial activity.

TABLE 49
Effect of Cationic Peptides on Salmonella Infection in BALB/c mice.

| Peptide | Viable Bacteria in the Spleen | Statistical Significance |
|---|---|---|
| Treatment | (CFU/ml) | (p value) |
| Control | 2.70 ± 0.84 × 10⁵ | |
| SEQ ID NO: 1 | 1.50 ± 0.26 × 10⁵ | 0.12 |
| SEQ ID NO: 6 | 2.57 ± 0.72 × 10⁴ | 0.03 |
| SEQ ID NO: 13 | 3.80 ± 0.97 × 10⁴ | 0.04 |
| SEQ ID NO: 17 | 4.79 ± 1.27 × 10⁴ | 0.04 |
| SEQ ID NO: 27 | 1.01 ± 0.26 × 10⁵ | 0.06 |

The BALB/c mice were injected IP with Salmonella and Peptide, and 24 h later the animals were euthanized, the spleen removed, homogenized, diluted in PBS and plate counts were done to determine bacteria viability.

TABLE 50
Effect of Cationic Peptides on Salmonella Infection in BALB/c mice.

| Peptide Treatment | Viable Bacteria in the Spleen (CFU/ml) |
|---|---|
| Control | 1.88 ± 0.16 × 10⁴ |
| SEQ ID NO: 48 | 1.98 ± 0.18 × 10⁴ |
| SEQ ID NO: 26 | 7.1 ± 1.37 × 10⁴ |
| SEQ ID NO: 30 | 5.79 ± 0.43 × 10³ |
| SEQ ID NO: 37 | 1.57 ± 0.44 × 10⁴ |
| SEQ ID NO: 5 | 2.75 ± 0.59 × 10⁴ |
| SEQ ID NO: 7 | 5.4 ± 0.28 × 10³ |
| SEQ ID NO: 9 | 1.23 ± 0.87 × 10⁴ |
| SEQ ID NO: 14 | 2.11 ± 0.23 × 10³ |
| SEQ ID NO: 20 | 2.78 ± 0.22 × 10⁴ |
| SEQ ID NO: 23 | 6.16 ± 0.32 × 10⁴ |

The BALB/c mice were injected intraperitoneally with Salmonella and Peptide, and 24 h later the animals were euthanized, the spleen removed, homogenized, diluted in PBS and plate counts were done to determine bacteria viability.

TABLE 51
Effect of Cationic Peptides in a Murine S. aureus infection model.

| | | # Mice Survived |
|---|---|---|
| Treatment | CFU/ml (blood) | (3 days)/Total mice in group |
| No Peptide | 7.61 ± 1.7 × 10³ | 6/8 |
| SEQ ID NO: 1 | 0 | 4/4 |
| SEQ ID NO: 27 | 2.25 ± 0.1 × 10² | 3/4 |
| SEQ ID NO: 30 | 1.29 ± 0.04 × 10² | 4/4 |
| SEQ ID NO: 37 | 9.65 ± 0.41 × 10² | 4/4 |
| SEQ ID NO: 5 | 3.28 ± 1.7 × 10³ | 4/4 |
| SEQ ID NO: 6 | 1.98 ± 0.05 × 10² | 3/4 |
| SEQ ID NO: 7 | 3.8 ± 0.24 × 10³ | 4/4 |
| SEQ ID NO: 9 | 2.97 ± 0.25 × 10² | 4/4 |
| SEQ ID NO: 13 | 4.83 ± 0.92 × 10³ | 3/4 |
| SEQ ID NO: 17 | 9.6 ± 0.41 × 10² | 4/4 |
| SEQ ID NO: 20 | 3.41 ± 1.6 × 10³ | 4/4 |
| SEQ ID NO: 23 | 4.39 ± 2.0 × 10³ | 4/4 |

CD-1 mice were given 1 × 10⁸ bacteria in 5% porcine mucin via intraperitoneal (IP) injection. Cationic peptide (200 μg) was given via a separate IP injection. The mice were monitored for 3 days at which point they were euthanized, blood removed and plated for viable counts. The following peptides were not effective in controlling S. aureus infection: SEQ ID NO: 48, SEQ ID NO: 26.

TABLE 52
Effect of Peptide in a Murine EHEC infection model.

| Treatment | Peptide | Survival (%) |
|---|---|---|
| control | none | 25 |
| SEQ ID NO: 23 | 200 μg | 100 |

CD-1 male mice (5 weeks old) were given 5.8 × 10⁶ CFU EHEC bacteria via intraperitoneal (IP) injection. Cationic peptide (200 μg) was given via a separate IP injection. The mice were monitored for 3 days.

TABLE 53
Up-regulation of patterns of gene expression in A549 epithelial cells
induced by peptides that are active in vivo.

| | | Fold Up regulation of | | | |
|---|---|---|---|---|---|
| | | Gene Expression | | | |
| | | relative to Untreated Cells | | | |
| | Unstimulated | SEQ ID | SEQ ID | SEQ ID | SEQ ID |
| Target (Accession number) | Cell Intensity | NO: 30 | NO: 7 | NO: 13 | NO: 37 |
| Zinc finger protein (AF061261) | 13 | 2.6 | 9.4 | 9.4 | 1.0 |
| Cell cycle gene (S70622) | 1.62 | 8.5 | 3.2 | 3.2 | 0.7 |
| IL-10 Receptor (U00672) | 0.2 | 2.6 | 9 | 4.3 | 0.5 |
| Transferase (AF038664) | 0.09 | 12.3 | 9.7 | 9.7 | 0.1 |
| Homeobox protein (AC004774) | 0.38 | 3.2 | 2.5 | 2.5 | 1.7 |
| Forkhead protein (AF042832) | 0.17 | 14.1 | 3.5 | 3.5 | 0.9 |
| Unknown (AL096803) | 0.12 | 4.8 | 4.3 | 4.3 | 0.6 |
| KIAA0284 Protein (AB006622) | 0.47 | 3.4 | 2.1 | 2.1 | 1.3 |
| Hypothetical Protein (AL022393) | 0.12 | 4.4 | 4.0 | 4.0 | 0.4 |
| Receptor (AF112461) | 0.16 | 2.4 | 10.0 | 10.0 | 1.9 |
| Hypothetical Protein (AK002104) | 0.51 | 4.7 | 2.6 | 2.6 | 1.0 |
| Protein (AL050261) | 0.26 | 3.3 | 2.8 | 2.8 | 1.0 |
| Polypeptide (AF105424) | 0.26 | 2.5 | 5.3 | 5.3 | 1.0 |
| SPR1 protein (AB031480) | 0.73 | 3.0 | 2.7 | 2.7 | 1.3 |
| Dehydrogenase (D17793) | 4.38 | 2.3 | 2.2 | 2.2 | 0.9 |
| Transferase (M63509) | 0.55 | 2.7 | 2.1 | 2.1 | 1.0 |
| Peroxisome factor (AB013818) | 0.37 | 3.4 | 2.9 | 2.9 | 1.4 |
| The peptides SEQ ID NO: 30, SEQ ID NO: 7 and SEQ ID NO: 13 at concentrations of 50 μg/ml were each shown to increase the expression of a pattern of genes after 4 h treatment. Peptide was incubated with the human A549 epithelial cells for 4 h and the RNA was isolated, converted into labelled cDNA probes and hybridised to Human Operon arrays (PRHU04). The intensity of polynucleotides in control, unstimulated cells are shown in the second columns for | | | | | |
| # labelling of cDNA (average of Cy3 and Cy5). The Fold Up regulation column refers to the intensity of polynucleotide expression in peptide-simulated cells divided by the intensity of unstimulated cells. | | | | | |

The SEQ ID NO: 37 peptide was included as a negative control that was not active in the murine infection models.

TABLE 54

| | MIC (μg/ml) | | | | | |
|---|---|---|---|---|---|---|
| Peptide | E. coli | S. aureus | P. aerug. | S. typhim. | C. rhod. | EHEC |
| Polymyxin | 0.25 | 16 | 0.25 | 0.5 | 0.25 | 0.5 |
| Gentamicin | 0.25 | 0.25 | 0.25 | 0.25 | 0.25 | 0.5 |
| SEQ ID NO: 1 | 32 | > | 96 | 64 | 8 | 4 |
| SEQ ID NO: 5 | 128 | > | > | > | 64 | 64 |
| SEQ ID NO: 6 | 128 | > | > | 128 | 64 | 64 |
| SEQ ID NO: 7 | > | > | > | > | > | > |
| SEQ ID NO: 8 | > | > | > | > | > | > |
| SEQ ID NO: 9 | > | > | > | > | > | > |
| SEQ ID NO: 10 | > | > | > | > | > | 64 |
| SEQ ID NO: 12 | > | > | > | > | > | > |
| SEQ ID NO: 13 | > | > | > | > | > | > |
| SEQ ID NO: 14 | > | > | > | > | > | > |
| SEQ ID NO: 15 | 128 | > | > | > | 128 | 64 |
| SEQ ID NO: 16 | > | > | > | > | > | > |
| SEQ ID NO: 17 | > | > | > | > | > | > |
| SEQ ID NO: 19 | 8 | 16 | 16 | 64 | 4 | 4 |
| SEQ ID NO: 2 | 4 | 16 | 32 | 16 | 64 | |
| SEQ ID NO: 20 | 8 | 8 | 8 | 8 | 16 | 8 |
| SEQ ID NO: 21 | 64 | 64 | 96 | 64 | 32 | 32 |
| SEQ ID NO: 22 | 8 | 12 | 24 | 8 | 4 | 4 |
| SEQ ID NO: 23 | 4 | 8 | 8 | 16 | 4 | 4 |
| SEQ ID NO: 24 | 16 | 16 | 4 | 16 | 16 | 4 |
| SEQ ID NO: 26 | 0.5 | 32 | 64 | 2 | 2 | 0.5 |
| SEQ ID NO: 27 | 8 | 64 | 64 | 16 | 2 | 4 |
| SEQ ID NO: 28 | > | > | > | 64 | 64 | 128 |
| SEQ ID NO: 29 | 2 | > | > | 16 | 32 | 4 |
| SEQ ID NO: 30 | 16 | > | 128 | 16 | 16 | 4 |
| SEQ ID NO: 31 | > | > | 128 | > | > | 64 |
| SEQ ID NO: 33 | 16 | 32 | > | 16 | 64 | 8 |
| SEQ ID NO: 34 | 8 | > | > | 32 | 64 | 8 |
| SEQ ID NO: 35 | 4 | 128 | 64 | 8 | 8 | 4 |
| SEQ ID NO: 36 | 32 | > | > | 32 | 32 | 16 |
| SEQ ID NO: 37 | > | > | > | > | > | > |
| SEQ ID NO: 38 | 0.5 | 32 | 64 | 4 | 8 | 4 |
| SEQ ID NO: 40 | 4 | 32 | 8 | 4 | 4 | 2 |
| SEQ ID NO: 41 | 4 | 64 | 8 | 8 | 2 | 2 |
| SEQ ID NO: 42 | 1.5 | 64 | 4 | 2 | 2 | 1 |
| SEQ ID NO: 43 | 8 | 128 | 16 | 16 | 8 | 4 |
| SEQ ID NO: 44 | 8 | > | 128 | 128 | 64 | 64 |
| SEQ ID NO: 45 | 8 | > | 128 | 128 | 16 | 16 |
| SEQ ID NO: 47 | 4 | > | 16 | 16 | 4 | 4 |
| SEQ ID NO: 48 | 16 | > | 128 | 16 | 1 | 2 |
| SEQ ID NO: 49 | 4 | > | 16 | 8 | 4 | 4 |
| SEQ ID NO: 50 | 8 | > | 16 | 16 | 16 | 8 |
| SEQ ID NO: 51 | 4 | > | 8 | 32 | 4 | 8 |
| SEQ ID NO: 52 | 8 | > | 32 | 8 | 2 | 2 |
| SEQ ID NO: 53 | 4 | > | 8 | 8 | 16 | 8 |
| SEQ ID NO: 54 | 64 | > | 16 | 64 | 16 | 32 |

Most cationic peptides studied here and especially the cationic peptides effective in infection models are not significantly antimicrobial. A dilution series of peptide was incubated with the indicated bacteria overnight in a 96-well plate. The lowest concentration of peptide that killed the bacteria was used as the MIC. The symbol > indicates the MIC is too large to measure. An MIC of 4 μg/ml or less was considered clinically meaningful activity.
Abbreviations:
E. coli, Escherichia coli;
S. aureus, Staphylococcus aureus;
P. aerug, Pseudomonas aeruginosa;
S. Typhim, Salmonella enteritidis ssp. typhimurium;
C. rhod, Citobacter rhodensis;
EHEC, Enterohaemorrhagic E.coli.

### EXAMPLE 9

### Use of Polynucleotides Induced by Bacterial Signalling Molecules in Diagnostic/Screening

S. typhimurium LPS and E. coli 0111:B4 LPS were purchased from Sigma Chemical Co. (St. Louis, Mo.). LTA (Sigma) from S. aureus, was resuspended in endotoxin free water (Sigma). The Limulus amoebocyte lysate assay (Sigma) was performed on LTA preparations to confirm that lots were not significantly contaminated by endotoxin (i.e. <1 ng/ml, a concentration that did not cause significant cytokine production in the RAW cell assay). The CpG oligodeoxynucleotides were synthesized with an Applied Biosystems Inc., Model 392 DNA/RNA Synthesizer, Mississauga, ON., then purified and resuspended in endotoxin-free water (Sigma). The following sequences were used CpG: 5′-TCATGACGTTCCTGACGTT-3′ (SEQ ID NO: 57) and nonCpG: 5′-TTCAGGACTTTCCTCAGGTT-3′(SEQ ID NO: 58). The nonCpG oligo was tested for its ability to stimulate production of cytokines and was found to cause no significant production of TNF-α or IL-6 and therefore was considered as a negative control. RNA was isolated from RAW 264.7 cells that had been incubated for 4 h with medium alone, 100 ng/ml S. typhimurium LPS, 1 μg/ml S. aureus LTA, or 1 μM CpG (concentrations that led to optimal induction of tumor necrosis factor (TNF-α) in RAW cells). The RNA was used to polynucleotiderate cDNA probes that were hybridized to Clontech Atlas polynucleotide array filters, as described above. The hybridization of the cDNA probes to each immobilized DNA was visualized by autoradiography and quantified using a phosphorimager. Results from at least 2 to 3 independent experiments are summarized in Tables 55-59. It was found that LPS treatment of RAW 264.7 cells resulted in increased expression of more than 60 polynucleotides including polynucleotides encoding inflammatory proteins such as IL-1β, inducible nitric oxide synthase (iNOS), MIP-1α, MIP-1β, MIP-2α, CD40, and a variety of transcription factors. When the changes in polynucleotide expression induced by LPS, LTA, and CpG DNA were compared, it was found that all three of these bacterial products increased the expression of pro-inflammatory polynucleotides such as iNOS, MIP-1α, MIP-2α, IL-1β, IL-15, TNFR1 and NF-κB to a similar extent (Table 57). Table 57 describes 19 polynucleotides that were up-regulated by the bacterial products to similar extents in that their stimulation ratios differed by less than 1.5 fold between the three bacterial products. There were also several polynucleotides that were down-regulated by LPS, LTA and CpG to a similar extent. It was also found that there were a number of polynucleotides that were differentially regulated in response to the three bacterial products (Table 58), which includes many of these polynucleotides that differed in expression levels by more than 1.5 fold between one or more bacterial products). LTA treatment differentially influenced expression of the largest subset of polynucleotides compared to LPS or CpG, including hyperstimulation of expression of Jun-D, Jun-B, Elk-1 and cyclins G2 and A1. There were only a few polynucleotides whose expression was altered more by LPS or CpG treatment. Polynucleotides that had preferentially increased expression due to LPS treatment compared to LTA or CpG treatment included the cAMP response element DNA-binding protein 1 (CRE-BPI), interferon inducible protein 1 and CACCC Box-binding protein BKLF. Polynucleotides that had preferentially increased expression after CpG treatment compared to LPS or LTA treatment included leukemia inhibitory factor (LIF) and protease nexin 1 (PN-1). These results indicate that although LPS, LTA, and CpG DNA stimulate largely overlapping polynucleotide expression responses, they also exhibit differential abilities to regulate certain subsets of polynucleotides.

The other polynucleotide arrays used are the Human Operon arrays (identification number for the genome is PRHU04-S1), which consist of about 14,000 human oligos spotted in duplicate. Probes were prepared from 5 μg of total RNA and labeled with Cy3 or Cy5 labeled dUTP. In these experiments, A549 epithelial cells were plated in 100 mm tissue culture dishes at 2.5×10⁶ cells/dish, incubated overnight and then stimulated with 100 ng/ml E. coli O111:B4 LPS for 4 h. Total RNA was isolated using RNAqueous (Ambion). DNA contamination was removed with DNA-free kit (Ambion). The probes prepared from total RNA were purified and hybridized to printed glass slides overnight at 42° C. and washed. After washing, the image was captured using a Perkin Elmer array scanner. The image processing software (Imapolynucleotide 5.0, Marina Del Rey, Calif.) determines the spot mean intensity, median intensities, and background intensities. An “in house” program was used to remove background. The program calculates the bottom 10% intensity for each subgrid and subtracts this for each grid. Analysis was performed with Polynucleotidespring software (Redwood City, Calif.). The intensities for each spot were normalized by taking the median spot intensity value from the population of spot values within a slide and comparing this value to the values of all slides in the experiment. The relative changes seen with cells treated with LPS compared to control cells can be found in the Tables below. A number of previously unreported changes that would be useful in diagnosing infection are described in Table 60.

To confirm and assess the functional significance of these changes, the levels of selected mRNAs and proteins were assessed and quantified by densitometry. Northern blots using a CD14, vimentin, and tristetraprolin-specific probe confirmed similar expression after stimulation with all 3 bacterial products (Table 60). Similarly measurement of the enzymatic activity of nitric oxide synthetase, iNOS, using Griess reagent to assess levels of the inflammatory mediator NO, demonstrated comparable levels of NO produced after 24 h, consistent with the similar up-regulation of iNOS expression (Table 59). Western blot analysis confirmed the preferential stimulation of leukaemia inhibitory factor (LIF, a member of the IL-6 family of cytokines) by CpG (Table 59). Other confirmatory experiments demonstrated that LPS up-regulated the expression of TNF-α and IL-6 as assessed by ELISA, and the up-regulated expression of MIP-2α, and IL-1β mRNA and down-regulation of DP-1 and cyclin D mRNA as assessed by Northern blot analysis. The analysis was expanded to a more clinically relevant ex vivo system, by examining the ability of the bacterial elements to stimulate pro-inflammatory cytokine production in whole human blood. It was found that E. coli LPS, S. typhimurium LPS, and S. aureus LTA all stimulated similar amounts of serum TNF-α, and IL-1β. CpG also stimulated production of these cytokines, albeit to much lower levels, confirming in part the cell line data.

TABLE 55
Polynucleotides Up-regulated by E. coli O11:B4
LPS in A549 Epithelial Cells.

| | | Control: | |
|---|---|---|---|
| Accession | | Media only | Ratio: |
| Number | Gene | Intensity | LPS/control |
| D87451 | ring finger protein 10 | 715.8 | 183.7 |
| AF061261 | C3H-type zinc finger protein | 565.9 | 36.7 |
| D17793 | aldo-keto reductase family 1, | 220.1 | 35.9 |
| | member C3 | | |
| M14630 | prothymosin, alpha | 168.2 | 31.3 |
| AL049975 | Unknown | 145.6 | 62.3 |
| L04510 | ADP-ribosylation factor | 139.9 | 213.6 |
| | domain protein 1, 64 kD | | |
| U10991 | G2 protein | 101.7 | 170.3 |
| U39067 | eukaryotic translation | 61.0 | 15.9 |
| | initiation factor 3, subunit 2 | | |
| X03342 | ribosomal protein L32 | 52.6 | 10.5 |
| NM_004850 | Rho-associated, coiled-coil | 48.1 | 11.8 |
| | containing protein kinase 2 | | |
| AK000942 | Unknown | 46.9 | 8.4 |
| AB040057 | serine/threonine protein | 42.1 | 44.3 |
| | kinase MASK | | |
| AB020719 | KIAA0912 protein | 41.8 | 9.4 |
| AB007856 | FEM-1-like death receptor | 41.2 | 15.7 |
| | binding protein | | |
| J02783 | procollagen-proline, 2- | 36.1 | 14.1 |
| | oxoglutarate 4-dioxygenase | | |
| AL137376 | Unknown | 32.5 | 17.3 |
| AL137730 | Unknown | 29.4 | 11.9 |
| D25328 | phosphofructokinase, platelet | 27.3 | 8.5 |
| AF047470 | malate dehydrogenase 2, | 25.2 | 8.2 |
| | NAD | | |
| M86752 | stress-induced- | 22.9 | 5.9 |
| | phosphoprotein 1 | | |
| M90696 | cathepsin S | 19.6 | 6.8 |
| AK001143 | Unknown | 19.1 | 6.4 |
| AF038406 | NADH dehydrogenase | 17.7 | 71.5 |
| AK000315 | hypothetical protein | 17.3 | 17.4 |
| | FLJ20308 | | |
| M54915 | pim-1 oncogene | 16.0 | 11.4 |
| D29011 | proteasome subunit, beta | 15.3 | 41.1 |
| | type, 5 | | |
| AK000237 | membrane protein of | 15.1 | 9.4 |
| | cholinergic synaptic vesicles | | |
| AL034348 | Unknown | 15.1 | 15.8 |
| AL161991 | Unknown | 14.2 | 8.1 |
| AL049250 | Unknown | 12.7 | 5.6 |
| AL050361 | PTD017 protein | 12.6 | 13.0 |
| U74324 | RAB interacting factor | 12.3 | 5.2 |
| M22538 | NADH dehydrogenase | 12.3 | 7.6 |
| D87076 | KIAA0239 protein | 11.6 | 6.5 |
| NM_006327 | translocase of inner | 11.5 | 10.0 |
| | mitochondrial membrane 23 | | |
| | (yeast) homolog | | |
| AK001083 | Unknown | 11.1 | 8.6 |
| AJ001403 | mucin 5, subtype B, | 10.8 | 53.4 |
| | tracheobronchial | | |
| M64788 | RAP1, GTPase activating | 10.7 | 7.6 |
| | protein 1 | | |
| X06614 | retinoic acid receptor, alpha | 10.7 | 5.5 |
| U85611 | calcium and integring binding | 10.3 | 8.1 |
| | protein | | |
| U23942 | cytochrome P450, 51 | 10.1 | 10.2 |
| AL031983 | Unknown | 9.7 | 302.8 |
| NM_007171 | protein-O- | 9.5 | 6.5 |
| | mannosyltransferase 1 | | |
| AK000403 | hypothetical protein | 9.5 | 66.6 |
| | FLJ20396 | | |
| NM_002950 | ribophorin I | 9.3 | 35.7 |
| L05515 | cAMP response element- | 8.9 | 6.2 |
| | binding protein CRE-BPa | | |
| X83368 | phosphoinositide-3-kinase, | 8.7 | 27.1 |
| | catalytic, gamma polypeptide | | |
| M30269 | nidogen (enactin) | 8.7 | 5.5 |
| M91083 | chromosome 11 open reading | 8.2 | 6.6 |
| | frame 13 | | |
| D29833 | salivary proline-rich protein | 7.7 | 5.8 |
| AB024536 | immunoglobulin superfamily | 7.6 | 8.0 |
| | containing leucine-rich repeat | | |
| U39400 | chromosome 11 open reading | 7.4 | 7.3 |
| | frame 4 | | |
| AF028789 | unc119 (C. elegans) homolog | 7.4 | 27.0 |
| NM_003144 | signal sequence receptor, | 7.3 | 5.9 |
| | alpha (translocon-associated | | |
| | protein alpha) | | |
| X52195 | arachidonate 5-lipoxygenase- | 7.3 | 13.1 |
| | activating protein | | |
| U43895 | human growth factor- | 6.9 | 6.9 |
| | regulated tyrosine kinase | | |
| | substrate | | |
| L25876 | cyclin-dependent kinase | 6.7 | 10.3 |
| | inhibitor 3 | | |
| L04490 | NADH dehydrogenase | 6.6 | 11.1 |
| Z18948 | S100 calcium-binding protein | 6.3 | 11.0 |
| D10522 | myristoylated alanine-rich | 6.1 | 5.8 |
| | protein kinase C substrate | | |
| NM_014442 | sialic acid binding Ig-like | 6.1 | 7.6 |
| | lectin 8 | | |
| U81375 | solute carrier family 29 | 6.0 | 6.4 |
| AF041410 | malignancy-associated | 5.9 | 5.3 |
| | protein | | |
| U24077 | killer cell immunoglobulin- | 5.8 | 14.4 |
| | like receptor | | |
| AL137614 | hypothetical protein | 4.8 | 6.8 |
| NM_002406 | mannosyl (alpha-1,3-)- | 4.7 | 5.3 |
| | glycoprotein beta-1,2-N- | | |
| | acetylglucosaminyltransferase | | |
| AB002348 | KIAA0350 protein | 4.7 | 7.6 |
| AF165217 | tropomodulin 4 (muscle) | 4.6 | 12.3 |
| Z14093 | branched chain keto acid | 4.6 | 5.4 |
| | dehydrogenase E1, alpha | | |
| | polypeptide | | |
| U82671 | caltractin | 3.8 | 44.5 |
| AL050136 | Unknown | 3.6 | 5.0 |
| NM_005135 | solute carrier family 12 | 3.6 | 5.0 |
| AK001961 | hypothetical protein | 3.6 | 5.9 |
| | FLJ11099 | | |
| AL034410 | Unknown | 3.2 | 21.3 |
| S74728 | antiquitin 1 | 3.1 | 9.2 |
| AL049714 | ribosomal protein L34 | 3.0 | 19.5 |
| | pseudogene 2 | | |
| NM_014075 | PRO0593 protein | 2.9 | 11.5 |
| AF189279 | phospholipase A2, group IIE | 2.8 | 37.8 |
| J03925 | integrin, alpha M | 2.7 | 9.9 |
| NM_012177 | F-box protein Fbx5 | 2.6 | 26.2 |
| NM_004519 | potassium voltage-gated | 2.6 | 21.1 |
| | channel, KQT-like subfamily, | | |
| | member 3 | | |
| M28825 | CD1A antigen, a polypeptide | 2.6 | 16.8 |
| X16940 | actin, gamma 2, smooth | 2.4 | 11.8 |
| | muscle, enteric | | |
| X03066 | major histocompatibility | 2.2 | 36.5 |
| | complex, class II, DO beta | | |
| AK001237 | hypothetical protein | 2.1 | 18.4 |
| | FLJ10375 | | |
| AB028971 | KIAA1048 protein | 2.0 | 9.4 |
| AL137665 | Unknown | 2.0 | 7.3 |
| E. coli O111:B4 LPS (100 ng/ml) increased the expression of many polynucleotides in A549 cells as studied by polynucleotide microarrays. LPS was incubated with the A549 cells for 4 h and the RNA was isolated. 5 μg total RNA was used to make Cy3/Cy5 labelled cDNA probes and hybridised onto Human Operon arrays (PRHU04). The intensity of unstimulated cells is shown in the second column of Table 55. The “Ratio: LPS/control” column refers to the intensity of | | | |
| # polynucleotide expression in LPS simulated cells divided by in the intensity of unstimulated cells. | | | |

TABLE 56
Polynucleotides Down-regulated by E. coli O111:B4 LPS in A549 Epitheial Cells.

| | | Control: | |
|---|---|---|---|
| Accession | | Media only | Ratio: |
| Number | Gene | Intensity | LPS/control |
| NM_017433 | myosin IIIA | 167.8 | 0.03 |
| X60484 | H4 histone family member E | 36.2 | 0.04 |
| X60483 | H4 histone family member D | 36.9 | 0.05 |
| AF151079 | hypothetical protein | 602.8 | 0.05 |
| M96843 | inhibitor of DNA binding 2, dominant | 30.7 | 0.05 |
| | negative helix-loop-helix protein | | |
| S79854 | deiodinase, iodothyronine, type III | 39.4 | 0.06 |
| AB018266 | matrin 3 | 15.7 | 0.08 |
| M33374 | NADH dehydrogenase | 107.8 | 0.09 |
| AF005220 | Homo sapiens mRNA for NUP98-HOXD13 | 105.2 | 0.09 |
| | fusion protein, partial cds | | |
| Z80783 | H2B histone family, member L | 20.5 | 0.10 |
| Z46261 | H3 histone family, member A | 9.7 | 0.12 |
| Z80780 | H2B histone family, member H | 35.3 | 0.12 |
| U33931 | erythrocyte membrane protein band 7.2 | 18.9 | 0.13 |
| | (stomatin) | | |
| M60750 | H2B histone family, member A | 35.8 | 0.14 |
| Z83738 | H2B histone family, member E | 19.3 | 0.15 |
| Y14690 | collagen, type V, alpha 2 | 7.5 | 0.15 |
| M30938 | X-ray repair complementing defective | 11.3 | 0.16 |
| | repair in Chinese hamster cells 5 | | |
| L36055 | eukaryotic translation initiation factor 4E | 182.5 | 0.16 |
| | binding protein 1 | | |
| Z80779 | H2B histone family, member G | 54.3 | 0.16 |
| AF226869 | 5(3)-deoxyribonucleotidase; RB-associated | 7.1 | 0.18 |
| | KRAB repressor | | |
| D50924 | KIAA0134 gene product | 91.0 | 0.18 |
| AL133415 | vimentin | 78.1 | 0.19 |
| AL050179 | tropomyosin 1 (alpha) | 41.6 | 0.19 |
| AJ005579 | RD element | 5.4 | 0.19 |
| M80899 | AHNAK nucleoprotein | 11.6 | 0.19 |
| NM_004873 | BCL2-associated athanogene 5 | 6.2 | 0.19 |
| X57138 | H2A histone family, member N | 58.3 | 0.20 |
| AF081281 | lysophospholipase I | 7.2 | 0.22 |
| U96759 | von Hippel-Lindau binding protein 1 | 6.6 | 0.22 |
| U85977 | Human ribosomal protein L12 pseudogene, | 342.6 | 0.22 |
| | partial cds | | |
| D13315 | glyoxalase I | 7.5 | 0.22 |
| AC003007 | Unknown | 218.2 | 0.22 |
| AB032980 | RU2S | 246.6 | 0.22 |
| U40282 | integrin-linked kinase | 10.1 | 0.22 |
| U81984 | endothelial PAS domain protein 1 | 4.7 | 0.23 |
| X91788 | chloride channel, nucleotide-sensitive, 1A | 9.6 | 0.23 |
| AF018081 | collagen, type XVIII, alpha 1 | 6.9 | 0.24 |
| L31881 | nuclear factor I/X (CCAAT-binding | 13.6 | 0.24 |
| | transcription factor) | | |
| X61123 | B-cell translocation gene 1, anti- | 5.3 | 0.24 |
| | proliferative | | |
| L32976 | mitogen-activated protein kinase kinase | 6.3 | 0.24 |
| | kinase 11 | | |
| M27749 | immunoglobulin lambda-like polypeptide 3 | 5.5 | 0.24 |
| X57128 | H3 histone family, member C | 9.0 | 0.25 |
| X80907 | phosphoinositide-3-kinase, regulatory | 5.8 | 0.25 |
| | subunit, polypeptide 2 | | |
| Z34282 | H. sapiens (MAR11) MUC5AC mRNA for | 100.6 | 0.26 |
| | mucin (partial) | | |
| X00089 | H2A histone family, member M | 4.7 | 0.26 |
| AL035252 | CD39-like 2 | 4.6 | 0.26 |
| X95289 | PERB11 family member in MHC class I | 27.5 | 0.26 |
| | region | | |
| AJ001340 | U3 snoRNP-associated 55-kDa protein | 4.0 | 0.26 |
| NM_014161 | HSPC071 protein | 10.6 | 0.27 |
| U60873 | Unknown | 6.4 | 0.27 |
| X91247 | thioredoxin reductase 1 | 84.4 | 0.27 |
| AK001284 | hypothetical protein FLJ10422 | 4.2 | 0.27 |
| U90840 | synovial sarcoma, X breakpoint 3 | 6.6 | 0.27 |
| X53777 | ribosomal protein L17 | 39.9 | 0.27 |
| AL035067 | Unknown | 10.0 | 0.28 |
| AL117665 | DKFZP586M1824 protein | 3.9 | 0.28 |
| L14561 | ATPase, Ca++ transporting, plasma | 5.3 | 0.28 |
| | membrane 1 | | |
| L19779 | H2A histone family, member O | 30.6 | 0.28 |
| AL049782 | Unknown | 285.3 | 0.28 |
| X00734 | tubulin, beta, 5 | 39.7 | 0.29 |
| AK001761 | retinoic acid induced 3 | 23.7 | 0.29 |
| U72661 | ninjurin 1 | 4.4 | 0.29 |
| S48220 | deiodinase, iodothyronine, type I | 1,296.1 | 0.29 |
| AF025304 | EphB2 | 4.5 | 0.30 |
| S82198 | chymotrypsin C | 4.1 | 0.30 |
| Z80782 | H2B histone family, member K | 31.9 | 0.30 |
| X68194 | synaptophysin-like protein | 7.9 | 0.30 |
| AB028869 | Unknown | 4.2 | 0.30 |
| AK000761 | Unknown | 4.3 | 0.30 |
| E. coli O111 :B4 LPS (100 ng/ml) decreased the expression of many polynucleotides in A549 cells as studied by polynucleotide microarrays. LPS was incubated with the A549 cells for 4 h and the RNA was isolated. 5 μg total RNA was used to make Cy3/Cy5 labeled cDNA probes and hybridized onto Human Operon arrays (PRHU04). The intensity of unstimulated cells is shown in the second column of the Table. The “Ratio: LPS/control” column refers to the intensity of | | | |
| # polynuclebtide expression in LPS simulated cells divided by in the intensity of unstimulated cells. | | | |

TABLE 57
Polynucleotides expressed to similar extents after stimulation by the bacterial
products LPS, LTA, and CpG DNA.

| | Control | | | | |
|---|---|---|---|---|---|
| Accession | Unstim. | Ratio | Ratio | Ratio | |
| number | Intensity | LPS:Control | LTA:Control | CpG:Control | Protein/polynucleotide |
| M15131 | 20 | 82 | 80 | 55 | IL-1β |
| M57422 | 20 | 77 | 64 | 90 | tristetraprolin |
| X53798 | 20 | 73 | 77 | 78 | MIP-2α |
| M35590 | 188 | 50 | 48 | 58 | MIP-1β |
| L28095 | 20 | 49 | 57 | 50 | ICE |
| M87039 | 20 | 37 | 38 | 45 | iNOS |
| X57413 | 20 | 34 | 40 | 28 | TGFβ |
| X15842 | 20 | 20 | 21 | 15 | c-rel proto-oncopolynucleotide |
| X12531 | 489 | 19 | 20 | 26 | MIP-1α |
| U14332 | 20 | 14 | 15 | 12 | IL-15 |
| M59378 | 580 | 10 | 13 | 11 | TNFR1 |
| U37522 | 151 | 6 | 6 | 6 | TRAIL |
| M57999 | 172 | 3.8 | 3.5 | 3.4 | NF-κB |
| U36277 | 402 | 3.2 | 3.5 | 2.7 | I-κB (alpha subunit) |
| X76850 | 194 | 3 | 3.8 | 2.5 | MAPKAP-2 |
| U06924 | 858 | 2.4 | 3 | 3.2 | Stat 1 |
| X14951 | 592 | 2 | 2 | 2 | CD18 |
| X60671 | 543 | 1.9 | 2.4 | 2.8 | NF-2 |
| M34510 | 5970 | 1.6 | 2 | 1.4 | CD14 |
| X51438 | 2702 | 1.3 | 2.2 | 2.0 | vimentin |
| X68932 | 4455 | 0.5 | 0.7 | 0.5 | c-Fms |
| Z21848 | 352 | 0.5 | 0.6 | 0.6 | DNA polymerase |
| X70472 | 614 | 0.4 | 0.6 | 0.5 | B-myb |

Bacterial products (100 ng/ml S. typhimurium LPS, 1 μg/ml S. aureus LTA or 1 μM CpG) were shown to potently induce the expression of several polynucleotides. Peptide was incubated with the RAW cells for 4 h and the RNA was isolated, converted into labeled cDNA probes and hybridized to Atlas arrays. The intensity of control, unstimulated cells is shown in the second column.
The “Ratio LPS/LTA/CpG:Control” column refers to the intensity of polynucleotide expression in bacterial product-simulated cells divided by the intensity of unstimulated cells.

TABLE 58
Polynucleotides that were differentially regulated by the bacterial products
LPS, LTA, and CpG DNA.

| | Unstim. | | | | |
|---|---|---|---|---|---|
| Accession | Control | Ratio | Ratio | Ratio | |
| number | Intensity | LPS:Contrl | LTA:Contrl | CpG:Contrl | Protein/polynucleotide |
| X72307 | 20 | 1.0 | 23 | 1.0 | hepatocyte growth factor |
| L38847 | 20 | 1.0 | 21 | 1.0 | hepatoma transmembrane kinase |
| | | | | | ligand |
| L34169 | 393 | 0.3 | 3 | 0.5 | thrombopoietin |
| J04113 | 289 | 1 | 4 | 3 | Nur77 |
| Z50013 | 20 | 7 | 21 | 5 | H-ras proto-oncopolynucleotide |
| X84311 | 20 | 4 | 12 | 2 | Cyclin A1 |
| U95826 | 20 | 5 | 14 | 2 | Cyclin G2 |
| X87257 | 123 | 2 | 4 | 1 | Elk-1 |
| J05205 | 20 | 18 | 39 | 20 | Jun-D |
| J03236 | 20 | 11 | 19 | 14 | Jun-B |
| M83649 | 20 | 71 | 80 | 42 | Fas 1 receptor |
| M83312 | 20 | 69 | 91 | 57 | CD40L receptor |
| X52264 | 20 | 17 | 23 | 9 | ICAM-1 |
| M13945 | 573 | 2 | 3 | 2 | Pim-1 |
| U60530 | 193 | 2 | 3 | 3 | Mad related protein |
| D10329 | 570 | 2 | 3 | 2 | CD7 |
| X06381 | 20 | 55 | 59 | 102 | Leukemia inhibitory factor (LIF) |
| X70296 | 20 | 6.9 | 13 | 22 | Protease nexin 1 (PN-1) |
| U36340 | 20 | 38 | 7 | 7 | CACCC Box-binding protein |
| | | | | | BKLF |
| S76657 | 20 | 11 | 6 | 7 | CRE-BPI |
| U19119 | 272 | 10 | 4 | 4 | interferon inducible protein 1 |

Bacterial products (100 ng/ml S. typhimurium LPS, 1 μg/ml S. aureus LTA or 1 μM CpG) were shown to potently induce the expression of several polynucleotides. Peptide was incubated with the RAW cells for 4 h and the RNA was isolated, converted into labeled cDNA probes and hybridized to Atlas arrays. The intensity of control, unstimulated cells is shown in the second column.
The “Ratio LPS/LTA/CpG:Control” column refers to the intensity of polynucleotide expression in bacterial product-simulated cells divided by the intensity of unstimulated cells.

TABLE 59
Confirmation of Table 57 and 58 Array Data.

| | Relative levels | | | |
|---|---|---|---|---|
| Product | Untreated | LPS | LTA | CpG |
| CD14^{a} | 1.0 | 2.2 ± 0.4 | 1.8 ± 0.2 | 1.5 ± 0.3 |
| Vimentin^{a} | 1.0 | 1.2 ± 0.07 | 1.5 ± 0.05 | 1.3 ± 0.07 |
| Tristetraprolin^{a} | 1.0 | 5.5 ± 0.5 | 5.5 ± 1.5 | 9.5 ± 1.5 |
| LIF^{b} | 1.0 | 2.8 ± 1.2 | 2.7 ± 0.6 | 5.1 ± 1.6 |
| NO^{c} | 8 ± 1.5 | 47 ± 2.5 | 20 ± 3 | 21 ± 1.5 |
| ^{a}Total RNA was isolated from unstimulated RAW macrophage cells and cells treated for 4 hr with 100 ng/ml S. typhimurium LPS, 1 μg/ml S. aureus LTA, 1 μM CpG DNA or media alone and Northern blots were performed the membrane was probed for GAPDH, CD14, vimentin, and tristetraprolin as described previously [Scott et al]. The hybridization intensities of the Northern blots were compared to GAPDH to look for inconsistencies in loading. These experiments were | | | | |
| # repeated at least three times and the data shown is the average relative levels of each condition compared to media (as measured by densitometry) ± standard error. | | | | |

^{b}RAW 264.7 cells were stimulated with 100 ng/ml S. typhimurium LPS, 1 μg/ml S. aureus LTA, 1 μM CpG DNA or media alone for 24 hours. Protein lysates were prepared, run on SDS PAGE gels and western blots wre performed to detect LIF (R&D Systems). These experiments were repeated at least three times and the data shown is the relative levels of LIF compared to media (as measured by densitometry) ± standard error.
^{c}Supernatant was collected from RAW macrophage cells treated with 100 ng/ml S. typhimurium LPS, 1 μg/ml S. aureus LTA, 1 μM CpG DNA, or media alone for 24 hours and tested for the amount of NO formed in the supematant as estimated from the accumulation of the stable NO metabolite nitrite with the Griess reagent as described previously [Scott, et al]. The data shown is the average of three experiments ± standard error.

TABLE 60
Pattern of Gene expression in A549 Human Epithelial cells
up-regulated by bacterial signalling molecules (LPS).

| Accession Number | Gene |
|---|---|
| AL050337 | interferon gamma receptor 1 |
| U05875 | interferon gamma receptor 2 |
| NM_002310 | leukemia inhibitory factor receptor |
| U92971 | coagulation factor II (thrombin) receptor-like 2 |
| Z29575 | tumor necrosis factor receptor superfamily |
| | member 17 |
| L31584 | Chemokine receptor 7 |
| J03925 | cAMP response element-binding protein |
| M64788 | RAP1, GTPase activating protein |
| NM_004850 | Rho-associated kinase 2 |
| D87451 | ring finger protein 10 |
| AL049975 | Unknown |
| U39067 | eukaryotic translation initiation factor 3, subunit 2 |
| AK000942 | Unknown |
| AB040057 | serine/threonine protein kinase MASK |
| AB020719 | KIAA0912 protein |
| AB007856 | FEM-1-like death receptor binding protein |
| AL137376 | Unknown |
| AL137730 | Unknown |
| M90696 | cathepsin S |
| AK001143 | Unknown |
| AF038406 | NADH dehydrogenase |
| AK000315 | hypothetical protein FLJ20308 |
| M54915 | pim-1 oncogene |
| D29011 | proteasome subunit, beta type, 5 |
| AL034348 | Unknown |
| D87076 | KIAA0239 protein |
| AJ001403 | mucin 5, subtype B, tracheobronchial |
| J03925 | integrin, alpha M |
| E. coli 0111: B4 LPS (100 ng/ml) increased the expression of many polynucleotides in A549 cells as studied by polynucleotide microarrays. LPS was incubated with the A549 cells for 4 h and the RNA was isolated. 5 μg total RNA was used to make Cy3/Cy5 labelled cDNA probes and hybridised onto Human Operon arrays (PRHU04). The examples of polynucleotide expression changes in LPS simulated cells represent a greater than | |
| # 2-fold intensity level change of LPS treated cells from untreated cells. | |

### EXAMPLE 10

### Altering Signaling to Protect Against Bacterial Infections

The Salmonella Typhimurium strain SL 1344 was obtained from the American Type Culture Collection (ATCC; Manassas, Va.) and grown in Luria-Bertani (LB) broth. For macrophage infections, 10 ml LB in a 125 mL flask was inoculated from a frozen glycerol stock and cultured overnight with shaking at 37° C. to stationary phase. RAW 264.7 cells (1×10⁵ cells/well) were seeded in 24 well plates. Bacteria were diluted in culture medium to give a nominal multiplicity of infection (MOI) of approximately 100, bacteria were centrifuged onto the monolayer at 1000 rpm for 10 minutes to synchronize infection, and the infection was allowed to proceed for 20 min in a 37° C., 5% CO₂ incubator. Cells were washed 3 times with PBS to remove extracellular bacteria and then incubated in DMEM +10% FBS containing 100 μg/ml gentamicin (Sigma, St. Louis, Mo.) to kill any remaining extracellular bacteria and prevent re-infection. After 2 h, the gentamicin concentration was lowered to 10 μg/ml and maintained throughout the assay. Cells were pretreated with inhibitors for 30 min prior to infection at the following concentrations: 50 μM PD 98059 (Calbiochem), 50 μM U 0126 (Promega), 2 mM diphenyliodonium (DPI), 250 μM acetovanillone (apocynin, Aldrich), 1 mM ascorbic acid (Sigma), 30 mM N-acetyl cysteine (Sigma), and 2 mM N^{G}-L-monomethyl arginine (L-NMMA, Molecular Probes) or 2 mM N^{G}-D-monomethyl arginine (D-NMMA, Molecular Probes). Fresh inhibitors were added immediately after infection, at 2 h, and 6-8 h post-infection to ensure potency. Control cells were treated with equivalent volumes of dimethylsulfoxide (DMSO) per mL of media. Intracellular survival/replication of S. Typhimurium SL1344 was determined using the gentamicin-resistance assay, as previously described. Briefly, cells were washed twice with PBS to remove gentamicin, lysed with 1% Triton X-100/0.1% SDS in PBS at 2 h and 24 h post-infection, and numbers of intracellular bacteria calculated from colony counts on LB agar plates. Under these infection conditions, macrophages contained an average of 1 bacterium per cell as assessed by standard plate counts, which permitted analysis of macrophages at 24 h post-infection. Bacterial filiamentation is related to bacterial stress. NADPH oxidase and iNOS can be activated by MEK/ERK signaling. The results (Table 61) clearly demonstrate that the alteration of cell signaling is a method whereby intracellular Salmonella infections can be resolved. Thus since bacteria to up-regulate multiple genes in human cells, this strategy of blocking signaling represents a general method of therapy against infection.

TABLE 61
Effect of the Signaling Molecule MEK on Intracellular Bacteria in IFN-γ-
primed RAW cells.

| Treatment^{a} | Effect^{b} |
|---|---|
| 0 | None |
| MEK inhibitor | Decrease bacterial filamentation (bacterial stress)^{c} |
| U 0126 | Increase in the number of intracellular S. Typhimurium |
| MEK inhibitor | Decrease bacterial filamentation (bacterial stress)^{c} |
| PD 98059 | Increase in the number of intracellular S. Typhimurium |
| NADPH oxidase | Decrease bacterial filamentation (bacterial stress)^{c} |
| inhibitor^{d} | Increase in the number of intracellular S. Typhimurium |

### EXAMPLE 11

### Anti-Viral Activity

SDF-1, a C-X-C chemokine is a natural ligand for HIV-1 coreceptor-CXCR4. The chemokine receptors CXCR4 and CCR5 are considered to be potential targets for the inhibition of HIV-1 replication. The crystal structure of SDF-1 exhibits antiparallel β-sheets and a positively charged surface, features that are critical in binding to the negatively charged extracellular loops of CXCR4. These findings suggest that chemokine derivatives, small-size CXCR4 antagonists, or agonists mimicking the structure or ionic property of chemokines may be useful agents for the treatment of X4 HIV-1 infection. It was found that the cationic peptides inhibited SDF-1 induced T-cell migration suggesting that the peptides may act as CXCR4 antagonists. The migration assays were performed as follows. Human Jurkat T cells were resuspended to 5×10⁶/ml in chemotaxis medium (RPMI 1640/10 mM Hepes/0.5% BSA). Migration assays were performed in 24 well plates using 5 μm polycarbonate Transwell inserts (Costar). Briefly, peptide or controls were diluted in chemotaxis medium and placed in the lower chamber while 0.1 ml cells (5×10⁶/ml) was added to the upper chamber. After 3 hr at 37° C., the number of cells that had migrated into the lower chamber was determined using flow cytometry. The medium from the lower chamber was passed through a FACscan for 30 seconds, gating on forward and side scatter to exclude cell debris. The number of live cells was compared to a “100% migration control” in which 5×10⁵/ml cells had been pipetted directly into the lower chamber and then counted on the FACscan for 30 seconds. The results demonstrate that the addition of peptide results in an inhibition of the migration of Human Jurkat T-cells (Table 62) probably by influencing CXCR4 expression (Tables 63 and 64).

TABLE 62
Peptide inhibits the migration of human Jurkat-T cells:

| | Migration (%) | | | |
|---|---|---|---|---|
| | Positive | SDF-1 | SDF-1 + SEQ 1D | Negative |
| Experiment | control | (100 ng/ml) | 1 (50 μg/ml) | control |
| 1 | 100% | 32% | 0% | <0.01% |
| 2 | 100% | 40% | 0% | 0% |

TABLE 63
Corresponding polynucleotide array data to Table 56:

| Poly- | | | | |
|---|---|---|---|---|
| nucle- | Poly- | | | Acces- |
| otide/ | nucleotide | Unstimulated | Ratio | sion |
| Protein | Function | Intensity | peptide:Unstimulated | Number |
| CXCR-4 | Chemokine | 36 | 4 | D87747 |
| | receptor | | | |

TABLE 64
Corresponding FACs data to Tables 62 and 63:

| | | Fold Increase in Protein |
|---|---|---|
| | Concentration | Expression |
| Peptide | (μg/ml) | CXCR-4 |
| SEQ ID NO: 1 | 10 | No change |
| SEQ ID NO: 1 | 50 | 1.3 ± 0.03 |
| SEQ ID NO: 1 | 100 | 1.6 ± 0.23 |
| SEQ ID NO: 3 | 100 | 1.5 ± 0.2 |

### EXAMPLE 12

### Synergistic Combinations

Methods And Materials

S. aureus was prepared in phosphate buffered solution (PBS) and 5% porcine mucin (Sigma) to a final expected concentration of 1-4×10⁷ CFU/ml. 100 μl of S. aureus (mixed with 5% porcine mucin) was injected intraperitoneally (IP) into each CD-1 mouse (6˜8 weeks female weighing 20-25 g (Charles River)). Six hours after the onset of infection, 100 μl of the peptide was injected (50-200 μg total) IP along with 0.1 mg/kg Cefepime. After 24 hours, animals were sacrificed and heart puncture was performed to remove 100 μl of blood. The blood was diluted into 1 ml PBS containing Heparin. This was then further diluted and plated for viable colony counts on Mueller-Hinton agar plates (10⁻¹, 10⁻², 10⁻³, & 10⁻⁴). Viable colonies, colony-forming units (CFU), were counted after 24 hours. Each experiment was carried out a minimum of three times. Data is presented as the average CFU±standard error per treatment group (8-10 mice/group).

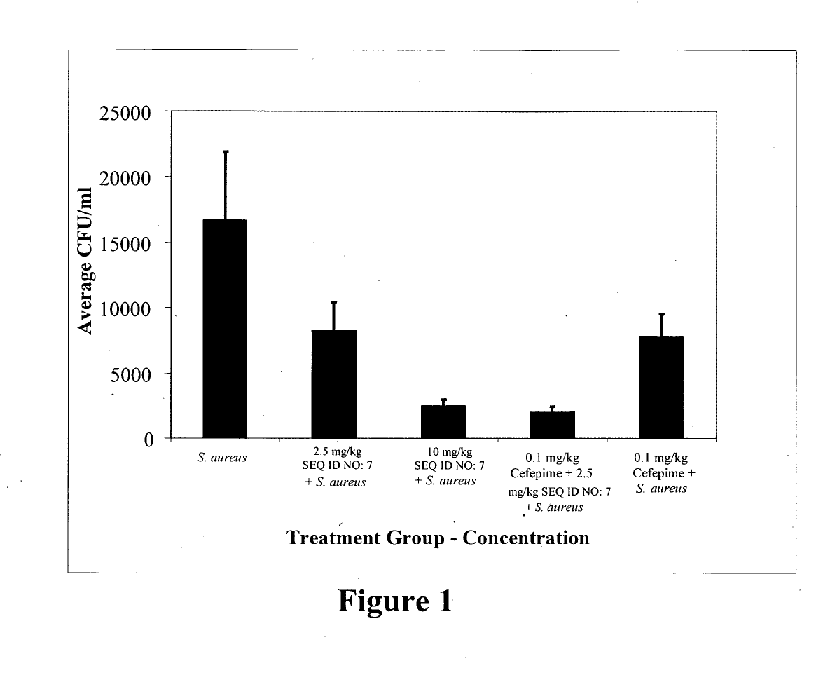

Experiments were carried out with peptide and sub-optimal Cefepime given 6 hours after the onset of systemic S. aureus infection (FIG. 1). The data in FIG. 1 is presented as the mean±standard error of viable counts from blood taken from the mice 24 hrs after the onset of infection. The combination of sub optimal antibiotic (cefepime) dosing and SEQ ID NO: 7 resulted in improved therapeutic efficacy. The ability of the peptides to work in combination with sub-optimal concentrations of an antibiotic in a murine infection model is an important finding. It suggests the potential for extending the life of antibiotics in the clinic and reducing incidence of antibiotic resistance.

SEQ ID NO: 1, as an example, induced phosphorylation and activation of the mitogen activated protein kinases, ERK1/2 and p38 in human peripheral blood-derived monocytes and a human bronchial epithelial cell line but not in B- or T-lymphocytes. Phosphorylation was not dependent on the G-protein coupled receptor, FPRL-1, which was previously proposed to be the receptor for SEQ ID NO: 1-induced chemotaxis on human monocytes and T cells. Activation of ERK1/2 and p38 was markedly increased by the presence of granulocyte macrophage-colony stimulating factor (GM-CSF), but not macrophage-colony stimulating factor (M-CSF). Exposure to SEQ ID NO: 1 also led to the activation of Elk-1, a transcription factor that is downstream of and activated by phosphorylated ERK1/2, as well as the up-regulation of various Elk-1 controlled genes. The ability of SEQ ID NO: 1 to signal through these pathways has broad implications in immunity, monocyte activation, proliferation and differentiation.

SEQ ID NO: 1 (sequence LGDFFRKSKEKIGKEFKRIVQRIKDFLRNLVPRTES), was synthesized by Fmoc [(N-(9-fluorenyl)methoxycarbonyl)] chemistry at the Nucleic Acid/Protein Synthesis (NAPS) Unit at UBC. Human recombinant granulocyte-macrophage colony-stimulating factor (GM-CSF), interleukin-4 (IL-4) and macrophage colony-stimulating factor (M-CSF) were purchased from Research Diagnostics Inc. (Flanders, N.J., USA). Pertussis toxin was supplied by List Biological Laboratories Inc. (Campbell, Calif., USA).

Blood monocytes were prepared using standard techniques. Briefly, 100 ml of fresh human venous blood was collected in sodium heparin Vacutainer collection tubes (Becton Dickinson, Mississauga, ON, Canada) from volunteers according to UBC Clinical Research Ethics Board protocol C02-0091. The blood was mixed, at a 1:1 ratio, with RPMI 1640 media [supplemented with 10% v/v fetal calf serum (FBS), 1% L-glutamine, 1 nM sodium pyruvate] in an E-toxa-clean (Sigma-Aldrich, Oakville, ON, Canada) washed, endotoxin-free bottle. PBMC were separated using Ficoll-Paque Plus (Amersham Pharmacia Biotech, Baie D'Urfé, PQ, Canada) at room temperature and washed with phosphate buffered saline (PBS). Monocytes were enriched with the removal of T-cells by rosetting with fresh sheep red blood cells (UBC animal care unit) pre-treated with Vibrio cholerae neuraminidase (Calbiochem Biosciences Inc., La Jolla, Calif., USA) and repeat separation by Ficoll Paque Plus. The enriched monocytes were washed with PBS, then cultured (approximately 2-3×10⁶ per well) for 1 hour at 37° C. followed by the removal of non-adherent cells; monocytes were >95% pure as determined by flow cytometry (data not shown). B-lymphocytes were isolated by removing non-adherent cells and adding them to a new plate for one hour at 37° C. This was repeated a total of three times. Any remaining monocytes adhered to the plates, and residual non-adherent cells were primarily B cells. Cells were cultured in Falcon tissue culture 6-well plates (Becton Dickinson, Mississauga, ON, Canada). The adherent monocytes were cultured in 1 ml media at 37° C. in which SEQ ID NO: 1 and/or cytokines dissolved in endotoxin-free water (Sigma-Aldrich, Oakville, ON, Canada) were added. Endotoxin-free water was added as a vehicle control. For studies using pertussis toxin the media was replaced with 1 ml of fresh media containing 100 ng/ml of toxin and incubated for 60 min at 37° C. SEQ ID NO: 1 and cytokines were added directly to the media containing pertussis toxin. For the isolation of T lymphocytes, the rosetted T cells and sheep red blood cells were resuspended in 20 ml PBS and 10 ml of distilled water was added to lyse the latter. The cells were then centrifuged at 1000 rpm for 5 min after which the supernatant was removed. The pelleted T cells were promptly washed in PBS and increasing amounts of water were added until all sheep red blood cells had lysed. The remaining T cells were washed once in PBS, and viability was confirmed using a 0.4% Trypan blue solution. Primary human blood monocytes and T cells were cultured in RPMI 1640 supplemented with 10% v/v heat-inactivated FBS, 1% v/v L-glutamine, 1 nM sodium pyruvate (GIBCO Invitrogen Corporation, Burlington, ON, Canada). For each experiment between two and eight donors were used.

The simian virus 40-transformed, immortalized 16HBE4o-bronchial epithelial cell line was a generous gift of Dr. D. Gruenert (University of California, San Francisco, Calif.). Cells were routinely cultured to confluence in 100% humidity and 5% CO₂ at 37° C. They were grown in Minimal Essential media with Earles'salts (GIBCO Invitrogen Corporation, Burlington, ON, Canada) containing 10% FBS (Hyclone), 2 mM L-glutamine. For experiments, cells were grown on Costar Transwell inserts (3-μm pore size, Fischer Scientific) in 24-well plates. Cells were seeded at 5×10⁴ cells per 0.25 ml of media on the top of the inserts while 0.95 ml of media was added to the bottom of the well and cultured at 37° C. and 5% CO₂. Transmembrane resistance was measured daily with a Millipore voltohmeter and inserts were used for experiments typically after 8 to 10 days, when the resistance was 500-700 ohms. The cells were used between passages 8 and 20.

Western Immunoblotting—After stimulation, cells were washed with ice-cold PBS containing 1 mM vanadate (Sigma). Next 125 μl of RIPA buffer (50 mM Tris-HCl, pH 7.4, NP-40 1%, sodium deoxycholate 0.25%, NaCl 150 mM, EDTA 1 mM, PMSF 1 mM, Aprotinin, leupeptin, pepstatin 1 μg/ml each, sodium orthovanadate 1 mM, NaF 1 mM) was added and the cells were incubated on ice until they were completely lysed as assessed by visual inspection. The lysates were quantitated using a BCA assay (Pierce). 30 μg of lysate was loaded onto 1.5 mm thick gels, which were run at 100 volts for approximately 2 hours. Proteins were transferred to nitrocellulose filters for 75 min at 70 V. The filters were blocked for 2 hours at room temperature with 5% skim milk in TBST (10 mM Tris-HCl pH 8, 150 mM NaCl, 0.1% Tween-20). The filters were then incubated overnight at 4° C. with the anti-ERK1/2-P or anti-p38-P (Cell Signaling Technology, Ma) monoclonal antibodies. Immunoreactive bands were detected using horseradish peroxidase-conjugated sheep anti-mouse IgG antibodies (Amersham Pharmacia, New Jersey) and chemiluminescence detection (Sigma, Mo). To quantify bands, the films were scanned and then quantified by densitometry using the software program, ImageJ. The blots were reprobed with a β-actin antibody (ICN Biomedical Incorporated, Ohio) and densitometry was performed to allow correction for protein loading.

Kinase Assay—An ERK1/2 activity assay was performed using a non-radioactive kit (Cell Signaling Technology). Briefly, cells were treated for 15 min and lysed in lysis buffer. Equal amounts of proteins were immunoprecipitated with an immobilized phospho-ERK1/2 antibody that reacts only with the phosphorylated (i.e. active) form of ERK1/2. The immobilized precipitated enzymes were then used for the kinase assay using Elk-1 followed by Western blot analysis with antibodies that allow detection and quantitation of phosphorylated substrates.

Quantification of IL-8—Human IL-8 from supernatants of 16HBE40-cells was measured by using the commercially available enzyme-linked immunosorbent assay kit (Biosource) according to the manufacturer's instructions.

Semiquantitative RT-PCR—Total RNA from two independent experiments was isolated from 16HBE4o-cells using RNaqueous (Ambion) as described by the manufacturer. The samples were DNase treated, and then cDNA synthesis was accomplished by using a first-strand cDNA synthesis kit (Gibco). The resultant cDNAs were used as a template in PCRs for various cytokine genes:

| | |
|---|---|
| MCP-1 | |
| 5′-TCATAGCAGCCACCTTCATTC-3′; | (SEQ ID NO: 59) |
| 5′-TAGCGCAGATTCTTGGGTTG-3; | (SEQ ID NO: 60) |
| MCP-3 | |
| 5′-TGTCCTTTCTCAGAGTGGTTCT-3′; | (SEQ ID NO: 61) |
| 5′-TGCTTCCATAGGGACATCATA-3′ | (SEQ ID NO: 62) |
| IL-6 | |
| 5′-ACCTGAACCTTCCAAAGATGG-3′; | (SEQ ID NO: 63) |
| 5′-GCGCAGAATGAGATGAGTTG-3′; | (SEQ ID NO: 64) |
| and | |
| IL-8 | |
| 5′-GTGCAGAGGGTTGTGGAGAAG-3′; | (SEQ ID NO: 65) |
| 5′-TTCTCCCGTGCAATATCTAGG-3′ | (SEQ ID NO: 66) |

Each RT-PCR reaction was performed in at least duplicate. Results were analysed in the linear phase of amplification and normalized to the housekeeping control, glyceraldehyde-3-phosphate dehydrogenase. Reactions were verified for RNA amplification by including controls without reverse transcriptase.

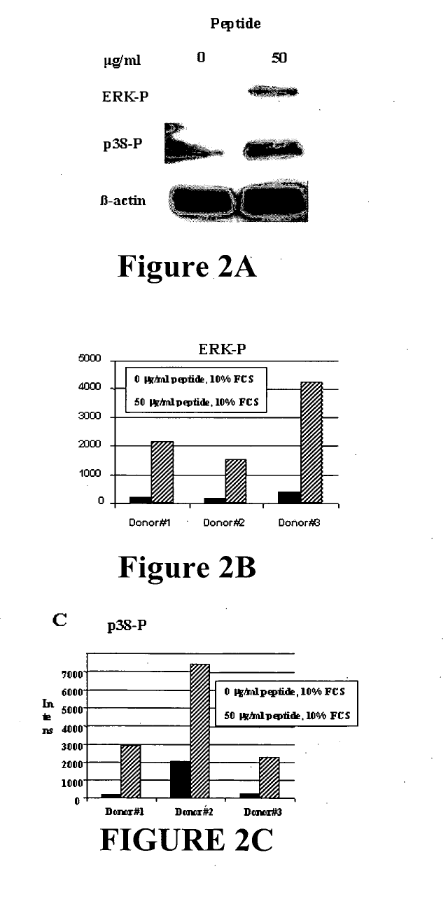

Peptides induce ERK1/2 and p38 phosphorylation in peripheral blood derived monocytes. To determine if peptide induced the activation of the MAP kinases, ERK1/2 and/or p38, peripheral blood derived monocytes were treated with 50 μg/ml SEQ ID NO: 1 or water (as a vehicle control) for 1.5 min. To visualize the activated (phosphorylated) form of the kinases, Western blots were performed with antibodies specific for the dually phosphorylated form of the kinases (phosphorylation on Thr202+Tyr204 and Thr180+Tyr182 for ERK1/2 and p38 respectively). The gels were re-probed with an antibody for β-actin to normalize for loading differences. In all, an increase in phosphorylation of ERK1/2 (n=8) and p38 (n=4) was observed in response to SEQ ID NO: 1 treatment (FIG. 2).

FIG. 2 shows exposure to SEQ ID NO: 1 induces phosphorylation of ERK1/2 and p38. Lysates from human peripheral blood derived monocytes were exposed to 50 μg/ml of SEQ ID NO: 1 for 15 minutes. A) Antibodies specific for the phosphorylated forms of ERK and p38 were used to detect activation of ERK1/2 and p38. All donors tested showed increased phosphorylation of ERK1/2 and p38 in response to SEQ ID NO: 1 treatment. One representative donor of eight. Relative amounts of phosphorylation of ERK (B) and p38(C) were determined by dividing the intensities of the phosphorylated bands by the intensity of the corresponding control band as described in the Materials and Methods.

Peptide induced activation of ERK1/2 is greater in human serum than in fetal bovine serum. It was demonstrated that SEQ ID NO: 1 induced phosphorylation of ERK1/2 did not occur in the absence of serum and the magnitude of phosphorylation was dependent upon the type of serum present such that activation of ERK1/2 was far superior in human serum (HS) than in fetal bovine serum (FBS).

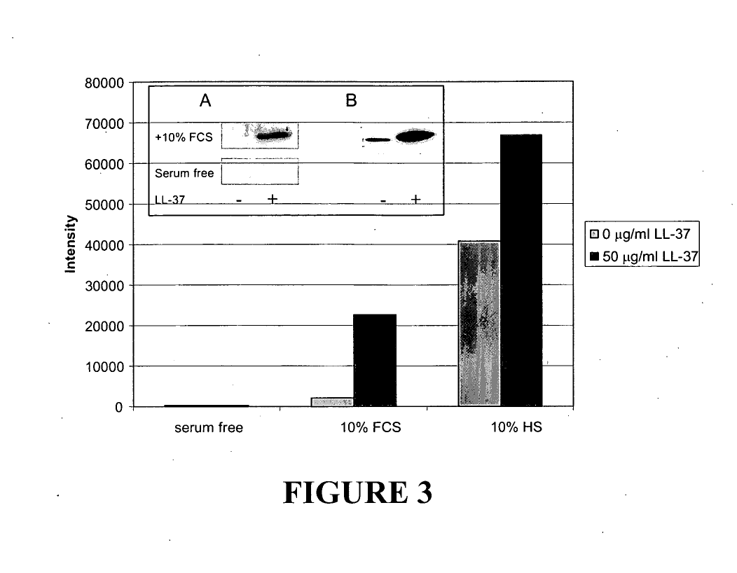

FIG. 3 shows SEQ ID NO: 1 induced phosphorylation of ERK1/2 does not occur in the absence of serum and the magnitude of phosphorylation is dependent upon the type of serum present. Human blood derived monocytes were treated with 50 μg/ml of SEQ ID NO: 1 for 15 minutes. Lysates were run on a 12% acrylamide gel then transferred to nitrocellulose membrane and probed with antibodies specific for the phosphorylated (active) form of the kinase. To normalize for protein loading, the blots were reprobed with β-actin. Quantification was done with ImageJ software. The FIG. 3 inset demonstrates that SEQ ID NO: 1 is unable to induce MAPK activation in human monocytes under serum free conditions. Cells were exposed to 50 mg/ml of SEQ ID NO: 1 (+), or endotoxin free water (−) as a vehicle control, for 15 minutes. (A) After exposure to SEQ ID NO: 1 in media containing 10% fetal calf serum, phosphorylated ERK1/2 was detectable, however, no phosphorylation of ERK1/2 was detected in the absence of serum (n=3). (B) Elk-1, a transcription factor downstream of ERK1/2, was activated (phosphorylated) upon exposure to 50 μg/ml of SEQ ID NO: 1 in media containing 10% fetal calf serum, but not in the absence of serum (n=2).

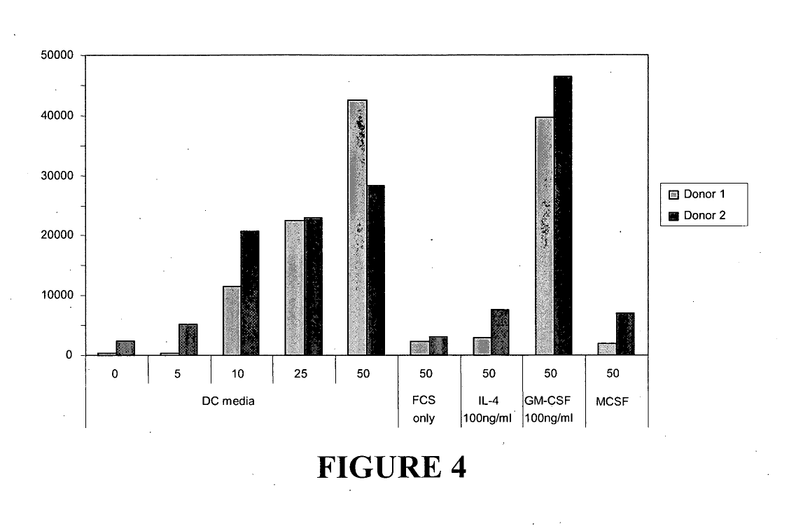

Peptide induced activation of ERK1/2 and p38 is dose dependent and demonstrates synergy with GM-CSF. GM-CSF, IL-4, or M-CSF (each at 100 ng/ml) was added concurrently with SEQ ID NO: 1 and phosphorylation of ERK1/2 was measured in freshly isolated human blood monocytes. ERK1/2 phosphorylation was evident when cells were treated with 50 μg/ml of SEQ ID NO: 1 (8.3 fold increase over untreated, n=9) but not at lower concentrations (n=2). In the presence of 100 ng/ml GM-CSF, SEQ ID NO: 1-induced ERK1/2 phosphorylation increased markedly (58 fold greater than untreated, n=5). Furthermore, in the presence of GM-CSF, activation of ERK1/2 occurred in response to concentrations of 5 and 10 μg/ml of SEQ ID NO: 1, respectively, in the two donors tested (FIG. 4). This demonstrates that SEQ ID NO: 1 induced activation of ERK1/2 occurred at a lower threshold in the presence of GM-CSF, a cytokine found locally at sites of infection.

FIG. 4 shows SEQ ID NO: 1 induced activation of ERK1/2 occurs at lower concentrations and is amplified in the presence of certain cytokines. When freshly isolated monocytes were stimulated in media containing both GM-CSF (100 ng/ml) and IL-4 (100 ng/ml) SEQ ID NO: 1 induced phosphorylation of ERK1/2 was apparent at concentrations as low as 5 μg/ml. This synergistic activation of ERK1/2 seems to be due primarily to GM-CSF.

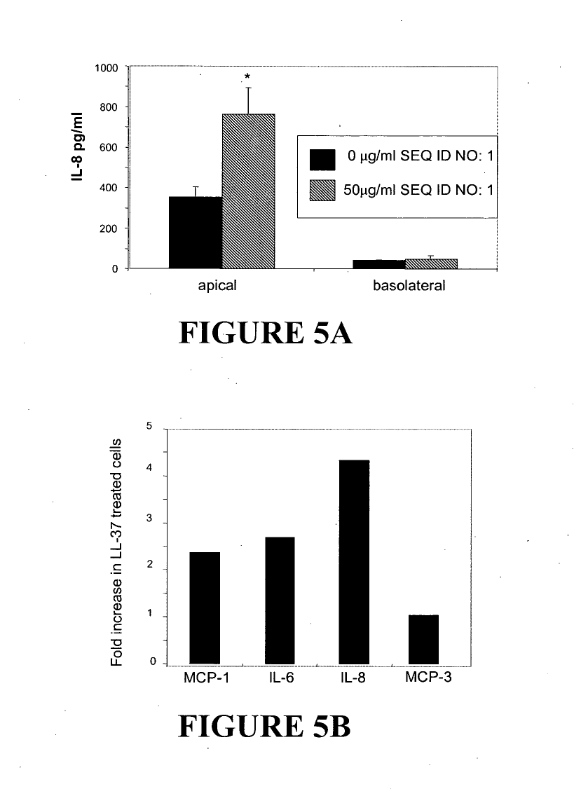

Activation of ERK1/2 leads to transcription of Elk-1 controlled genes and secretion of IL-8. IL-8 release is governed, at least in part, by activation of the ERK1/2 and p38 kinases. In order to determine if peptide could induce IL-8 secretion the human bronchial cell line, 16HBE4o-, was grown to confluency in Transwell filters, which allows for cellular polarization with the creation of distinct apical and basal surfaces. When the cells were stimulated with 50 μg/ml of SEQ ID NO: 1 on the apical surface for four hours a statistically significant increase in the amount of IL-8 released into the apical supernatant was detected (FIG. 5). To determine the downstream transcriptional effects of peptide-induced MAP kinase activation, the expression of genes known to be regulated by ERK1/2 or p38 was assessed by RT-PCR. RT-PCR was performed on RNA isolated from 16HBE4o-cells, treated for four hours with 50 μg/ml of SEQ ID NO: 1 in the presence of serum, from two independent experiments. MCP-1 and IL-8 have been demonstrated to be under the transcriptional control of both ERK1/2 and p38, consistent with this they are up-regulated 2.4 and 4.3 fold respectively. Transcription of MCP-3 has not previously been demonstrated to be influenced by the activation of the mitogen activated protein kinases, consistent with this, expression is not affected by peptide treatment. (FIG. 5). These data are consistent with the hypothesis that activation of the activation of the ERK1/2 and p38 signaling pathways has functional effects on transcription of cytokine genes with immunomodulatory functions. The inset to FIG. 3B also demonstrates that peptide induced the phosphorylation of transcription facor Elk-1 in a serum dependent manner.

FIG. 5 shows peptide affects both transcription of various cytokine genes and release of IL-8 in the 16HBE4o-human bronchial epithelial cell line. Cells were grown to confluency on a semi-permeable membrane and stimulated on the apical surface with 50 μg/ml of SEQ ID NO: 1 for four hours. A) SEQ ID NO: 1 treated cells produced significantly more IL-8 than controls, as detected by ELISA in the supernatant collected from the apical surface, but not from the basolateral surface. Mean±SE of three independent experiments shown, asterisk indicates p=0.002. B) RNA was collected from the above experiments and RT-PCR was performed. A number of cytokine genes known to be regulated by either ERK1/2 or p38 were up-regulated upon stimulation with peptide. The average of two independent experiments is shown.

### EXAMPLE 13

### Modulation of an Inflammatory Response

The innate immune response is a dynamic system since it can be triggered by receptor recognition of conserved bacterial components, initiating a broad inflammatory response to infectious agents, but must be able maintain homeostasis in the presence of commensal organisms, which contain many of these same conserved components. A delicate balance of pro- and anti-inflammatory mediators is vital for efficient functioning of the immune system under these disparate circumstances. In recent years, there has been speculation and some evidence implicating the sole human cathelicidin, SEQ ID NO: 1, in maintaining homeostasis, combating pathogenic challenge, and protecting against endotoxemia, an extreme inflammation-like condition (Devine DA, et al. Cationic peptides: distribution and mechanisms of resistance. Curr Pharm Des 2002; 8:703-14; Ciomei CD, et al. Antimicrobial and chemoattractant activity, Lipopolysaccharide neutralization, cytotoxicity, and inhibition by serum of analogs of human cathelicidin LL-37. Antimicrob Agents Chemother 2005; 49:2845-50). The data presented herein demonstrate that SEQ ID NO: 1 is an important component of human immunity that regulates the balance of pro- and anti-inflammatory molecules both under homeostatic conditions and during endotoxin challenge (i.e., infection situations).

Materials and Methods

Cell Isolation and Cell Lines—Human monocytic cells, THP-1 (Tsuchiya S, et al. Establishment and characterization of a human acute monocytic leukemia cell line (THP-1). Int J Cancer 1980; 26:171-6), were obtained from American type culture collection, ATCC® (TIB-202) and were grown in suspension in RPMI-1640 media (Gibco®, Invitrogen™ Life technologies, Burlington, ON), supplemented with 10% (v/v) heat inactivated fetal bovine serum (FBS), 2 mM L-glutamine and 1 mM sodium pyruvate (all from Invitrogen Life Technologies). Cultures were maintained at 37° C. in a humidified 5% (v/v) CO₂ incubator up to a maximum of six passages. THP-1 cells at a density of 1×10⁶ cells/ml were treated with 0.3 μg/ml phorbol 12-myristate 13-acetate (PMA; Sigma-Aldrich Canada, Oakville ON) for 24 hr (Tsuchiya S, et al. Induction of maturation in cultured human monocytic leukemia cells by a phorbol diester. Cancer Res 1982; 42:1530-6), inducing plastic-adherent cells that were further rested in complete RPMI-1640 medium for an additional 24 hr prior to stimulations with various treatments. Venous blood (20 ml) from healthy volunteers was collected in Vacutainer® collection tubes containing sodium heparin as an anticoagulant (Becton Dickinson, Mississauga, ON) in accordance with UBC ethical approval and guidelines. Blood was diluted 1:1 with complete RPMI 1640 medium and separated by centrifugation over a Ficoll-Paque® Plus (Amersham Biosciences, Piscataway, N.J., USA) density gradient. White blood cells were isolated from the buffy coat, washed twice in RPMI 1640 complete medium, and the number of peripheral blood mononuclear cells (PBMC) was determined by trypan blue exclusion. PBMC (5×10⁵) were seeded into 12-well tissue culture dishes (Falcon; Becton Dickinson) at 1×10⁶ cells/ml at 37° C. in 5% CO₂. All experiments using human THP-1 cells or PBMCs involved at least three biological replicates.

Stimulants, Reagents and Antibodies—LPS was isolated from P. aeruginosa H103 using the Darveau-Hancock method as previously described (Darveau RP, et al. Procedure for isolation of bacterial lipopolysaccharides from both smooth and rough Pseudomonas aeruginosa and Salmonella typhimurium strains. J Bacteriol 1983; 155:831-8). Briefly, P. aeruginosa was grown overnight in LB broth at 37° C. Cells were collected and washed and the isolated LPS pellets were extracted with a 2:1 chloroform:methanol solution to remove contaminating lipids. Purified LPS samples were quantitated using an assay for the specific sugar 2-keto-3-deoxyoctosonic acid (KDO assay) and then resuspended in endotoxin-free water (Sigma-Aldrich).

TLR2 agonists lipoteichoic acid (LTA) from S. aureus and a synthetic tripalmitoylated lipopeptide, Pam₃CSK4, were purchased from InvivoGen (San Diego, Calif., USA). TLR9 agonist CpG oligodeoxynucleotide # 2007 (Krieg AM. CpG motifs in bacterial DNA trigger direct B-cell activation. Nature 1995; 374:546-9) was a gift from Dr. Lorne Babuik (Vaccine and Infectious Disease org., SK, Canada). Recombinant human TNFα and recombinant human IL1 β were obtained from Research Diagnostics Inc., (Flanders, N.J., USA). All reagents were tested for endotoxin and reconstituted in endotoxin-free water. LTA from S. aureus used in this study had 1.25 EU of endotoxin/μg of LTA. Polymyxin B was purchased from InvivoGen, Actinomycin D (transcriptional inhibitor) was purchased from Calbiochem-Novabiochem Corporation (La Jolla, Calif.) and Monensin (inhibitor of protein secretion) was purchased from eBiosciences., CA, USA. A cationic peptide, SEQ ID NO: 1, was synthesized using F-moc chemistry at the Nucleic Acid/Protein Synthesis Unit, University of British Columbia (Vancouver, BC, Canada). The synthetic peptide was re-suspended in endotoxin-free water and stored at −20° C. until further use.

Rabbit polyclonal antibodies against the NFκB subunits p105/p50, p65 and Re1B were purchased from Cell Signaling Technologies (Mississauga, ON, Canada). Rabbit polyclonal antibody against the NFκB subunit c-Rel was purchased from Chemicon International (Temecula, Calif., USA) and mouse IgG2a monoclonal antibody against NFκB subunit p100/p52 was purchased from Upstate Cell Signaling Solutions (Lake Placid, N.Y., USA). HRP-conjugated goat anti-rabbit and anti-mouse IgG antibodies were purchased from Cell Signaling Technologies and Amersham Biosciences respectively.

Treatment with inflammatory stimuli, peptide or inhibitors—THP-1 cells or PBMC were stimulated with LPS (10 or 100 ng/ml), LTA (1 μg/ml), Pam₃CSK4 (100 ng/ml), CpG-ODN 2007 (2 μg/ml), recombinant human TNFα (50 ng/ml) or recombinant human IL1β (50 ng/ml) for 1, 2, 4, or 24 hours as indicated in the results section. SEQ ID NO: 1 (0.5-50 μg/ml) was added simultaneously or 30 min after addition of the stimulants as indicated in the results. Alternatively, cells were stimulated with SEQ ID NO: 1 (20 μg/ml) for 30 min, washed with RPMI complete media to remove the peptide and then stimulated with LPS (100 ng/ml). Polymyxin B (0.1 mg/ml), actinomycin D (4 μg/ml), or monensin (working concentration as per the manufacturer's instructions) were added to the THP-1 cells 30 min prior to stimulants.

Detection of cytokines—Following incubation of the cells under various treatment regimens, the tissue culture supernatants were centrifuged at 1000×g for 5 min, then at 10,000×g for 2 min to obtain cell-free samples. Supernatants were aliquoted and then stored at −20° C. prior to assay for various cytokines. TNFα and IL8 secretion were detected with a capture ELISA (eBioscience and BioSource International Inc., CA, USA respectively) using either tissue culture supernatants or the nuclear and cytoplasmic extracts (see below) as per the experimental design. All assays were performed in triplicate. The concentration of the cytokines in the culture medium was quantified by establishing a standard curve with serial dilutions of the recombinant human TNFα or IL8 respectively. Alternatively, five cytokines (GMCSF, IL1β, IL6, IL8 and TNFα) were measured simultaneously using the Human Cytokine 5-Plex kit from Biosource International Inc., (Medicorp Inc., Montreal, Canada) as per the manufacturer's instructions. The multiplex bead immunoassays were analyzed using Luminex 100™ StarStation software (Applied Cytometry Systems, Sacramento, Calif., USA).

RNA extraction, amplification and hybridization to DNA microarrays—RNA was isolated from THP-1 cells with RNeasy Mini kit, treated with RNase-Free DNase (Qiagen Inc., Canada) and eluted in RNase-free water (Ambion Inc., Austin, Tex., USA) as per the manufacturer's instructions. RNA concentration, integrity and purity were assessed by Agilent 2100 Bioanalyzer using RNA 6000 Nano kits (Agilent Technologies, USA). RNA was (reverse) transcribed with incorporation of amino-allyl-UTP (aa-UTP) using the MessageAmpII™ amplification kit, according to the manufacturer's instructions, then column purified and eluted in nuclease-free water. Column purified samples were labeled with mono-functional dyes, Cyanine-3 and Cyanine-5 (Amersham Biosciences), according to manufacturer's instructions, and then purified using the Mega Clear kit (Ambion). Yield and fluorophore incorporation was measured using Lambda 35 UV/VIS fluorimeter (PerkinElmer Life and Analytical Sciences, Inc., USA). Microarray slides were printed with the human genome 21K Array-Ready Oligo Set™ (Qiagen Inc., USA) at The Jack Bell Research Center (Vancouver, BC, Canada). The slides were pre-hybridized for 45 min at 48° C. in pre-hybridization buffer containing 5×SSC (Ambion), 0.1% (w/v) SDS and 0.2% (w/v) BSA. Equivalent (20 pmol) cyanine labeled samples from control and treated cells were then mixed and hybridized on the array slides, in Ambion SlideHyb™ buffer #2 (Ambion) for 18 hr at 37° C. in a hybridization oven. Following hybridization, the slides were washed twice in 1×SSC/0.1% sodium dodecyl sulphate (SDS) for 5 min at 65° C., then twice in 1×SSC and 0.1×SSC for 3 min each at 42° C. Slides were centrifugated for 5 min at 1000×g, dried and scanned using ScanArray™ Express software/scanner (scanner and software by Packard BioScience BioChip Technologies) and the images were quantified using ImaGene™ (BioDiscovery Inc., El Segundo, Calif., USA).

Analysis of DNA Microarrays—Assessment of slide quality, normalization, detection of differential gene expression and statistical analysis was carried out with ArrayPipe (version 1.6), a web-based, semi-automated software specifically designed for processing of microarray data (Hokamp K, et al. ArrayPipe: a flexible processing pipeline for microarray data. Nucleic Acids Res 2004; 32(Web Server issue):W457-9) (www.pathogenomics.ca/arraypipe). The following processing steps were applied: 1) flagging of markers, 2) subgrid-wise background correction, using the median of the lower 10% foreground intensity as an estimate for the background noise, 3) data-shifting, to rescue negative spots, 4) printTip LOESS normalization, 5) merging of technical replicates, 6) two-sided one-sample Student t-test on the log₂-ratios within each treatment group, 7) averaging of biological replicates to yield overall fold-changes for each treatment group. Further, the gene expression data was overlaid on molecular interaction networks using Cytoscape (Shannon P, et al. Cytoscape: a software environment for integrated models of biomolecular interaction networks. Genome Res 2003; 13:2498-504). Interactions networks were custom built from manually curated data and information contained within the Transpath pathway database (Krull M, et al. TRANSPATH: an integrated database on signal transduction and a tool for array analysis. Nucleic Acids Res 2003; 31:97-100). The false discovery rate of selecting differentially expressed genes from microarray analysis was estimated at 35%, based on Beta Uniform Mixture model (Pounds S, et al. Estimating the occurrence of false positives and false negatives in microarray studies by approximating and partitioning the empirical distribution of p-values. Bioinformatics 2003; 19:1236-42) and Q-Value model (Storey J D. A direct approach to false discovery rates. Journal of the Royal Statistical Society 2002; 64:479-498). This was consistent with the confirmation, using qPCR, at 4 different time points, of array results for 14 of 20 genes (70%) selected for follow-up.

Quantitative real-time PCR (qPCR)—Differential gene expression identified by microarray analysis was validated using quantitative real-time PCR (qPCR) using SuperScript™ III Platinum® Two-Step qRT-PCR Kit with SYBR® Green (Invitrogen Life Technologies), as per the manufacturer's instructions, in the ABI PRISM® 7000 sequence detection system (Applied Biosystems, Foster city, Calif., USA). Briefly, 1 μg of total RNA was reverse transcribed in a 20 μl reaction volume for 50 min at 42° C., the reaction was terminated by incubating for 5 min at 85° C. and then digested for 30 min at 37° C. with RNAse H. The PCR reaction was carried out in a 12.5 μl reaction volume containing 2.5 μl of 1/10 diluted cDNA template. A melting curve was performed to ensure that any product detected was specific to the desired amplicon. Fold changes were calculated after normalization to endogenous GAPDH and using the comparative Ct method (Pfaffl MW. A new mathematical model for relative quantification in real-time RT-PCR. Nucleic Acids Res 2001; 29:No. 9 e45). The primers used for qRT-PCR are reported in Table 65.

TABLE 65

Sequence of primers (human) used for qPCR

| | | |
|---|---|---|
| Gene | Forward primer (5′-3′) | Reverse Primer (5′-3′) |

| | | |
|---|---|---|
| CCL4 | CTTTTCTTACACCGCGAGGAA | GCAGAGGCTGCTGGTCTCAT |
| | (SEQ ID NO: 67) | (SEQ ID NO: 68) |
| CCL20 | TGACTGCTGTCTTGGATAGACAGA | TGATAGCATTGATGTCACAGCCT |
| | (SEQ ID NO: 69) | (SEQ ID NO: 70) |
| CXCL1 | GCCAGTGCTTGCAGACCCT | GGCTATGACTTCGGTTTGGG |
| | (SEQ ID NO: 71) | (SEQ ID NO: 72) |
| IL-8 | GACCACACTGCGCCAACAC | CTTCTCCACAACCCTCTGCAC |
| | (SEQ ID NO: 73) | (SEQ ID NO: 74) |
| GAPDH | GTCGCTGTTGAAGTCAGAGG | GAAACTGTGGCGTGATGG |
| | (SEQ ID NO: 75) | (SEQ ID NO: 76) |
| IL-10 | GGTTGCCAAGCCTTGTCTGA | AGGGAGTTCACATGCGCCT |
| | (SEQ ID NO: 77) | (SEQ ID NO: 78) |
| TNF-α | TGGAGAAGGGTGACCGACTC | TCCTCACAGGGCAATGATCC |
| | (SEQ ID NO: 79) | (SEQ ID NO: 80) |
| TNFAIP2 | CTACCAGCGCGCCTTTAATG | TCCGGAAGGACAGGCAGTT |
| | (SEQ ID NO: 81) | (SEQ ID NO: 82) |
| TNFAIP3 | CTGCCCAGGAATGCTACAGATAC | CAGGGTCACCAAGGGTACAAA |
| | (SEQ ID NO: 83) | (SEQ ID NO: 84) |
| TNIP3 | TGAAAGAAAGGTAGCAGAGCTGAA | CCGCGTGCTGAGGAATCT |
| | (SEQ ID NO: 85) | (SEQ ID NO: 86) |
| BIRC3 | AAAGCGCCAACACGTTTGA | AGGAACCCCAGCAGGAAAAG |
| | (SEQ ID NO: 87) | (SEQ ID NO: 88) |
| NF-κB1 | CTTAGGAGGGAGAGCCCACC | TTGTTCAGGCCTTCCCAAAT |
| | (SEQ ID NO: 89) | (SEQ ID NO: 90) |
| RELA | TAGGAAAGGACTGCCGGGAT | CCGCTTCTTCACACACTGGA |
| | (SEQ ID NO: 91) | (SEQ ID NO: 92) |
| RELB | TGGGCATTGACCCCTACAAC | TGGGTCCCTGAAGAACCATCAGGAAGTAGA |
| | (SEQ ID NO: 93) | (SEQ ID NO: 94) |
| NF-κBIA | GGTGAAGGGAGACCTGGCTT | GTGCCTCAGCAATTTCTGGC |
| | (SEQ ID NO: 95) | (SEQ ID NO: 96) |

Nuclear and Cytoplasmic Extracts—THP-1 cells (3×10⁶) seeded into 60 mm² petri dishes (VWR International, Mississauga, ON) were pre-treated with inhibitors for 30 min, and then stimulated with agonists or peptide for 30 min or 60 min. Cells were subsequently treated with Versene for 10 min at 37° C. in 5% CO₂ (to detach adherent cells) then washed twice with ice-cold phosphate buffered saline. Cytoplasmic and nuclear extracts were isolated using NE-PER® Nuclear and Cytoplasmic Extraction Reagents Kit (Pierce Biotechnology, Rockford, Ill., USA) according to the manufacturer's instructions. The protein concentration of the extracts was quantified using a Bicinchoninic Acid (BCA) Protein Assay (Pierce Biotechnology) and the extracts were stored at −80° C. until further use.

Translocation of NFκB subunits—Equivalent nuclear extracts (5-10 μg) were resolved on a 7.5% SDS-polyacrylamide gel (SDS-PAGE) and transferred to polyvinylidene difluoride (PVDF) Immobilon-P membranes (Millipore Canada Ltd., Mississauga, ON). Equivalent protein loading was verified by staining PVDF membranes with Blot-Fast-Stain™ (Chemicon International) according to the manufacturer's instructions. Subsequently, the PVDF membranes were incubated with anti-p105/p50, anti-p65, anti-c-Rel, anti-Rel B or anti-p100/p52 antibodies at 1/1000 dilution in TBST (20 mM Tris pH 7.5, 150 mM NaCl, 0.1% Tween 20) containing 5% skimmed milk powder (TBST/milk) for 1 hr. Membranes were washed for 1 hour in TBST and then incubated with a 1/5000 dilution of HRP-conjugated goat anti-mouse or anti-rabbit Ab (in TBST/milk) for 30 min. The membranes were incubated for 30 to 60 min in TBST and developed with chemiluminescence peroxidase substrate (Sigma-Aldrich), according to manufacturer's instructions. Alternatively, equivalent nuclear extracts (2.5-10 μg) were analyzed for NFκB subunits p50 or p65 by StressXpress NFκB p50 or p65 ELISA kits (Stressgen Bioreagents, Victoria, BC, Canada) according to manufacturer's instructions. Luminescence was detected with SpectraFluor Plus Multifunction Microplate Reader (Tecan Systems Inc., SJ, USA).

Results

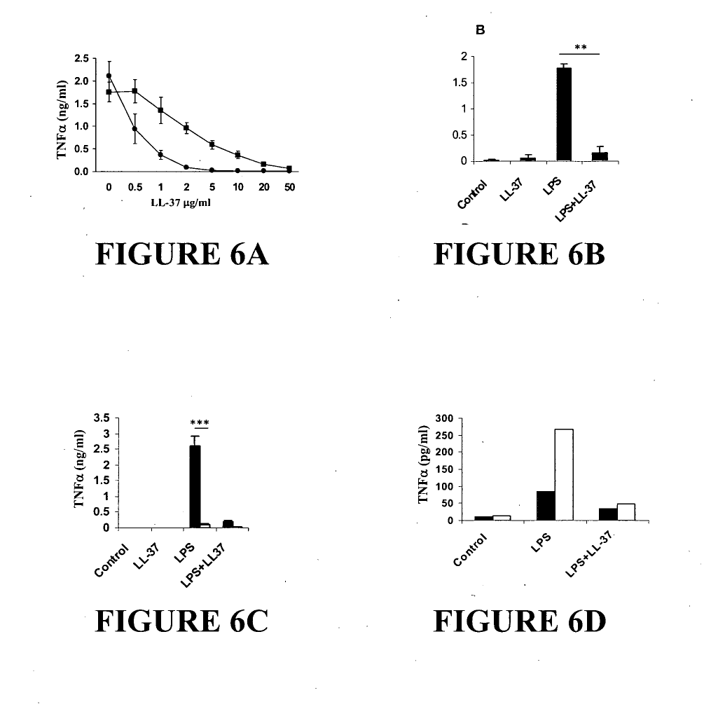

Low, physiological concentrations of SEQ ID NO: 1 suppress LPS-induced secretion of the pro-inflammatory cytokine TNFα. SEQ ID NO: 1 is found at mucosal surfaces at concentrations of around 2.5 to 5 μg/ml in adults and up to 20 μg/ml in infants (Schaller-Bals S, et al. Increased levels of antimicrobial peptides in tracheal aspirates of newborn infants during infection. Am J Respir Crit Care Med 2002; 165:992-5). Previous studies indicated that it has the ability to down-regulate pro-inflammatory cytokines in isolated monocytic cells (Bowdish DM, et al. Immunomodulatory activity of small host defense peptides. Antimicrob Agents Chemother 2005; 49:1727-32). To determine the lowest dose of SEQ ID NO: 1 that exhibited anti-endotoxin activity, THP-1 cells were stimulated with LPS (10 and 100 ng/ml) in the absence or presence of SEQ ID NO: 1 added simultaneously at concentrations ranging from 0.5 to 50 μg/ml for a period of 4 hours in complete RPMI cell culture media (i.e., which contains physiological salt concentrations). Tissue culture supernatants were assayed by ELISA for the presence of the pro-inflammatory cytokine TNFα (FIG. 6A). Very low concentrations (≦1 μg/ml) of SEQ ID NO: 1 inhibited TNFα release from LPS-induced cells, demonstrating that physiological concentrations of SEQ ID NO: 1 exhibit anti-endotoxin activity. The anti-endotoxin effect of SEQ ID NO: 1 was more pronounced when the cells were stimulated with 10 ng/ml of LPS, a concentration at the lower level of concentrations used by investigators to mimic TLR signalling responses, but considerably higher than circulating endotoxin concentrations in septic patients (Opal SM, et al. Relationship between Plasma Levels of Lipopolysaccharide (LPS) and LPS-Binding Protein in Patients with Severe Sepsis and Septic Shock http://www joumals.uchicago.edu/JID/journal/issues/v180n5/990373/990373.text.html-fn1#fn1 J Infect Dis 1999; 180:1584-9). Under these conditions, 0.5 μg/ml of SEQ ID NO: 1 inhibited 50% of LPS-induced TNFα release. This inhibitory effect increased to ≧80% with a dose of 1 μg/ml of SEQ ID NO: 1, and TNFα was reduced to background levels with 2 μg/ml of SEQ ID NO: 1. In the presence of LPS at a higher concentration (100 ng/ml), 2 μg/ml of SEQ ID NO: 1 was required to inhibit 50% of TNFα released into the tissue culture supernatant. Higher concentrations (20 μg/ml) of SEQ ID NO: 1 caused ≧95% inhibition of TNFα release. These results indicated that physiological concentrations of SEQ ID NO: 1 exhibit an anti-endotoxin effect on LPS present at low and high concentrations. The anti-endotoxin effect of SEQ ID NO: 1 was similarly observed in PBMCs (FIG. 6B), for which SEQ ID NO: 1 (20 μg/ml) inhibited >91% of LPS (100 ng/ml) induced TNF-α. Subsequent mechanistic studies employed 100 ng/ml of LPS, at which concentrations more robust transcriptional up-regulation responses were observed, and 20 μg/ml of SEQ ID NO: 1, which was not cytotoxic to primary cells (Bowdish D, et al. The human cationic peptide LL-37 induces activation of the extracellular signal-regulated kinase and p38 kinase pathways in primary human monocytes. J Immunol 2004; 172:3758-65) or THP-1 cells as determined by LDH (lactose dehydrogenase) release and MTT (3-(4,5-dimethylthiazol-2-yl)-2,5-diphenyltetrazolium bromide) assay (data not shown).

To gain further insight into the mode of inhibition exerted by SEQ ID NO: 1, TNFα production and release was monitored in the supernatants of LPS-stimulated THP-1 cells treated with the transcriptional inhibitor actinomycin D. Four μg/ml of actinomycin D was used since this concentration was required for inhibition, by more than 96% within 1 hour of treatment, of LPS-induced transcription of the genes for both the cytokine TNFα and the pro-inflammatory TNFα-inducible protein 2 (TNFAIP2) (monitored by qPCR, data not shown). Actinomycin D reduced the level of TNFα release by 97.6% (FIG. 6C), indicating that LPS largely induced de novo expression of TNFα as opposed to processing and release of intracellular pools of pro-form TNFα. Moreover, the use of monensin as an inhibitor of TNFα secretion led to accumulation of TNFα within cells after LPS stimulation for 60 min (FIG. 6D). However SEQ ID NO: 1 by itself did not similarly lead to the accumulation of TNFα inside cells, indicating that it also prevented TNFα expression at the protein level rather than blocking secretion.

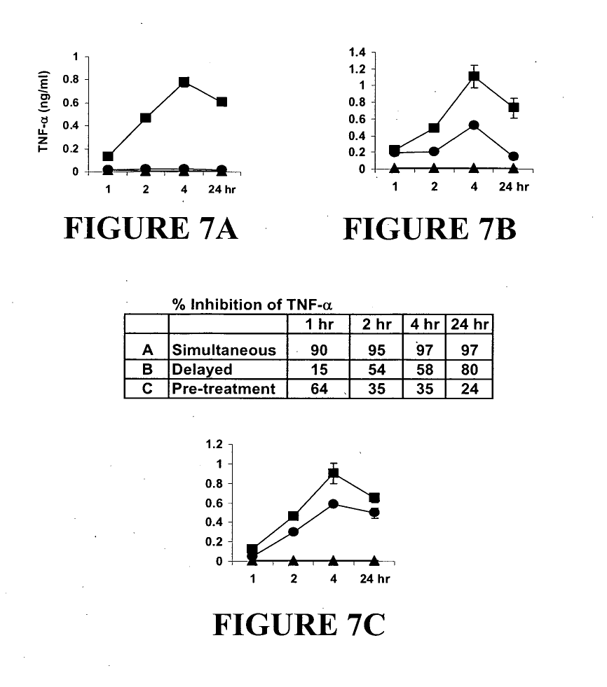

The sustained presence of SEQ ID NO: 1 inhibits TNFα release. To determine the kinetics of the anti-endotoxin effect, the supernatant from THP-1 cells was monitored for TNFα after 1, 2, 4 and 24 hr of stimulation with LPS (100 ng/ml) in absence or presence of SEQ ID NO: 1 (20 μg/ml). When the peptide and LPS were added simultaneously, the release of TNFα was substantially inhibited (90 to 97%) by SEQ ID NO: 1 at all time points (FIG. 7A). When SEQ ID NO: 1 was added 30 min after LPS addition, TNFα secretion was reduced more than 50% at 2 and 4 hr post LPS treatment and by 80% after 24 hr (FIG. 7B) consistent with previous observations in mouse macrophages (Scott MG, et al. The human antimicrobial peptide SEQ ID NO: 1 is a multifunctional modulator of innate immune responses. J Immunol 2002; 169:3883-91). In contrast, when the cells were pre-treated with SEQ ID NO: 1 for 30 min, washed and stimulated with LPS, TNFα secretion was substantially (64%) reduced after 1 hr, but this declined to only 24 to 35% at subsequent time points (FIG. 7C). This indicated that a sustained presence of SEQ ID NO: 1 was required to exhibit a maximal anti-endotoxin effect.

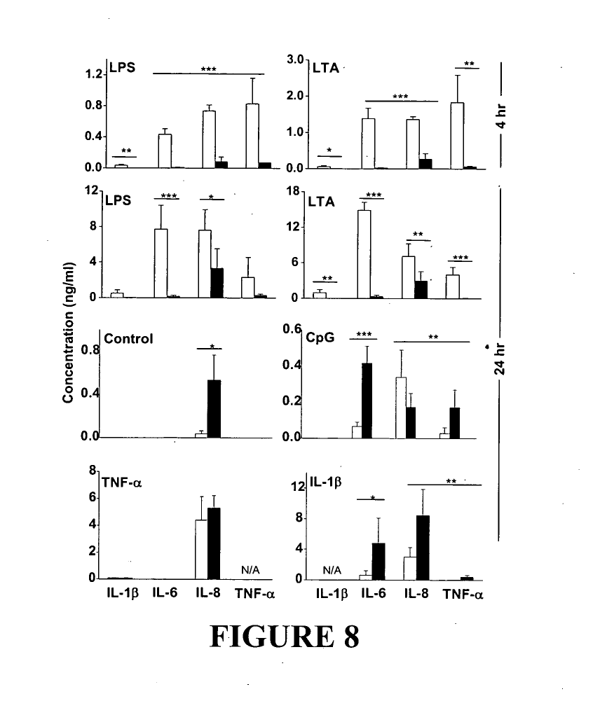

SEQ ID NO: 1 suppresses TLR-induced cytokine secretion by PBMC. PBMC were treated with agonists of TLR2 (LTA, PAM₃CSK4), TLR4 (LPS), TLR9 (CpG), and the inflammatory cytokines TNFα and IL1β, to determine if SEQ ID NO: 1 could suppress cytokine secretion induced by inflammatory stimuli LPS and other agonists in primary cells. Cytokine production was analyzed by Luminex 100™ StarSystem using the human 5-Plex cytokine kit to monitor IL1β, IL6, IL8 and TNFα in the culture supernatants. The cytokine profile of stimulated PBMC in the presence or absence of SEQ ID NO: 1 was monitored after 4 or 24 hours of treatment. The release of all 4 cytokines was significantly reduced by SEQ ID NO: 1 in both LPS- and LTA-stimulated cells after 4 hr of treatment, and this anti-inflammatory activity was sustained over 24 hr (FIG. 8). Effects on IL8 production were more modest, as anticipated, since SEQ ID NO: 1 has the ability to induce IL8 production (Scott M G, et al. The human antimicrobial peptide LL-37 is a multifunctional modulator of innate immune responses. J Immunol 2002; 169:3883-91). In addition, SEQ ID NO: 1 reduced IL1β, IL6, IL8 and TNFα production by TLR2-agonist PAM₃CSK4-stimulated PBMC after 4 or 24 hr of treatment, by approximately 30-50%, (Table 66). These data show that SEQ ID NO: 1 significantly reduced the production of pro-inflammatory cytokines resulting from activation of TLR2 or TLR4 (Table 66). SEQ ID NO: 1 also reduced, by ˜50%, IL8 secretion by PBMC stimulated with the TLR9 agonist CpG for 24 hr (FIG. 8; Table 66).

In contrast, SEQ ID NO: 1 enhanced TNFα and IL6 production by CpG-stimulated PBMC and IL6, IL8 and (modestly) TNFα by PBMC stimulated with IL1β (FIG. 8; Table 66). Conversely, SEQ ID NO: 1 had no effect on TNFα induced cytokine production. These results indicate that the SEQ ID NO: 1 was anti-inflammatory in response to selected TLR ligands, and that it was likely modulating innate immune pathways rather than simply suppressing some step in the main TLR to NFκB pathway.

Table 66 lists percent inhibition or enhancement of agonist-induced cytokine production by SEQ ID NO: 1. PBMC were incubated alone or with TLR agonists (LPS, LTA, CpG) or inflammatory cytokines (TNFα, IL1β) for 4 or 24 hr in the presence or absence of SEQ ID NO: 1. The concentration of IL1β, IL6, IL8 and TNFα released in the tissue culture supernatant is reported. The percent inhibition of IL1β, IL6, IL8 and TNFα in the presence of SEQ ID NO: 1±the standard deviation of 3 biological repeats is reported, as well as the fold enhancement of cytokine production in the presence of SEQ ID NO: 1±the standard deviation of 3 biological repeats.

TABLE 66

| | Agonist | | | | | | | | | | | |
|---|---|---|---|---|---|---|---|---|---|---|---|---|
| | Cells Only | | | TNF-α | | | IL-1β | | | LPS | | |
| | Ave pg/ml | | | Ave pg/ml | | | Ave pg/ml | | | Ave pg/ml | | |
| | −SEQ | +SEQ | Fold Inc. or | −SEQ | +SEQ | Fold Inc. | −SEQ | +SEQ | Fold Inc. | −SEQ | +SEQ | Fold Inc. |
| | ID | ID | % Inh. | ID | ID | or % Inh. | ID | ID | or % Inh. | ID | ID | or % Inh. |
| | NO: 1 | NO: 1 | (Ave ± SD) | NO: 1 | NO: 1 | (Ave ± SD) | NO: 1 | NO: 1 | (Ave ± SD) | NO: 1 | NO: 1 | (Ave ± SD) |
| Release by 4 hr | | | | | | | | | | | | |
| IL-1β | <9 | | — | 74 | 71 | 1.0 ± 0.1 | N/A | | | 34 | <9 | >81.9 ± 17.7 |
| IL-6 | <7 | | — | <7 | | — | 39 | 53 | 1.2 ± 0.4 | 435 | <7 | >98.4 ± 0.8 |
| IL-8 | 15 | 32 | 2.1 ± 0.1 | 54 | 90 | 1.9 ± 0.7 | 124 | 333 | 2.4 ± 1.2 | 738 | 84 | 89.1 ± 7.7 |
| TNF-α | <16 | | — | N/A | | | 97 | 89 | N/A | 830 | 73 | 96.2 ± 2.2 |
| Release by | | | | | | | | | | | | |
| 24 hr | | | | | | | | | | | | |
| IL-1β | <9 | | — | 94 | 95 | 1.0 ± 0.2 | N/A | | | 512 | 14 | 99.1 ± 1.3 |
| IL-6 | <7 | | — | 9 | 13 | 1.7 ± 0.6 | 645 | 4815 | 8.1 ± 1.4 | 7734 | 170 | 97.9 ± 1.3 |
| IL-8 | 37 | 532 | 10.3 ± 4.0 | 4410 | 5320 | 1.4 ± 0.6 | 3034 | 8452 | 2.9 ± 0.9 | 7620 | 3332 | 74.7 ± 15.8 |

| | | | | | | | | | |
|---|---|---|---|---|---|---|---|---|---|
| TNF-α | <16 | — | N/A | 20 | 451 | 23.6 ± 12.6 | 2334 | 303 | 78.9 ± 18.0 |

Agonist

CpG

| | | |
|---|---|---|
| | | Fold Inc. |
| LTA | PAM3 | or % |

| | | |
|---|---|---|
| Ave pg/ml | Ave pg/ml | Inh. (Ave ± SD) |

| | −SEQ | +SEQ | Fold Inc. or | −SEQ | +SEQ | Fold Inc. | −SEQ | +SEQ | Fold Inc. |
|---|---|---|---|---|---|---|---|---|---|
| | ID | ID | % Inh. | ID | ID | or % Inh. | ID | ID | or % Inh. |
| | NO: 1 | NO: 1 | (Ave ± SD) | NO: 1 | NO: 1 | (Ave ± SD) | NO: 1 | NO: 1 | (Ave ± SD) |
| Release by 4 hr | | | | | | | | | |
| IL-1β | 53 | <9 | >87.0 ± 14.5 | 34 | <9 | >81.9 ± 17.7 | <9 | | — |
| IL-6 | 1391 | 24 | 98.3 ± 0.8 | 435 | <9 | >98.4 ± 0.8 | <7 | 17.0 | — |
| IL-8 | 1366 | 273 | 79.8 ± 11.8 | 738 | 84 | 89.1 ± 7.7 | 20 | 34 | 1.7 ± 0.6 |
| TNF-α | 1836 | 66 | 96.3 ± 0.6 | 830 | 73 | 96.2 ± 2.2 | 28 | 34 | 3.5 ± 2.6 |
| Release by 24 hr | | | | | | | | | |
| IL-1β | 969 | <9 | >99.4 ± 0.6 | 512 | 14 | 99.1 ± 1.3 | <9 | | 6.6 ± 1.7 |
| IL-6 | 14887 | 318 | 97.9 ± 1.5 | 7734 | 170 | 97.9 ± 1.3 | 66 | 417 | — |
| IL-8 | 7108 | 2928 | 58.8 ± 23.1 | 7620 | 3332 | 74.7 ± 15.8 | 339 | 174 | 48.6 ± 1.0 |
| TNF-α | 4040 | 39 | 99.2 ± 0.6 | 2334 | 303 | 78.9 ± 18.0 | 28 | 171 | 17.6 ± 20.5 |

LPS-induced gene expression profile is altered by SEQ ID NO: 1. Human 21K oligo-based DNA microarrays were probed to elucidate the impact of SEQ ID NO: 1 on LPS stimulation of gene responses in human monocytic cells. Transcriptional responses were analyzed following 1, 2, 4 and 24 hr of stimulation to provide a temporal profile of gene expression in monocytes equivalent to the early, intermediate and late stages of innate immune responses. Microarray analyses were performed in duplicate from three independent biological replicates. Statistically significant, differentially expressed genes were defined as those with a fold change of at least 1.5 with a Student's t-test p-value ≦0.05 (MIAME compliant data was deposited to ArrayExpress). The number of differentially expressed genes was greatest at the 2 and 4 hr time points. Over the monitored time period, 561 and 410 genes were differentially regulated in the presence of LPS, without or with SEQ ID NO: 1 respectively. Of the 561 genes that were differentially expressed in LPS-stimulated cells, only 39 (˜7%) were identified as being up-regulated in cells stimulated with LPS in the presence of SEQ ID NO: 1 (Table 67). At least 163 genes that were upregulated in cells stimulated with LPS (i.e., proinflammatory genes) were suppressed in the presence of SEQ ID NO: 1 (Table 68). This indicates that SEQ ID NO: 1 effectively suppressed the induction of a large subset of LPS-responsive genes, but maintained a modest subset of genes that function in promoting some aspects of inflammation or anti-inflammatory response.

TABLE 67
List of 39 genes differentially expressed upon stimulation by LPS and
remaining up-regulated in the presence of SEQ ID NO: 1, as detected by microarray
analysis at one or more time points.

| | LPS_1 hr | | LPS_2 hr | | LPS_4 hr | | LPS_24 hr | |
|---|---|---|---|---|---|---|---|---|
| | Fold | | Fold | | Fold | | Fold | |
| Gene Name | change | p-value | change | p-value | change | p-value | change | p-value |
| ZNF83 | 4.21 | 0.01 | 1.59 | 0.59 | −1.42 | 0.73 | −1.20 | 0.92 |
| NFKBIA | 1.71 | 0.01 | 2.22 | 0.35 | 1.88 | 0.11 | 1.53 | 0.05 |
| Q9P188 | 1.69 | 0.02 | 1.13 | 0.24 | 1.66 | 0.09 | 3.30 | 0.24 |
| INVS | 1.69 | 0.02 | −1.36 | 0.60 | 1.51 | 0.73 | 1.44 | 0.87 |
| DIAPH1 | 1.77 | 0.02 | −1.49 | 0.87 | 1.81 | 0.13 | −1.15 | 0.58 |
| IER3 | 1.58 | 0.03 | 2.26 | 0.10 | 1.99 | 0.03 | 2.92 | 0.12 |
| Q9H640 | 1.62 | 0.04 | 1.43 | 0.44 | −1.45 | 0.53 | −1.93 | 0.36 |
| GBP2 | 1.32 | 0.05 | 2.10 | 0.01 | 2.38 | 0.02 | 1.04 | 0.34 |
| NANS | 1.13 | 0.05 | 1.65 | 0.04 | 1.62 | 0.07 | 1.81 | 0.00 |
| Q86XN7; | 2.67 | 0.06 | 8.01 | 0.04 | 7.45 | 0.03 | −1.02 | 0.20 |
| Q9H9M1 | | | | | | | | |
| TNFAIP3 | 2.47 | 0.07 | 3.35 | 0.05 | 3.71 | 0.04 | 1.33 | 0.23 |
| Q96MJ8; | 1.74 | 0.08 | 4.01 | 0.01 | 1.90 | 0.58 | 1.65 | 0.05 |
| Q9BSE2 | | | | | | | | |
| Q9H753 | 2.29 | 0.08 | 3.91 | 0.02 | 2.55 | 0.77 | 1.02 | 0.75 |
| NTNG1 | 3.75 | 0.08 | −1.46 | 0.27 | 1.05 | 0.41 | 1.52 | 0.02 |
| INHBE | 1.58 | 0.09 | 1.84 | 0.05 | −1.07 | 0.64 | 1.07 | 0.73 |
| BCL6 | 1.76 | 0.12 | 1.67 | 0.03 | 1.73 | 0.04 | 1.05 | 0.25 |
| CXCL1 | 2.54 | 0.12 | 4.26 | 0.05 | 1.98 | 0.11 | 1.30 | 0.39 |
| EHD1 | 1.80 | 0.13 | 3.42 | 0.05 | 3.17 | 0.02 | 1.88 | 0.08 |
| RELB | 1.16 | 0.14 | 2.16 | 0.05 | 2.80 | 0.02 | 1.42 | 0.22 |
| HRK | 1.82 | 0.15 | 1.58 | 0.23 | 3.15 | 0.50 | 2.72 | 0.05 |
| CCL4 | 2.03 | 0.15 | 2.43 | 0.01 | 1.71 | 0.09 | 1.20 | 0.15 |
| SESN2 | 1.26 | 0.17 | 2.47 | 0.05 | 2.66 | 0.03 | −1.33 | 0.57 |
| NAB1 | 1.22 | 0.17 | 1.67 | 0.05 | 2.46 | 0.06 | 1.17 | 0.31 |
| EBI3 | 1.18 | 0.19 | 5.59 | 0.06 | 1.78 | 0.12 | −1.06 | 0.40 |
| DDX21 | 1.26 | 0.23 | 1.51 | 0.06 | 2.74 | 0.15 | −1.08 | 0.35 |
| XBP1 | 1.76 | 0.23 | 1.80 | 0.05 | 1.32 | 0.05 | 1.39 | 0.08 |
| SLURP1; ARS | 1.56 | 0.25 | 2.10 | 0.17 | 1.33 | 0.23 | 1.80 | 0.05 |
| HDAC10 | 2.19 | 0.31 | 1.35 | 0.19 | 1.60 | 0.06 | 1.13 | 0.25 |
| MEP1A | −1.23 | 0.39 | 1.08 | 0.72 | −1.16 | 0.59 | 2.47 | 0.02 |
| RAP2C | 1.34 | 0.43 | 1.70 | 0.03 | 2.61 | 0.04 | 1.37 | 0.09 |
| GYS1 | −1.30 | 0.47 | −1.01 | 0.54 | 2.17 | 0.03 | 2.26 | 0.51 |
| RARRES3 | 1.29 | 0.48 | −2.19 | 0.57 | 1.01 | 0.66 | 1.77 | 0.05 |
| PPY | 1.19 | 0.49 | 1.71 | 0.61 | 1.58 | 1.00 | 4.28 | 0.02 |
| NFKB1 | 1.16 | 0.75 | 1.72 | 0.01 | 1.89 | 0.03 | −1.12 | 0.97 |
| MTL4_HUMAN | 1.10 | 0.81 | 1.52 | 0.04 | 2.22 | 0.23 | −1.07 | 0.88 |
| Q9H040 | −1.62 | 0.82 | −1.02 | 0.72 | 1.58 | 0.01 | 1.71 | 0.43 |
| Q9NUP6 | 1.51 | 0.99 | 1.31 | 0.28 | 1.25 | 0.12 | 6.86 | 0.06 |
| | LPS + SEQ | | LPS + SEQ | | LPS + SEQ | | LPS + SEQ | |
| | ID NO: 1 | | ID NO: 1 | | ID NO: 1 | | ID NO: 1 | |
| | 1 hr | | 2 hr | | 4 hr | | 24 hr | |
| | Fold | | Fold | | Fold | | Fold | |
| Gene Name | change | p-value | change | p-value | change | p-value | change | p-value |
| ZNF83 | 2.02 | 0.03 | 1.08 | 0.65 | 1.17 | 0.41 | −1.37 | 0.38 |
| NFKBIA | 1.94 | 0.03 | 2.36 | 0.01 | 1.50 | 0.23 | 1.30 | 0.02 |
| Q9P188 | 1.58 | 0.04 | 1.87 | 0.32 | 2.14 | 0.02 | 2.05 | 0.15 |
| INVS | 1.55 | 0.02 | −2.95 | 0.08 | 1.77 | 0.96 | 1.44 | 0.08 |
| DIAPH1 | 2.07 | 0.01 | −1.52 | 0.96 | 2.77 | 0.04 | 1.78 | 0.13 |
| IER3 | 1.51 | 0.04 | 2.15 | 0.02 | 1.55 | 0.43 | 1.35 | 0.36 |
| Q9H640 | 1.77 | 0.02 | 1.48 | 0.17 | −1.99 | 0.21 | −1.97 | 0.10 |
| GBP2 | 1.72 | 0.08 | −1.29 | 0.36 | 1.51 | 0.06 | 1.33 | 0.33 |
| NANS | 1.02 | 0.76 | 1.01 | 0.51 | −1.41 | 0.27 | 1.70 | 0.04 |
| Q86XN7; | 1.67 | 0.20 | 3.71 | 0.04 | 1.08 | 0.41 | 1.78 | 0.14 |
| Q9H9M1 | | | | | | | | |
| TNFAIP3 | 2.50 | 0.14 | 3.45 | 0.02 | 2.34 | 0.04 | 1.20 | 0.67 |
| Q96MJ8; | 1.63 | 0.03 | 1.86 | 0.26 | 1.69 | 0.89 | 2.62 | 0.00 |
| Q9BSE2 | | | | | | | | |
| Q9H753 | 1.15 | 0.21 | 2.32 | 0.00 | 1.12 | 0.77 | 1.31 | 0.24 |
| NTNG1 | 1.55 | 0.11 | 1.29 | 0.27 | 1.09 | 0.53 | 3.39 | 0.06 |
| INHBE | −1.01 | 0.67 | 2.57 | 0.01 | −1.06 | 0.56 | −1.24 | 0.39 |
| BCL6 | 1.02 | 0.22 | 1.95 | 0.01 | 1.20 | 0.48 | 1.20 | 0.81 |
| CXCL1 | 1.93 | 0.12 | 4.56 | 0.03 | 2.08 | 0.63 | 1.09 | 0.49 |
| EHD1 | 1.64 | 0.13 | 3.48 | 0.00 | 1.55 | 0.15 | 1.73 | 0.07 |
| RELB | −1.02 | 0.25 | 2.58 | 0.00 | 2.00 | 0.93 | 1.11 | 0.20 |
| HRK | 3.46 | 0.08 | 2.01 | 1.00 | 2.28 | 0.87 | 2.09 | 0.05 |
| CCL4 | 1.36 | 0.19 | 1.88 | 0.05 | 1.80 | 0.05 | 1.14 | 0.86 |
| SESN2 | −1.05 | 0.88 | 1.30 | 0.16 | 1.62 | 0.01 | 1.12 | 0.45 |
| NAB1 | −1.09 | 0.47 | 2.42 | 0.00 | 1.41 | 0.03 | −1.20 | 0.66 |
| EBI3 | −1.25 | 0.54 | 1.96 | 0.02 | 1.89 | 0.47 | 2.44 | 0.26 |
| DDX21 | 1.21 | 0.37 | 1.55 | 0.00 | 1.60 | 0.01 | 1.31 | 0.05 |
| XBP1 | 1.12 | 0.09 | 1.58 | 0.00 | −1.02 | 0.32 | 1.02 | 0.68 |
| SLURP1; ARS | 2.62 | 0.46 | 1.20 | 0.30 | 1.39 | 0.51 | 1.85 | 0.02 |
| HDAC10 | 1.22 | 0.24 | 1.32 | 0.86 | 1.97 | 0.01 | 1.32 | 0.32 |
| MEP1A | −1.85 | 0.11 | 2.05 | 0.10 | 1.22 | 0.75 | 1.89 | 0.06 |
| RAP2C | 1.27 | 0.29 | 1.54 | 0.03 | 1.31 | 0.50 | 1.08 | 0.22 |
| GYS1 | −1.15 | 0.75 | −1.18 | 0.17 | 1.96 | 0.05 | −1.02 | 0.46 |
| RARRES3 | −1.13 | 0.46 | 1.15 | 0.70 | 1.24 | 0.13 | 2.62 | 0.05 |
| PPY | −4.35 | 0.48 | 2.50 | 0.26 | 1.13 | 0.69 | 5.65 | 0.04 |
| NFKB1 | 1.20 | 0.78 | 1.65 | 0.05 | 1.45 | 0.93 | 1.02 | 0.44 |
| MTL4_HUMAN | −1.26 | 0.87 | 1.52 | 0.01 | 1.18 | 0.08 | 1.03 | 0.41 |
| Q9H040 | −1.19 | 0.89 | −1.26 | 0.52 | 1.51 | 0.00 | −1.53 | 0.22 |
| Q9NUP6 | 1.31 | 0.59 | 1.29 | 0.90 | −1.27 | 0.64 | 1.90 | 0.01 |

TABLE 68
Genes that are upregulated by the Toll-like receptor 4 ligand LPS and
downregulated by LL-37.

| | | | LPS + | |
|---|---|---|---|---|
| | | LPS | LL37 | LL37 |
| | | fold | fold | fold |
| Gene Name | Gene Description | change | change | change |
| LC2A6 | Facilitative glucose transporter; binds cytochalasin B with low affinity | 7.04 | 1.13 | 1.41 |
| SLC4A5 | HCO3-transporter; Na+/HCO3-co-transporter | 6.80 | 1.52 | 4.72 |
| MCL1 | Apoptosis regulator Bcl-2 protein, BH | 6.31 | 1.73 | 1.72 |
| Q86XN7; Q9H9M1 | Aldehyde dehydrogenase; Proline-rich extensin; Proline-rich region | 6.00 | 1.41 | 2.29 |
| Q86UU3; Q8NAA1 | Proline-rich extensin; Proline-rich region | 5.41 | −1.08 | 1.16 |
| C15orf2 | low complexity | 5.24 | −2.56 | −1.29 |
| TNFRSF5 | Receptor for TNFSF5/CD40L | 5.24 | −1.30 | 1.82 |
| FACL6 | Activation of long-chain fatty acids for both synthesis of cellular lipids, and degradation | 5.09 | 1.50 | 2.61 |
| | via beta-oxidation. | | | |
| Q8IW99; Q96AU7 | Thymic Stromal Lymphopoietin Isoform 2. | 4.92 | −1.12 | −1.20 |
| PRB4 | Salivary proline-rich protein II-1 | 4.9 | −1.02 | −1.29 |
| Q9NWP0 | low complexity | 4.89 | −1.20 | −1.06 |
| Q8NF24; Q8TEE5 | β-Ig-H3/Fasciclin domain; Proline-rich extensin | 4.60 | 1.45 | 1.06 |
| PDE4DIP | Similar to Rat Myomegalin. | 4.56 | 1.27 | −1.42 |
| NUDT4 | Nudix hydrolase | 4.55 | −1.33 | −1.39 |
| DUSP2 | Regulates mitogenic signal transduction by dephosphorylating both Thr and Tyr | 4.42 | 1.35 | 1.46 |
| | residues on MAP kinases ERK1 and ERK2 | | | |
| LMAN2 | Intracellular lectin in the early secretory pathway; transport and sorting of high | 4.38 | −1.41 | −1.37 |
| | mannose-type glycoproteins | | | |
| RELB | Stimulates promoter activity in the presence of p49- and p50-NFκB. Neither associates | 4.30 | 1.96 | 1.23 |
| | with DNA nor with p65-NFκB | | | |
| SNF1LK | Probable serine/threonine-protein kinase SNF1LK | 4.27 | 1.25 | 1.93 |
| TNFα | Cytokine that binds to TNFRSF1A/TNFR1 and TNFRSF1B/TNFBR. | 4.25 | 1.14 | 2.64 |
| GHRHR | G protein-coupled receptor for growth hormone GRF. | 4.11 | −3.22 | 1.01 |
| TNFSF6 | Cytokine that binds to TNFRSF6/FAS, a receptor that transduces the apoptotic signal | 3.79 | 1.32 | 1.69 |
| | into cells. | | | |
| ENSG00000181873 | Glycine cleavage T protein (aminomethyl transferase) | 3.78 | −1.18 | 1.96 |
| IRAK2 | Required for IL1R-induced NFκB activation. Proximal mediators of IL-1 signalling | 3.71 | 1.41 | 1.46 |
| CKB | Reversibly catalyzes the transfer of phosphate between ATP and various phosphogens | 3.60 | 1.39 | 1.57 |
| | (e.g. creatine phosphate). | | | |
| CASR | Senses changes in the extracellular concentration of calcium ions. | 3.51 | 1.01 | −1.47 |
| KRTAP4-10 | Keratin, high sulfur B2 protein; von Willebrand factor, type C | 3.45 | 1.69 | −3.16 |
| ARHGEF3 | DH domain; Pleckstrin-like | 3.43 | 1.01 | 1.10 |
| CYP3A4; CYP3A7 | P450 Cytochrome. | 3.43 | −4.24 | −1.00 |
| GPR27 | Orphan receptor. Possible candidate for amine-like G-protein coupled receptor | 3.41 | 1.25 | −1.83 |
| PAX8 | Transcription factor for the thyroid-specific expression of the genes. | 3.37 | −1.95 | −5.99 |
| GAP43 | Associated with nerve growth. Major component of the motile & growth cones | 3.36 | 1.87 | −1.81 |
| Q96M75; Q9H568 | Actin/actin-like | 3.31 | −2.73 | 1.50 |
| AGTRL1 | Receptor for apelin coupled to G proteins that inhibit adenylate cyclase activity. | 3.24 | 2.00 | 1.24 |
| | Alternative co-receptor with CD4 for HIV-1 infection. | | | |
| C1orf22 | Putative α-mannosidase C1orf22 | 3.21 | 1.17 | 1.11 |
| EHD1 | EH-domain containing protein 1; Testilin; hPAST1 | 3.20 | 1.58 | 1.6 |
| ADRA1B | G protein-coupled α-adrenergic receptor | 3.17 | 1.62 | −1.60 |
| SSTR2 | G protein-coupled receptor for somatostatins-14 and -28. | 3.17 | 1.09 | 1.27 |
| SYNE1 | Involved in the maintenance of nuclear organization and structural integrity. Connects | 3.16 | 1.37 | −1.30 |
| | nuclei to the cytoskeleton. | | | |
| ENSG00000139977 | Bipartite nuclear localization signal; GCN5-related N-acetyltransferase | 3.15 | −1.94 | −1.20 |
| PTPRK | Regulator of processes involving cell contact and adhesion such as growth control, | 3.13 | 1.33 | 1.19 |
| | tumor invasion, and metastasis. | | | |
| O15059; Q9NZ16 | Guanine-nucleotide dissociation stimulator CDC25; Pleckstrin-like | 3.13 | 1.28 | 3.43 |
| N4BP3; KIAA0341 | Nedd4-binding protein 3; N4BP3 | 3.11 | −1.28 | 1.60 |
| Q8IVT2 | coiled-coil; low complexity | 3.10 | 1.32 | −1.73 |
| Q9NV39 | low complexity | 3.08 | −1.39 | −1.72 |
| HIP1R; HIP12; | Component of clathrin-coated pits and vesicles, may link the endocytic machinery to | 3.06 | −1.22 | 1.21 |
| KIAA0655 | actin cytoskeleton | | | |
| IL6 | Cytokine with a wide variety of biological functions | 3.04 | 1.11 | 1.46 |
| TNFAIP2 | May play a role as a mediator of inflammation and angiogenesis; Probably function in | 2.97 | 1.54 | 1.0 |
| | nuclear protein import as nuclear transport receptor. | | | |
| RCV1 | Seems to be implicated in the pathway from retinal rod guanylate cyclase to rhodopsin. | 2.95 | −1.38 | −1.69 |
| FBLN2 | Its binding to fibronectin and some other ligands is calcium dependent | 2.95 | 1.14 | −1.04 |
| TWIST2 | Inhibits transcriptional activation by MYOD1, MYOG, MEF2A and MEF2C. Represses | 2.92 | 1.80 | 2.05 |
| | expression of proinflammatory cytokines such as TNFα and IL1β. | | | |
| PARD6B | Adapter protein involved in asymmetrical cell division and polarization processes and | 2.88 | −3.02 | 1.46 |
| | formation of epithelial tight junctions. | | | |
| DCK | Required for the phosphorylation of several deoxyribonucleosides. | 2.84 | 1.23 | 1.65 |
| TULP4 | Tubby-like protein 4; Tubby superfamily protein | 2.83 | −2.18 | 1.07 |
| KLK10 | Has a tumor-suppressor role for NES1 in breast and prostate cancer | 2.81 | 1.40 | 1.25 |
| SPAP1 | Immunoglobulin-like | 2.80 | 1.23 | 2.35 |
| IBRDC2 | Zn-finger, RING; Zn-finger, cysteine-rich C6HC | 2.79 | −1.64 | 1.03 |
| JAM2 | May play a role in the processes of lymphocyte homing to secondary lymphoid organs | 2.77 | −2.6 | −1.44 |
| NRG2 | Direct ligand for ERBB3 and ERBB4 tyrosine kinase receptors. May also promote the | 2.74 | −1.44 | 2.31 |
| | heterodimerization with the EGF receptor | | | |
| CBARA1 | Bipartite nuclear localization signal; Calcium-binding EF-hand | 2.74 | 1.5 | 1.74 |
| DLG2 | Interacts with the cytoplasmic tail of NMDA receptor subunits as well as potassium | 2.66 | 1.55 | −1.0 |
| | channels | | | |
| PRKCBP1 | Protein kinase C binding protein 1 | 2.66 | −3.68 | −1.42 |
| MGLL | Alpha/beta hydrolase; Alpha/beta hydrolase fold; Esterase/lipase/thioesterase, active | 2.65 | 1.56 | 1.07 |
| | site; Lipase | | | |
| Q9BYE1 | Chymotrypsin serine protease, family S1; Low density lipoprotein-receptor, class A; | 2.60 | −2.52 | −3.84 |
| MARCKS | MARCKS is the most prominent cellular substrate for protein kinase C. Binds | 2.60 | 1.33 | 1.13 |
| | calmodulin, actin, and synapsin and is an F-actin cross-linking protein | | | |
| Q96N98 | Amidase | 2.60 | 1.25 | 1.07 |
| Q8NBY1; Q96AF2; | Bipartite nuclear localization signal; Protein kinase; Tyrosine protein kinase | 2.60 | 1.28 | 1.30 |
| Q9BS16 | Soxlz/Sox6-binding protein SolT. | 2.58 | −2.57 | 1.82 |
| PPP2CA | Protein phosphatase PP2A can modulate the activity of MAP-2 kinase and other | 2.58 | −1.47 | 1.19 |
| | kinases. | | | |
| RAB38 | May be involved in melanosomal transport and docking. Involved in the proper sorting | 2.54 | −1.778 | 1.62 |
| | of TYRP1 | | | |
| VCAM1 | Important in cell-cell recognition. VCAM1/VLA4 interaction may play a role in | 2.53 | 1.46 | 2.21 |
| | immune responses and in leukocyte emigration to inflammation sites | | | |
| TTTY8 | Transcript Y 8 protein | 2.52 | 1.22 | −1.13 |
| HTR2A | One of the several different serotonin G protein-coupled receptors | 2.51 | −1.20 | −1.35 |
| SERPINB10 | May play a role in the regulation of protease activities during hematopoiesis | 2.51 | 1.51 | −5.00 |
| O75121; Q9BVE1 | Immunoglobulin-like | 2.51 | −2.15 | −1.07 |
| ZCCHC2 | Phox-like; Zn-finger, CCHC type | 2.50 | −1.04 | 1.60 |
| CXCL2 | Chemokine produced by activated monocytes & neutrophils and expressed at | 2.50 | 1.38 | 1.42 |
| | inflammation sites | | | |
| GADD45B | Involved in the regulation of growth & apoptosis. Mediates activation of | 2.48 | 1.29 | 1.17 |
| | MTK1/MEKK4 MAPKKK | | | |
| KARS | Lysyl-tRNA synthetase LysRS | 2.43 | 1.29 | −2.94 |
| SCG2 | Secretogranin II; a neuroendocrine secretory granule protein, biologically active peptide | 2.42 | −1.83 | 1.45 |
| | precursor | | | |
| SLC17A2 | May be involved in actively transporting phosphate into cells via Na(+) cotransport | 2.41 | 1.03 | 1.08 |
| FLT4 | Receptor for VEGFC. Has a tyrosine-protein kinase activity | 2.41 | 1.41 | 2.48 |
| Q9NXT0 | KRAB box; Zn-finger, C2H2 type | 2.38 | 1.01 | −1.22 |
| Q96L19 | L-lactate dehydrogenase; | 2.38 | 1.00 | 1.12 |
| BICD1 | Drosophila Bicaudal D Homolog 1 | 2.34 | −1.66 | −4.36 |
| HCK | May also contribute to neutrophil migration and may regulate the neutrophil | 2.32 | 1.72 | 1.11 |
| | degranulation | | | |
| Q8N9T8; Q9H978 | Krr1 | 2.31 | −1.26 | −2.64 |
| PPP1R1A | Inhibitor of protein-phosphatase 1. | 2.31 | −3.64 | 1.33 |
| PAX7 | Probable transcription factor. May have a role in myogenesis | 2.31 | −1.01 | 1.52 |
| EBI3 | Cytokine receptor | 2.29 | 1.69 | 2.00 |
| THRA | Nuclear hormone receptor. High affinity receptor for triiodothyronine | 2.29 | −3.93 | −1.63 |
| SLC16A10 | Solute carrier family 16 (Monocarboxylate transporters), member 10 | 2.25 | −1.72 | 6.63 |
| INPP5E | Endonuclease/exonuclease/phosphatase family; Prenyl group binding site (CAAX box) | 2.25 | 1.16 | 2.82 |
| Q9H967 | Bipartite nuclear localization signal; G-protein beta WD-40 repeat | 2.23 | 1.50 | 3.75 |
| NFKB1 | NFκB1 p105 and p50 subunits involved in immune response and acute phase reactions. | 2.21 | 1.36 | 1.09 |
| MKL1 | Antiapoptotic transcriptional factor that acts as a cofactor of serum response factor | 2.21 | 1.24 | −1.08 |
| | (SRF). | | | |
| SS18L2 | SS18-like protein 2; SYT homolog-2 | 2.17 | 1.16 | 1.09 |
| TNFRSF9 | Receptor for TNFSF14/4-1BBL. Possibly active during T cell activation | 2.16 | 1.02 | −1.37 |
| TNFAIP6 | Possibly involved in cell-cell and cell-matrix interactions during inflammation & | 2.16 | 1.55 | −1.17 |
| | tumorgenesis | | | |
| Q9Y2K2 | Protein kinase; Serine/Threonine protein kinase; Tyrosine protein kinase | 2.14 | 1.16 | 1.12 |
| ING5 | Zn-finger-like, PHD finger | 2.11 | 1.77 | 1.12 |
| IL1A | Pro-inflammatory cytokine. | 2.11 | 1.35 | −2.22 |
| TMH | unknown | 2.10 | −1.15 | 1.38 |
| HDAC4 | Histone deacetylase acts on lysine residues on the N-terminus of core histones. | 2.10 | −1.44 | −1.02 |
| KPTN | Kaptin actin-binding protein. | 2.10 | 1.41 | 2.98 |
| SEC61G | Necessary for protein translocation in the endoplasmic reticulum | 2.07 | −1.14 | 4.02 |
| Q9Y484 | G-protein beta WD-40 repeat | 2.07 | 1.08 | −2.49 |
| FRAS1 | von Willebrand factor, type C Cytochrome c heme-binding site; Signal peptidase; | 2.05 | −3.27 | 2.13 |
| IER5 | Immediate early response 5. | 2.01 | −1.06 | 1.37 |
| Q8N137; Q8NCB8 | LysT-interacting protein Lip8. | 2.01 | −1.16 | 2.01 |
| Q96HQ0; Q9H5P0 | ATP/GTP-binding site motif A (P-loop); KRAB box; Zn-finger, C2H2 subtype; | 2.00 | −1.31 | 1.94 |
| TXNRD1 | Thioredoxin reductase, cytoplasmic precursor; TR; TR1 | 1.99 | 1.17 | 1.06 |
| CAV2 | Caveolin-2; May act as a scaffolding protein within caveolar membranes. | 1.98 | −1.17 | −1.48 |
| SCARB1 | CD36 antigen | 1.97 | −1.16 | 2.25 |
| MAP3K5 | Phosphorylates and activates two different subgroups of MAP kinase kinases. | 1.96 | 1.16 | 1.375 |
| PDHX | Required for anchoring dihydrolipoamide dehydrogenase (E3) to pyruvate | 1.96 | 1.32 | 1.23 |
| | dehydrogenase | | | |
| TCEB3 | SIII, or elongin, is a general transcription elongation factor. | 1.95 | 1.07 | 2.51 |
| C21orf55 | May have a role in protein folding or as a chaperone | 1.95 | 1.07 | 2.03 |
| MPHOSPH10 | Component of U3 nucleolar small nuclear ribonucleoprotein. Processing preribosomal | 1.94 | 1.19 | 1.22 |
| | RNA | | | |
| PDE8A | Phosphodiesterase plays a role in signal transduction by regulating the intracellular | 1.93 | −1.33 | 1.17 |
| | concentration of cyclic nucleotides. | | | |
| TFR2 | Transferrin receptor 2. Cellular iron uptake o | 1.92 | −1.57 | 1.60 |
| FARP1 | Band 4.1 domain; DH domain; Pleckstrin-like | 1.92 | 1.26 | 10.39 |
| SERPINA1 | Inhibitor of serine proteases. Primary target is elastase. Moderate affinity for plasmin, | 1.92 | 1.30 | 1.23 |
| | thrombin | | | |
| MYO15A | Myosins-15A; Unconventional myosins serve in intracellular movements. | 1.91 | 1.32 | −1.59 |
| RABGGTA | Catalyzes the transfer of a geranyl-geranyl moiety from geranyl-geranyl pyrophosphate | 1.89 | 1.27 | −1.22 |
| | to both cysteines in certain Rab proteins. | | | |
| KCNMB4 | Calcium-activated BK potassium channel, beta subunit | 1.89 | 1.12 | 1.56 |
| Q9BR02 | Bipartite nuclear localization signal; Ribosomal protein L23, N-terminal domain | 1.89 | −1.08 | 1.54 |
| APOB | Apolipoprotein B; Recognition signal for the cellular binding and internalization of | 1.88 | 1.39 | −1.48 |
| | LDL. | | | |
| MYC | Binds DNA both in a non-specific manner and activates transcription of growth-related | 1.87 | 1.23 | 1.1 |
| | genes | | | |
| FARP2 | Band 4.1 domain; DH domain; Pleckstrin-like | 1.85 | 1.32 | 1.12 |
| TFAP2BL1 | Transcription factor AP-2 | 1.84 | 1.22 | 2.04 |
| Q86U90; Q9H5F8 | SUA5/yciO/yrdC, N-terminal | 1.82 | 1.07 | −1.01 |
| USH1C | May be involved in protein-protein interaction | 1.81 | −1.29 | 1.22 |
| SOX2 | Transcription factor SOX-2 | 1.78 | 1.32 | −1.19 |
| Q9NVC3 | Amino acid/polyamine transporter, family II | 1.78 | −1.57 | 2.57 |
| NEIL2 | Formamidopyrimidine-DNA glycolase | 1.76 | −1.21 | 1.91 |
| TNIP1 | Interacts with TNFAIP3 and inhibits TNF-induced NFκB-dependent gene expression | 1.75 | 1.41 | 1.09 |
| ADRA1D | This alpha-adrenergic receptor mediates its effect through the influx of extracellular | 1.72 | −1.96 | −1.0792 |
| | calcium | | | |
| PCDHB9 | Potential calcium-dependent cell-adhesion protein. | 1.72 | −2.70 | 1.96 |
| Q12987 | Bipartite nuclear localization signal | 1.71 | −1.06 | 1.18 |
| TNFRSF6 | Receptor for TNFSF6/FASL. | 1.71 | 1.49 | 1.75 |
| C20orf72 | Protein C20orf72 | 1.70 | 1.14 | 1.67 |
| DNAJA3 | Modulates apoptotic signal transduction or effector structures within the mitochondrial | 1.69 | −1.20 | −1.26 |
| | matrix. | | | |
| MAB21L1 | Guanylate kinase; Mab-21 protein | 1.67 | −3.06 | −1.43 |
| BIRC2 | Apoptotic suppressor. Interacts with TRAF1 and TRAF2. | 1.67 | 1.34 | 1.12 |
| MYST1 | MOZ/SAS-like protein | 1.66 | 1.32 | 3.50 |
| CNN3 | Thin filament-associated protein | 1.66 | 1.00 | 1.12 |
| CXCL3 | Chemokine: May play a role in inflammation. | 1.65 | −2.13 | −1.215 |
| CD80; CSRP2; | Involved in the costimulatory signal essential for T lymphocytes activation. | 1.65 | −1.07 | 1.13 |
| RAD51L1 | | | | |
| ADARB1; TNFSF8 | Cytokine that binds to TNFRSF8/CD30. Induces proliferation of T cells; | 1.64 | −1.04 | −3.34 |
| Q81W74 | unknown | 1.62 | 1.09 | −1.02 |
| UXS1 | NAD-dependent epimerase/dehydratase | 1.62 | 1.11 | −1.04 |
| ENSG00000182364; | Phosphatidylinositol 3- and 4-kinase | 1.61 | −1.46 | −1.19 |
| TNFRSF7 | Receptor for TNFSF7/CD27L. May play a role in survival of activated T-cells. | 1.60 | 1.29 | −1.25 |
| MYBL2 | Transcription factor involved in the regulation of cell survival, proliferation, and | 1.60 | −1.07 | −1.22 |
| | differentiation. | | | |
| RAB33A | Ras-related protein Rab-33A; Small GTP-binding protein S10 | 1.60 | −1.30 | 1.15 |
| ATIC | Bifunctional purine biosynthesis protein PURH; | 1.59 | −1.36 | −1.166 |
| CAMK1 | Phosphorylates synapsin 1 | 1.59 | 1.26 | 1.53 |
| CCNT1 | Regulatory subunit of the cyclin-dependent kinase pair (CDK9/cyclin T) complex | 1.58 | 1.17 | 1.97 |
| KCNE4 | β subunit of voltage-gated potassium channel complex of pore-forming alpha subunits. | 1.57 | −1.20 | 1.41 |
| BOK | Apoptosis regulator Bcl-2 protein, | 1.56 | −1.21 | 1.12 |
| NF2 | Probably acts as a membrane stabilizing protein | 1.56 | 1.27 | 1.36 |
| PDP2; KIAA1348 | Catalyzes the dephosphorylation/reactivation of the α-subunit of pyruvate | 1.51 | −2.13 | −1.12 |
| | dehydrogenase E1 component | | | |

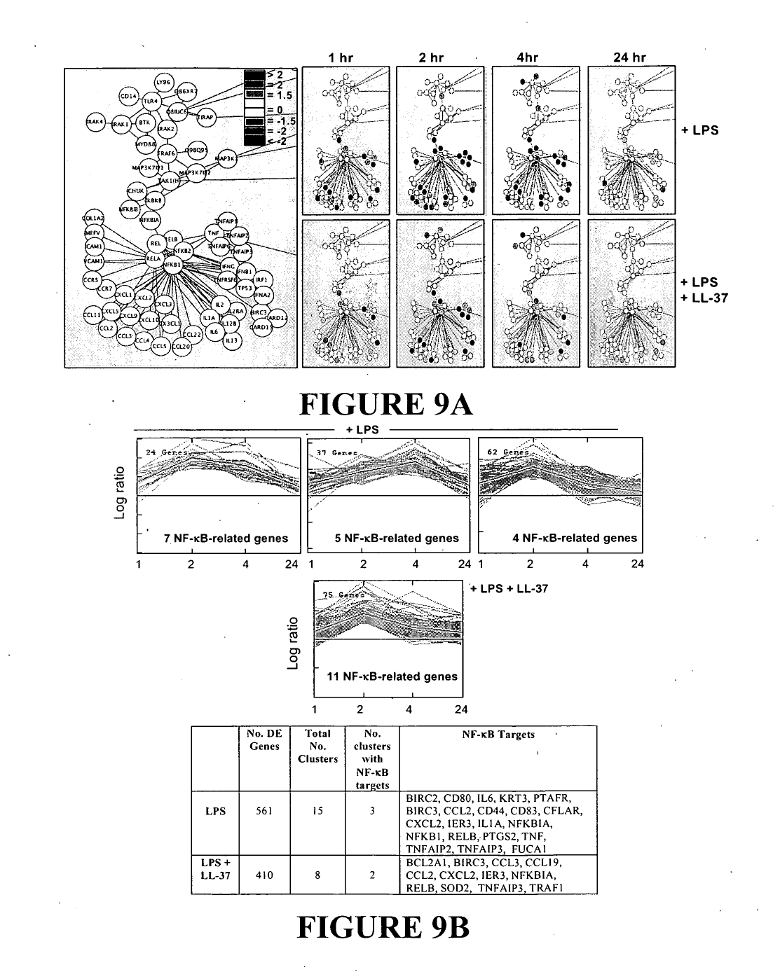

Given that LPS has been known to induce inflammatory responses via the TLR4 to NFκB pathway (Chow JC, et al. Toll-like receptor-4 mediates lipopolysaccharide-induced signal transduction. J Biol Chem 1999; 274:10689-92) and the product of certain differentially expressed genes in the microarray analysis were associated with this pathway, we analyzed in more detail the NFκB-regulated genes and the TLR4 pathway. This pathway was first mapped by integrating protein:protein interaction, signal transduction and regulatory data from the literature into Cytoscape (www.cytoscape.org), an open-source bioinformatics software platform for visualizing molecular interaction networks and integrating these interactions with other data. The microarray expression data was then overlaid onto this signal transduction protein network by colour coding the individual nodes (equivalent to specific genes/proteins) according to the extent of regulation (ranging from red to green, where the intensity of colour demonstrated the extent of up- to down regulation respectively). This then provided a graphic illustration of the genes with altered expression in response to LPS in the absence or presence of SEQ ID NO: 1 at each of the time points (FIG. 9A), and indicated that LPS generally up-regulated genes encoding elements of the TLR4→NFκB pathway, with a peak response at 2-4 hours, and that SEQ ID NO: 1 generally dampened this up-regulation.

To investigate further whether a defined portion of the LPS-responsive genes were likely co-regulated by NFκB, LPS-responsive, differentially-expressed genes with similar temporal expression profiles were clustered using the K-means procedure, a non-hierarchical algorithm, with an affinity threshold of 85% (FIG. 9B). Each cluster thus represented a set of potential co-regulated genes (based on their similar expression profiles over time). Based on this method, the LPS-induced genes Were divided into 15 clusters. Three of these clusters, containing a total of 123 genes with peak expression at 2 hr, 4 hr or both, contained 21 genes that are known from the literature to be NFκB-regulated (FIG. 9B). On the other hand, the temporal expression patterns of the 410 genes induced by LPS in the presence of SEQ ID NO: 1 fell into 8 clusters, one of which contained 11 of the 12 differentially expressed NFκB gene targets; six of these NFκB target genes were also included in the subset of LPS-stimulated genes and demonstrated modestly to substantially decreased expression in the presence of SEQ ID NO: 1. Many p50/p65 target genes (Tian B, et al. Identification of direct genomic targets downstream of the NF-kappa B transcription factor mediating TNF signaling. J Biol Chem 2005) were found in the clusters containing the NFκB genes. Thus SEQ ID NO: 1 clearly resulted in the suppression of LPS-stimulation of a substantial number of known NFκB target genes, and clustering data indicated that many other genes that might be NFκB regulated were similarly suppressed. However the data also suggested that the effect observed was selective in that some known NFκB regulated genes were still apparently differentially expressed in the presence of the combination of LPS and SEQ ID NO: 1. To confirm these observations, genes with significant differential expression in response to LPS, and that were differentially affected (remained up-regulated or abrogated) by the presence of the peptide, were selected for validation by quantitative real-time PCR.

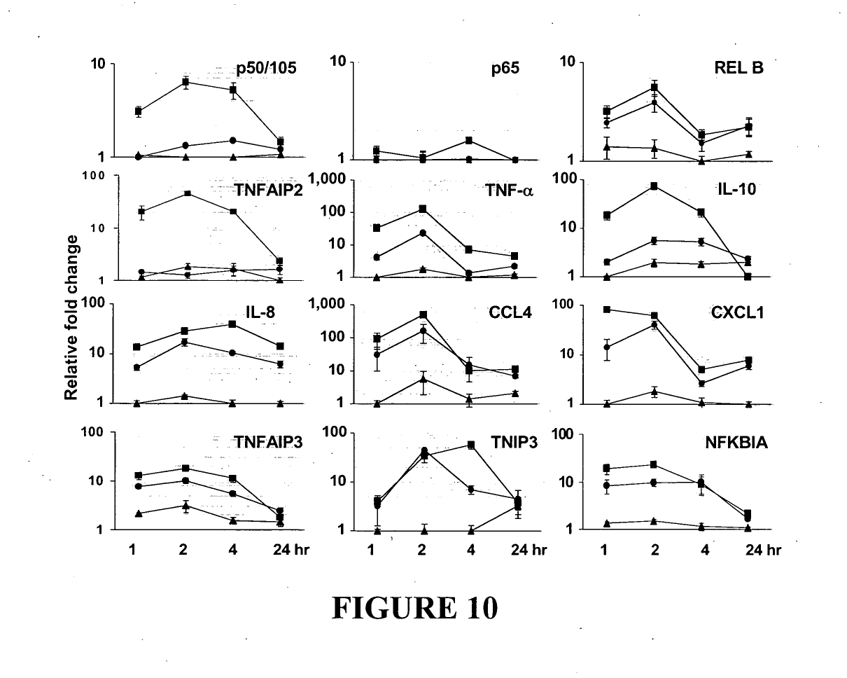

SEQ ID NO: 1 selectively modulates the transcription of specific LPS-induced inflammatory genes. Using qPCR, the expression profiles were validated for 14 of 20 selected genes differentially expressed according to the microarray analysis (FIG. 10). Several known “pro-inflammatory” genes were up-regulated after 2 and 4 hr of treatment with LPS, and this expression level invariably decreased after 24 hr of stimulation. Further, the expression of several LPS-induced genes was confirmed to be altered by the presence of SEQ ID NO: 1. Even though the peptide had a dampening effect on selected LPS-induced expression of inflammatory genes, not all genes up-regulated by LPS were suppressed by the presence of SEQ ID NO: 1, indicating that the effect of SEQ ID NO: 1 on LPS-induced inflammation was selective (FIG. 10). The expression of pro-inflammatory genes such as NFκB1 (p105/p50) and TNFAIP2 were substantially reduced (90-97%) in LPS-stimulated cells in the presence of SEQ ID NO: 1 at all time points. Also, LPS-induced transcription of TNFα was reduced in the presence of SEQ ID NO: 1 by 87% after 1 hr and around 80% at 2 and 4 hr, but at 24 hr only 58% reduction was observed. Similarly, LPS-induced transcription of IL10 was reduced by more than 90% after 1 and 2 hr in presence of SEQ ID NO: 1, and this effect decreased to 77% after 4 hr. In contrast, the expression of chemoattractants such as IL8, CCL4, and CXCL1, was slightly reduced by SEQ ID NO: 1 in LPS-stimulated cells but not completely eliminated. Likewise, the expressions of certain anti-inflammatory genes, that are negative regulators of the TLR4 to NFκB pathway were only slightly reduced in the presence of SEQ ID NO: 1. These genes included TNFAIP3 (TNFα-inducible Protein 3) and its interacting partner TNIP3 (TNFAIP3-interacting protein 3), as well as the NFκB-inhibitor, NFκBIA. LPS-induced transcription of NFκB subunit NFκB1 (p105/p50), but not RelB, was completely abrogated by SEQ ID NO: 1, whereas RelA (p65) did not show significant differential expression in response to LPS or SEQ ID NO: 1.

From the temporal transcriptional profiling of LPS-induced genes, it was concluded that SEQ ID NO: 1 did not substantially affect the LPS-induced expression of selected genes that are required for cell recruitment and movement (chemokines) or negative regulators of NFκB. In contrast, SEQ ID NO: 1 neutralized the expression of genes coding for inflammatory cytokines, NFκB1 (p105/p50) and TNFα-induced pro-inflammatory genes such as TNFAIP2.

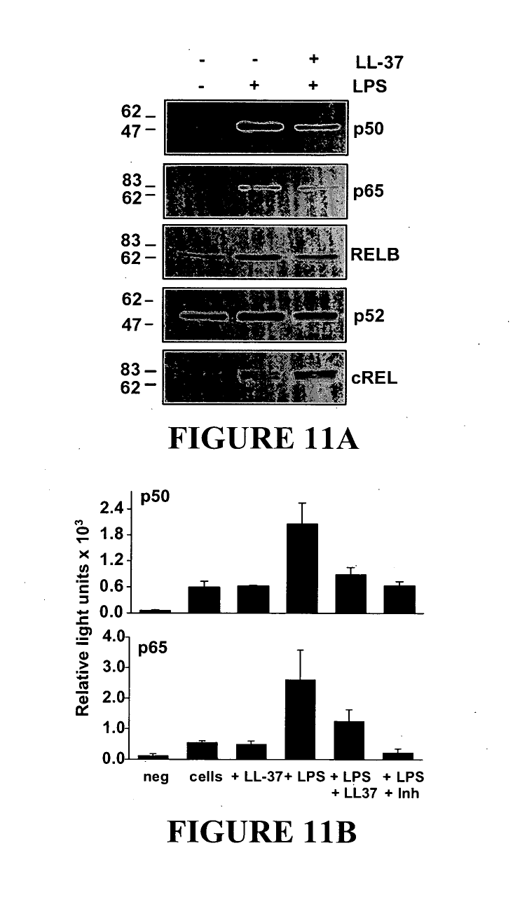

SEQ ID NO: 1 significantly inhibits LPS-induced translocation of the NFκB subunits p50 and p65. The above data indicated that although LL-37 reduced TNFα secretion by more than 95% at all time points, it had a lesser effect (58-87%) in reducing TNFα transcription. To study this in more detail we investigated the key transcription factor NFκB. TLR activation results in nuclear translocation of NFκB, the key transcription factor required for expression of many innate immunity and inflammatory genes (Bonizzi G, et al. The two NF-B activation pathways and their role in innate and adaptive immunity. Trends Immunol 2004; 25:280-8; Li ZW, et al. Genetic dissection of antigen receptor induced-NF-kappaB activation. Mol Immunol 2004; 41:701-14). Although NFκB has a number of subunits with different primary transcriptional regulatory functions, the p50/p65 NFκB heterodimer is most commonly implicated in the regulation of immunity genes. Nevertheless, transcriptionally active NFκB heterodimers other than p50/p65 have important functions as it has been shown that they can influence gene responses to bacterial molecules as well as susceptibility to a variety of infections (Tato CM, et al. Host-Pathogen interactions: Subversion and utilization of the NF-κB pathway during infection. Infect Immunity 2002; 70:3311-7; Mason N, et al. Cutting edge: identification of c-Rel-dependent and -independent pathways of IL-12 production during infectious and inflammatory stimuli. J Immunol 2002; 168:2590-4). To determine if SEQ ID NO: 1 suppressed LPS-induced changes in gene expression by affecting NFκB translocation into the nucleus, the nuclear localization of five NFκB subunits was assessed by Western blots. All monitored subunits of NF-κB (p105/50, p65, c-Rel, Rel B and p100/52) were detected in the nuclear extracts of THP-1 cells (FIG. 11A). The nuclear localization of p50, p65, c-Rel and Rel B, and to a lesser extent p100/52, was increased in THP-1 cells stimulated with LPS for 30 and 60 min (by 60 mins, LPS had induced a 3.5 fold increase in nuclear p50, a 4.5 fold increase in p65, a 1.7 fold increase in RELB and c-REL, and a 1.2 fold increase in p100/52 as assessed by densitometry). The LPS-induced translocation of p50, p65 and Rel B was clearly suppressed in the presence of SEQ ID NO: 1 as there was around a 35-70% decrease in subunit translocation after 60 min (FIG. 11A), while p100/52 and c-Rel did not appear to be affected.

To more accurately quantify the translocation of p50 or p65, the nuclear extracts were analyzed by ELISA-based immunoassays specific for these subunits (FIG. 11B). SEQ ID NO: 1 suppressed, by slightly more than 50%, LPS-induced p50 and p65 translocation at 30 and 60 min (54±4% and 56±4% inhibition of p50 at 30 and 60 min respectively and 57±8% and 54±3% inhibition of p65 at 30 and 60 min respectively). As a control, it was demonstrated that polymyxin B, a known inhibitor of LPS-LBP (LPS-binding protein) engagement, more substantially inhibited the translocation of NFκB subunits p50 and p65 (82±5% and 80±9% respectively at 60 min; data not shown), demonstrating that TLR4 to NFκB activation can be blocked significantly by agents acting at the cell surface. Although SEQ ID NO: 1 has been reported to activate signal transduction pathways including MAPK in human monocytes and lung epithelial cells (Bowdish D, et al. The human cationic peptide LL-37 induces activation of the extracellular signal-regulated kinase and p38 kinase pathways in primary human monocytes. J Immunol 2004; 172:3758-65), SEQ ID NO: 1 did not promote translocation of NF-κB subunits in human THP-1 cells. Together, these data demonstrate that SEQ ID NO: 1 can moderately alter the LPS-induced translocation of NFκB subunits, thereby providing one mechanism by which SEQ ID NO: 1 suppressed pro-inflammatory cytokine production.

To evaluate the anti-endotoxic activity of SEQ ID NO: 1, two different concentrations of LPS, 10 ng/ml and 100 ng/ml respectively, were used to stimulate human monocytic cells in the presence or absence of this host defense peptide, in an attempt to reflect concentrations of endotoxin ranging from the presumably low concentrations secreted by the normal flora (homeostatic conditions) and early in infection, to those observed in septic infections. To date there has been considerable controversy concerning the role of SEQ ID NO: 1 in human infections, particularly at physiological concentrations. Direct antimicrobial action will certainly occur at low salt concentrations but in the presence of more physiological concentrations of Na⁺ (130 mM) and Mg²⁺/Ca²⁺ (1-2 mM) found in tissues and in tissue culture medium (as employed here), SEQ ID NO: 1 has weak or no direct antimicrobial action at the peptide concentrations (1-5 μg/ml) apparently present at mucosal surfaces (Bowdish D M, et al. Impact of SEQ ID NO: 1 on anti-infective immunity. J Leukoc Biol 2005; 77:451-9). Nevertheless there is clear evidence of an anti-infective role (Scott M G, et al. The human antimicrobial peptide LL-37 is a multifunctional modulator of innate immune responses. J Immunol 2002; 169:3883-91; Bowdish D M, et al. Impact of LL-37 on anti-infective immunity. J Leukoc Biol 2005; 77:451-9; Kirikae T, et al. Protective effects of human 18-kilodalton cationic antimicrobial protein (CAP-18)-derived peptide against murine endotoxemia. Infect Immun 1998; 66:1861-8; Fukumoto K, et al. Effect of antibacterial cathelicidin peptide CAP18/LL-37 on sepsis in neonatal rats. Pediatr Surg Int 2005; 21:20-4; Ciomei C D, et al. Antimicrobial and chemoattractant activity, Lipopolysaccharide neutralization, cytotoxicity, and inhibition by serum of analogs of human cathelicidin LL-37. Antimicrob Agents Chemother 2005; 49:2845-50), which could be explained if SEQ ID NO: 1 has a role in modulating innate immunity. Consistent with this concept, at physiological concentrations SEQ ID NO: 1 is able to mediate chemotaxis (Agerberth B, et al. The human antimicrobial and chemotactic peptides LL-37 and alpha-defensins are expressed by specific lymphocyte and monocyte populations. Blood 2000; 96:3086-93; Yang D, et al. Participation of mammalian defensins and cathelicidins in anti-microbial immunity: receptors and activities of human defensins and cathelicidin (LL-37). J Leukoc Biol 2001; 69:691-7; Niyonsaba F, et al. A cathelicidin family of human antibacterial peptide LL-37 induces mast cell chemotaxis. Immunology 2002;106:20-6), MAP kinase phosphorylation (Scott M G, et al. The human antimicrobial peptide LL-37 is a multifunctional modulator of innate immune responses. J Immunol 2002; 169:3883-91; Tjabringa G S, et al. The antimicrobial peptide LL-37 activates innate immunity at the airway epithelial surface by transactivation of the epidermal growth factor receptor. J Immunol 2003; 171:6690-6; Bowdish D, et al. The human cationic peptide LL-37 induces activation of the extracellular signal-regulated kinase and p38 kinase pathways in primary human monocytes. J Immunol 2004; 172:3758-65; Lau Y E, et al. Interaction and cellular localization of the human host defense peptide LL-37 with lung epithelial cells. Infect Immun 2005; 73:583-91), Ca²⁺ mobilization (Niyonsaba F, et al. Evaluation of the effects of peptide antibiotics human beta-defensins-1/-2 and LL-37 on histamine release and prostaglandin D(2) production from mast cells. Eur J Immunol; 2001; 31:1066-75) and IL8 release in GMCSF treated monocytes (Bowdish D M, et al. Impact of LL-37 on anti-infective immunity. J Leukoc Biol 2005; 77:451-9), and as shown herein, anti-endotoxic activity.

The sole human cathelicidin peptide, SEQ ID NO: 1, has been shown to protect animals against endotoxemia/sepsis. Low, physiological concentrations of SEQ ID NO: 1 (≦1 μg/ml) are able to modulate inflammatory responses by inhibiting the release of the pro-inflammatory cytokine TNFα in LPS-stimulated human monocytic cells. Microarray studies established a temporal transcriptional profile, and identified differentially expressed genes in LPS-stimulated monocytes in the presence or absence of SEQ ID NO: 1. SEQ ID NO: 1 significantly inhibited the expression of specific pro-inflammatory genes upregulated by NFκB in the presence of LPS, including NFκB1 (p105/p50) and TNFα-induced protein 2 (TNFAIP2). In contrast, SEQ ID NO: 1 did not significantly inhibit LPS-induced genes that antagonize inflammation, such as TNFα-induced protein 3 (TNFAIP3) and the NFκB inhibitor, NFκBIA, or certain chemokine genes that are classically considered pro-inflammatory. Nuclear translocation, in LPS-treated cells, of the NFκB subunits p50 and p65 was reduced ≧50% in the presence of SEQ ID NO: 1, demonstrating that the peptide altered gene expression in part by acting directly on the TLR to NFκB pathway. SEQ ID NO: 1 almost completely prevented the release of TNFα and other cytokines by human peripheral blood mononuclear cells (PBMC) following stimulation with LPS and other TLR2/4 and TLR9 agonists, but not with cytokines TNFα or IL1β. Biochemical and inhibitor studies were consistent with a model whereby SEQ ID NO: 1 modulated the inflammatory response to LPS/endotoxin and other agonists of TLRs by a complex mechanism involving multiple points of intervention.

The data presented herein conclusively demonstrates that endotoxin-induced inflammatory gene responses and cytokine secretion in monocytes were suppressed by low, physiological concentrations of SEQ ID NO: 1, implicating SEQ ID NO: 1 in the regulation and control of pro-inflammatory responses associated with pathogenic assault and, by extension, with homeostatic levels of TLR agonists secreted by commensals. The data further demonstrates that SEQ ID NO: 1 can suppress LPS-induced NFκB translocation, and exert an anti-inflammatory effect that is not restricted to endotoxin-induced inflammation. In the human THP-1 monocytic cell line as well as in human PBMC, SEQ ID NO: 1 suppressed pro-inflammatory cytokine production induced by LPS as well as other agonists of TLR2 (LTA, PAM₃CSK4) and in part TLR9 (CpG), but selectively enhanced responses to the pro-inflammatory cytokines IL1β and TNFα. To gain mechanistic insight, transcriptional responses were profiled using microarrays and real time PCR over the course of 1 to 24 hr to study the effects of SEQ ID NO: 1 on LPS-stimulated monocytes. While the transcription of LPS-induced pro-inflammatory cytokines peaked at 2-4 hr and waned by 24 hr, a single, low dose of SEQ ID NO: 1 suppressed pro-inflammatory cytokine secretion by 1 hr, and this effect was sustained for 24 hr.

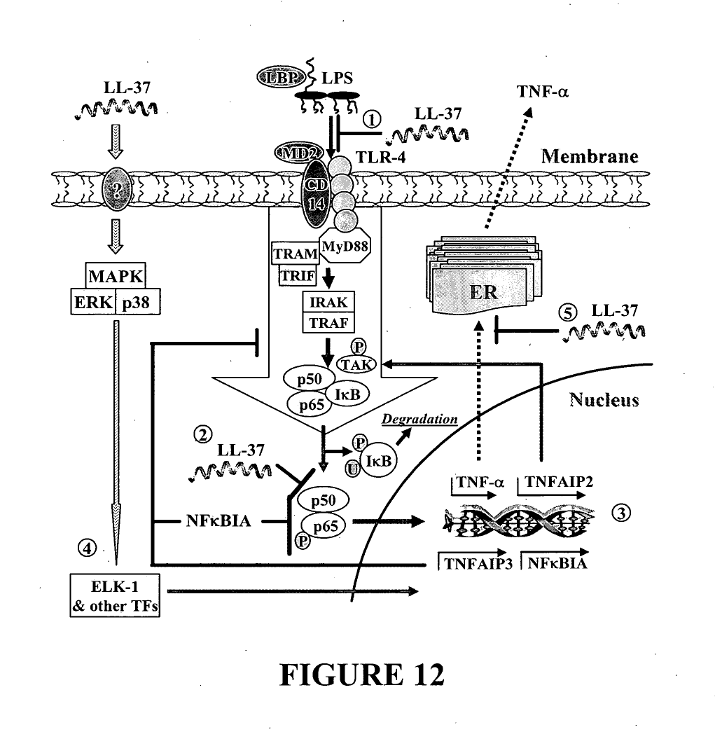

Overall, the data provides evidence that SEQ ID NO: 1 can manipulate both pre- and post-transcriptional events to modulate the TLR-induced inflammatory response in monocytes. A model consistent with the data in this manuscript is outlined in FIG. 12.

LPS-induced activation of NFκB is mediated by TLR4, a receptor containing TIR domain. It is known that receptors with TIR domains are potent activators of NFκB, as well as several other transcription factors such as AP-1, NF-IL6 and IRF3/7 (Takeda K, et al. Toll receptors and pathogen resistance. Cell Microbiol 2003;5:143-53). Mice deficient in TLR4 or MD2 are hyposensitive to LPS, moreover expression of some NFκB target genes is defective without MD2 (Poltorak A, et al. Defective LPS signaling in C3H/HeJ and C57BL/10ScCr mice: mutations in Tlr4 gene. Science 1998; 282:2085-8; Hoshino K, et al. Cutting edge: Toll-like receptor 4 (TLR4)-deficient mice are hyporesponsive to lipopolysaccharide: evidence for TLR4 as the Lps gene product. J Immunol 1999;162:3749-52; Nagai Y, et al. Essential role of MD-2 in LPS responsiveness and TLR4 distribution. Nat Immunol 2002;3:667-72). NFκB is known to play a central role in pathogenesis resulting in sepsis (Brown M A, et al. NF-kappaB action in sepsis: the innate immune system and the heart. Front Biosci 2004; 9:1201-17; Xiao C, et al. NF-kappaB, an evolutionarily conserved mediator of immune and inflammatory responses. Adv Exp Med Biol 2005; 560:41-5) as well as innate immunity to infections (Alcamo E, et al. Targeted mutation of TNF receptor I rescues the RelA-deficient mouse and reveals a critical role for NF-kappa B in leukocyte recruitment. J Immunol 2001; 167:1592-600; Senftleben U, et al. IKKbeta is essential for protecting T cells from TNFalpha-induced apoptosis. Immunity 2001;14:217-30). NFκB transcription factor is a dimeric complex of various subunits that belong to the Rel family; p105/50 (NFκB1), p100/52 (NFκB2), p65 (RelA), RelB, and c-Rel. NFκB proteins share a 300-amino acid Rel homology domain (RHD) that contains a nuclear localization sequence (NLS) and is involved in dimerization, sequence-specific DNA binding and interaction with the inhibitory IkB proteins (Ghosh S, et al. NF-kappa B and Rel proteins: evolutionarily conserved mediators of immune responses. Annu Rev Immunol 1998; 16:225-26). The NFκB proteins form numerous homo- and hetero-dimers that are associated with specific biological responses that stem from their ability to regulate target gene transcription differentially, e.g., p50/p52 dimers function as repressors, whereas Rel A or c-Rel dimers are transcriptional activators. In contrast, RelB does not form homodimers, but instead forms stable heterodimers with either p50 or p52 to exhibit a greater regulatory flexibility, and can be either an activator (Ryseck R P, et al. RelB, a new Rel family transcription activator that can interact with p50-NF-kappa B. Mol Cell Biol 1992;12:674-84) or a repressor (Ruben S M, et al. I-Rel: a novel rel-related protein that inhibits NF-kappa B transcriptional activity. Genes Dev 1992; 6:745-60). Many inflammatory stimuli trigger signal transduction pathways that result in nuclear localization of NFκB and subsequent transcription of inflammatory and immunity genes encoding for cytokines, chemokines, acute phase reactants, and cell adhesion molecules. The NFκB heterodimer comprising of p50 and p65 subunits has been strongly implicated in transcriptional events triggered by the activation of pro-inflammatory cytokine receptors or TLRs (Ghosh S, et al. NF-kappa B and Rel proteins: evolutionarily conserved mediators of immune responses. Annu Rev Immunol 1998; 16:225-260; Wang T, et al. NF-kappa B and Sp1 elements are necessary for maximal transcription of toll-like receptor 2 induced by Mycobacterium avium. J Immunol 2001; 167:6924-32). The activation and nuclear translocation of NFκB p50/p65 heterodimer is associated with increased transcription of genes encoding chemokines, cytokines, adhesion molecules such as intercellular adhesion molecule 1 (ICAM-1), vascular cell adhesion molecule-1 (VCAM-1), and endothelial-leukocyte adhesion molecule 1 (ELAM), as well as enzymes that produce secondary inflammatory mediators and inhibitors of apoptosis (Ghosh S, et al. NF-kappa B and Rel proteins: evolutionarily conserved mediators of immune responses. Annu Rev Immunol 1998; 16:225-260). These molecules are important components of the innate immune responses to invading pathogens and are required for migration of inflammatory mediators and phagocytic cells to tissues where NFκB has been activated in response to infection or injury (Pande V, et al. NF-kappaB in human disease: current inhibitors and prospects for de novo structure based design of inhibitors. Curr Med Chem 2005; 12:357-74).

The present invention provides evidence that the host defense peptide, SEQ ID NO: 1, can partially (˜50%) reduce LPS-induced p50/p65 translocation to the nucleus, indicating that this is one mechanism whereby SEQ ID NO: 1 suppressed LPS-induced gene transcription and exerted an anti-endotoxin effect. However if SEQ ID NO: 1 were merely blocking the binding of LPS to the TLR4 receptor through inhibiting its interaction with LBP and/or the LPS receptor complex (Scott M G, et al. Cutting edge: cationic antimicrobial peptides block the binding of lipopolysaccharide (LPS) to LPS binding protein. J Immunol 2000; 164, 549-53), it would be expected that NFκB translocation, and all NFκB-dependent transcriptional events would be inhibited to the same extent as TNFα release, that is >95%; however, this was not observed here. Instead, the effects of SEQ ID NO: 1 on NFκB subunit translocation were selective and relatively modest, and effects on LPS-stimulated transcription of NFκB-regulated genes ranged from very high, e.g., >95% for TNFAIP2 and p105/p50, to moderate (˜80%) for TNFα itself, through to almost no inhibition for other NFκB-regulated genes like TNFAIP3. Similarly SEQ ID NO: 1 can protect against sepsis in animal models when administered shortly after endotoxin (Bowdish D, et al. The human cationic peptide LL-37 induces activation of the extracellular signal-regulated kinase and p38 kinase pathways in primary human monocytes. J Immunol 2004; 172:3758-65). In unpublished mouse model experiments (K. Lee, M. G. Scott and R. E. W. Hancock), it was demonstrated that 200 μg of SEQ ID NO: 1 could protect against an 80% lethal dose (400 μg) of E. coli LPS administered peritoneally. Under such circumstances, the LPS would be in 5-fold molar excess and it seems unlikely that in this situation LPS neutralization alone could explain the protection exhibited by SEQ ID NO: 1.

The data presented herein indicates that the host defense peptide SEQ ID NO: 1 can selectively regulate genes that modulate inflammatory responses by suppressing NFκB translocation leading to dysregulation (modulation) of TLR-triggered transcriptional responses. SEQ ID NO: 1 caused inhibition of LPS-triggered pro-inflammatory gene TNFAIP2, but did not neutralize the LPS-induced expression of some of the known negative regulators of NFκB such as TNFAIP3, TNIP3 and NFκBIA (IκBα). Conversely, the transcription of known LPS-induced genes that are regulated by p50/p65 (FIG. 9B) were also inhibited >90% in the presence of SEQ ID NO: 1. However, although NFκB transcription factor activity is influenced by changes in nuclear concentration and subunit composition, the observed ˜50% inhibition of p50/p65 translocation in LPS-induced cells by SEQ ID NO: 1 seems unlikely to completely account for the observed 80% reduction in TNFα gene transcription at 2-4 hr or the >95% reduction in TNFα protein production and release. Rather, this nearly complete inhibition of pro-inflammatory cytokine release, without an equivalent abrogation of gene transcription, implies that mechanisms other than inhibition of NFκB are also required for SEQ ID NO: 1 to regulate TLR-induced inflammation. Such anomalies demonstrate that SEQ ID NO: 1 influences post-transcriptional events to modulate the inflammatory response. It is therefore shown that SEQ ID NO: 1 affects components of protein translation, maturation or secretion directly and/or indirectly via SEQ ID NO: 1-activated effectors or SEQ ID NO: 1-induced gene transcription (FIG. 12). It is known that SEQ ID NO: 1 can activate components of the MAPK pathway, in particular, p38 (which can influence post-transcriptional events) and ERK, and can promote the activity of the transcription factor, Elk-1 (Bowdish D, et al. The human cationic peptide LL-37 induces activation of the extracellular signal-regulated kinase and p38 kinase pathways in primary human monocytes. J Immunol 2004; 172:3758-65). The putative receptors for SEQ ID NO: 1, including FPRL-1, P2X7, and EGRFR, do not appear to be responsible for SEQ ID NO: 1 induced activation of the MAPK pathway in monocytes (Bowdish D, et al. The human cationic peptide LL-37 induces activation of the extracellular signal-regulated kinase and p38 kinase pathways in primary human monocytes. J Immunol 2004; 172:3758-65). In Drosophila, the LPS or PGN mediated up-regulation of expression of NFκB dependent genes is reported to be suppressed by a MAPK-regulated transcription factor, AP-1 (Kim T, et al. Downregulation of lipopolysaccharide response in drosophila by negative crosstalk between the AP1 and NF-κB signaling modules Nature Immunology 6, 211-218 (2005)). SEQ ID NO: 1 also demonstrates synergy with inflammatory stimuli such as GMCSF (Bowdish D M, et al. Impact of LL-37 on anti-infective immunity. J Leukoc Biol 2005; 77:451-9; Devine D A, et al. Cationic peptides: distribution and mechanisms of resistance. Curr Pharm Des 2002; 8:703-14) and IL1β (FIG. 8; Table 66) that likely reflect activation of co-operative signal transduction pathways or transcription of genes whose products contribute to a stabilized, enhanced or prolonged response. Thus, SEQ ID NO: 1 probably works alone or synergistically with other effector molecules of innate immunity, potentially via the MAPK pathway, to modulate TLR activation and enhance host defense mechanisms.

Accordingly, the data demonstrates that SEQ ID NO: 1 selectively suppresses the pro-inflammatory response in monocytes, particularly the TLR-induced secretion of pro-inflammatory cytokines. The ability of SEQ ID NO: 1 to dampen pro-inflammatory (septic) responses would be valuable for maintaining homeostasis in the face of natural shedding of microflora-associated TLR agonist molecules, as well as limiting the induction of systemic inflammatory syndrome/septic shock in response to moderate pathogen challenge. The anti-inflammatory effects of SEQ ID NO: 1 were observed at physiologically relevant concentrations of the peptide, and small changes in peptide concentration led to substantial impact on the cellular response to bacterial components such as LPS. SEQ ID NO: 1 thus appears to manifest multiple, complex mechanisms of action, including direct and indirect inhibition of TLR activation and transcription. The improved understanding of the mechanism(s) utilized by SEQ ID NO: 1 to selectively modulate inflammation, and thereby balance the TLR response to commensal or pathogenic bacteria indicates that endogenous cationic host defense peptides are important players in limiting over-active inflammation.

Although the invention has been described with reference to the presently preferred embodiment, it should be understood that various modifications can be made without departing from the spirit of the invention. Accordingly, the invention is limited only by the following claims.

What is claimed is:

1. A method of identifying an agent that selectively enhances innate immunity comprising:
contacting a cell containing a polynucleotide or polynucleotides that encode a polypeptide involved in innate immunity, with an agent of interest, wherein the agent suppresses inflammation and sepsis while increasing the expression of an anti-inflammatory gene encoding the polynucleotide as compared with expression of the anti-inflammatory gene in the absence of the agent and wherein the modulated expression results in enhancement of innate immunity.

2. The method of claim 1, wherein the anti-inflammatory gene is selected from the group consisting of ZNF83, NFKBIA, Q9P188, INVS, DIAPH1, IER3, Q9H640, GBP2, NANS, Q86XN7, Q9H9M1, TNFAIP3, Q96MJ8, Q9BSE2, Q9H753, NTNG1, INHBE, BCL6, CXCL1, EHD1, RELB, HRK, CCL4, SESN2, NAB1, EBI3, DDX21, XBP1, SLURP1, ARS, HDAC10, MEP1A, RAP2C, GYS1, RARRES3, PPY, NFKB1, MTL4_HUMAN, Q9H040, and Q9NUP6.

3. The method of claim 1, wherein the agent inhibits the inflammatory or septic response.

4. The method of claim 1, wherein the agent blocks the inflammatory or septic response.

5. The method of claim 1, wherein the agent inhibits the expression of TNF-α, IL1-β, IL-6, TNFAIP2, or p50 or p65 subunits of transcription factor NFκB.

6. The method of claim 1, wherein the agent inhibits the expression of proinflammatory molecules.

7. The method of claim 1, wherein the agent is a peptide.

8. The method of claim 7, wherein the peptide is selected from SEQ ID NO:4-54.

9. The method of claim 1, wherein the inflammation is induced by a microbe or a microbial ligand acting on a Toll-like receptor.

10. The method of claim 9, wherein the Toll-like receptor is Toll-like receptor-2, Toll-like receptor-4, or Toll-like receptor-9.

11. The method of claim 9, wherein the microbial ligand is a bacterial endotoxin, lipopolysaccharide, lipoteichoic acid or CpG DNA.

12. An agent identified by the method of claim 1.

13. An agent of claim 12, wherein the agent is a peptide, peptidomimetic, chemical compound, or a nucleic acid molecule.

14. A method of identifying an agent that selectively supresses sepsis comprising:
contacting a cell containing a polynucleotide or polynucleotides that encode a polypeptide involved in innate immunity, with an agent of interest, wherein the agent suppresses expression of a proinflammatory gene while maintaining expression of an anti-inflammatory gene encoding the polynucleotide as compared with expression of the anti-inflammatory gene in the absence of the agent, thereby suppressing sepsis.

15. The method of claim 14, wherein the anti-inflammatory gene is selected from the group consisting of ZNF83, NFKBIA, Q9P188, INVS, DIAPH1, IER3, Q9H640, GBP2, NANS, Q86XN7, Q9H9M1, TNFAIP3, Q96MJ8, Q9BSE2, Q9H753, NTNG1, INHBE, BCL6, CXCL1, EHD1, RELB, HRK, CCL4, SESN2, NAB1, EBI3, DDX21, XBP1, SLURP1, ARS, HDAC10, MEP1A, RAP2C, GYS1, RARRES3, PPY, NFKB1, MTL4_HUMAN, Q9H040, and Q9NUP6.

16. The method of claim 14, wherein the agent inhibits the expression of TNF-α, IL1-β, IL-6, TNFAIP2, or p50 or p65 subunits of transcription factor NFκB.

17. The method of claim 14, wherein the agent is a peptide selected from SEQ ID NO:4-54.

18. The method of claim 14, wherein the inflammation is induced by a microbe or a microbial ligand acting on a Toll-like receptor.

19. The method of claim 18, wherein the Toll-like receptor is Toll-like receptor-2, Toll-like receptor-4, or Toll-like receptor-9.

20. The method of claim 18, wherein the microbial ligand is a bacterial endotoxin, lipopolysaccharide, lipoteichoic acid or CpG DNA.

21. The method of claim 14, wherein the proinflammatory gene is selected from the group consisting of LC2A6, SLC4A5, MCL1, Q86XN7, Q9H9M1, Q86UU3, Q8NAA1, C15orf2, TNFRSF5, FACL6, Q8IW99, Q96AU7, PRB4, Q9NWP0, Q8NF24, Q8TEE5, PDE4DIP, NUDT4, DUSP2, LMAN2, RELB, SNF1LK, TNFα, GHRHR, TNFSF6, ENSG00000181873, IRAK2, CKB, CASR, KRTAP4-10, ARHGEF3, CYP3A4, CYP3A7, GPR27, PAX8, GAP43, Q96M75, Q9H568, AGTRL1, C1orf22, EHD1, ADRA1B, SSTR2, SYNE1, ENSG00000139977, PTPRK, O15059, Q9NZ16, N4BP3, KIAA0341, Q8IVT2, Q9NV39, HIP1R, HIP12, KIAA0655, IL6, TNFAIP2, RCV1, FBLN2, TWIST2, PARD6B, DCK, TULP4, LK10, SPAP1, IBRDC2, JAM2, NRG2, CBARA1, DLG2, PRKCBP1, MGLL, Q9BYE1, MARCKS, Q96N98, Q8NBY1, Q96AF2, Q9BS16, PPP2CA, RAB38, VCAM1, TTTY8, HTR2A, SERPINB10, O75121, Q9BVE1, ZCCHC2, CXCL2, GADD45B, KARS, SCG2, SLC17A2, FLT4, Q9NXT0, Q96L19, BICD1, HCK, Q8N9T8, Q9H978, PPP1R1A, PAX7, EBI3, THRA, SLC16A10, INPP5E, Q9H967, NFKB1, MKL1, SS18L2, TNFRSF9, TNFAIP6, Q9Y2K2, ING5, IL1A, TMH, HDAC4, KPTN, SEC61G, Q9Y484, FRAS1, IER5, Q8N137, Q8NCB8, Q96HQ0, Q9H5P0, TXNRD1 CAV2, SCARB1, MAP3K5, PDHX, TCEB3, C21orf55, MPHOSPH10, PDE8A, TFR2, FARP1, SERPINA1, MYO15A, RABGGTA, KCNMB4, Q9BR02, APOB, MYC, FARP2, TFAP2BL1, Q86U90, Q9H5F8, USH1C, IL8, SOX2, Q9NVC3, NEIL2, TNIP1, ADRA1D, PCDHB9, Q12987, TNFRSF6, C20orf72, DNAJA3, MAB21L1, BIRC2, MYST1, CNN3, CXCL3, CD80, CSRP2, RAD51L1, ADARB1, TNFSF8, Q8IW74, UXS1, ENSG00000182364, TNFRSF7, MYBL2, RAB33A, ATIC, CAMK1, CCNT1, KCNE4, BOK, NF2, PDP2, and KIAA1348.


SEQUENCE LISTING (96 sequences)

SEQ ID NO: 1 - protein, 37 aa, Homo sapiens
LLGDFFRKSKEKIGKEFKRIVQRIKDFLRNLVPRTES

SEQ ID NO: 2 - protein, 13 aa, Bovine
ILPWKWPWWPWRR

SEQ ID NO: 3 - protein, 12 aa, Bovine
RLARIVVIRVAR

SEQ ID NO: 4 - protein, 14 aa, Artificial sequence
  Cationic peptide
  MISC_FEATURE (1)..(2): Xaa is independently R, L or K and one or both may be present
  MISC_FEATURE (3)..(3): Xaa is one of C, S or A
  MISC_FEATURE (4)..(4): Xaa is one of R or P
  MISC_FEATURE (6)..(6): Xaa is one of A or V
  MISC_FEATURE (8)..(8): Xaa is one of A or V
  MISC_FEATURE (11)..(11): Xaa is one of V or W
  MISC_FEATURE (12)..(12): Xaa is one of C, S or A
  MISC_FEATURE (13)..(14): Xaa is independently R, L or K and one or both may be present
XXXXIXPXIPXXXX

SEQ ID NO: 5 - protein, 13 aa, Artificial sequence
  Cationic peptide
LLCRIVPVIPWCK

SEQ ID NO: 6 - protein, 14 aa, Artificial sequence
  Cationic peptide
LRCPIAPVIPVCKK

SEQ ID NO: 7 - protein, 13 aa, Artificial sequence
  Cationic peptide
KSRIVPAIPVSLL

SEQ ID NO: 8 - protein, 13 aa, Artificial sequence
  Cationic peptide
KKSPIAPAIPWSR

SEQ ID NO: 9 - protein, 14 aa, Artificial sequence
  Cationic peptide
RRARIVPAIPVARR

SEQ ID NO: 10 - protein, 13 aa, Artificial sequence
  Cationic peptide
LSRIAPAIPWAKL

SEQ ID NO: 11 - protein, 16 aa, Artificial sequence
  Cationic peptide
  MISC_FEATURE (1)..(2): Xaa is independently D, E, S, T or N and one or both may be present
  MISC_FEATURE (4)..(5): Xaa is P, G or D and one or both may be present
  MISC_FEATURE (6)..(6): Xaa is one of G, A, V, L, I or Y
  MISC_FEATURE (8)..(8): Xaa is one of R, K or H
  MISC_FEATURE (9)..(10): Xaa is P, G or D and one or both may be present
  MISC_FEATURE (11)..(11): Xaa is one of S, T, C, M or R
  MISC_FEATURE (12)..(12): Xaa is one of G, A, V, L, I or Y
  MISC_FEATURE (14)..(14): Xaa is one of G, A, V, L, I or Y
  MISC_FEATURE (15)..(16): Xaa is D, E, S, T or N and one or both may be present
XXLXXXKXXXXXPXXX

SEQ ID NO: 12 - protein, 13 aa, Artificial sequence
  Cationic peptide
DLPAKRGSAPGST

SEQ ID NO: 13 - protein, 14 aa, Artificial sequence
  Cationic peptide
SELPGLKHPCVPGS

SEQ ID NO: 14 - protein, 14 aa, Artificial sequence
  Cationic peptide
TTLGPVKRDSIPGE

SEQ ID NO: 15 - protein, 13 aa, Artificial sequence
  Cationic peptide
SLPIKHDRLPATS

SEQ ID NO: 16 - protein, 12 aa, Artificial sequence
  Cationic peptide
ELPLKRGRVPVE

SEQ ID NO: 17 - protein, 14 aa, Artificial sequence
  Cationic peptide
NLPDLKKPRVPATS

SEQ ID NO: 18 - protein, 19 aa, Artificial sequence
  Cationic peptide
  MISC_FEATURE (1)..(4): Xaa is chosen from A, P or R and one, two, three or all four maybe present
  MISC_FEATURE (5)..(6): Xaa is an aromatic amino acid (F, Y and W)
  MISC_FEATURE (7)..(7): Xaa is one of P or K
  MISC_FEATURE (8)..(9): Xaa is chosen from A, P, Y or W and one, both or none may be present
  MISC_FEATURE (11)..(12): Xaa is chosen from A, P, Y or W and one, both or none may be present
  MISC_FEATURE (14)..(15): Xaa is chosen from A, P, Y or W and one, both or none may be present
  MISC_FEATURE (16)..(18): Xaa is chosen from R or P and one, two or three may be present
XXXXXXXXXWXXWXXXXXK

SEQ ID NO: 19 - protein, 16 aa, Artificial sequence
  Cationic peptide
RPRYPWWPWWPYRPRK

SEQ ID NO: 20 - protein, 13 aa, Artificial sequence
  Cationic peptide
RRAWWKAWWARRK

SEQ ID NO: 21 - protein, 14 aa, Artificial sequence
  Cationic peptide
RAPYWPWAWARPRK

SEQ ID NO: 22 - protein, 16 aa, Artificial sequence
  Cationic peptide
RPAWKYWWPWPWPRRK

SEQ ID NO: 23 - protein, 14 aa, Artificial sequence
  Cationic peptide
RAAFKWAWAWWRRK

SEQ ID NO: 24 - protein, 12 aa, Artificial sequence
  Cationic peptide
RRRWKWAWPRRK

SEQ ID NO: 25 - protein, 20 aa, Artificial sequence
  Cationic peptide
  MISC_FEATURE (1)..(2): Xaa is R or K and one or both may be present
  MISC_FEATURE (3)..(3): Xaa is a polar or charged amino acid (S, T, M, N, Q, D, E, K, R and H)
  MISC_FEATURE (4)..(4): Xaa is C, S, M, D or A
  MISC_FEATURE (5)..(5): Xaa is F, I, V, M or R
  MISC_FEATURE (6)..(7): Xaa is R or K and one or both may be present
  MISC_FEATURE (9)..(9): Xaa is C, S, M, D or A
  MISC_FEATURE (10)..(10): Xaa is F, I, V, M or R
  MISC_FEATURE (13)..(13): Xaa is F, I, V, M or R
  MISC_FEATURE (14)..(14): Xaa is C, S, M, D or A
  MISC_FEATURE (15)..(15): Xaa is F, I, V, M or R
  MISC_FEATURE (16)..(17): Xaa is R or K and one or both may be present
  MISC_FEATURE (18)..(18): Xaa is C, S, M, D or A
  MISC_FEATURE (19)..(20): Xaa is R or K and one or both may be present
XXXXXXXVXXRGXXXXXXXX

SEQ ID NO: 26 - protein, 19 aa, Artificial sequence
  Cationic peptide
RRMCIKVCVRGVCRRKCRK

SEQ ID NO: 27 - protein, 18 aa, Artificial sequence
  Cationic peptide
KRSCFKVSMRGVSRRRCK

SEQ ID NO: 28 - protein, 19 aa, Artificial sequence
  Cationic peptide
KKDAIKKVDIRGMDMRRAR

SEQ ID NO: 29 - protein, 18 aa, Artificial sequence
  Cationic peptide
RKMVKVDVRGIMIRKDRR

SEQ ID NO: 30 - protein, 17 aa, Artificial sequence
  Cationic peptide
KQCVKVAMRGMALRRCK

SEQ ID NO: 31 - protein, 20 aa, Artificial sequence
  Cationic peptide
RREAIRRVAMRGRDMKRMRR

SEQ ID NO: 32 - protein, 17 aa, Artificial sequence
  Cationic peptide
  MISC_FEATURE (1)..(1): Xaa is R or K
  MISC_FEATURE (2)..(2): Xaa is a polar or charged amino acid (S, T, M, N, Q, D, E, K, R and H)
  MISC_FEATURE (3)..(3): Xaa is one of C, S, M, D or A
  MISC_FEATURE (4)..(4): Xaa is one of F, I, V, M or R
  MISC_FEATURE (5)..(6): Xaa is R or K and one or both may be present
  MISC_FEATURE (8)..(8): Xaa is one of A, I, S, M, D or R
  MISC_FEATURE (9)..(9): Xaa is one of F, I, V, M or R
  MISC_FEATURE (12)..(12): Xaa is one of F, I, V, M or R
  MISC_FEATURE (13)..(13): Xaa is one of A, I, S, M, D or R
  MISC_FEATURE (14)..(14): Xaa is one of F, I, V, M or R
  MISC_FEATURE (15)..(15): Xaa is R or K
  MISC_FEATURE (16)..(16): Xaa is one of C, S, M, D or A
  MISC_FEATURE (17)..(17): Xaa is R or K
XXXXXXVXXRGXXXXXX

SEQ ID NO: 33 - protein, 18 aa, Artificial sequence
  Cationic peptide
RTCVKRVAMRGIIRKRCR

SEQ ID NO: 34 - protein, 19 aa, Artificial sequence
  Cationic peptide
KKQMMKRVDVRGISVKRKR

SEQ ID NO: 35 - protein, 17 aa, Artificial sequence
  Cationic peptide
KESIKVIIRGMMVRMKK

SEQ ID NO: 36 - protein, 17 aa, Artificial sequence
  Cationic peptide
RRDCRRVMVRGIDIKAK

SEQ ID NO: 37 - protein, 19 aa, Artificial sequence
  Cationic peptide
KRTAIKKVSRRGMSVKARR

SEQ ID NO: 38 - protein, 18 aa, Artificial sequence
  Cationic peptide
RHCIRRVSMRGIIMRRCK

SEQ ID NO: 39 - protein, 31 aa, Artificial sequence
  Cationic peptide
  MISC_FEATURE (2)..(2): Xaa is a polar amino acid (C, S, T, M, N and Q)
  MISC_FEATURE (4)..(4): Xaa is one of A, L, S or K
  MISC_FEATURE (6)..(6): Xaa is one of A, L, S or K
  MISC_FEATURE (11)..(11): Xaa is one of A, L, S or K
  MISC_FEATURE (16)..(16): Xaa is one of A, L, S or K
  MISC_FEATURE (16)..(31): Xaa is amino acids chosen from G, A, V, L, I, P, F, S, T, K and H and one to seventeen may be present
KXKXFXKMLMXALKKXXXXXXXXXXXXXXXX

SEQ ID NO: 40 - protein, 28 aa, Artificial sequence
  Cationic peptide
KCKLFKKMLMLALKKVLTTGLPALKLTK

SEQ ID NO: 41 - protein, 26 aa, Artificial sequence
  Cationic peptide
KSKSFLKMLMKALKKVLTTGLPALIS

SEQ ID NO: 42 - protein, 27 aa, Artificial sequence
  Cationic peptide
KTKKFAKMLMMALKKVVSTAKPLAILS

SEQ ID NO: 43 - protein, 32 aa, Artificial sequence
  Cationic peptide
KMKSFAKMLMLALKKVLKVLTTALTLKAGLPS

SEQ ID NO: 44 - protein, 25 aa, Artificial sequence
  Cationic peptide
KNKAFAKMLMKALKKVTTAAKPLTG

SEQ ID NO: 45 - protein, 26 aa, Artificial sequence
  Cationic peptide
KQKLFAKMLMSALKKKTLVTTPLAGK

SEQ ID NO: 46 - protein, 26 aa, Artificial sequence
  Cationic peptide
  MISC_FEATURE (1)..(26): Xaa at residues 4, 7, 8, 10, 11,14, 15 is a hydrophobic aminoacid
  MISC_FEATURE (1)..(26): Xaa at residues 5, 6, 9, 12, 13 is a hydrophilic amino acid
KWKXXXXXXXXXXXXIFHTALKPISS

SEQ ID NO: 47 - protein, 26 aa, Artificial sequence
  Cationic peptide
KWKSFLRTFKSPVRTIFHTALKPISS

SEQ ID NO: 48 - protein, 26 aa, Artificial sequence
  Cationic peptide
KWKSYAHTIMSPVRLIFHTALKPISS

SEQ ID NO: 49 - protein, 26 aa, Artificial sequence
  Cationic peptide
KWKRGAHRFMKFLSTIFHTALKPISS

SEQ ID NO: 50 - protein, 26 aa, Artificial sequence
  Cationic peptide
KWKKWAHSPRKVLTRIFHTALKPISS

SEQ ID NO: 51 - protein, 26 aa, Artificial sequence
  Cationic peptide
KWKSLVMMFKKPARRIFHTALKPISS

SEQ ID NO: 52 - protein, 26 aa, Artificial sequence
  Cationic peptide
KWKHALMKAHMLWHMIFHTALKPISS

SEQ ID NO: 53 - protein, 26 aa, Artificial sequence
  Cationic peptide
KWKSFLRTFKSPVRTVFHTALKPISS

SEQ ID NO: 54 - protein, 26 aa, Artificial sequence
  Cationic peptide
KWKSYAHTIMSPVRLVFHTALKPISS

SEQ ID NO: 55 - DNA, 20 bp, Artificial sequence
  Primer
gtccctgtatgcctctggtc

SEQ ID NO: 56 - DNA, 19 bp, Artificial sequence
  Primer
gatgtcacgcacgatttcc

SEQ ID NO: 57 - DNA, 19 bp, Artificial sequence
  CpG oligonucleotide
tcatgacgttcctgacgtt

SEQ ID NO: 58 - DNA, 20 bp, Artificial sequence
  nonCpG oligonucleotide
ttcaggactttcctcaggtt

SEQ ID NO: 59 - DNA, 21 bp, Homo sapiens
tcatagcagccaccttcattc

SEQ ID NO: 60 - DNA, 20 bp, Homo sapiens
tagcgcagattcttgggttg

SEQ ID NO: 61 - DNA, 22 bp, Homo sapiens
tgtcctttctcagagtggttct

SEQ ID NO: 62 - DNA, 21 bp, Homo sapiens
tgcttccatagggacatcata

SEQ ID NO: 63 - DNA, 21 bp, Homo sapiens
acctgaaccttccaaagatgg

SEQ ID NO: 64 - DNA, 20 bp, Homo sapiens
gcgcagaatgagatgagttg

SEQ ID NO: 65 - DNA, 21 bp, Homo sapiens
gtgcagagggttgtggagaag

SEQ ID NO: 66 - DNA, 21 bp, Homo sapiens
ttctcccgtgcaatatctagg

SEQ ID NO: 67 - DNA, 21 bp, Artificial sequence
  Primer
cttttcttacaccgcgaggaa

SEQ ID NO: 68 - DNA, 20 bp, Artificial sequence
  Primer
gcagaggctgctggtctcat

SEQ ID NO: 69 - DNA, 24 bp, Artificial sequence
  Primer
tgactgctgtcttggatacacaga

SEQ ID NO: 70 - DNA, 23 bp, Artificial sequence
  Primer
tgatagcattgatgtcacagcct

SEQ ID NO: 71 - DNA, 19 bp, Artificial sequence
  Primer
gccagtgcttgcagaccct

SEQ ID NO: 72 - DNA, 20 bp, Artificial sequence
  Primer
ggctatgacttcggtttggg

SEQ ID NO: 73 - DNA, 19 bp, Artificial sequence
  Primer
gaccacactgcgccaacac

SEQ ID NO: 74 - DNA, 21 bp, Artificial sequence
  Primer
cttctccacaaccctctgcac

SEQ ID NO: 75 - DNA, 20 bp, Artificial sequence
  Primer
gtcgctgttgaagtcagagg

SEQ ID NO: 76 - DNA, 18 bp, Artificial sequence
  Primer
gaaactgtggcgtgatgg

SEQ ID NO: 77 - DNA, 20 bp, Artificial sequence
  Primer
ggttgccaagccttgtctga

SEQ ID NO: 78 - DNA, 19 bp, Artificial sequence
  Primer
agggagttcacatgcgcct

SEQ ID NO: 79 - DNA, 20 bp, Artificial sequence
  Primer
tggagaagggtgaccgactc

SEQ ID NO: 80 - DNA, 20 bp, Artificial sequence
  Primer
tcctcacagggcaatgatcc

SEQ ID NO: 81 - DNA, 20 bp, Artificial sequence
  Primer
ctaccagcgcgcctttaatg

SEQ ID NO: 82 - DNA, 19 bp, Artificial sequence
  Primer
tccggaaggacaggcagtt

SEQ ID NO: 83 - DNA, 23 bp, Artificial sequence
  Primer
ctgcccaggaatgctacagatac

SEQ ID NO: 84 - DNA, 21 bp, Artificial sequence
  Primer
cagggtcaccaagggtacaaa

SEQ ID NO: 85 - DNA, 24 bp, Artificial sequence
  Primer
tgaaagaaaggtagcagagctgaa

SEQ ID NO: 86 - DNA, 18 bp, Artificial sequence
  Primer
ccgcgtgctgaggaatct

SEQ ID NO: 87 - DNA, 19 bp, Artificial sequence
  Primer
aaagcgccaacacgtttga

SEQ ID NO: 88 - DNA, 20 bp, Artificial sequence
  Primer
aggaaccccagcaggaaaag

SEQ ID NO: 89 - DNA, 20 bp, Artificial sequence
  Primer
cttaggagggagagcccacc

SEQ ID NO: 90 - DNA, 20 bp, Artificial sequence
  Primer
ttgttcaggccttcccaaat

SEQ ID NO: 91 - DNA, 20 bp, Artificial sequence
  Primer
taggaaaggactgccgggat

SEQ ID NO: 92 - DNA, 20 bp, Artificial sequence
  Primer
ccgcttcttcacacactgga

SEQ ID NO: 93 - DNA, 20 bp, Artificial sequence
  Primer
tgggcattgacccctacaac

SEQ ID NO: 94 - DNA, 30 bp, Artificial sequence
  Primer
tgggtccctgaagaaccatcaggaagtaga

SEQ ID NO: 95 - DNA, 20 bp, Artificial sequence
  Primer
ggtgaagggagacctggctt

SEQ ID NO: 96 - DNA, 20 bp, Artificial sequence
  Primer
gtgcctcagcaatttctggc